(19) United States
(12) Patent Application Publication
(10) Pub. No.: US 2025/0082777 A1
(43) Pub. Date: Mar. 13, 2025

(54) METHODS OF EX VIVO DOSING AND ADMINISTRATION OF LIPID PARTICLES OR VIRAL VECTORS AND RELATED SYSTEMS AND USES

(71) Applicant: Sana Biotechnology, Inc., Seattle, WA (US)

(72) Inventors: Aaron Edward FOSTER, Bainbridge Island, WA (US); Steven A. GOLDMAN, Tucson, AZ (US); Kutlu ELPEK, Arlington, MA (US); Jesse GREEN, Seattle, WA (US); Terry FRY, Seattle, WA (US); De-Kuan CHANG, Seattle, WA (US); Hosein KOUROS-MEHR, Seattle, WA (US); Paul BRUNETTA, San Francisco, CA (US); Matthew Abraham SCOTT-SKANDERA, Seattle, WA (US)

(21) Appl. No.: 18/727,595

(22) PCT Filed: Jan. 10, 2023
(86) PCT No.: PCT/US2023/060409

§ 371 Date: Jul. 9, 2024

Related U.S. Application Data
(60) Provisional application No. 63/431,647, filed on Dec. 9, 2022.
(60) Provisional application No. 63/426,253, filed on Nov. 17, 2022.
(60) Provisional application No. 63/415,971, filed on Oct. 13, 2022.
(60) Provisional application No. 63/393,803, filed on Jul. 29, 2022.
(60) Provisional application No. 63/326,783, filed on Apr. 1, 2022.
(60) Provisional application No. 63/300,633, filed on Jan. 18, 2022.
(60) Provisional application No. 63/298,196, filed on Jan. 10, 2022.

(51) Int. Cl. A61K 48/00; A61K 31/194; A61K 38/17; A61K 39/00; A61P 35/00; C07K 16/28; C12N 5/0783; C12N 15/86

(52) U.S. Cl. CPC A61K 48/005; A61K 31/194; A61K 38/1793; A61K 40/11; A61K 40/31; A61K 40/4211; A61K 40/4221; A61K 48/0041; A61K 48/0075; A61K 48/0083; A61P 35/00; C07K 16/2803; C07K 16/2887; C12N 5/0636; C12N 15/86; A61K 2239/13; A61K 2239/17; C12N 2740/15022; C12N 2740/15043

(57) ABSTRACT

Provided herein are methods of ex vivo administration of a lipid particle or a payload gene, to a subject. In some embodiments, the methods are in-line methods of administration of a lipid particle or payload gene that are performed in a closed fluid circuit. Also provided are related compositions, containers, and systems in connection with the provided methods.

### CROSS-REFERENCE TO RELATED APPLICATIONS

This application claims priority to U.S. provisional application 63/298,196 entitled “METHODS OF EX VIVO DOSING AND ADMINISTRATION OF LIPID PARTICLES OR VIRAL VECTORS AND RELATED SYSTEMS AND USES”, filed Jan. 10, 2022, and to U.S. provisional application 63/300,633 entitled “METHODS OF EX VIVO DOSING AND ADMINISTRATION OF LIPID PARTICLES OR VIRAL VECTORS AND RELATED SYSTEMS AND USES”, filed Jan. 18, 2022, and to U.S. provisional application 63/326,783 entitled “METHODS OF EX VIVO DOSING AND ADMINISTRATION OF LIPID PARTICLES OR VIRAL VECTORS AND RELATED SYSTEMS AND USES”, filed Apr. 1, 2022, and to U.S. provisional application 63/393,803 entitled “METHODS OF EX VIVO DOSING AND ADMINISTRATION OF LIPID PARTICLES OR VIRAL VECTORS AND RELATED SYSTEMS AND USES”, filed Jul. 29, 2022, and to U.S. provisional application 63/415,971 entitled “METHODS OF EX VIVO DOSING AND ADMINISTRATION OF LIPID PARTICLES OR VIRAL VECTORS AND RELATED SYSTEMS AND USES”, filed Oct. 13, 2022, and to U.S. provisional application 63/426,253 entitled “METHODS OF EX VIVO DOSING AND ADMINISTRATION OF LIPID PARTICLES OR VIRAL VECTORS AND RELATED SYSTEMS AND USES”, filed Nov. 17, 2022, and to U.S. provisional application 63/431,647 entitled “METHODS OF EX VIVO DOSING AND ADMINISTRATION OF LIPID PARTICLES OR VIRAL VECTORS AND RELATED SYSTEMS AND USES”, filed Dec. 9, 2022, the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 18615_2006140_SeqList.XML created Jan. 9, 2023 which is 412,992 bytes in size. The information in the electronic format of the Sequence Listing is incorporated by reference in its entirety.

### FIELD

Provided herein are methods of ex vivo administration of a lipid particle or viral vector, such as for delivery of a payload gene, to a subject. In some embodiments, the methods are in-line methods of administration of a lipid particle or viral vector, such as for the delivery of a payload gene. that are performed in a closed fluid circuit. Also provided are related compositions, containers, and systems in connection with the provided methods.

### SUMMARY

Provided herein is a method for administration of a lipid particle or viral vector to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lipid particles or viral vectors to create a transfection mixture; and d) reinfusing the contacted PBMCs or subset and/or the transfection mixture to the subject, thereby administering the lipid particle to the subject, wherein steps (a)-(d) are performed in-line in a closed fluid circuit. In some of any of the provided embodiments, the lipid particle or viral vectors comprises a nucleic acid encoding a payload gene.

Provided herein is an in-line method for administration of a lipid particle or viral vector to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lipid particles or viral vectors to create a transfection mixture; and d) reinfusing the contacted PBMCs or subset and/or the transfection mixture to the subject, thereby administering the lipid particle to the subject, wherein steps (a)-(d) are performed in-line in a closed fluid circuit. In some of any of the provided embodiments, the lipid particle or viral vectors comprises a nucleic acid encoding a payload gene.

Provided herein is a method for administration of a payload gene to a subject, the method comprising: a) obtaining whole blood from a subject; b) collecting the fraction of blood containing peripheral blood mononuclear cells (PBMCs) or subset thereof; c) contacting the PBMCs or subset with a composition comprising lipid particles or viral vectors comprising a nucleic acid encoding a payload gene to create a transfection mixture; and d) reinfusing the contacted PBMCs or subset and/or the transfection mixture to the subject, thereby administering the payload gene to the subject, wherein steps (a)-(d) are performed in-line in a closed fluid circuit.

Provided herein is an in-line method for administration of a payload gene to a subject, the method comprising: a) obtaining whole blood from a subject; b) collecting the fraction of blood containing peripheral blood mononuclear cells (PBMCs) or subset thereof; c) contacting the PBMCs or subset with a composition comprising lipid particles or viral vectors comprising a nucleic acid encoding a payload gene to create a transfection mixture; and d) reinfusing the contacted PBMCs or subset and/or the transfection mixture to the subject, thereby administering the payload gene to the subject, wherein steps (a)-(d) are performed in-line in a closed fluid circuit.

Provided herein is a method for administration of a lipid particle or viral vector to a subject, the method comprising: a) obtaining whole blood from a subject; b) collecting the fraction of blood containing peripheral blood mononuclear cells (PBMCs) subset thereof; c) contacting the PBMCs or subset thereof with a composition comprising lipid particles or viral vectors to create a transfection mixture; and d) reinfusing the contacted PBMCs or leukocyte components and/or the transfection mixture to the subject, thereby administering the lipid particle or viral vector to the subject, wherein the method is characterized by one or more of: (i) wherein the method is characterized by the collecting in (b) not comprising a selection step (e.g. immunoaffinity selection) for target cells; (ii) the contacting in step (c) is initiated within 24 hours after collecting the fraction of blood containing PBMCs or subset thereof; (iii) the contacting in step (c) is for no more than 24 hours prior to the reinfusing in step (d); (iv) the whole blood, collected PBMCs or subset thereof, and transfection mixture are not subjected to cryopreservation or freezing; and/or (v) steps (a)-(d) are carried out for a time that is no more than 24 hours. In some of any of the provided embodiments, the lipid particle or viral vector comprises a nucleic acid encoding a payload gene.

Provided herein is a method for delivering a payload gene to a subject, the method comprising: a) obtaining whole blood from a subject; b) collecting the fraction of blood containing peripheral blood mononuclear cells (PBMCs) or leukocyte components; c) contacting the PBMCs or subset thereof with a composition comprising lipid particles or viral vectors comprising a nucleic acid encoding a payload gene to create a transfection mixture; and d) reinfusing the contacted PBMCs or subset thereof and/or the transfection mixture to the subject, thereby administering the lipid particle or viral vector to the subject, wherein the method is characterized by one or more of: (i) wherein the method is characterized by the collecting in (b) not comprising a selection step (e.g. immunoaffinity selection) for target cells.; (ii) the contacting in step (c) is initiated within 24 hours after collecting the fraction of blood containing PBMCs or subset thereof; (iii) the contacting in step (c) is for no more than 24 hours prior to the reinfusing in step (d); (iv) the whole blood, PBMCs or subset thereof, and transfection mixture are not subjected to cryopreservation or freezing; and/or (v) steps (a)-(d) are carried out for a time that is no more than 24 hours.

Provided herein is a method for administration of a lentiviral vector encoding a chimeric antigen receptor (CAR) to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lentiviral vectors comprising a nucleic acid encoding a CAR to create a transfection mixture; and d) reinfusing the contacted PBMCs or subset and/or the transfection mixture to the subject, thereby administering the lentiviral particle to the subject, wherein steps (a)-(d) are performed in-line in a closed fluid circuit.

Provided herein is an in-line method for administration of a lentiviral vector encoding a chimeric antigen receptor (CAR) to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lentiviral vectors comprising a nucleic acid encoding a CAR to create a transfection mixture; and d) reinfusing the contacted PBMCs or subset and/or the transfection mixture to the subject, thereby administering the lentiviral particle to the subject, wherein steps (a)-(d) are performed in-line in a closed fluid circuit.

Also provided herein is a method for delivering a lentiviral vector encoding a chimeric antigen receptor (CAR) to a subject, the method comprising: a) obtaining whole blood from a subject; b) collecting the fraction of blood containing peripheral blood mononuclear cells (PBMCs) or leukocyte components; c) contacting the PBMCs or subset thereof with a composition comprising lentiviral vectors comprising a nucleic acid encoding a CAR to create a transfection mixture; and d) reinfusing the contacted PBMCs or subset thereof and/or the transfection mixture to the subject, thereby administering the lentiviral particle to the subject, wherein the method is characterized by one or more of (i) wherein the method is characterized by the collecting in (b) not comprising a selection step (e.g. immunoaffinity selection) for target cells; (ii) the contacting in step (c) is initiated within 24 hours after collecting the fraction of blood containing PBMCs or subset thereof; (iii) the contacting in step (c) is for no more than 24 hours prior to the reinfusing in step (d); (iv) the whole blood, PBMCs or subset thereof, and transfection mixture are not subjected to cryopreservation or freezing; and/or (v) steps (a)-(d) are carried out for a time that is no more than 24 hours.

In some of any of the provided embodiments, the lentiviral vector is pseudotyped for targeting to a T cell. In some of any of the provided embodiments, the T cell is a CD3+ T cell, a CD4+T cell or a CD8+ T cell. In some of any of the provided embodiments, the T cell is a CD8+ T cell. In some of any of the provided embodiments, the CAR is an anti-CD19 CAR, an anti-CD22 CAR, an anti-CD20 CAR, or an anti-BCMA CAR. In some of any of the provided embodiments, the CAR is an anti-CD19 CAR. In some of any of the provided embodiments, the anti-CD19 CAR comprises an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36, and a CD3zeta signaling domain set forth in SEQ ID NO: 38.

In some of any of the provided embodiments, the method is carried out in a single in-line procedure to maintain a closed or functionally closed fluid circuit. In some of any of the provided embodiments, two or more of steps (a)-(d) are carried out in-line in a closed fluid circuit. In some of any of the provided embodiments, three or more of steps (a)-(d) are carried out in-line in a closed fluid circuit. In some of any of the provided embodiments, between at least two steps, the method includes separating the subject from the in-line closed fluid circuit and then reconnecting the subject prior to the next step. In some of any of the provided embodiments, steps (a)-(c) are carried out in-line in a closed fluid circuit, and wherein the method comprises separating the subject from the closed fluid circuit after step (c) and reconnecting the subject to the closed fluid circuit before step (d). In some of any of the provided embodiments, all of steps (a)-(d) are carried out in-line in a closed fluid circuit

In some of any of the provided embodiments, the method is characterized by at least two of (i)-(v). In some of any of the provided embodiments, the method is characterized by at least three of (i)-(v). In some of any of the provided embodiments, the method is characterized by at least four of (i)-(v). In some of any of the provided embodiments, the method is characterized by (i)-(v).

In some of any of the provided embodiments, the method is characterized by the collecting in (b) not comprising a selection step (e.g. immunoaffinity selection) for target cells. In some of any of the provided embodiments, the target cells are T cells and the method does not include a selection step for T cells positive for a T cell marker (e.g. CD3, CD4 or CD8). In some of any of the provided embodiments, the target cells are CD34+cells and the method does not include a selection step for cells positive for CD34. In some of any of the provided embodiments, the method is characterized by the contacting in step (c) being initiated within 24 hours after collecting the fraction of blood containing PBMCs or subset thereof. In some of any of the provided embodiments, the contacting in step (c) is initiated immediately after collecting the fraction of blood containing PBMCs or subset thereof following transfer to a contacting chamber.

In some of any of the provided embodiments, the contacting in step (c) is initiated 0 to 12 hours, 0 to 6 hours, 0 to 4 hours, 0 to 2 hours or 0 to 1 hour, or 0 to 30 minutes after collecting the fraction of blood containing PBMCs or subset thereof. In some of any of the provided embodiments, the contacting in step (c) is initiated within at or about 12 hours, within at or about 6 hours, within at or about 2 hours, within at or about 1 hour, within at or about 30 minutes or within at or about 15 minutes after collecting the fraction of blood containing PBMCs or subset thereof. In some of any of the provided embodiments, the method is characterized by the contacting in step (c) being no more than 24 hours prior to the reinfusing in step (d).

In some of any of the provided embodiments, the contacting in step (c) is for 15 minutes to 24 hours, 15 minutes to 12 hours, 15 minutes to 6 hours, 15 minutes to 4 hours, 15 minutes to 2 hours, 15 minutes to 1 hour, 15 minutes to 30 minutes, 30 minutes to 24 hours, 30 minutes to 12 hours, 30 minutes to 6 hours, 30 minutes to 4 hours, 30 minutes to 2 hours, 30 minutes to 1 hour, 1 hour to 24 hours, 1 hour to 12 hours, 1 hour to 6 hours, 1 hour to 4 hours, 1 hour to 2 hours, 2 hours to 24 hours, 2 hours to 12 hours, 2 hours to 6 hours, 2 hours to 4 hours, 4 hours to 24 hours, 4 hours to 12 hours, 4 hours to 6 hours, 6 hours to 24 hours, 6 hours to 12 hours or 12 hours to 24 hours. In some of any of the provided embodiments, the contacting in step (c) is for at or about 15 minutes, at or about 30 minutes, at or about 1 hour, or at or about 2 hours, or any value between any of the foregoing. In some of any of the provided embodiments, at least a portion of the contacting in (c) is carried out under centrifugation.

In some of any of the provided embodiments, the method is characterized by the whole blood, PBMCs or subset thereof, and transfection mixture having not been subjected to cryopreservation or freezing. In some of any of the provided embodiments, the fraction of blood, PBMCs or subset thereof, and transfection mixture are not formulated with a cryoprotectant (e.g. DMSO). In some of any of the provided embodiments, the transfection mixture is directly reinfused to the subject, optionally without any further processing or washing steps. In some of any of the provided embodiments, the method is characterized by steps (a)-(d) being carried out for a time that is no more than 24 hours. In some of any of the provided embodiments, the steps (a)-(d) are carried out for a time that is between 1 hour and 24 hours, between 1 hour and 12 hours, between 1 hour and 6 hours, between 1 hour and 4 hours, between 1 hour and 2 hours, between 2 hours and 24 hours, between 2 hours and 12 hours, between 2 hours and 6 hours, between 2 hours and 4 hours, between 2 hours and 24 hours, between 2 hours and 12 hours, between 2 hours and 6 hours, between 2 hours and 4 hours, between 4 hours and 24 hours, between 4 hours and 12 hours, between 4 hours and 6 hours, between 6 hours and 24 hours, between 6 hours and 12 hours, or between 12 hours and 24 hours. In some of any of the provided embodiments, the steps (a)-(d) are carried out for a time that is between 2 hours and 6 hours. In some of any of the provided embodiments, the steps (a)-(d) are carried out for a time that is between 2 hours and 4 hours or between 3 hours and 4 hours.

In some of any of the provided embodiments, the closed fluid circuit comprises one or more of a blood processing set for obtaining the whole blood from the subject, a separation chamber for the separating the blood, a contacting container for the contacting the PBMCs or subset thereof with the composition comprising lipid particles or viral vectors, and a transfer container containing the contacted PBMCs or subset thereof and/or the transfection mixture for reinfusion to the subject. In some of any of the provided embodiments, the closed fluid circuit comprises one or more of a blood processing set for obtaining the whole blood from the subject, a separation chamber for the separating the blood, a contacting container for the contacting the PBMCs or subset thereof with the composition comprising lipid particles or viral vectors, and a transfer container containing the contacted PBMCs or subset thereof and/or the transfection mixture for reinfusion to the subject. In some of any of the provided embodiments, the blood processing set, the separation chamber, the contacting chamber and/or the transfer container are operably connected via at least one connector set comprising at least one tubing line and optionally one or more connectors. In some of any of the provided embodiments, the transfer container is separably connected form the closed fluid circuit for reinfusion.

In some of any of the provided embodiments, the transfer container is not disengaged from the closed fluid circuit during reinfusion to the subject. In some of any of the provided embodiments, the transfer container is part of a return processing unit comprised by the closed fluid circuit, said return processing unit configured to reinfuse the PBMCs or subset thereof or the transfection mixture to the subject. In some of any of the provided embodiments, the operable connection is via at least one connector selected from the group consisting of valves, luer ports and spikes. In some of any of the provided embodiments, the connector set is disposable. In some of any of the provided embodiments, the connector set is sterile. In some of any of the provided embodiments, the closed fluid circuit is sterile. In some of any of the provided embodiments, the transfer container is operably connected to the closed fluid circuit and/or donor subject, optionally via one or more tubing lines, during the reinfusion to the subject. In some embodiments, the contacting chamber comprises a centrifuge.

In some of any of the provided embodiments, the closed fluid circuit further comprises a collection container operably connected to the separation chamber to collect the PBMCs or subset, optionally wherein the collection container is a bag, more optionally a sterile bag. In some of any of the provided embodiments, the contacting chamber and the transfer container are the same container, optionally wherein the container is a bag, more optionally a sterile bag. In some of any of the provided embodiments, the collecting container, the contacting chamber, and the transfer container are the same container, wherein the container is a bag, more optionally a sterile bag. In some of any of the provided embodiments, the closed fluid circuit comprises one or more of a blood processing set for obtaining the whole blood from the subject, a separation chamber for the separating the PBMCs or subset from the blood to collect the PBMCs or subset, and a container, wherein the container is configured as a collection container for collecting the PBMCs or subset from the separation chamber, a contacting chamber for contacting with the lipid particles (e.g. lentiviral vector) to create a transfection mixture, and a transfer container for reinfusing the transfer mixture to the subject. In some of any of the provided embodiments, the container is a bag, optionally a sterile bag. In some of any of the provided embodiments, the blood processing set, the separation chamber, and the container are operably connected via at least one connector set comprising at least one tubing line and optionally one or more connectors. In some of any of the provided embodiments, the container or the collecting container is operably connected to a source container comprising the composition comprising lipid particles (e.g. lentiviral vector), optionally wherein the operable connection is via at least one connector set comprising at least one tubing line and optionally one or more connectors. In some of any of the provided embodiments, the container or the transfer container is operably connected to a return processing unit for reinfusion of contacted PBMCs or the transfection mixture to the subject. In some of any of the provided embodiments, the operably connection is via at least one connector set comprising at least one tubing line and optionally one or more connectors.

In some embodiments, the collected fraction of blood contains PBMCs or subset thereof separated from other blood components. In some embodiments, the PBMCs or wherein collecting the fraction of blood is by apheresis. In some embodiments, the apheresis device comprises membrane apheresis or centrifugal apheresis. In some embodiments, the collected fraction comprises leukocytes or precursors thereof. In some embodiments, the precursors thereof comprise hematopoietic stem cells or CD34+progenitors.

In some embodiments, collecting the fraction of blood is by leukapheresis. In some embodiments, the collected fraction of blood contains leukocytes. In some embodiments, the collected fraction is a leukapheresis composition obtained from whole blood by leukapheresis.

In some embodiments, the transfection mixture is reinfused into the subject. In some embodiments, the transfection mixture comprises an anticoagulant. In some embodiments, the anticoagulant is a citrate.

In some embodiments, the viability of cells of the collected fraction is greater than 95%, e.g. between 95% and 100%, between 96% and 100%, between 97% and 100%, between 98% and 100% or between 99% and 100%. In some embodiments, the viability of cells of the contacted PBMCs or subset thereof or of cell in the transfection mixture is greater than 95%, e.g. between 95% and 100%, between 96% and 100%, between 97% and 100%, between 98% and 100% or between 99% and 100%.

In some embodiments, the lipid particle is a viral vector or viral-like particle. In some embodiments, the viral vector or viral-like particle is a retroviral vector or retroviral-like particle. In some embodiments, the viral vector or viral-like particle is a lentiviral vector or lentiviral-like particle. In some embodiments, the viral vector or viral-like particle comprises a fusogen embedded in the lipid bilayer.

In some embodiments, the fusogen is a viral fusogen selected from a Class I viral membrane fusion protein, a Class II viral membrane protein, a Class II viral membrane fusion protein, a viral membrane glycoprotein, or a viral envelope protein. In some embodiments, the fusogen is endogenous to the virus. In some embodiments, the fusogen is a pseudotyped fusogen. In some embodiments, the fusogen is a viral envelope protein. In some embodiments, the fusogen is a vesicular stomatitis virus envelope glycoprotein (VSV-G). In some embodiments, the fusogen is a baboon endogenous virus (BaEV) envelope glycoprotein. In some embodiments, the fusogen is a Cocal virus envelope glycoprotein.

In some embodiments, the fusogen is an Alphavirus fusion protein (e.g. Sindbis virus) or a functional variant thereof. In some embodiments, the fusogen is a Paramyxoviridae fusion protein (e.g., a Morbillivirus or a Henipavirus) or a functional variant thereof. In some embodiments, the fusogen is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some embodiments, the fusogen is a Henipavirus fusion protein (e.g., Nipah virus, Hendra virus, Cedar virus, Kumasi virus, Mójiāng virus) or a functional variant thereof. In some embodiments, the fusogen is a Nipah virus fusion protein or a functional variant thereof.

In some embodiments, the lipid particle comprises a paramyxovirus F protein, or a biologically active portion thereof. In some embodiments, the lipid particle comprises a paramyxovirus G, paramyxovirus H, and/or paramyxovirus HN protein, or a biologically active portion thereof. In some embodiments, the paramyxovirus G, paramyxovirus H, and/or paramyxovirus HN protein further comprises a targeting moiety.

In some embodiments, the lipid particle comprises a nucleic acid encoding a payload gene. In some embodiments, the nucleic acid encoding a payload gene encodes a chimeric antigen receptor (CAR). In some embodiments, the targeting moiety comprises a binding agent. In some embodiments, the binding agent is targeted against CD3, CD4, CD8, CD34, CD90, CD19, CD20, CD22, CD16, or CD56.

In some embodiments, the fusogen comprises an F protein molecule or a biologically active portion thereof from a Paramyxovirus and/or a glycoprotein G (G protein) or a biologically active portion thereof from a Paramyxovirus. In some embodiments, the Paramyxovirus is a henipavirus. In some embodiments, the Paramyxovirus is Nipah virus. In some embodiments, the fusogen comprises a Nipah virus F glycoprotein (NiV-F) or a biologically active portion thereof and a Nipah virus G glycoprotein (NiV-G) or a biologically active portion thereof. In some embodiments, the Paramyxovirus is Hendra virus.

In some embodiments, the G protein or the biologically active portion thereof is a mutant NiV-G protein or biologically active portion thereof that exhibits reduced binding to Ephrin B2 or Ephrin B3. In some embodiments, the mutant NiV-G protein comprises one or more amino acid substitutions corresponding to amino acid substitutions selected from the group consisting of E501A, W504A, Q530A and E533A with reference to numbering set forth in SEQ ID NO:14. In some embodiments, the G protein or biologically active portion is a biologically active portion of wild-type NiV-G that has a deletion of up to 40 amino acids at or near the N-terminus, optionally not including the initial methionine. In some embodiments, the G protein is a biologically active portion that is a truncated NiV-G that has a deletion of amino acids 2-34 at or near the N-terminus of wild-type NiV-G set forth in SEQ ID NO:14. In some embodiments, the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

In some embodiments, the F protein or the biologically active portion thereof is a NiV-F protein or a biologically active portion thereof. In some embodiments, the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine. In some embodiments, the F protein or the biologically active portion is a truncated NiV-F that lacks amino acids 523-546 of SEQ ID NO:2. In some embodiments, the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12. In some embodiments, the Niv-G protein comprises the amino acid sequence set forth in SEQ ID NO: 19, and the Niv-F protein comprises the amino acid sequence set forth in SEQ ID NO:12.

In some embodiments, the fusogen is a re-targeted fusogen that binds to a target cell. In some embodiments, the fusogen comprises a targeting moiety that binds to the target cell. In some embodiments, the target cell is a T cell, a B cell, an NK cell, a macrophage, a monocyte, a dendritic cell, a hematopoietic stem cell or a CD34+progenitor cell. In some embodiments, the target cell is a T cell. In some embodiments, the targeting moiety binds to CD4 or CD8. In some embodiments, the targeting moiety is a Design ankyrin repeat proteins (DARPin), a single domain antibody (sdAb), a single chain variable fragment (scFv), or an antigen-binding fibronectin type III (Fn3) scaffold. Provided herein is an in-line method for administration of a lentiviral vector to a subject, the method comprising: a) obtaining whole blood from a subject; b) collecting the fraction of blood containing peripheral blood mononuclear cells (PBMCs) or subset thereof; c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and d) reinfusing the contacted PBMCs or subset and/or the transfection mixture to the subject, thereby administering the lentiviral vector to the subject, wherein steps (a)-(d) are performed in-line in a closed fluid circuit.

Provided herein is a method for administrating a lentiviral vector to a subject, the method comprising: a) obtaining whole blood from a subject; b) collecting the fraction of blood containing peripheral blood mononuclear cells (PBMCs) or leukocyte components; c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and d) reinfusing the contacted PBMCs or subset thereof and/or the transfection mixture to the subject, thereby administering the lentiviral particle to the subject, wherein the method is characterized by one or more of: (i) wherein the method is characterized by the collecting in (b) not comprising a selection step (e.g. immunoaffinity selection) for target cells; (ii) the contacting in step (c) is initiated within 24 hours after collecting the fraction of blood containing PBMCs or subset thereof; (iii) the contacting in step (c) is for no more than 24 hours prior to the reinfusing in step (d); (iv) the whole blood, PBMCs or subset thereof, and transfection mixture are not subjected to cryopreservation or freezing; and/or (v) steps (a)-(d) are carried out for a time that is no more than 24 hours.

Provided herein is am in-line method for administrating a lentiviral vector to a subject, the method comprising: a) obtaining whole blood from a subject; b) collecting the fraction of blood containing peripheral blood mononuclear cells (PBMCs) or leukocyte components; c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and d) reinfusing the contacted PBMCs or subset thereof and/or the transfection mixture to the subject, thereby administering the lentiviral particle to the subject, wherein the method is characterized by one or more of: (i) wherein the method is characterized by the collecting in (b) not comprising a selection step (e.g. immunoaffinity selection) for target cells; (ii) the contacting in step (c) is initiated within 24 hours after collecting the fraction of blood containing PBMCs or subset thereof; (iii) the contacting in step (c) is for no more than 24 hours prior to the reinfusing in step (d); (iv) the whole blood, PBMCs or subset thereof, and transfection mixture are not subjected to cryopreservation or freezing; and/or (v) steps (a)-(d) are carried out for a time that is no more than 24 hours.

In some embodiments, the CAR binds to or recognizes a protein or antigen expressed by or on cells associated with a disease or condition. In some embodiments, the disease or condition is a cancer.

Provided herein is an in-line method for treating cancer in a subject, the method comprising: a) obtaining whole blood from a subject; b) collecting the fraction of blood containing peripheral blood mononuclear cells (PBMCs) or subset thereof; c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and d) reinfusing the contacted PBMCs or subset and/or the transfection mixture to the subject, thereby administering the lentiviral vector to the subject, wherein steps (a)-(d) are performed in-line in a closed fluid circuit.

Provided herein is a method for treating cancer in a subject, the method comprising: a) obtaining whole blood from a subject; b) collecting the fraction of blood containing peripheral blood mononuclear cells (PBMCs) or subset thereof; c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and d) reinfusing the contacted PBMCs or subset and/or the transfection mixture to the subject, thereby administering the lentiviral vector to the subject, wherein steps (a)-(d) are performed in-line in a closed fluid circuit.

Provided herein is a method for treating a cancer in a subject, the method comprising: a) obtaining whole blood from a subject; b) collecting the fraction of blood containing peripheral blood mononuclear cells (PBMCs) or leukocyte components; c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and d) reinfusing the contacted PBMCs or subset thereof and/or the transfection mixture to the subject, thereby administering the lentiviral particle to the subject, wherein the method is characterized by one or more of: (i) wherein the method is characterized by the collecting in (b) not comprising a selection step (e.g. immunoaffinity selection) for target cells; (ii) the contacting in step (c) is initiated within 24 hours after collecting the fraction of blood containing PBMCs or subset thereof; (iii) the contacting in step (c) is for no more than 24 hours prior to the reinfusing in step (d); (iv) the whole blood, PBMCs or subset thereof, and transfection mixture are not subjected to cryopreservation or freezing; and/or (v) steps (a)-(d) are carried out for a time that is no more than 24 hours.

In some of any of the provided embodiments, the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker. In some of any of the provided embodiments, the CD8 binding agent is a VHH having the sequence set forth in SEQ ID NO: 128. In some of any of the provided embodiments, the CD8 binding agent is linked to the C-terminus of a truncated NiV-G set forth in SEQ ID NO: 19 for retargeting of the lipid particle (e.g. lentiviral vector) to CD8+ T cells). In some of any of the provided embodiments the lipid particle (e.g. lentiviral vector) comprising the retargeted NiV-G is pseudotyped with the a NiV-F of a biologically active portion thereof, optionally wherein the NiV-F or biologically active portion is set forth in SEQ ID NO:12.

In some of any of the provided embodiments, the composition comprising the lipid particle is a viral vector and the composition comprising the lipid particle or the composition comprising the lentiviral vector comprises from 1×10⁸ to 1×10¹¹ infectious units (IU), 1×10⁸ to 1×10¹⁰ IU, 1×10⁸ to 1×10⁹ IU, 1×10⁹ to 1×10¹¹ IU, 1×10⁹ to 1×10¹⁰IU, 1×10¹⁰ to 1×10¹¹ IU. In some of any of the provided embodiments, the volume of the composition comprising lipid particles is between 100 mL and 400 mL, inclusive. In some of any of the provided embodiments, the collected PBMCs or subset thereof comprises from 1×108 to 1×1010 nucleated cells, 1×108 to 5×109 nucleated cells, 1×108 to 2×109 nucleated cells, 1×108 to 1×109 nucleated cells, 1×108 to 5×108 nucleated cells, 5×108 to 1×1010 nucleated cells, 5×108 to 5×109 nucleated cells, 5×108 to 2×109 nucleated cells, 5×108 to 1×109 nucleated cells, 1×109 to 1×1010 nucleated cells, 1×108 to 5×109 nucleated cells, 1×108 to 2×109 nucleated cells, 2×109 to 1×1010 nucleated cells, 2×108 to 5×109 nucleated cells, or 5×109 to 1 x 1010 nucleated cells. In some of any of the provided embodiments, the volume of the collected PBMCs or subset thereof is between 100 mL and 400 mL, inclusive. In some of any of the provided embodiments, the concentration of the PBMCs or subset during the contacting is from 1×106 cells/mL to lx 108 cells/mL, from 1×106 cells/mL to 5×107 cells/mL, from 1×106 cells/mL to 1×107 cells/mL, from 1×106 cells/mL to 5×106 cells/mL, from 5×106 cells/mL to 1×108 cells/mL, from 5×106 cells/mL to 5×107 cells/mL, from 5×106 cells/mL to 1×107 cells/mL, from 1×107 cells/mL to 1×108 cells/mL, from 1×107 cells/mL to 5×107 cells/mL, from 5×107 cells/mL to 1×108 cells/mL.

In some of any of the provided embodiments, the method does not include a lymphodepleting regimen prior to obtaining the whole blood from the subject. In some of any of the provided embodiments, the method does not include a lymphodepleting regimen prior to reinfusing the contacted PBMCs or subset or the transfection mixture to the subject. In some of any of the provided embodiments, the subject has not been subjected to a lymphodepleting regimen within 30 days prior, optionally within one week, prior to reinfusing the contacting PBMCs or subset of the transfection mixture to the subject.

In some of any of the provided embodiments, the payload agent is or encodes a therapeutic agent. In some of any of the provided embodiments, the payload agent is a nucleic acid comprising a gene for correcting a genetic deficiency. In some of any of the provided embodiments, the payload agent encodes a membrane protein. In some of any of the provided embodiments, the membrane protein is an antigen receptor for targeting cells expressed by or associated with a disease or condition. In some of any of the provided embodiments, the membrane protein is a chimeric antigen receptor (CAR). In some embodiments, the CAR is an anti-CD19 CAR, an anti-CD22 CAR or an anti-CD22 CAR. In some embodiments, the CAR is an anti-CD19 CAR. In some embodiments, the anti-CD19 CAR comprises an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36, and a CD3zeta signaling domain set forth in SEQ ID NO: 38.

In some embodiments, the method further comprises administering a cytokine receptor agonist to the subject. In some embodiments, the cytokine receptor agonist is a recombinant protein, a chemically synthesized protein or a conjugate. In some embodiments, the cytokine receptor agonist binds to a cytokine receptor on a T cell. In some embodiments, the cytokine receptor is selected from the group consisting of an IL-2 receptor (IL-2R), an IL-15 receptor (IL-15R), an IL-7 receptor (IL-7R), or an IL-21 receptor (IL-21R). In some embodiments, the cytokine receptor agonist comprises a T cell stimulating cytokine or a T cell stimulating cytokine mutein, a T cell stimulating cytokine mimetic, an antibody or antigen-binding fragment that binds a cytokine receptor on a T cell, or an antibody or antigen-binding fragment that binds a T cell stimulating cytokine. In some embodiments, the cytokine receptor agonist is a conjugate comprising (1) a T cell stimulating cytokine, a T cell stimulating cytokine mutein or a T cell stimulating cytokine mimetic and (2) a water soluble polymer. In some embodiments, the cytokine receptor agonist is a fusion protein comprising (1) a T cell stimulating cytokine, a T cell stimulating cytokine mutein or a T cell stimulating cytokine mimetic and (2) a half-life extending moiety.

In some embodiments, the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21), or a T cell stimulating cytokine mutein of any of the foregoing. In some embodiments, the T cell stimulating cytokine mutein comprises at least one amino acid modification relative to a wild-type T cell stimulating cytokine. In some embodiments, the T cell stimulating cytokine mutein is an IL-2 cytokine mutein comprising one or more amino acid modifications relative to wild-type human IL-2. In some embodiments, the IL-2 mutein exhibits increased affinity for the IL-2 Rβ, relative to wild-type human IL-2. In some embodiments, the IL-2 mutein exhibits increased IL-2 activity for the intermediate affinity IL-2 receptor composed of IL-2Rbeta and IL-2Rgamma (IL-2R R/y), relative to wild-type human IL-2. In some embodiments, the IL-2 mutein exhibits reduced binding to IL-2Ralpha, relative to wild-type human IL-2. In some embodiments, the IL-2 mutein exhibits reduced IL-2 activity for the high-affinity IL-2 receptor composed of IL-2Ralpha, IL-2Rbeta and IL-2Rgamma (IL-2R a/p/y). In some embodiments, the T cell stimulating cytokine or cytokine mutein is IL-15 or an IL-15 cytokine mutein and the IL-15 or IL-15 cytokine mutein is bound to IL-15Ra or a portion thereof comprising the sushi domain. In some embodiments, the T cell stimulating cytokine mutein is a IL-15 cytokine mutein comprising one more amino acid modifications relative to human IL-15. In some embodiments, the IL-15 mutein exhibits reduced binding to IL-15Ra. In some embodiments, the cytokine receptor agonist comprises a T cell stimulating cytokine mimetic and the mimetic is a IL-2Rα ligand, a IL-2Rβ ligand, a IL-2Rγ ligand, a common γc receptor (Rγc) ligand, and/or IL-7Rα ligand.

In some embodiments, the one, two, three, four, five or six water-soluble polymers are attached to the T cell stimulating cytokine. In some embodiments, the water soluble polymer is a polymer selected from the group consisting of poly(alkylene oxide), poly(vinyl pyrrolidone), poly(vinyl alcohol), polyoxazoline, and poly(acryloylmorpholine). In some embodiments, the water-soluble polymer has a weight-average molecular weight in a range of from about 500 Daltons to about 100,000 Daltons.

In some embodiments, the water-soluble polymer is a poly(alkylene oxide). In some embodiments, the poly(alkylene oxide) is a poly(ethylene glycol). In some embodiments, the cytokine receptor agonist is a human IL-2 or IL-2 mutein covalently attached to one or more poly(ethylene glycol) polymers. In some embodiments, the cytokine receptor agonist is a human IL-15 or IL-15 mutein covalently attached to one or more poly(ethylene glycol) polymers. In some embodiments, the cytokine receptor agonist is a human IL-7 or IL-7 mutein covalently attached to one or more poly(ethylene glycol) polymers. In some embodiments, the cytokine receptor agonist is a human IL-21 or IL-21 mutein covalently attached to one or more poly(ethylene glycol) polymers. In some embodiments, the half-life extending moiety is an Fc region of an immunoglobulin, human serum albumin, an albumin binding moiety, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof. In some embodiments, the half-life extending moiety is an albumin binding moiety. In some embodiments, the cytokine receptor agonist is a fusion protein comprising a human IL-2 or IL-2 mutein fused an albumin binding moiety. In some embodiments, the cytokine receptor agonist is a fusion protein comprising a human IL-15 or IL-15 mutein fused to an albumin binding moiety. In some embodiments, the cytokine receptor agonist is a fusion protein comprising a human IL-7 or IL-7 mutein fused to an albumin binding moiety. In some embodiments, the cytokine receptor agonist is a fusion protein comprising a human IL-21 or IL-21 mutein fused to an albumin binding moiety. In some embodiments, the albumin binding moiety is a single domain antibody (sdAb) that specifically binds to albumin.

In some embodiments, the half-life extending moiety is an Fc region of an immunoglobulin. In some embodiments, the cytokine receptor agonist is a fusion protein comprising a human IL-2 or IL-2 mutein fused an Fc region of an immunoglobulin. In some embodiments, the cytokine receptor agonist is a fusion protein comprising a human IL-15 or IL-15 mutein fused to an Fc region of an immunoglobulin. In some embodiments, the cytokine receptor agonist is a fusion protein comprising a human IL-7 or IL-7 mutein fused to an Fc region of an immunoglobulin. In some embodiments, the cytokine receptor agonist is a fusion protein comprising a human IL-21 or IL-21 mutein fused to an Fc region of an immunoglobulin. In some embodiments, the Fc of an immunoglobulin is an Fc of human IgG1. In some embodiments, the Fc of an immunoglobulin is an Fc of human IgG4. In some embodiments, the T cell stimulating cytokine or mutein is an IL-7 or IL-7 mutein that is glycosylated. In some embodiments, the T cell stimulating cytokine or mutein is hyperglycosylated, relative to wild-type human IL-7. In some embodiments, the T cell stimulating cytokine or mutein is produced from Chinese Hamster Ovary (CHO) cells. In some embodiments, the T cell stimulating cytokine or mutein is an IL-7 conformer, wherein said conformer comprises the following three disulfide bridges: Cys: 1-4 (Cys2-Cys92); 2-5 (Cys34-Cys129) and 3-6 (Cys47-Cys14l). In some embodiments, the cytokine receptor agonist is an antibody or antigen-binding fragment that binds a T cell stimulating cytokine and the T cell stimulating cytokine is human IL-2. In some embodiments, the antibody or antigen binding fragment inhibits binding of IL-2 with an IL-2 receptor alpha (IL-2 Rα) subunit, inhibits IL-2 signaling through IL-2 Rαβγ and through IL-2 Rβγ and/or inhibits IL-2 signaling through IL-2 Rαβγ to a greater extent than through IL-2 Rβγ. In some embodiments, the cytokine receptor agonist is an antibody or antigen-binding fragment that binds a T cell stimulating agent and the T cell stimulating agent is human IL-21. In some embodiments, the antibody or antigen binding fragment enhances human IL-21 activity through the IL-21 receptor.

In some embodiments, the cytokine receptor agonist is selected from the group consisting of NL-201, SAR444245 (IL-2 Synthorin™), STK-012, BPT-143, AU-007, IL-15 Synthorin™, P10-001, bempegaldesleukin (NKTR-214), SHR-1916, ARK102, 8MW-2311, NKTR-255, Exenokine-2, MDNA-11, GX-17/NT-17, SHR-1501, ASKG-215, BCD-225, Exenokine-21, MK-1169, Hu-Mikβ1, JS08-1, CYT-107, AM0015 and KW-007.

In some embodiments, the cytokine receptor agonist is administered by the in-line method of administration. In some embodiments, the transfection mixture further comprises the cytokine receptor agonist, and wherein reinfusing the transfection mixture to the subject further administers the cytokine receptor agonist to the subject by the in-line method of administration. In some embodiments, the collected PBMCs or subset are contacted with the cytokine receptor agonist to produce the transfection mixture comprising the cytokine receptor agonist, wherein the contacting with the cytokine receptor agonist is carried out prior to the reinfusing of step (d). In some embodiments, the contacting with the cytokine receptor agonist is carried out prior to, concurrently with or after the contacting with the composition comprising lipid particles or lentiviral vector. In some embodiments, the contacting with the cytokine receptor agonist is performed in-line in the closed fluid circuit.

In some embodiments, the amount of the cytokine receptor agonist is from or from about 0.05 mg to 10 mg, from or from about 0.05 mg to 7.5 mg, from or from about 0.05 mg to 5 mg, from or from about 0.05 mg to 2.5 mg, from or from about 0.05 mg to 1 mg, from or from about 0.05 mg to 0.5 mg, from or from about 0.05 mg to 0.25 mg, from or from about 0.05 mg to 0.1 mg, from or from about 0.05 mg to 0.075 mg, from or from about 0.075 mg to 10 mg, from or from about 0.075 mg to 7.5 mg, from or from about 0.075 mg to 5 mg, from or from about 0.075 mg to 2.5 mg, from or from about 0.075 mg to 1 mg, from or from about 0.075 mg to 0.5 mg, from or from about 0.075 mg to 0.25 mg, from or from about 0.075 mg to 0.1 mg, from or from about 0.1 mg to 10 mg, from or from about 0.1 mg to 7.5 mg, from or from about 0.1 mg to 5 mg, from or from about 0.1 mg to 2.5 mg, from or from about 0.1 mg to 1 mg, from or from about 0.1 mg to 0.5 mg, from or from about 0.1 mg to 0.25 mg, from or from about 0.25 mg to 10 mg, from or from about 0.25 mg to 7.5 mg, from or from about 0.25 mg to 5 mg, from or from about 0.25 mg to 2.5 mg, from or from about 0.25 mg to 1 mg, from or from about 0.25 mg to 0.5 mg, from or from about 0.5 mg to 10 mg, from or from about 0.5 mg to 7.5 mg, from or from about 0.5 mg to 5 mg, from or from about 0.05 mg to 2.5 mg, from or from about 0.05 mg to 1 mg, from or from about 1 mg to 10 mg, from or from about 1 mg to 7.5 mg, from or from about 1 mg to 5 mg, from or from about 1 mg to 2.5 mg, from or from about 2.5 mg to 10 mg, from or from about 2.5 mg to 7.5 mg, from or from about 2.5 mg to 5 mg, from or from about 5 mg to 10 mg, from or from about 5 mg to 7.5 mg, or from or from about 7.5 mg to 10 mg. In some embodiments, the volume of the transfection mixture is between 100 mL and 1000 mL, inclusive. In some embodiments, the volume of the transfection mixture is between 100 mL and 400 mL. In some embodiments, the method further comprises administering one or more doses of the cytokine receptor agonist to the subject after the in-line administration of the lipid particle or lentiviral vector. In some embodiments, the one or more doses of the cytokine receptor agonist is administered to the subject separate from the in-line administration of the lentiviral vector. In some embodiments, each of the one or more doses of the cytokine receptor agonist is from at or about 0.001 mg/kg to at or about 0.1 mg/kg, at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg. In some embodiments, each of the one or more doses of the cytokine receptor agonist is from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

In some embodiments, the cytokine receptor agonist is administered daily, once a week (Q1W), once every two weeks (Q2W), once every three weeks (Q3W) or once every four weeks (Q4W). In some embodiments, the cytokine receptor agonist is administered one time. In some embodiments, the cytokine receptor agonist is administered for one week, two weeks, three weeks, four weeks, five weeks, six weeks, seven weeks or eight weeks. In some embodiments, the cytokine receptor agonist is administered subcutaneously. In some embodiments, the cytokine receptor agonist is administered intravenously. In some embodiments, the cytokine receptor agonist is administered intramuscularly. In some embodiments, a first dose of the cytokine receptor agonist is administered prior to the in-line administration of the lipid particle or the lentiviral vector. In some embodiments, the first dose of the cytokine receptor agonist is administered within one month, within one week or within three days of the in-line administration of the lipid particle or the lentiviral vector. In some embodiments, the first dose of the cytokine receptor agonist is administered on the same day as the in-line administration of the lipid particle or the lentiviral vector. In some embodiments, the first dose of the cytokine receptor agonist is administered after the in-line administration of the lipid particle or the lentiviral vector. In some embodiments, the first dose of the cytokine receptor agonist is administered no more than one month, no more than 21 days, no more than 14 days or no more than 7 days after the in-line administration of the lipid particle or the lentiviral vector.

Provided herein is a system for infusion of lipid particles or viral vectors into a subject, the system comprising: (a) an incoming processing unit for obtaining whole blood from the circulatory system of a subject; (b) a separation chamber for collecting peripheral blood mononuclear cells (PBMCs) or subset thereof from the blood fraction; and (c) a contacting chamber container for transfection of the PBMCs or subset thereof with a composition comprising lipid particles or viral vectors to create a transfection mixture; and (d) a transfer container for reinfusing the contacted PBMCs or subset thereof or the transfection mixture to the same subject.

In some of any of the provided embodiments, the payload agent is or encodes a therapeutic agent. In some of any of the provided embodiments, the payload agent is a nucleic acid comprising a gene for correcting a genetic deficiency. In some of any of the provided embodiments, the payload agent encodes a membrane protein, optionally wherein the membrane protein is an antigen receptor for targeting cells expressed by or associated with a disease or condition. In some of any of the provided embodiments, the membrane protein is a chimeric antigen receptor (CAR).

Provided herein is a system for infusion of lipid particles or viral vectors into a subject, the system comprising: (a) an incoming processing unit for obtaining whole blood from the circulatory system of a subject; (b) a separation chamber for collecting peripheral blood mononuclear cells (PBMCs) or subset thereof from the blood fraction; and (c) a contacting chamber container for transfection of the PBMCs or subset thereof with a composition comprising lipid particles or viral vectors to create a transfection mixture; and (d) a transfer container for reinfusing the contacted PBMCs or subset thereof or the transfection mixture to the same subject.

Provided herein is a system for delivering a payload agent into a subject, the system comprising: (a) an incoming processing unit for obtaining whole blood from the circulatory system of a subject; (b) a separation chamber for collecting peripheral blood mononuclear cells (PBMCs) or subset thereof from the blood fraction; and (c) a contacting chamber container for transfection of the PBMCs or subset thereof with a composition comprising lipid particles or viral vectors to create a transfection mixture; and (d) a transfer container for reinfusing the contacted PBMCs or subset thereof or the transfection mixture to the same subject.

In some of any of the provided embodiments, the lipid particle is a viral vector. In some of any of the provided embodiments, the viral vector is a lentiviral vector. In some of any of the provided embodiments, the blood processing set, the separation chamber, the contacting chamber and/or the transfer container are operably connected via at least one connector set comprising at least one tubing line and optionally one or more connectors. In some of any of the provided embodiments, the separation chamber is operably connected to a collection container that collects the PBMCs or subset. In some of any of the provided embodiments, the contacting chamber and the transfer container are configured as part of the same container or are the same container. In some of any of the provided embodiments, the collecting container, the contacting chamber and the transfer container are configured as part of the same container or are the same container. In some of any of the provided embodiments, the container is a bag, optionally sterile bag.

In some of any of the provided embodiments, the system is a closed fluid circuit to operate in-line. In some of any of the provided embodiments, the transfer container configured to be separably connected form the closed fluid circuit for reinfusion. In some of any of the provided embodiments, the transfer container configured not to be disengaged from the closed fluid circuit during reinfusion to the subject. In some of any of the provided embodiments, the transfer container is part of a return processing unit comprised by the system, optionally the closed fluid circuit, said return processing unit configured to reinfuse the PBMCs or subset thereof or the transfection mixture to the subject.

In some of any of the provided embodiments, the separation chamber is operably connected to a collection container that collects the PBMCs or subset. In some of any of the provided embodiments, the contacting chamber and the transfer container are configured as part of the same container. In some of any of the provided embodiments, the collecting container, the contacting chamber and the transfer container are configured as part of the same container. In some of any of the provided embodiments, the container is a bag, optionally sterile bag.

In some of any of the provided embodiments, the operable connection is via at least one connector selected from the group consisting of valves, luer ports and spikes. In some of any of the provided embodiments, the connector set is disposable. In some of any of the provided embodiments, the connector set is sterile.

In some of any of the provided embodiments, the transfer container is configured to be operably connected to the closed fluid circuit and/or donor subject, optionally via one or more tubing lines, during the reinfusion to the subject. In some of any of the provided embodiments, the separation chamber is an apheresis device. In some of any of the provided embodiments, the separation chamber is a leukapheresis device. In some of any of the provided embodiments, the contacting chamber comprises a centrifuge. In some of any of the provided embodiments, the contacting chamber is configured to be operably connected, optionally via a sterile connector set, to a container comprising the composition comprising the lipid particles.

In some of any of the provided embodiments, the composition comprising the lipid particles comprises a cytokine receptor agonist. In some of any of the provided embodiments, the contacting container is configured to be operably connected, optionally via a sterile connector set, to a container comprising a cytokine receptor agonist. In some of any of the provided embodiments, the transfer container is configured to be operably connected, optionally via a sterile connector set, to a container comprising a cytokine receptor agonist.

Provided herein is a combination comprising the system of any embodiment provided and a container comprising the composition comprising the lipid particles. In some of any of the provided embodiments, the lipid particle is a viral vector. In some of any of the provided embodiments, the viral vector is a lentiviral vector. In some of any of the provided embodiments, the viral vector comprises a fusogen embedded in the lipid bilayer. In some of any of the provided embodiments, the fusogen is a viral fusogen selected from a Class I viral membrane fusion protein, a Class II viral membrane protein, a Class II viral membrane fusion protein, a viral membrane glycoprotein, or a viral envelope protein. In some of any of the provided embodiments, the fusogen is endogenous to the virus. In some of any of the provided embodiments, the fusogen is a pseudotyped fusogen. In some of any of the provided embodiments, the fusogen is a viral envelope protein. In some of any of the provided embodiments, the fusogen is a vesicular stomatitis virus envelope glycoprotein (VSV-G). In some of any of the provided embodiments, the fusogen is a baboon endogenous virus (BaEV) envelope glycoprotein. In some of any of the provided embodiments, the fusogen is a Cocal virus envelope glycoprotein. In some of any of the provided embodiments, the fusogen is an Alphavirus fusion protein (e.g. Sindbis virus) or a functional variant thereof. In some of any of the provided embodiments, the fusogen is a Paramyxoviridae fusion protein (e.g., a Morbillivirus or a Henipavirus) or a functional variant thereof. In some of any of the provided embodiments, the fusogen is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some of any of the provided embodiments, the fusogen is a Henipavirus fusion protein (e.g., Nipah virus, Hendra virus, Cedar virus, Kumasi virus, Mójiāng virus) or a functional variant thereof. In some of any of the provided embodiments, the fusogen is a Nipah virus fusion protein or a functional variant thereof. In some of any of the provided embodiments, the fusogen comprises a paramyxovirus F protein, or a biologically active portion thereof. In some of any of the provided embodiments, the fusogen comprises a paramyxovirus G, paramyxovirus H, and/or paramyxovirus HN protein, or a biologically active portion thereof. In some of any of the provided embodiments, the lipid particle or lentiviral vector comprises a nucleic acid encoding a payload gene. In some of any of the provided embodiments, the nucleic acid encoding a payload gene encodes a chimeric antigen receptor (CAR). In some of any of the provided embodiments, the fusogen comprises an F protein molecule or a biologically active portion thereof from a Paramyxovirus and a glycoprotein G (G protein) or a biologically active portion thereof from a Paramyxovirus. In some of any of the provided embodiments, the Paramyxovirus is a henipavirus. In some of any of the provided embodiments, the Paramyxovirus is Nipah virus. In some of any of the provided embodiments, the fusogen comprises a Nipah virus F glycoprotein (NiV-F) or a biologically active portion thereof and a Nipah virus G glycoprotein (NiV-G) or a biologically active portion thereof. In some of any of the provided embodiments, the Paramyxovirus is Hendra virus. In some of any of the provided embodiments, the G protein or the biologically active portion thereof is a mutant NiV-G protein or biologically active portion thereof that exhibits reduced binding to Ephrin B2 or Ephrin B3. In some of any of the provided embodiments, the mutant NiV-G protein comprises one or more amino acid substitutions corresponding to amino acid substitutions selected from the group consisting of E501A, W504A, Q530A and E533A with reference to numbering set forth in SEQ ID NO:14. In some of any of the provided embodiments, the G protein or biologically active portion is a biologically active portion of wild-type NiV-G that has a deletion of up to 40 amino acids at or near the N-terminus, optionally not including the initial methionine. In some of any of the provided embodiments, the G protein is a biologically active portion that is a truncated NiV-G that has a deletion of amino acids 2-34 at or near the N-terminus of wild-type NiV-G set forth in SEQ ID NO:14. In some of any of the provided embodiments, the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

In some of any of the provided embodiments, the F protein or the biologically active portion thereof is a NiV-F protein or a biologically active portion thereof. In some of any of the provided embodiments, the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine. In some of any of the provided embodiments, the F protein or the biologically active portion is a truncated NiV-F that lacks amino acids 525-546 of SEQ ID NO:2. In some of any of the provided embodiments, the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12. In some of any of the provided embodiments, the Niv-G protein comprises the amino acid sequence set forth in SEQ ID NO: 19, and the Niv-F protein comprises the amino acid sequence set forth in SEQ ID NO:12.

In some of any of the provided embodiments, the fusogen is a re-targeted fusogen that binds to a target cell. In some of any of the provided embodiments, the fusogen comprises a targeting moiety that binds to the target cell. In some of any of the provided embodiments, the paramyxovirus G, paramyxovirus H, and/or paramyxovirus HN protein further comprises a targeting moiety. In some of any of the provided embodiments, the targeting moiety comprises a binding agent. In some of any of the provided embodiments, the binding agent is targeted against CD3, CD4, CD8, CD34, CD90, CD19, CD20, CD22, CD16, or CD56. In some of any of the provided embodiments, the target cell is a T cell, a B cell, an NK cell, a macrophage, a monocyte, a dendritic cell, a hematopoietic stem cell or a CD34+progenitor cell. In some of any of the provided embodiments, the target cell is a T cell. In some of any of the provided embodiments, the targeting moiety binds to CD4 or CD8. In some of any of the provided embodiments, the targeting moiety is a Design ankyrin repeat proteins (DARPin), a single domain antibody (sdAb), a single chain variable fragment (scFv), or an antigen-binding fibronectin type III (Fn3) scaffold. In some of any of the provided embodiments, the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker. In some of any of the provided embodiments, the CD8 binding agent is a VHH having the sequence set forth in SEQ ID NO: 128. In some of any of the provided embodiments, the CD8 binding agent is linked to the C-terminus of a truncated NiV-G set forth in SEQ ID NO: 19 for retargeting of the lipid particle or lentiviral vector to CD8+ T cells. In some of any of the provided embodiments, the lipid particle or lentiviral vector comprising the retargeted NiV-G is pseudotyped with the a NiV-F of a biologically active portion thereof, optionally wherein the NiV-F or biologically active portion is set forth in SEQ ID NO:12. In some of any of the provided embodiments, the lipid particle is a viral vector and the composition comprises from 1×10⁸ to 1×10¹¹ infectious units (IU), 1×10⁸ to 1×10¹⁰ IU, 1×10⁸ to 1×10⁹ IU, 1×10⁹ to 1×10¹¹ IU, 1×10⁹ to 1×10¹⁰ IU, 1×101⁰ to 1×10¹¹ IU. In some of any of the provided embodiments, the volume of the composition comprising lipid particles is between 100 mL and 1000 mL, inclusive. In some embodiments, the volume of the composition comprising lipid particles is between 100 mL and 400 mL, inclusive.

In some of any of the provided embodiments, the combination further comprises a cytokine receptor agonist. In some of any of the provided embodiments, the cytokine receptor agonist is a recombinant protein, a chemically synthesized protein or a conjugate. In some of any of the provided embodiments, the cytokine receptor agonist binds to a cytokine receptor on a T cell. In some of any of the provided embodiments, the cytokine receptor is selected from the group consisting of an IL-2 receptor (IL-2R), an IL-15 receptor (IL-15R), an IL-7 receptor (IL-7R), or an IL-21 receptor (IL-21R). In some of any of the provided embodiments, the cytokine receptor agonist comprises a T cell stimulating cytokine or a T cell stimulating cytokine mutein, a T cell stimulating cytokine mimetic, an antibody or antigen-binding fragment that binds a cytokine receptor on a T cell, or an antibody or antigen-binding fragment that binds a T cell stimulating cytokine. In some of any of the provided embodiments, the cytokine receptor agonist is a conjugate comprising (1) a T cell stimulating cytokine, a T cell stimulating cytokine mutein or a T cell stimulating cytokine mimetic and (2) a water soluble polymer. In some of any of the provided embodiments, the cytokine receptor agonist is a fusion protein comprising (1) a T cell stimulating cytokine, a T cell stimulating cytokine mutein or a T cell stimulating cytokine mimetic and (2) a half-life extending moiety. In some of any of the provided embodiments, the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21), or a T cell stimulating cytokine mutein of any of the foregoing. In some of any of the provided embodiments, the T cell stimulating cytokine mutein comprises at least one amino acid modification relative to a wild-type T cell stimulating cytokine. In some of any of the provided embodiments, the T cell stimulating cytokine mutein is an IL-2 cytokine mutein comprising one or more amino acid modifications relative to wild-type human IL-2. In some of any of the provided embodiments, the IL-2 mutein exhibits increased affinity for the IL-2 Rβ, relative to wild-type human IL-2. In some of any of the provided embodiments, the IL-2 mutein exhibits increased IL-2 activity for the intermediate affinity IL-2 receptor composed of IL-2Rbeta and IL-2Rgamma (IL-2R β/γ), relative to wild-type human IL-2. In some of any of the provided embodiments, the IL-2 mutein exhibits reduced binding to IL-2Ralpha, relative to wild-type human IL-2. In some of any of the provided embodiments, the IL-2 mutein exhibits reduced IL-2 activity for the high-affinity IL-2 receptor composed of IL-2Ralpha, IL-2Rbeta and IL-2Rgamma (IL-2R α/β/γ). In some of any of the provided embodiments, the T cell stimulating cytokine or cytokine mutein is IL-15 or an IL-15 cytokine mutein and the IL-15 or IL-15 cytokine mutein is bound to IL-15Rα or a portion thereof comprising the sushi domain. In some of any of the provided embodiments, the T cell stimulating cytokine mutein is a IL-15 cytokine mutein comprising one more amino acid modifications relative to human IL-15. In some of any of the provided embodiments, the IL-15 mutein exhibits reduced binding to IL-15Rα. In some of any of the provided embodiments, the cytokine receptor agonist comprises a T cell stimulating cytokine mimetic and the mimetic is a IL-2Rα ligand, a IL-2Rβligand, a IL-2Rγ ligand, a common γc receptor (Rγc) ligand, and/or IL-7Rα ligand. In some of any of the provided embodiments, the one, two, three, four, five or six water-soluble polymers are attached to the T cell stimulating cytokine.

In some of any of the provided embodiments, the water soluble polymer is a polymer selected from the group consisting of poly(alkylene oxide), poly(vinyl pyrrolidone), poly(vinyl alcohol), polyoxazoline, and poly(acryloylmorpholine). In some of any of the provided embodiments, the water-soluble polymer has a weight-average molecular weight in a range of from about 500 Daltons to about 100,000 Daltons. In some of any of the provided embodiments, the water-soluble polymer is a poly(alkylene oxide). In some of any of the provided embodiments, the poly(alkylene oxide) is a poly(ethylene glycol). In some of any of the provided embodiments, the cytokine receptor agonist is a human IL-2 or IL-2 mutein covalently attached to one or more poly(ethylene glycol) polymers. In some of any of the provided embodiments, the cytokine receptor agonist is a human IL-15 or IL-15 mutein covalently attached to one or more poly(ethylene glycol) polymers. In some of any of the provided embodiments, the cytokine receptor agonist is a human IL-7 or IL-7 mutein covalently attached to one or more poly(ethylene glycol) polymers. In some of any of the provided embodiments, the cytokine receptor agonist is a human IL-21 or IL-21 mutein covalently attached to one or more poly(ethylene glycol) polymers. In some of any of the provided embodiments, the half-life extending moiety is an Fc region of an immunoglobulin, human serum albumin, an albumin binding moiety, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof. In some of any of the provided embodiments, the half-life extending moiety is an albumin binding moiety. In some of any of the provided embodiments, the cytokine receptor agonist is a fusion protein comprising a human IL-2 or IL-2 mutein fused an albumin binding moiety. In some of any of the provided embodiments, the cytokine receptor agonist is a fusion protein comprising a human IL-15 or IL-15 mutein fused to an albumin binding moiety. In some of any of the provided embodiments, the cytokine receptor agonist is a fusion protein comprising a human IL-7 or IL-7 mutein fused to an albumin binding moiety. In some of any of the provided embodiments, the cytokine receptor agonist is a fusion protein comprising a human IL-21 or IL-21 mutein fused to an albumin binding moiety. In some of any of the provided embodiments, the albumin binding moiety is a single domain antibody (sdAb) that specifically binds to albumin.

In some of any of the provided embodiments, the half-life extending moiety is an Fc region of an immunoglobulin. In some of any of the provided embodiments, the cytokine receptor agonist is a fusion protein comprising a human IL-2 or IL-2 mutein fused an Fc region of an immunoglobulin. In some of any of the provided embodiments, the cytokine receptor agonist is a fusion protein comprising a human IL-15 or IL-15 mutein fused to an Fc region of an immunoglobulin. In some of any of the provided embodiments, the cytokine receptor agonist is a fusion protein comprising a human IL-7 or IL-7 mutein fused to an Fc region of an immunoglobulin. In some of any of the provided embodiments, the cytokine receptor agonist is a fusion protein comprising a human IL-21 or IL-21 mutein fused to an Fc region of an immunoglobulin. In some of any of the provided embodiments, the Fc of an immunoglobulin is an Fc of human IgG1. In some of any of the provided embodiments, the Fc of an immunoglobulin is an Fc of human IgG4. In some of any of the provided embodiments, the T cell stimulating cytokine or mutein is an IL-7 or IL-7 mutein that is glycosylated. In some of any of the provided embodiments, the T cell stimulating cytokine or mutein is hyperglycosylated, relative to wild-type human IL-7. In some of any of the provided embodiments, the T cell stimulating cytokine or mutein is produced from Chinese Hamster Ovary (CHO) cells. In some of any of the provided embodiments, the T cell stimulating cytokine or mutein is an IL-7 conformer, wherein said conformer comprises the following three disulfide bridges: Cys: 1-4 (Cys2-Cys92); 2-5 (Cys34-Cys129) and 3-6 (Cys47-Cys141). In some of any of the provided embodiments, the cytokine receptor agonist is an antibody or antigen-binding fragment that binds a T cell stimulating cytokine and the T cell stimulating cytokine is human IL-2. In some of any of the provided embodiments, the antibody or antigen binding fragment inhibits binding of IL-2 with an IL-2 receptor alpha (IL-2 Rα) subunit, inhibits IL-2 signaling through IL-2 Rαβγ and through IL-2 Rβγ and/or inhibits IL-2 signaling through IL-2 Rαβγ to a greater extent than through IL-2 Rβγ. In some of any of the provided embodiments, the cytokine receptor agonist is an antibody or antigen-binding fragment that binds a T cell stimulating agent and the T cell stimulating agent is human IL-21. In some of any of the provided embodiments, the antibody or antigen binding fragment enhances human IL-21 activity through the IL-21 receptor. In some of any of the provided embodiments, the cytokine receptor agonist is selected from the group consisting of NL-201, SAR444245 (IL-2 Synthorin™), STK-012, BPT-143, AU-007, IL-15 Synthorin™, PIO-001, bempegaldesleukin (NKTR-214), SHR-1916, ARK102, 8MW-2311, NKTR-255, Exenokine-2, MDNA-11, GX-I7/NT-I7, SHR-1501, ASKG-215, BCD-225, Exenokine-21, MK-1169, Hu-Mikβ1, JS08-1, CYT-107, AM0015 and KW-007.

Provided herein is a container comprising a leukapheresis composition for delivering a viral vector to a subject, wherein the leukapheresis composition comprises at least 5-1×10⁶ cells/mL to 1×10⁸ cells/mL (100-400 ml leukapheresis product) and viral vector composition consisting of 1×108 to 1 x 10¹¹ infectious units (IU) (100-400 ml viral particle product). In some of any of the provided embodiments, the container is a bag.

Provided herein is a sterile composition comprising between 1×10⁶ cells/mL to 1×108 cells/mL and viral vector composition consisting of 1×10⁸ to 1×10¹¹ infectious units (IU).

Provided herein is a sterile composition comprising between 5-1×106 cells/mL to 1×108 cells/mL of peripheral blood mononuclear cells (PBMCs) or subset thereof and viral vector composition consisting of 1×108 to 1×1011 infectious units (IU).

In some of any of the provided embodiments, the composition has a volume of between 100 mL to 1000 mL.

Provided herein is a sterile composition comprising peripheral blood mononuclear cells (PBMCs) from a 100 mL to 400 mL leukapheresis product and a viral vector composition comprising 1 x 108 to 1×10¹¹ infectious units (IU).

In some of any of the provided embodiments, the volume of the composition is between 100 mL to 1000 mL. In some of any of the provided embodiments, the viability of cells of the collected fraction is greater than 95%, e.g. between 95% and 100%, between 96% and 100%, between 97% and 100%, between 98% and 100% or between 99% and 100%. In some of any of the provided embodiments, the viability of cells of the contacted PBMCs or subset thereof or of cells in the transfection mixture is greater than 95%, e.g. between 95% and 100%, between 96% and 100%, between 97% and 100%, between 98% and 100% or between 99% and 100%.

In some of any of the provided embodiments, the virial vector is a lentiviral vector. In some of any of the provided embodiments, the viral vector comprises a fusogen embedded in the lipid bilayer. In some of any of the provided embodiments, the fusogen is a viral fusogen selected from a Class I viral membrane fusion protein, a Class II viral membrane protein, a Class II viral membrane fusion protein, a viral membrane glycoprotein, or a viral envelope protein. In some of any of the provided embodiments, the fusogen is endogenous to the virus. In some of any of the provided embodiments, the fusogen is a pseudotyped fusogen. In some of any of the provided embodiments, the fusogen is a viral envelope protein. In some of any of the provided embodiments, the fusogen is a vesicular stomatitis virus envelope glycoprotein (VSV-G). In some of any of the provided embodiments, the fusogen is a baboon endogenous virus (BaEV) envelope glycoprotein. In some of any of the provided embodiments, the fusogen is a Cocal virus envelope glycoprotein. In some of any of the provided embodiments, the fusogen is an Alphavirus fusion protein (e.g. Sindbis virus) or a functional variant thereof. In some of any of the provided embodiments, the fusogen is a Paramyxoviridae fusion protein (e.g., a Morbillivirus or a Henipavirus) or a functional variant thereof. In some of any of the provided embodiments, the fusogen is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof. In some of any of the provided embodiments, the fusogen is a Henipavirus fusion protein (e.g., Nipah virus, Hendra virus, Cedar virus, Kumasi virus, Mójiang virus) or a functional variant thereof. In some of any of the provided embodiments, the fusogen is a Nipah virus fusion protein or a functional variant thereof. In some of any of the provided embodiments, the fusogen comprises a paramyxovirus F protein, or a biologically active portion thereof. In some of any of the provided embodiments, the fusogen comprises a paramyxovirus G, paramyxovirus H, and/or paramyxovirus HN protein, or a biologically active portion thereof. In some of any of the provided embodiments, the paramyxovirus G, paramyxovirus H, and/or paramyxovirus HN protein further comprises a targeting moiety. In some of any of the provided embodiments, the lipid particle or lentiviral vector comprises a nucleic acid encoding a payload gene. In some of any of the provided embodiments, the nucleic acid encoding a payload gene encodes a chimeric antigen receptor (CAR). In some of any of the provided embodiments, the targeting moiety comprises a binding agent. In some of any of the provided embodiments, the binding agent is targeted against CD3, CD4, CD8, CD34, CD90, CD19, CD20, CD22, CD16, or CD56.

In some of any of the provided embodiments, the fusogen comprises an F protein molecule or a biologically active portion thereof from a Paramyxovirus and a glycoprotein G (G protein) or a biologically active portion thereof from a Paramyxovirus. In some of any of the provided embodiments, the Paramyxovirus is a henipavirus. In some of any of the provided embodiments, the Paramyxovirus is Nipah virus. In some of any of the provided embodiments, the fusogen comprises a Nipah virus F glycoprotein (NiV-F) or a biologically active portion thereof and a Nipah virus G glycoprotein (NiV-G) or a biologically active portion thereof. In some of any of the provided embodiments, the Paramyxovirus is Hendra virus. In some of any of the provided embodiments, the G protein or the biologically active portion thereof is a mutant NiV-G protein or biologically active portion thereof that exhibits reduced binding to Ephrin B2 or Ephrin B3. In some of any of the provided embodiments, the mutant NiV-G protein comprises one or more amino acid substitutions corresponding to amino acid substitutions selected from the group consisting of E501A, W504A, Q530A and E533A with reference to numbering set forth in SEQ ID NO:14. In some of any of the provided embodiments, the G protein or biologically active portion is a biologically active portion of wild-type NiV-G that has a deletion of up to 40 amino acids at or near the N-terminus, optionally not including the initial methionine. In some of any of the provided embodiments, the G protein is a biologically active portion that is a truncated NiV-G that has a deletion of amino acids 2-34 at or near the N-terminus of wild-type NiV-G set forth in SEQ ID NO:14. In some of any of the provided embodiments, the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

In some of any of the provided embodiments, the F protein or the biologically active portion thereof is a NiV-F protein or a biologically active portion thereof. In some of any of the provided embodiments, the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine. In some of any of the provided embodiments, the F protein or the biologically active portion is a truncated NiV-F that lacks amino acids 525-546 of SEQ ID NO:2. In some of any of the provided embodiments, the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12. In some of any of the provided embodiments, the Niv-G protein comprises the amino acid sequence set forth in SEQ ID NO: 19, and the Niv-F protein comprises the amino acid sequence set forth in SEQ ID NO:12.

In some of any of the provided embodiments, the fusogen is a re-targeted fusogen that binds to a target cell. In some of any of the provided embodiments, the fusogen comprises a targeting moiety that binds to the target cell. In some of any of the provided embodiments, the target cell is a T cell, a B cell, an NK cell, a macrophage, a monocyte, a dendritic cell, a hematopoietic stem cell or a CD34+progenitor cell. In some of any of the provided embodiments, the target cell is a T cell. In some of any of the provided embodiments, the targeting moiety binds to CD4 or CD8. In some of any of the provided embodiments, the targeting moiety is a Design ankyrin repeat proteins (DARPin), a single domain antibody (sdAb), a single chain variable fragment (scFv), or an antigen-binding fibronectin type III (Fn3) scaffold. In some of any of the provided embodiments, the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker. In some of any of the provided embodiments, the CD8 binding agent is a VHH having the sequence set forth in SEQ ID NO: 128. In some of any of the provided embodiments, the CD8 binding agent is linked to the C-terminus of a truncated NiV-G set forth in SEQ ID NO: 19 for retargeting of the lipid particle or lentiviral vector to CD8+ T cells. In some of any of the provided embodiments, the lipid particle or lentiviral vector comprising the retargeted NiV-G is pseudotyped with the a NiV-F of a biologically active portion thereof, optionally wherein the NiV-F or biologically active portion is set forth in SEQ ID NO:12. In some of any of the provided embodiments, the lipid particle is a viral vector and the composition comprises from 1×10⁸ to 1×10¹¹ infectious units (IU), 1×10⁸ to 1×10¹⁰ IU, 1×10⁸ to 1×10⁹ IU, 1×10⁹ to 1×10¹¹ IU, 1×10⁹ to 1×10¹⁰ IU, 1×10¹⁰ to 1×10¹¹ IU. In some of any of the provided embodiments, the volume of the composition comprising lipid particles is between 100 mL and 400 mL, inclusive.

In some of any of the provided embodiments, the method further comprises administering a cytokine receptor agonist to the subject. In some of any of the provided embodiments, the cytokine receptor agonist is a recombinant protein, a chemically synthesized protein or a conjugate. In some of any of the provided embodiments, the cytokine receptor agonist binds to a cytokine receptor on a T cell. In some of any of the provided embodiments, the cytokine receptor is selected from the group consisting of an IL-2 receptor (IL-2R), an IL-15 receptor (IL-15R), an IL-7 receptor (IL-7R), or an IL-21 receptor (IL-21R). In some of any of the provided embodiments, the cytokine receptor agonist comprises a T cell stimulating cytokine or a T cell stimulating cytokine mutein, a T cell stimulating cytokine mimetic, an antibody or antigen-binding fragment that binds a cytokine receptor on a T cell, or an antibody or antigen-binding fragment that binds a T cell stimulating cytokine. In some of any of the provided embodiments, the cytokine receptor agonist is a conjugate comprising (1) a T cell stimulating cytokine, a T cell stimulating cytokine mutein or a T cell stimulating cytokine mimetic and (2) a water soluble polymer. In some of any of the provided embodiments, the cytokine receptor agonist is a fusion protein comprising (1) a T cell stimulating cytokine, a T cell stimulating cytokine mutein or a T cell stimulating cytokine mimetic and (2) a half-life extending moiety. In some of any of the provided embodiments, the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21), or a T cell stimulating cytokine mutein of any of the foregoing. In some of any of the provided embodiments, the T cell stimulating cytokine mutein comprises at least one amino acid modification relative to a wild-type T cell stimulating cytokine. In some of any of the provided embodiments, the T cell stimulating cytokine mutein is an IL-2 cytokine mutein comprising one or more amino acid modifications relative to wild-type human IL-2. In some of any of the provided embodiments, the IL-2 mutein exhibits increased affinity for the IL-2 Rβ, relative to wild-type human IL-2. In some of any of the provided embodiments, the IL-2 mutein exhibits increased IL-2 activity for the intermediate affinity IL-2 receptor composed of IL-2Rbeta and IL-2Rgamma (IL-2R β/γ), relative to wild-type human IL-2. In some of any of the provided embodiments, the IL-2 mutein exhibits reduced binding to IL-2Ralpha, relative to wild-type human IL-2. In some of any of the provided embodiments, the IL-2 mutein exhibits reduced IL-2 activity for the high-affinity IL-2 receptor composed of IL-2Ralpha, IL-2Rbeta and IL-2Rgamma (IL-2R α/β/γ). In some of any of the provided embodiments, the T cell stimulating cytokine or cytokine mutein is IL-15 or an IL-15 cytokine mutein and the IL-15 or IL-15 cytokine mutein is bound to IL-15Rα or a portion thereof comprising the sushi domain. In some of any of the provided embodiments, the T cell stimulating cytokine mutein is a IL-15 cytokine mutein comprising one more amino acid modifications relative to human IL-15. In some of any of the provided embodiments, the IL-15 mutein exhibits reduced binding to IL-15Rα. In some of any of the provided embodiments, the cytokine receptor agonist comprises a T cell stimulating cytokine mimetic and the mimetic is a IL-2Rα ligand, a IL-2Rβligand, a IL-2Rγ ligand, a common γc receptor (Rγc) ligand, and/or IL-7Rα ligand. In some of any of the provided embodiments, the one, two, three, four, five or six water-soluble polymers are attached to the T cell stimulating cytokine. In some of any of the provided embodiments, the water soluble polymer is a polymer selected from the group consisting of poly(alkylene oxide), poly(vinyl pyrrolidone), poly(vinyl alcohol), polyoxazoline, and poly(acryloylmorpholine). In some of any of the provided embodiments, the water-soluble polymer has a weight-average molecular weight in a range of from about 500 Daltons to about 100,000 Daltons.

In some of any of the provided embodiments, the water-soluble polymer is a poly(alkylene oxide). In some of any of the provided embodiments, the poly(alkylene oxide) is a poly(ethylene glycol). In some of any of the provided embodiments, the cytokine receptor agonist is a human IL-2 or IL-2 mutein covalently attached to one or more poly(ethylene glycol) polymers. In some of any of the provided embodiments, the cytokine receptor agonist is a human IL-15 or IL-15 mutein covalently attached to one or more poly(ethylene glycol) polymers. In some of any of the provided embodiments, the cytokine receptor agonist is a human IL-7 or IL-7 mutein covalently attached to one or more poly(ethylene glycol) polymers. In some of any of the provided embodiments, the cytokine receptor agonist is a human IL-21 or IL-21 mutein covalently attached to one or more poly(ethylene glycol) polymers. In some of any of the provided embodiments, the half-life extending moiety is an Fc region of an immunoglobulin, human serum albumin, an albumin binding moiety, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a sterile composition thereof. In some of any of the provided embodiments, the half-life extending moiety is an albumin binding moiety. In some of any of the provided embodiments, the cytokine receptor agonist is a fusion protein comprising a human IL-2 or IL-2 mutein fused an albumin binding moiety. In some of any of the provided embodiments, the cytokine receptor agonist is a fusion protein comprising a human IL-15 or IL-15 mutein fused to an albumin binding moiety. In some of any of the provided embodiments, the cytokine receptor agonist is a fusion protein comprising a human IL-7 or IL-7 mutein fused to an albumin binding moiety. In some of any of the provided embodiments, the cytokine receptor agonist is a fusion protein comprising a human IL-21 or IL-21 mutein fused to an albumin binding moiety. In some of any of the provided embodiments, the albumin binding moiety is a single domain antibody (sdAb) that specifically binds to albumin. In some of any of the provided embodiments, the half-life extending moiety is an Fc region of an immunoglobulin. In some of any of the provided embodiments, the cytokine receptor agonist is a fusion protein comprising a human IL-2 or IL-2 mutein fused an Fc region of an immunoglobulin. In some of any of the provided embodiments, the cytokine receptor agonist is a fusion protein comprising a human IL-15 or IL-15 mutein fused to an Fc region of an immunoglobulin. In some of any of the provided embodiments, the cytokine receptor agonist is a fusion protein comprising a human IL-7 or IL-7 mutein fused to an Fc region of an immunoglobulin. In some of any of the provided embodiments, the cytokine receptor agonist is a fusion protein comprising a human IL-21 or IL-21 mutein fused to an Fc region of an immunoglobulin. In some of any of the provided embodiments, the Fc of an immunoglobulin is an Fc of human IgG1. In some of any of the provided embodiments, the Fc of an immunoglobulin is an Fc of human IgG4. In some of any of the provided embodiments, the T cell stimulating cytokine or mutein is an IL-7 or IL-7 mutein that is glycosylated. In some of any of the provided embodiments, the T cell stimulating cytokine or mutein is hyperglycosylated, relative to wild-type human IL-7. In some of any of the provided embodiments, the T cell stimulating cytokine or mutein is produced from Chinese Hamster Ovary (CHO) cells. In some of any of the provided embodiments, the T cell stimulating cytokine or mutein is an IL-7 conformer, wherein said conformer comprises the following three disulfide bridges: Cys: 1-4 (Cys2-Cys92); 2-5 (Cys34-Cys129) and 3-6 (Cys47-Cys141).

In some of any of the provided embodiments, the cytokine receptor agonist is an antibody or antigen-binding fragment that binds a T cell stimulating cytokine and the T cell stimulating cytokine is human IL-2. In some of any of the provided embodiments, the antibody or antigen binding fragment inhibits binding of IL-2 with an IL-2 receptor alpha (IL-2 Rα) subunit, inhibits IL-2 signaling through IL-2 Rαβγ and through IL-2 Rβγ and/or inhibits IL-2 signaling through IL-2 Rαβγto a greater extent than through IL-2 Rβγ. In some of any of the provided embodiments, the cytokine receptor agonist is an antibody or antigen-binding fragment that binds a T cell stimulating agent and the T cell stimulating agent is human IL-21. In some of any of the provided embodiments, the antibody or antigen binding fragment enhances human IL-21 activity through the IL-21 receptor. In some of any of the provided embodiments, the cytokine receptor agonist is selected from the group consisting of NL-201, SAR444245 (IL-2 Synthorin™), STK-012, BPT-143, AU-007, IL-15 Synthorin™, PIO-001, bempegaldesleukin (NKTR-214), SHR-1916, ARK102, 8MW-2311, NKTR-255, Exenokine-2, MDNA-11, GX-17/NT-17, SHR-1501, ASKG-215, BCD-225, Exenokine-21, MK-1169, Hu-Mikβ1, JS08-1, CYT-107, AM0015 and KW-007.

In some of any of the provided embodiments, the composition comprises an anticoagulant. In some of any of the provided embodiments, the anticoagulant is a citrate. In some of any of the provided embodiments, the container is a bag.

Provided herein is a lipid particle or viral vector therapy for use in treating a subject having a disease or condition in need of treatment, wherein the lipid particle or viral vector therapy comprises a leukapheresis cell composition contacted with a composition comprising lipid particles or viral vectors, and wherein the lipid particle therapy is delivered to the subject with an apheresis device.

Provided herein is a lipid particle or viral vector therapy for use in treating a subject having a disease or condition in need of treatment, wherein the lipid particle or viral vector therapy comprises a leukapheresis cell composition contacted with a composition comprising lipid particles or viral vectors, and wherein the lipid particle therapy is delivered to the subject with an apheresis device.

Provided herein is a method of treating a disease or condition comprising infusing the lentiviral vector encoding the chimeric antigen receptor (CAR) by the provided methods to a subject having a disease or condition in need of treatment thereof. In some of any of the provided embodiments, the CAR comprises an extracellular antigen binding domain specific for an antigen associated with the disease or condition. In some of any of the provided embodiments, the CAR is an anti-CD19 CAR, an anti-CD22 CAR, and anti-CD20 CAR, or an anti-BCMA CAR. In some of any of the provided embodiments, the CAR is an anti-CD19 CAR.

Provided herein is a method of treating a disease or condition comprising infusing the any of the provided compositions into a subject in need thereof.

In some of any of the provided embodiments, the administration is by in-line infusion of the composition to the subject. In some of any of the provided embodiments, the in-line infusion comprises an apheresis device.

In some of any of the provided embodiments, the disease or disorder is treatable by administration of the lipid particle or viral vector or the payload agent. In some of any of the provided embodiments, the disease or condition is a cancer. In some of any of the provided embodiments, the cancer is a solid tumor, a lymphoma or a leukemia. In some of any of the provided embodiments, the cancer is a B cell Lymphoma. In some of any of the provided embodiments, the B cell lymphoma is a Non-Hodgkin lymphoma, DLBCL, or follicular lymphoma. In some of any of the provided embodiments, the cancer is a relapsed/refractory cancer. In some embodiments, the cancer is a relapsed and/or refractory Large B-cell Lymphoma (LBCL). In some embodiments, the LBCL comprises Non-Hodgkin's lymphoma (NHL). In some embodiments, the NHL comprises a lymphoma selected from the group consisting of diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma.

In some of any of the provided embodiments, the subject has not received a lymphodepleting regimen or therapy. In some of any of the provided embodiments, the subject has not received a lymphodepleting regimen or therapy within 30 days prior to the use or administration.

In some of any of the provided embodiments, the subject has received 2 prior lines of systemic therapy for treating the cancer. In some of any of the provided embodiments, the subject has received 2 prior chemotherapy regimens comprising 1 or more regimens comprising anthracycline and/or one or more regimens comprising anti-CD20 antibody. In some of any of the provided embodiments, the subject has received an autologous stem cell transplant (ASCT).

In some of any of the provided embodiments, prior to performing the therapy or method, the provided method further comprises administering to the subject an agent to mobilize peripheral blood hematopoietic stem cells or CD34+progenitor cells. In some embodiments, the agent to mobilize the cells is G-CSF. In some embodiments, the agent to mobilize the cells includes the combination of G-CSF and Plerixafor.

Provided herein is a lipid particle therapy for use in treating a subject having a disease or condition in need of treatment, wherein the lipid particle therapy particle therapy is administered to the subject by any of the provided methods.

Provided herein is a lipid particle therapy for use in treating a subject having a disease or condition in need of treatment, wherein the lipid particle therapy comprises a leukapheresis cell composition contacted with a composition comprising lipid particles, and wherein the lipid particle therapy is administered to the subject via an apheresis device.

Provided herein is a lentiviral vector therapy for use in treating a subject having a disease or condition in need of treatment, wherein the lentiviral vector therapy is for administration to the subject by any of the provided methods.

In some of any of the provided embodiments, the methods or therapies for use are by in-line infusion comprising an apheresis device.

In some of any of the provided embodiments, the disease or condition is a cancer. In some of any of the provided embodiments, the cancer is a solid tumor, a lymphoma or a leukemia. In some of any of the provided embodiments, the cancer is a B cell Lymphoma. In some of any of the provided embodiments, the B cell lymphoma is a Non-Hodgkin lymphoma, DLBCL, or follicular lymphoma. In some of any of the provided embodiments, the cancer is a relapsed/refractory cancer. In some of any of the provided embodiments, the cancer is a relapsed and/or refractory Large B-cell Lymphoma (LBCL). In some of any of the provided embodiments, the LBCL comprises Non-Hodgkin's lymphoma (NHL). In some of any of the provided embodiments, the subject has received 2 prior lines of systemic therapy for treating the cancer. In some of any of the provided embodiments, the subject has received 2 prior chemotherapy regimens comprising 1 or more regimens comprising anthracycline and/or one or more regimens comprising anti-CD20 antibody. In some of any of the provided embodiments, the subject has received an autologous stem cell transplant (ASCT). In some of any of the provided embodiments, the NHL comprises a lymphoma selected from the group consisting of diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma. In some of any of the provided embodiments, the subject has not received a lymphodepleting regimen or therapy. In some of any of the provided embodiments, the subject has not received a lymphodepleting regimen or therapy within 30 days prior to the use or administration. In some of any of the provided embodiments, prior to performing the therapy or method, further comprising administering to the subject an agent to mobilize peripheral blood hematopoietic stem cells or CD34+progenitor cells. In some embodiments, the agent to mobilize the cells is G-CSF. In some embodiments, the agent to mobilize the cells includes the combination of G-CSF and Plerixafor..

In some embodiments of any of the methods or the uses or products for use (e.g. lipid particle therapy for use, the lentiviral vector therapy for use or the sterile composition for use), the one or more agents that stimulate mobilization are selected from the group consisting of stem cell factor (SCF), small molecule VLA-4 inhibitor BI05192, BOP (N-(benzenesulfonyl)-L-prolyl-L-0-(1-pyrrolidinylcarbonyl)tyrosine), heparin, granulocyte colony-stimulating factor (G-CSF), MGTA-145, and plerixafor (AMD3100).

In some embodiments of any of the methods or the uses or products for use (e.g. lipid particle therapy for use, the lentiviral vector therapy for use or the sterile composition for use), the one or more agents that stimulate mobilization comprise G-CSF. In some embodiments, the G-CSF is administered to the subject daily on the two days, three days, four days, or five days prior to obtaining the whole blood. In some embodiments, the G-CSF is administered to the subject on the day of obtaining the whole blood. In some embodiments, the G-CSF is administered to the subject on the day of reinfusing the contacted PBMCs to the subject.

In some embodiments of any of the methods or the uses or products for use (e.g. lipid particle therapy for use, the lentiviral vector therapy for use or the sterile composition for use), the one or more agents that stimulate mobilization comprise plerixafor. In some embodiments, the plerixafor is administered to the subject on the day of reinfusing the contacted PBMCs to the subject. In some embodiments, the one or more agents that stimulate mobilization are G-CSF and plerixafor. In some embodiments, the G-CSF is administered to the subject daily on the four days prior to obtaining the blood; and the plerixafor is administered to the subject on the day of reinfusing the contacted PBMCs to the subject.

### BRIEF DESCRIPTION OF THE DRAWINGS

FIG. 1 depicts an exemplary flow diagram of one embodiment of the provided method of administering a lipid nanoparticle (e.g. viral vector) to a subject.

FIG. 2 depicts an exemplary flow diagram outlining an alternative embodiment of the method in FIG. 1 in which one or more various optional features can be additionally incorporated into the method.

FIG. 3 depicts tumor growth for several doses of a viral vector encoding a CD19-targeted chimeric antigen receptor (CAR) administered ex-vivo to CD19-tumor bearing mice.

FIG. 4 depicts flow cytometric analysis of the CD19-targeted (CAR+CD8+ T cells (FMC63-derived scFv anti-CD19 binding domain) in the peripheral blood following ex vivo administration to CD19-tumor tumor-bearing mice.

FIG. 5A depicts tumor growth over time for an in vivo delivery condition, FIG. 5B depicts tumor growth following extracorporeal delivery (ECD), and FIG. 5C depicts tumor growth following a combined injection.

FIG. 6 depicts flow cytometric analysis of the CD19-targeted CAR+CD8+ T cells (FMC63-derived scFv anti-CD19 binding domain) in the peripheral blood following in vivo, extracorporeal, and combined administration to CD19-tumor tumor-bearing mice.

Tumor bioluminescence following extracorporeal administration of an exemplary CD8-retargeted viral vector by ECD, or PBMCs only (untreated) or Nalm6 tumor cells only is shown in FIG. 7A. FIG. 7B depicts radiance of individual mice at indicated time points. CAR T cell frequency is shown in FIG. 7C, with tumor size as a function of area under the curve depicted in FIG. 7D.

FIG. 8A shows an exemplary protocol for in vivo administration of PBMCs incubated with CD8-targeted CD19 CAR lentiviral vector (LV). Tumor bioluminescence (BLI) in tumor-bearing animals is shown in FIG. 8B, and quantification via total flux (photon/sec) is depicted in FIG. 8C.

FIG. 9 depicts results from transduced cells analyzed for CAR expression by flow cytometry and the presence of transgene by VCN analysis. Cells pre-treated with exemplary cytokine treatment for 3 days are shown with spinfection in FIG. 9A and without spinfection in FIG. 9B. Similarly, cells pre-treated with exemplary cytokine treatment for 6 days are shown with spinfection in FIG. 9C and without spinfection in FIG. 9D.

Binding of an exemplary lentiviral vector encoding a GFP transgene or a CD19 CAR transgene is shown for CD4+ and CD8+cells in FIG. 10A. Binding is also reflected in FIG. 10B for incubations of 1, 2, or 4 hours.

GFP and CAR expression was analyzed by flow cytometry post transduction in FIG. 11A. Target cell killing by CD8+cells transduced with an exemplary CD8/CD19 CAR vector is shown in FIG. 11B.

Transduction as assessed by CD19 CAR expression in both activated and resting cells is shown in FIG. 12.

The percentage of CAR+CD8+ T cells is depicted in FIG. 13A, while Vector Copy Number (VCN) is depicted in FIG. 13B. CAR expression was analyzed by flow cytometry post transduction in FIG. 13C. Target cell killing by CD8+cells transduced with an exemplary CD8/CD19 CAR vector is shown in FIG. 13D.

The percentage of CAR+cells in the CD8+ T cell population is shown in FIG. 14A and Nalm6 target cell killing initiated at day 8 of culture is shown in FIG. 14B.

### DETAILED DESCRIPTION

Provided herein is a method for ex vivo administration of a lipid particle or viral vector to a subject, the method comprising a) obtaining whole blood from a subject; b) collecting the fraction of blood containing a blood component via apheresis; c) contacting the blood component with a composition comprising lipid particles or viral vectors; and d) reinfusing the contacted blood component to the subject, thereby administering the lipid particle or viral vector to the subject. Also provided herein is a method for administration of a payload gene to subject, the method comprising a) obtaining whole blood from a subject; b) collecting the fraction of blood containing a blood component via apheresis; c) contacting the blood component with a composition comprising a nucleic acid encoding a payload gene; and d) reinfusing the contacted blood component to the subject, thereby administering the payload gene to the subject. In any of the provided embodiments, the ex vivo administration provides for extracorporeal dosing (ECD) of the lipid particle or viral vector.

In some aspects, whole blood obtained from any subject is made up of various cellular and non-cellular components such as red blood cells, white blood cells (i.e., leukocytes) and platelets suspended in its liquid component, plasma. Whole blood can be separated into its components (cellular, liquid or other), and the separated component can be modified such as by being contacted by a lipid particle or viral vector or nucleic acid encoding a payload gene, and then administered to a patient and/or subject in need. The administration of lipid particles or viral vectors and/or payload genes via a blood component in some aspects can be used in treatment of patients and/or subjects suffering from disease. Thus, it is often desirable to separate and collect a desired blood component from whole blood, modify the blood component (i.e., such as with transfection, transduction, or other genetic modifications) and then treat the patient and/or subject with the payload gene or lipid particle or viral vector comprised in that specific blood component. The remaining components may be returned to the donor or retained for other uses.

In some embodiments, the provided methods provide for extracorporeal or ex vivo dosing of a lipid particle or viral vector including for delivery of a payload gene contained therein to a subject. In some embodiments, the viral vector may be a viral vector, such as a viral vector that is pseudotyped for targeting to a desired target cell (e.g. CD8-targeted viral vector for delivery to a T cell). Thus, in some embodiments, the provided methods provide for ex vivo transduction for delivery of a viral vector or payload gene to target cells of interest for therapy. In some embodiments, delivery of the payload gene to target cells may provide a therapeutic intervention or treatment for a disease or condition, such as cancer or a genetic deficiency.

In some embodiments, the methods provide for a strategy for administration of lipid particles or viral vectors, as carriers for therapeutic payloads. The provided methods can in some aspects increase efficiency of on-target transduction and reduce total amount of lipid particle or viral vector needed for treatment. For instance, ex vivo administration as provided allows for increased rate of transfection and/or transduction, and reduces the effective dose of the lipid particle or viral vector or nucleic acid encoding a payload gene required to treat a subject. Ex vivo administration therefore in some aspects also allows for smaller volumes, reducing the total viral particles needed for therapeutic composition manufacturing, transport, and delivery. In some cases, the methods permit delivery of a lipid particle (viral vector) at a defined, small volume, which can increase the certain of transduction events even at lower doses. In some embodiments, methods of ex vivo dosing in accord with the provided methods also can minimize off target toxicity, such as to organs, compared to methods involving systemic (e.g. intravenous) delivery of the lipid particles. The provided method also is short and convenient and can be carried out bedside. For instance, in some embodiments, the provided methods for dosing and administration include a short term exposure of PBMCs such as from a leukapheresis composition with a viral vector composition and then reinfusion back to the subject. In such methods, the PBMCs from the subject can be directly infused back to the subject (after ex vivo contacting with the lipid particle composition, e.g. viral vectors) without unhooking or disconnecting the container from an in-line system containing the container (e.g. bag) of the reinfused cells. Further, the provided methods do not involve ex vivo selection (e.g. immunoaffinity selection) of target cells from the whole blood or from the collected leukapheresis composition; instead, all collected cells separated from the whole blood fraction can be contacted with the lipid particle or viral vector and reinfused to the subject. In some embodiments, the process can be carried out entirely in a closed fluid circuit in which the system includes in-line the components used to obtain the whole blood, separate out PBMCs or subsets (e.g. leukapheresis), contact (e.g. transfect or transduce) the PBMCs or subsets with a composition comprising lipid particles or viral vectors to create a transfection mixture, and reinfuse the transfection mixture to the subject. Accordingly, the provided ex vivo delivery of the lipid particles or viral vectors, such as to deliver a payload gene, is such that the initial contact between the lipid particles (e.g. viral vector, such as containing nucleic acid encoding the payload gene) and cells is ex vivo but then all of the remaining processes are in vivo without all of the artificial conditions of in vitro engineering.

In some aspects, provided herein are in-line methods of administration of lipid particles or viral vectors and/or nucleic acids encoding payload genes. In some embodiments, the provided in-line methods are extracorporeal or ex vivo. In some aspects, in-line methods are closed systems of administration that are associated with lower risk of contamination to the subject, collected whole blood, lipid particles or viral vectors, nucleic acids, separated cells, contacted cells, and cells that are reinfused. In some aspects, in-line methods avoid any additional product labeling and/or traceable handling requirements because the lipid particles or viral vectors, nucleic acids encoding payload genes, and the cells never leave the in-line system. In some aspects, the in-line system remains connected to the subject during the entire procedure.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8;309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3. Table 1, below, lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-HI located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given VHH amino acid sequence, it is understood that such a CDR has a sequence of the corresponding CDR (e.g., CDR-H3) within the VHH, as defined by any of the aforementioned schemes. In some embodiments, specific CDR sequences are specified. Exemplary CDR sequences of provided antibodies are described using various numbering schemes (see e.g. Table 1), although it is understood that a provided antibody can include CDRs as described according to any of the other aforementioned numbering schemes or other numbering schemes known to a skilled artisan.

As used herein, a cytokine receptor agonist is a cytokine that interacts with a cytokine receptor to cause or promote an increase in the activation of the cytokine receptor. Thus, a cytokine receptor agonist acts to activate or stimulate cytokine receptor-mediated signaling. For instance, an IL-7 receptor agonist is a polypeptide capable of activating IL-7 receptor-mediated signaling. Typically a cytokine receptor agonist has comparable or increased biological activity compared to the wild-type cytokine. For instance, an IL-7 agonist has comparable or increased biological activity compared to wild-type IL-7.

As used herein, “cytokine mutein” refers to a cytokine polypeptide wherein specific amino acid modifications to the protein have been made relative to the wild-type cytokine. The cytokine muteins may be characterized by amino acid modifications that include amino acid insertions, deletions, substitutions at one or more sites of the native or wild-type cytokine polypeptide chain. In accordance with this disclosure, any such insertions, deletions, substitutions and modifications result in a cytokine mutein that binds to a cytokine receptor of the wild-type or native cytokine to stimulate the receptor as a cytokine receptor agonist. Exemplary muteins can include substitutions of 1, 2, 3, 4, 5, 6, 7, 8, 9, 10 or more amino acids. Muteins can also include conservative modifications and substitutions at other positions that have a minimal effect on the secondary or tertiary structure of the mutein. Such conservative substitutions include those described by Dayhoff in The Atlas of Protein Sequence and Structure 5 (1978), and by Argos in EMBO J, 8:779-785 (1989). For example, amino acids belonging to one of the following groups represent conservative changes: Group I: Ala, Pro, Gly, Gln, Asn Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. In particular embodiments, a cytokine mutein exhibits at least 85%, at least 90%, at least 95%, or at least 97% sequence identity to the wild-type or native cytokine, such as a wild-type human cytokine. Exemplary wild-type cytokines include IL-2, IL-7, IL-15 or IL-21, such as human wild-type sequences of the foregoing.

By “wild type” or “WT” or “native” herein is meant an amino acid sequence that is found in nature, including allelic variations. A wild type protein or polypeptide has an amino acid sequence that has not been intentionally modified.

As used herein, a “cytokine mimetic” is a protein, peptide or small molecule that is unrelated in topology or amino acid sequence to a wild-type cytokine but mimics or recapitulates activity of a wild-type cytokine to activate or stimulate cytokine receptor-mediated signaling. Hence, a cytokine mimetic may be a cytokine receptor agonist.

As used herein, “lipid particle” refers to any biological or synthetic particle that contains a bilayer of amphipathic lipids enclosing a lumen or cavity. Typically a lipid particle does not contain a nucleus. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. In some embodiments, the fusosome is derived from a source cell. A lipid particle also may include an exogenous agent or a nucleic acid encoding an exogenous agent, which may be present in the lumen of the lipid particle.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome. in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G proteins such as those from Nipah Virus (NiV) and biologically active portions or variants thereof including any as described.

As used herein, a “re-targeted fusogen,” such as a re-targeted G protein, refers to a fusogen that comprises a targeting moiety having a sequence that is not part of the naturally-occurring form of the fusogen in which the targeting moiety targets or binds a molecule on a desired cell type. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In some such embodiments, the attachment of the targeting moiety to a fusogen (e.g. G protein) may be directly or indirectly via a linker, such as a peptide linker. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a targeted lipid particle or viral vector delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a targeted lipid particle or viral vector delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity can include 10%-150% or more of the activity of a full-length or wild-type F protein or G protein. Examples of biologically active portions of F and G proteins include proteins with truncations of the cytoplasmic domain, such as any of the described NiV-F with a truncated cytoplasmic tail.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2. Amino acid substitutions may be introduced into an antibody of interest and the products screened for a desired activity, for example, retained/improved binding.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle or viral vector refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis- regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### Systems and Methods for Ex Vivo Dosing of Lipid Particles

Provided herein are methods for administration of a lipid particle or viral vector or a payload gene to a subject. In some embodiments the method comprises a) obtaining whole blood from the subject; b) collecting the fraction of blood containing PBMC or a subset (e.g. containing leukocyte components); c) contacting the collected PBMC or subset (e.g. leukocyte components) with a composition comprising lipid particles or viral vectors (e.g. that contain a nucleic acid encoding a payload gene for delivery to a cell or subject) to create a transfection mixture; and d) reinfusing the contacted PBMC or subset (e.g. leukocyte components) and/or transfection mixture to the subject, thereby administering the lipid particle or viral vectors and/or payload gene to the subject. In some embodiments, the method is performed ex vivo to the subject. In some embodiments, the method is performed extracorporeal or ex vivo to the subject. In some embodiments, a suitable device or devices to complete the provided method are comprised within a fluid circuit (e.g., in-line). In some embodiments, the in-line system is a closed system.

The method according to the present disclosure is capable of delivering a lipid particle or viral vector and/or payload gene to a system for administration, such as an extracorporeal system. The extracorporeal system for use in the provided method may include a combination of various machine hardware components (i.e., apheresis and blood processing machines), a software control module, and/or a sensor module in-line to ensure monitor the process such as to assess efficiency of transduction, cell health and other aspects related to accuracy and safety of the dosing, and the use of replacement fluids designed to fully exploit the design of the system according to the present methods. It is understood that components described for one system according to the present invention can be implemented within other systems according to the present invention as well. In some embodiments, the method is performed in-line. In some embodiments, the method is performed in a closed fluid circuit, or functionally closed fluid circuit. In some embodiments, various components of the system of administration for use in the provided embodiments are operably connected to the subject, and/or to each other.

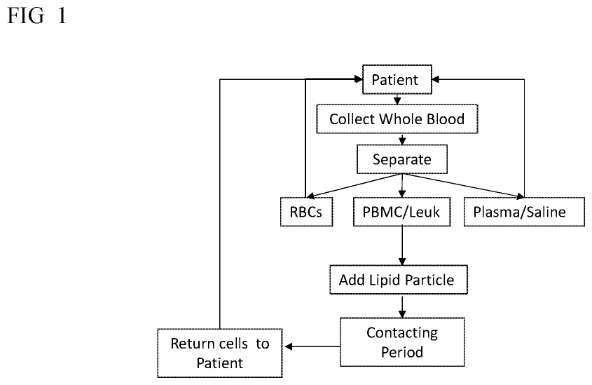

In some embodiments, the method for administration comprises the use of a blood processing set for obtaining the whole blood from the subject, a separation chamber for the separating the blood, a contacting container for the contacting the separated blood component with the composition comprising lipid particles or viral vectors, and a further fluid circuit for re-infusion of the contacted blood component to the patient and/or subject. (see e.g. FIG. 1). In some embodiments, the contacting chamber is for contacting the separated blood component with a composition of lipid particles or viral vectors comprising nucleic acids encoding a payload gene. In some embodiments, the method further comprises the use of any of i) a washing component for concentrating cells of the separated blood component (i.e., leukocytes), and/or a ii) a sensor and/or module for monitoring cell density and/or concentration, In some embodiments, the methods allow processing of blood directly from the patient and/or subject, transfection with the lipid particle or viral vector (e.g. transduction with a viral vector), and reinfusion directly to the patient and/or subject without any steps of selecting for the target cells to be transduced. For instance, if T cells are a desired target cell, the method does not include any step for selecting for T cells or for CD8+ T cells. As another example, if HSCs or CD34+progenitors are a desired target cell, the method does not include any step for selecting for HSCs or CD34+cells. Further the methods also can be carried out without cryopreserving or freezing any cells before or between any one or more of the steps, such that there is no step of formulating cells with a cryoprotectant, e.g. DMSO. In some embodiments, the provided methods also do not include a lymphodepletion regimen. In some embodiments, the method including steps (a)-(d) can be carried out for a time of no more than 24 hours, such as between 2 hours and 12 hours, for example 3 hours to 6 hours.

In some embodiments, the method is performed in-line. In some embodiments, the method is performed in a closed fluid circuit, or a functionally closed fluid circuit. In some embodiments, each of steps (a)-(d) are performed in-line in a closed fluid circuit in which all parts of the system are operably connected, such as via at least one tubing line. In some embodiments, the system is sterile. In some embodiments, the closed fluid circuit is sterile.

In some embodiments, operable connection of the system, for example such as via the blood processing unit, separation chamber, contacting chamber and reinfusion processing unit, is achieved by a connector set containing a least one tubing line and one or more optional connectors. The connector set may include at least one tubing line, such as a plurality of tubing lines, that provide for an operable connection of all containers or components of the system to provide for the closed fluid path. Thus, in some embodiments, the components of the provided system typically include at least one tubing line, and generally a set or system of tubing lines, and at least one connector. Exemplary connectors include valves, ports, spikes, welds, seals, and hose clamps. The connectors and/or other components may be aseptic, for example, to permit the entire process to be carried out in a closed, sterile system, which can eliminate or reduce the need for clean rooms, sterile cabinets, and/or laminar flow systems.

In some embodiments, the at least one tubing line includes a series of tubing lines. Tubing can be made of a plastic, such as polycarbonate, and may be of various sizes and/or volumes, generally designed to permit flow of the desired liquid compositions at the appropriate rate, and connection with the chamber and/or other components. The series of tubing lines generally allows for the flow of liquids between the chamber and/or one or more components of the system, such as the other containers, facilitated in some aspects by connectors. In some embodiments, the system includes tubing lines connecting each of the various components to at least one other of the components, where liquid is permitted to flow between each, and which may be permitted or stopped by the configuration of various connectors, such as valves, and/or clamps.

In some aspects, the connectors are such that they may be placed in or directed to alternative configurations, respectively blocking, allowing, and/or directing the flow of fluids through various components, such as between various containers and through certain tubing lines connecting various components, such as rotational and gate valves. In other embodiments, certain connectors and/or other components have a single configuration which permits, directs, or blocks passage of liquid or gas, such as seals, caps, and/or open ports or channels. Various components in the system may include valves, ports, seals, and clamps. Valves can include rotational valves, such as stopcocks, rotary valves, and gate valves. Valves can be arranged in a manifold array or as a single multiport rotational valve. Ports may include Luer ports or spike ports. Seals may include O-rings, gaskets, adhesive seals, and couplings. Clamps may include pinch clamps.

In some embodiments, the connector set (e.g. containing one or more tubing lines and/or connectors) is sterile. In some embodiments, the connection set is a disposable processing set that provides a sterile closed pathway between the blood processing unit (e.g. apheresis device) and the return processing unit. In some embodiments, the cells from the subject, including the separated leukocyte components, never leave the disposable set (except for closed system monitoring via the one or more monitoring modules) which, in some aspects, remains connected to the donor subject during the entire dosing administration procedure. Thus, the provided embodiments allow for an efficient process for harvesting leukocytes from whole blood, transfecting the leukocytes (or a subset or cell type therein) with a lipid particle or viral vector and reinfusing the transfection mixture (i.e. the leukocyte components contacted with the lipid particles or viral vectors) directly back to the subject, in which the connector set (e.g. disposable connector set) can provide for a sterile and closed fluid pathway between the blood processing unit (e.g. apheresis device) and the return processing unit so that the entire process occurs while the system is connected to the subject or patient.

Other components of a system include containers capable of holding or storing liquids. The containers can include bags, vials, boxes, syringes, bulbs, tanks, bottles, beakers, buckets, flasks, and tubing lines. Such components can hold compositions used in and produced by the methods, including byproducts and interim products and waste. Such compositions may include liquid, including buffers, growth media, transduction media, water, diluents, washes, and/or saline, and may also include the cells, lipid particles or viral vectors, and/or other agents for use in the processing steps, such as transfection (e.g. transduction).

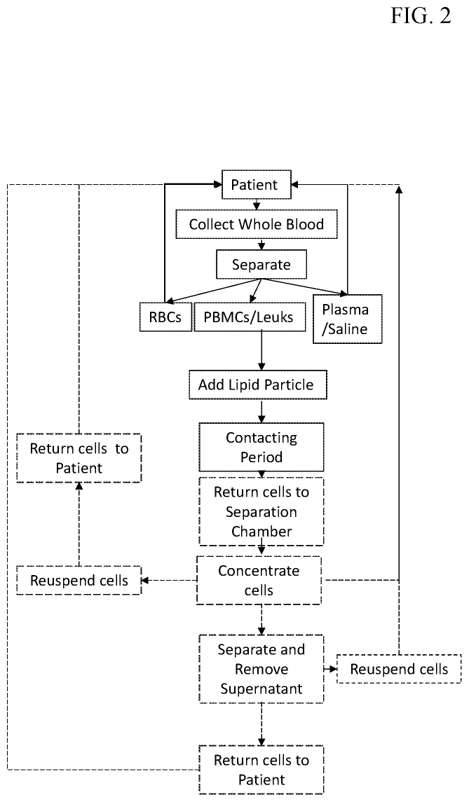

Also provided herein are systems for administration of lipid particle or viral vector comprising a payload gene for targeted delivery to a subject or a cell in the subject. In some embodiments, the methods and systems are for autologous administration to the subject. Exemplary systems for administration are shown in FIG. 1 and FIG. 2.

In some embodiments, the provided methods can be used to process about 3-8 liters (L) of blood by apheresis, such as leukapheresis, to separate leukocyte components or precursors from a whole blood sample. The leukocyte components or precursors thereof include peripheral blood mononuclear cells (PBMCs). In some embodiments, the collected (e.g. separated) leukocyte components or precursors thereof, such as PBMCs, are contacted with a lipid particle or viral vector to create a transfection mixture. In some embodiments, the amount to leukocyte components or precursors thereof, such as PBMCs, during the contacting is at or about 2×10⁶ to 6×10⁹ nucleated cells in which the cells are at a concentration of 5×10⁶ cells/mL to 1×10⁸ cells/mL, such as at or about 1×10⁷ cells/mL and/or are provided in a volume of 100-400 mL. In some embodiments, the lipid particle or viral vector composition is a viral vector composition (e.g. lentiviral vector) containing at or about 1×10⁸ to 1×10¹¹ infectious units (IU). In some embodiments, the transfection mixture is reinfused to the subject.

### A. Obtaining Whole Blood PMBCs

In some of any of the provided embodiments, the method comprises obtaining whole blood from a subject. In some embodiments, a method of collecting blood components is used. In some embodiments, the method includes inserting a venous-access device into a subject, and withdrawing whole blood from the subject. In some embodiments, the method withdraws the blood from the subject through a draw line, which is optionally operably connected to a blood processing set described below. In some embodiments, a draw line pump controls the flow through the draw line. In some embodiments, an anticoagulant is introduced into the withdrawn blood through an anticoagulant line. In some embodiments, the anticoagulant line pump controls the flow through the anticoagulant line.

In some of any of the provided embodiments, the collection of whole blood is performed in a blood processing set. A suitable blood processing set in some embodiments has at least one blood treatment device, such as a hemofilter or dialyzer. In some embodiments, the blood processing set has a blood chamber and a dialysate chamber separated from the blood chamber by a membrane.

In some embodiments, the method comprises obtaining whole blood from a patient and/or subject using a blood processing set that contains a priming solution. In some embodiments, the priming solution comprises citrate, and/or citrate with another suitable buffer. In some embodiments, the citrate is concentrated. In some embodiments, the priming solution is a composition of citrate and another suitable buffer (i.e., a dialysis or replacement solution). In some embodiments, the priming fluid is present in the tubes and/or connectors of the blood processing set at the time of obtaining the whole blood.

In some embodiments, the method comprises filling the blood processing set with whole blood, or a fluid comprising whole blood, from the patient and/or subject. In some embodiments, the blood processing set is filled prior to priming. In some embodiments, the blood processing set is filled following priming. In some embodiments, the filling of the blood processing set may be done within a closed fluid circuit (e.g., in-line). In some embodiments, the blood processing set may be isolated from the fluid circuit before or after collection and filling of the set. In some embodiments, the blood processing set may be connected to a fluid circuit following the filling of said blood processing set.

In some embodiments, the blood processing set comprises a dialysate compartment. In some of any embodiments, the method for collecting whole blood comprises filling the dialysate compartment of the blood processing set bypassing or passing over a membrane. Thus, in some embodiments, a portion of a priming solution described herein (which, as noted above, may include citrate) travels from the blood chamber of the blood processing set (e.g., hemofilter) to the side of the dialysate chamber. In some embodiments, the filling of the dialysate compartment can be through the fresh dialysate side with a solution having the same properties as the priming solution. In some embodiments, the dialysate solution comprises at least the same calcium concentration and/or citrate concentration as the priming solution.

In some embodiments, the blood processing set has at least one blood treatment device. In some embodiments, the blood treatment apparatus is a hemodialysis apparatus, a hemofiltration apparatus or a hemodiafiltration apparatus. In some embodiments, the venous line of the extracorporeal blood circuit is the section from which the blood of the ex vivo treatment patient and/or subject flows to the body of the patient and/or subject or from which it flows back after being treated in a blood treatment device (e.g. a dialyzer).

In some embodiments, the blood processing set has at least one sensor, module, control or regulating unit. In some embodiments, the at least one sensor, module, control or regulating unity is operably connected to one or more components disclosed herein with a fluid and/or signal connection.

In some of any of the provided embodiments, the at least one sensor, module, control or regulating unit is programmed to interact with a blood treatment device, such as a hemofilter or dialyzer as described herein, to perform a blood treatment or to control or regulate the blood processing set after priming according to one of the above-described embodiments. In some embodiments, no heparin or other anticoagulant and/or calcium is added to the ex vivo blood circuit and/or the patient and/or subject.

In some exemplary embodiments according to the invention, in the method of blood treatment after priming, the blood pump is initially set slower than later, and later set faster than earlier.

In some embodiments, a blood pump (e.g., such as a peristaltic pump) is positioned on the blood extraction tube to pump of the whole blood from the subject to a next chamber for use in the method, e.g., a separation chamber as described in Section II.B.2. In some embodiments, the blood extraction pump is positioned midway between the point at which blood is withdrawn from the subject (e.g., the venipuncture site) and the point at which the blood enters the blood processing set and/or separation chamber (e.g., the inlet). In some embodiments, a “distal segment” of the blood extraction tube carries the withdrawn blood from the subject to the blood pump. In some embodiments, a “proximal segment” of the blood extraction tube carries the blood from the blood pump to a next apparatus for use in the method, e.g.., a separation chamber.

In some aspects, it is common in the art to add a flow of anticoagulant solution (e.g. heparin-saline or warfarin-saline) into the “distal segment” of the blood extraction tube at a location close to the vascular access point. Such addition of anticoagulant solution near the vascular access point serves to prevent clotting or coagulation of the blood as it subsequently passes through the apheresis system. This addition of anticoagulant solution is typically accomplished by providing a bag or container of anticoagulant solution connected to the “distal segment” of the blood extraction tube by way of an anticoagulant solution delivery tube. An anticoagulant pump, such as a peristaltic pump, may be positioned on the anticoagulant delivery tube to pump a metered amount of anticoagulant solution through said anticoagulant delivery tube and into the distal end of the “distal segment” of the blood extraction tube to accomplish the desired anticoagulation effect.

The blood processing set may also have a plurality of lines including, but not limited to, a blood draw line, an anticoagulant line, and a return line. In some embodiments, a line specific pump controls the flow through each of these lines. In some embodiments, the blood draw line may be connected (e.g., via a fluid connection that may be closed) to the venous-access device and configured to transport the drawn whole blood to a separation chamber as described below. In some embodiments, a blood draw pump controls the flow through the blood draw line. An anticoagulant line may be connected to an anticoagulant source, and may introduce anticoagulant into the drawn whole blood, i.e., near the venous access device. In some embodiments, an anticoagulant pump controls the flow through the anticoagulant line. The return line may fluidly connect the venous-access device and the separation device, and may be used to return the first or second blood component or compensation fluid to the subject. A return pump may control the flow through the return line. In some embodiments, the return line fluidly connects to the venous-access device at a point between the blood draw pump and the venous-access device.

In some embodiments, the blood processing set is comprised in fluid circuit, optionally a closed in-line circuit. In some embodiments, the blood processing set can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the blood processing set is disposable. In some embodiments, the blood processing set is disposable.

B. Collecting Cells by Separation from the Blood Fraction

In some of any of the provided embodiments, the method further comprises the collection of one or more components from whole blood. In some embodiments, the method further comprises the collection of peripheral blood mononuclear cells (PBMCs) or precursors thereof from whole blood. In some embodiments, the method further comprises the collection of mononuclear cells or precursors thereof from whole blood. In some embodiments, the mononuclear cells are collected via apheresis from whole blood. In some embodiments, the PBMCs are collected via apheresis from whole blood. In some embodiments, the method further comprises the collection of leukocytes or precursors thereof from whole blood. In some embodiments, cells are collected via apheresis from whole blood. In some embodiments, leukocytes or precursors thereof are collected via apheresis from whole blood. In some embodiments, the leukocytes or precursors thereof are collected via leukapheresis. In some embodiments, the mononuclear cells or precursors thereof are collected via mononuclear collection (MNC) or continuous MNC (CMNC). In some embodiments, the leukocytes (white blood cells) include lymphocytes (e.g. T cells, NK cells and B cells), monocytes, macrophages and granulocytes (e.g. neutrophils, eosinophils and basophils). In some embodiments, the collected cells may also include red blood cells, such a hematocrit.

In some embodiments, the method comprises the collection of peripheral blood mononuclear cells (PBMCs). In some embodiments, the method comprises the collection of mononuclear cells. In some embodiments, PBMC's include peripheral blood cells having a round nucleus. In some embodiments, mononuclear cells include blood cells having a single spherical or near-spherical nucleus. In some embodiments, the collected cells are mononuclear cells and/or PBMC's that are lymphocytes (e.g. T cells, NK cells and B cells). In some embodiments, the collected cells are PBMC's that are monocytes. In some embodiments, the PBMC's include leukocyte precursors and/or hemapoietic stem cells. In some embodiments, the leukocyte precursors, such as hemapoietic stem cells, may be collected from the blood (i.e., wherein PBMC are collected). In some embodiments, leukocytes and precursors thereof (e.g. hematopoietic stem cells) are collected and separated from the blood fraction. In some embodiments, leukocyte components that are mature white blood cells are collected and separated from the blood fraction. In some embodiments, leukocyte precursor cells (e.g. hematopoietic stem cells) are collected and separated from the blood fraction.

In some aspects, apheresis is a process wherein whole blood is: (a) withdrawn (e.g.., as described above in Section II.A); (b) separated into two or more fractions (i.e., components); and (c) at least one of the separated blood components is retransfused (reinfused) into the subject. In some aspects, the most common type of apheresis procedure is known as “plasmapheresis”. In plasmapheresis a quantity of liquid plasma is separated from a “cell concentrate” comprising the remaining liquid and cellular constituents of the blood and such cell concentrate is, thereafter, retransfused into the subject. Other types of apheresis procedures include “leukapheresis” (wherein leukocytes are separated from the whole blood) and “thrombocytapheresis” (wherein platelets are separated from the whole blood). In some embodiments, the method comprises a step of leukapheresis. In some aspects, apheresis procedures are performed through the use of automated and/or electronically-controlled apheresis instruments.

Examples of commercially available automated apheresis instruments include the Autopheresis-C® system (Baxter Healthcare Corporation, Fenwal Division, 1425 Lake Cook Road, Deerfield, Ill. 60015), and the (Haemonetics Corporation, City, State). Other commercially available apheresis machines for use in collection of mononuclear cells and/or PBMCs include Spectra Optia® and COBE Spectra®. In some embodiments, the apheresis is a two-step Sepctra Optia® mononuclear cell (MNC apheresis) system. In some embodiments, the apheresis is a Spectra®Optia continuous mononuclear cell (CMNC apheresis) system. In some embodiments, the apheresis device (e.g. Spectra®Optia) includes three major sub-systems, 1) the apheresis machine itself (centrifuge, centrifuge filler, pumps, valves, computerized safety and control systems, etc.), 2) a sterile, single-use, disposable blood tubing set, and 3) embedded software. In some embodiments, such a system can be used to collect mononuclear cells (MNC) from the peripheral blood.

In some embodiments, apheresis uses one or more blood separation apparatus such as a rotation, membrane or centrifugal separator (i.e., a separation chamber as described further below). In some embodiments, the collection of a fraction of blood is via extracorporeal apheresis.

In some of any of the provided embodiments, the collecting of the fraction of blood is via separation into one or more blood components in a separation chamber. In some of any of the provided embodiments, the fraction of blood containing leukocyte components or precursors thereof is collected via a separation chamber. In some embodiments, the separation chamber is configured to separate the PBMCs from whole blood by filtration, such as by membrane filtration. In some embodiments, the separation chamber is configured to separate the PBMCs from whole blood by centrifugation. In some embodiments, the remaining blood components (e.g. plasma, red blood cells and/or platelets) may be returned into the blood stream of the subject.

In some embodiments, the separation chamber includes a centrifuge in which PBMCs are separated by centrifugation. With centrifugation, blood components are separated in order of increasing density as follows: plasma, platelets, lymphocytes and monocytes, granulocytes, and red blood cells. Once blood components are separated, outlet tubes placed within the separation chamber (e.g. apheresis system) allow specific components (e.g. PBMCs) to be selectively removed from the subject based on the density variation into a container. The other components can be returned to the subject and, optionally, are mixed with replacement fluids, such as colloids and crystalloids, during return. In some embodiments, a packing factor (PF) for centrifugation is chosen to achieve the desired separation of cells. The packing factor is characterized by the g-force associated with the centrifugations, the sedimentation velocity at 1 g, the residence time in the separation chamber, and the distance over which sedimentation occurs. The packing factor provides a measure of the radial migration compared to the width of the centrifuge chamber, with adequate cell separation obtained when P >1. In some embodiments, the rotational speed of the centrifuge is from 800 rpm to 2400 rpm, such as 1000 rpm to 2000 rpm, for example at or about 1500 rpm (about 100 g). It is within the level of a skilled artisan to determine the appropriate packing factor for separating cells. For instance, the packing factor can depend on factors such as the particular apheresis device being used, the centrifugal speed, the residence time of cells in the chamber and other factors. In some embodiments, the packing factor is between 2 and 20, such as between 2 and 16, between 2 and 12, between 2 and 8, between 2 and 4, between 4 and 20, between 4 and 16, between 4 and 12, between 4 and 8, between 8 and 20, between 8 and 16, between 8 and 12, between 12 and 20, between 12 and 16 or between 16 and 20. In some embodiments, the packing factor is between 4 and 5, such as at or about 4.5.

In some embodiments, the separation chamber separates the drawn blood into at least a first blood component, and a second blood component. In some embodiments, the separation chamber separates the drawn blood into at least a first blood component containing leukocytes or precursors thereof, and a second blood component (e.g. red blood cells and/or plasma). In some embodiments, the separation chamber may be configured such that the blood components are sent to a first and second blood bag, respectively. In some embodiments, the blood component separation device also has an outlet and may optionally alternate between discharging the first blood component (i.e., leukocytes or precursors thereof) and the second blood component (i.e. red blood cells and/or plasma) through the outlet.

In some embodiments, the separation chamber is a centrifuge, optionally a centrifuge bowl. In some embodiments, the centrifuge may separate the drawn blood into a third blood component in addition to the first blood component and the second blood component blood component. In some embodiments, the second and/or third blood component may be returned to the subject in addition to the first blood component via the return line. In some embodiments, The first blood component can be leukocytes or precursors thereof and/or the second blood component can be red blood cells, and/or the third blood component can be plasma and/or platelets. In some embodiments, the separation chamber separates the whole blood into a first blood component (e.g., containing leukocytes or precursors thereof) and a second blood component, optionally wherein the whole blood is separated into a first, second, and third blood component. In some embodiments, the separation chamber extracts the first blood component from the separation chamber. In some embodiments, the separation chamber extracts leukocytes or precursors thereof from the separation chamber. In some embodiments, the second blood (e.g. red blood cells) and/or third blood component (e.g. plasma and/or platelets) is returned to the subject through the return line. In some embodiments, the return line operably connects to the venous-access device at a point between the draw line pump and the venous-access device.

In some embodiments, the separation chamber is an apheresis device. In some embodiments, the separation chamber is an apheresis device which separates cells based on their respective density. For example, a device which uses differential centrifugation to separate the most dense red blood cells, from the less dense cell components of (i) plasma and (ii) the “buffy coat”. In some embodiments, the collecting cells by separation of the blood is collecting cells of the “buffy coat”. In some embodiments, the “buffy coat” layer comprises lymphocytes (e.g., T, B, and NK cells) as well as monocytes and granulocytes. In some embodiments, the “buffy coat” layer comprises and/or further comprises PBMCs. In some embodiments, the “buffy coat” layer comprises HSCs.

In some embodiments, the separation chamber is an apheresis device. In some embodiments, the separation chamber is an apheresis device that separates cells based on their respective density with the use of a density gradient reagent. For example, a device which uses differential centrifugation to separate the most dense red blood cells and gradient reagent, from the less dense cell components of (i) plasma, and (ii) PBMCs. In some embodiments, the collecting cells by separation of the blood is collecting the PBMC. In some embodiments, the cells of the PBMC layer comprises lymphocytes (e.g., T, B, and NK cells), optionally wherein the cells of the PBMCs layer further comprise monocytes. In some embodiments, the cells of the PBMC comprises HSCs. Any density reagent known in the art is suitable for use in the method, for example sucrose, Percoll, and/or Ficoll can be used to perform density based differential centrifugation in a separation chamber (i.e., apheresis device).

In some embodiments, the separation chamber is a leukapheresis device. In some embodiments, the separation chamber is an leukapheresis device that separates cells based on their respective density with the use of a density gradient reagent. For example, a device which uses differential centrifugation to separate the most dense red blood cells and gradient reagent, from the less dense cell components of (i) plasma, and (ii) leukocytes and/or precursors thereof. In some embodiments, the collecting cells by separation of the blood is collecting the leukocytes. In some embodiments, the cells of the leukocyte layer comprises lymphocytes (e.g., T, B, and NK cells), optionally wherein the cells of the leukocyte layer further comprise monocytes.

In some embodiments, the collected cells contain 20-60% T cells, 5-40% monocytes, 2.5-30% B cells, 2.5-30% NK cells, 0.5-10% granulocytes and 0.5-10% hematocrit. For instance, in some embodiments, the collected cells contain up to 50% T cells, 10-30% monocytes, 5-20% B cells, 5-20% NK cells, 2-5% granulocytes and 2-5% hematocrit. In some embodiments, the collected cells contain on average up to 50% T cells, 20% monocytes, 10% B cells, and 10% NK cells, 3% granulocytes, and 3% hematocrit.

In any of the provided embodiments, the separated cells are collected into a container (also called a “collection container”). The container may be a different forms, including a flexible bag, similar to an IV bag, or a rigid container similar to a cell culture vessel. In particular embodiments, the container is a collection bag. Generally, the composition of the container will be any suitable, biologically inert material, such as glass or plastic, including polypropylene, polyethylene, etc. In particular embodiments, the container is sterile, such as a sterile bag. In some embodiments, the container includes one or more ports such that the cells or reagents can be introduced into or transferred out of the container. For instance, the container may include one or more ports so that reagents for transfection of cells (e.g. composition containing viral particles) can be introduced to cells within the container. In some cases more than one port may be present for the introduction of one or more reagents, media, etc. and/or for transferring out the cells.

In some of any of the provided embodiments, the separation of cells is via apheresis, such as by leukapheresis. In some of any of the provided embodiments, the apheresis (e.g., leukapheresis) is for a set number of minutes. In some of any of the provided embodiments, the apheresis (e.g., leukapheresis) is for at most 100, at most 120, at most 140, at most 160, at most 180, at most 200, at most 220, at most 240, at most 260, at most 280, at most 300, at most 320, at most 340, at most 360, at most 380, or at most 400 minutes. In some of any of the provided embodiments, the apheresis (e.g., leukapheresis) is for 100-120, 120-140, 140-160, 160-180, 180-200, 200-220, 220-240, 240-260, 260-280, 280-300, 300-320, 320-340, 340-360, 360-380, or 380-400 minutes, each range inclusive. In some of any of the provided embodiments, the apheresis (e.g., leukapheresis) is for at most 200, 220, 240, 260, 280, or 300 minutes. In some of any of the provided embodiments, the apheresis (e.g., leukapheresis) is for 100, 120, 140, 160, 180, 200, 220, 240, 260, 280, 300, 320, 340, 360, 380, or 400 minutes.

In some embodiments, the collection device, such as the apheresis device (e.g. leukapheresis device) processes blood from a subject for separating the desired blood components (e.g. PBMCs). In some of any of the provided embodiments, the processed blood volume (i.e., the volume of blood obtained from whole blood as described in Section II.A) is at least 5, 6, 7, 8, 9, 10, 11, 12, 13, 14, 15, 16, 17, 18, 19, or 20 liters. In some of any of the provided embodiments, the processed blood volume is 5-20, 5-18, 5-16, 5-14, 5-12, 5-10, 10-20, 10-18, 10-16, 10-14, 10-12, 12-20, 12-18, 12-16, 2-14, 14-20, 14-18, 14-16, 16-20, 16-18 or 18-20 liters, each range inclusive. In some of any of the provided embodiments, the processed blood volume is 5, 6, 7, 8, 9, 10, 11, 12, 13, 14, 15, 16, 17, 18, 19, or 20 liters, or any value between any of the foregoing. In some of any of the provided embodiments, the processed blood volume is 5, 6, 7, 8, 9, 10, 11, 12, or 13 liters. In some of any of the provided embodiments, the processed blood volume is at most 10, 11, 12, 13, 14, or 15 liters.

In some of any of the provided embodiments, the processed blood volume is at least the total blood volume of the patient and/or subject. For example and in some embodiments, any of the below formulas may be used for calculating the total blood volume of a patient and/or subject.

### Formulas for Calculating Total Blood Volume (TBV) of Men and Women:

Female:183+(356×height³(meters)]+[33.1×weight(kg)]

Male:604+(367×height³(meters)]+[32.2×weight(kg)]

In some of any of the provided embodiments, the processed blood volume is at least 1, at least 2, at least 3, or at least 4 times the total blood volume of the patient and/or subject. In some of any of the provided embodiments, the processed blood volume is between 1 and 2 times the total blood volume, range inclusive. In some of any of the provided embodiments, the processed blood volume is between 2 and 3 times the total blood volume, range inclusive. In some of any of the provided embodiments, the processed blood volume is between 3 and 4 times the total blood volume, range inclusive. In some of any of the provided embodiments, the processed blood volume is or is about 1.5, 1.6, 1.7, 1.8, 1.9, 2.0, 2.1, 2.2, 2.3, 2.4, or 2.5 times the total blood volume.

In some embodiments, the separation chamber or device containing the same (e.g. apheresis device) is comprised in a fluid circuit, optionally a closed in-line circuit. In some embodiments, the separation chamber can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the separation chamber set is disposable. In some embodiments, the separation chamber is disposable.

In some of any of the provided embodiments, the cells of the whole blood are separated. In some of any of the provided embodiments, PBMCs or subsets thereof are separated from the whole blood. In some embodiments, the separated cells is or comprise PBMCs. In some embodiments, the separated cells include or are enriched leukocytes. In some of any of the provided embodiments, the leukocyte components or precursors thereof are separated from the whole blood. In some embodiments, the separated cells is or comprise leukocytes. In some embodiments, the separated cells are not leukocytes. In some embodiments, the separated cells are leukocyte precursors, such as hematopoietic stem cells. In some embodiments, the separated cells are stem cells. In some embodiments, the separated cells are hematopoietic stem cells (HSCs).

In some embodiments, the separated cells are or include T cells, such as CD4+ or CD8+T cells. In some embodiments, the separated cells are or include Natural Killer cells (NK cells). In some embodiments, the separated cells are or include B cells. In some embodiments, the separated cells are or include macrophages. In some embodiments, the separated cells are myeloid derived suppressor cells. In some embodiments, the separated cells are a leukocyte belonging to the group selected from monocytes, lymphocytes, neutrophils, eosinophils, basophils, and macrophages.

In some of any of the provided embodiments, the method does not comprise selection of cells. In some embodiments, the method comprises collecting a cell component from the whole blood without selecting for cell surface expression of any protein. In some embodiments, the method does not comprise selecting T cells positive for a T cell marker (e.g. CD3, CD4 or CD8)). In some embodiments, the method does not comprise selecting cells position for the CD34. In some embodiment, the provided methods do not include a step of immunoaffinity-based selection.

In some embodiments, the separated cells are nucleated. In some embodiments, the separated cells are or comprise peripheral blood mononuclear cells (PBMCs). In some embodiments, the number of nucleated cells (e.g. PBMCs) is 5-10×10⁸, 10-20×108, 20-30×10⁸, 30-40×10⁸, 40-50×10⁸, 50-60×10⁸, 60-70×10⁸, 70-80×10⁸, 80-90×10⁸, 100-150×10⁸, 150-200×10⁸, 200-300×10⁸, or 300-400×10⁸ cells, each range inclusive. In some embodiments, the number of nucleated cells (e.g. PBMCs) is at least 5×10⁸, 10×10⁸, 20×10⁸, 30×10⁸, 40×10⁸, 50×10⁸, 60×10⁸, 70×10⁸, 80×10⁸, 90×10⁸, 100×108, 150×10⁸, 200×10⁸, or 300×10⁸ cells. In some embodiments, the number of nucleated cells (e.g. PBMCs) is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of nucleated cells (e.g. PBMCs) is 1-5%, 5-10%, 10-20%, 20-30%, 30-40%, 40-50%, or 50-60% of the total number of separated cells, each range inclusive. In some embodiments, the number of nucleated cells (e.g. PBMCs) is at least 5%, 10%, 20%, 30%, 40%, 50%, 60%, 70%, 80%, or 90% of the total cell number of separated cells. In some embodiments, the number of nucleated cells (e.g. PBMCs) is 5%, 10%, 20%, 30%, 40%, 50%, 60%, 70%, 80%, 90%, or 100% of the total cell number of separated cells.

In some embodiments, the separated cells comprise CD3+cells. In some embodiments, the total number of CD3+cells is 5-10×10⁸, 10-20×108, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive.. In some embodiments, the number of CD3+cells is at least 5×10⁸, 10×10⁸, 20×10⁸, 30×10⁸, 40×10⁸, 50×10⁸, 60×10⁸, 70×10⁸, 80×10⁸, 90×10⁸, 100×108, 150×10⁸, 200×10⁸, or 300×10⁸ cells. In some embodiments, the number of CD3+cells is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of CD3+cells is 1-5%, 5-10%, 10-20%, 20-30%, 30-40%, 40-50%, or 50-60% of the total number of separated cells, each range inclusive. In some embodiments, the number of CD3+cells is at least 5%, 10%, 20%, 30%, 40%, 50%, 60%, 70%, 80%, or 90% of the total cell number of separated cells. In some embodiments, the number of CD3+cells is 5%, 10%, 20%, 30%, 40%, 50%, 60%, 70%, 80%, 90%, or 100% of the total cell number of separated cells.

In some embodiments, the separated cells comprise monocytes. In some embodiments, the total number of monocytes is 5-10×10⁸, 10-20×108, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the number of monocytes is at least 5×10⁸, 10×10⁸, 20×10⁸, 30×10⁸, 40×10⁸, 50×10⁸, 60×10⁸, 70×10⁸, 80×10⁸, 90×10⁸, 100×108, 150×10⁸, 200×10⁸, or 300×10⁸ cells. In some embodiments, the number of monocytes is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of monocytes is 1-5%, 5-10%, 10-20%, 20-30%, 30-40%, 40-50%, or 50-60% of the total number of separated cells, each range inclusive. In some embodiments, the number of monocytes is at least 5%, 10%, 20%, 30%, 40%, 50%, 60%, 70%, 80%, or 90% of the total cell number of separated cells. In some embodiments, the number of monocytes is 5%, 10%, 20%, 30%, 40%, 50%, 60%, 70%, 80%, 90%, or 100% of the total cell number of separated cells.

In some embodiments, the separated cells include certain PBMC subsets, such as hematopoietic stem cells. In some embodiments, the separated cells include or are enriched in stem cells. In some embodiments, the separated cells include or are enriched hematopoietic stem cells (HSCs).

In some embodiments, the separated cells comprise stem cells, optionally wherein the separated cells comprise HSCs. In some embodiments, the total number of stem cells is 5-10×10⁸, 10-20×108, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the number of stem cells is at least 5×10⁸, 10×10⁸, 20×10⁸, 30×10⁸, 40×10⁸, 50×10⁸, 60×10⁸, 70×10⁸, 80×10⁸, 90×10⁸, 100×108, 150×10⁸, 200×10⁸, or 300×10⁸ cells. In some embodiments, the number of stem cells is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of stem cells is 1-5%, 5-10%, 10-20%, 20-30%, 30-40%, 40-50%, or 50-60% of the total number of separated cells, each range inclusive. In some embodiments, the number of stem cells is at least 5%, 10%, 20%, 30%, 40%, 50%, 60%, 70%, 80%, or 90% of the total cell number of separated cells. In some embodiments, the number of stem cells is 5%, 10%, 20%, 30%, 40%, 50%, 60%, 70%, 80%, 90%, or 100% of the total cell number of separated cells.

In some embodiments, the separated cells comprise platelets. In some embodiments, the total number of platelets is 50-100×10⁸, 100-200×108, 200-300×10⁸, 300-400×10⁸, 400-500×10⁸, 500-600×10⁸, 600-700×10⁸, 700-800×10⁸, 800-900×10⁸, 1000-1250×10⁸, 1250-1500×10⁸, 1500-1750×10⁸, 1750-2000×10⁸ cells, or 2000-3000×10⁸ each range inclusive. In some embodiments, the number of platelets is at least 50×10⁸, 100×10⁸, 200×10⁸, 300×10⁸, 400×10⁸, 500×10⁸, 600×10⁸, 700×10⁸, 800×10⁸, 900×10⁸, 1000×108, 1500×10⁸, 2000×10⁸, or 3000×10⁸ cells. In some embodiments, the number of platelets is 50×10⁸, 100×10⁸, 200×10⁸, 300×10⁸, 400×10⁸, 500×10⁸, 600×10⁸, 700×10⁸, 800×10⁸, 900×10⁸, 1000×108, 1500×10⁸, 2000×10⁸, or 3000×10⁸ cells.

In some embodiments, the separated cells have a hematocrit reading of 1-5%, , range inclusive. In some embodiments, the hematocrit reading is at least 1%, 2%, 3%, 4%, or 5%. In some embodiments, the hematocrit reading is 1%, 2%, 3%, 4%, or 5%. In some embodiments, the hematocrit reading is at most 1%, 2%, 3%, 4%, or 5%.

In some embodiments, the separated cells are viable. In some embodiments, the percentage of viable cells within the separated cell component is 1-5%, 5-10%, 10-20%, 20-30%, 30-40%, 40-50%, 50-60%, 60-70%, 70-80%, 80-90%, 90-95%, or 95-100% of the total cell number each range inclusive. . In some embodiments, the number of viable cells is at least 5%, 10%, 20%, 30%, 40%, 50%, 60%, 70%, 80%, or 90% of the total cell number of separated cells. In some embodiments, the number of viable cells is 5%, 10%, 20%, 30%, 40%, 50%, 60%, 70%, 80%, 90%, or 100% of the total cell number of separated cells.

In some embodiments, the separated cells are comprised within the separation chamber in a volume (i.e., within the lumen of a separation chamber). In some embodiments, the separated cells are transferred to a collection container In some embodiments, the volume of separated cells is between 120-140 mL, 140-160 mL, 160-180 mL, 180-200 mL, 200-220 mL, 220-240 mL, 240-260 mL, 260-280 mL, or 280-300 mL, each range inclusive. In some embodiments, the volume of separated cells is at least 120 mL, 140 mL, 160 mL, 180 mL, 200 mL, 220 mL, 240 mL, 260 mL, 280 mL or 300 mL. In some embodiments, the volume of separated cells is 120 mL, 140 mL, 160 mL, 180 mL, 200 mL, 220 mL, 240 mL, 260 mL, 280 mL or 300 mL. In some embodiments, the volume of the separated cells is no more than 1000, 2000, 3000, 4000, or 5000 mL. In some embodiments, the volume of the separated cells is no more than 1000 mL.

In some embodiments, the concentration of separated cells is between 1×10⁷- 2×10⁷, 2×10⁷-3×10⁷ cells/mL, 3×10⁷- 4×10⁷, 4×10⁷- 5×10⁷, 5×10⁷- 6×10⁷, 6×10⁷- 7×10⁷, 7×10⁷- 8×10⁷, 8×10⁷- 9×10⁷,or 9×10⁷- 10×107 cells/mL, each range inclusive. In some embodiments, the concentration of separated cells is at least 1×10⁷, 2×10⁷, 3×10⁷, 4×10⁷, 5×10⁷, 6×10⁷, 8×10⁷, 9×10⁷ or 10×10⁷ cells/mL. In some embodiments, the concentration of separated cells is 1×10⁷, 2×10⁷, 3×10⁷, 4×10⁷, 5×10⁷, 6×10⁷, 8×10⁷, 9×10⁷ or 10×107 cells/mL. In some embodiments, the concentration of separated cells is between 1×107 and 2×10⁷.

In some embodiments, an apheresis device (e.g. Spectra Optia or COBE Spectra) processes 10-12L of blood and separates PBMCs containing leukocytes (while blood cells) by collection to a collection bag. In some embodiments, the volume of separated cells in the container is between 100 mL and 400 mL, inclusive, such as between 200 mL and 250 mL, inclusive, e.g. at or about 240 mL. In some embodiments, the number of collected nucleated cells is about 1×10⁸ to 30×10⁹. In some embodiments, the collected cells contain at or about 4×10⁸ to 20×10⁹ CD3+ T cells. In some embodiments, the T cells include CD8+ T cells. The exact number of nucleated cells, CD3+ T cells or CD8+ T cells will vary depending on the subject, which can be impacted or different depending on the particular disease or condition of the subject. For instance, an apheresis yield is generally lower in subjects with ALL/CLL compared to lymphoma. In some embodiments, the remaining blood components (e.g. plasma, red blood cells and/or platelets) may be returned into the blood stream of the subject.

In some embodiments, the method does not comprise cryopreservation of the separated cells. Therefore in some embodiments, the separated cells are not subject to cryopreservation. In some embodiments, the separated cells are not subject to cryopreservation further in the method. In some embodiments, the separated cells are not treated with any cryopreservation media, optionally wherein the separated cells are not treated with DMSO.

In some embodiments, the separated cells are not expanded. In some embodiments, the separated cells are not cultured for growth or expansion. In some embodiments, the separated cells are not treated with compositions for expansion, such as adjuvants of cell growth or activation.

In some embodiments, the container (e.g. bag) may contain an anti-coagulant to prevent clotting while the cells and sample are processed ex vivo such as in an extracorporeal in-line device. In some embodiments, the anti-coagulant is citrate or heparin.

In some embodiments, the collection container containing separated cells is a Leukopak. A Leukopak is a sterile bag containing a highly-enriched leukapheresis-derived product. In some embodiments, Leukopaks contain high concentrations of mononuclear cells, B cells, T cells, stem/progenitor cells, dendritic cells, and other cell types.

In some embodiments, the container containing the separated cells (e.g. sterile bag such as a blood bag) may be transferred to a contacting chamber for contacting the cells with a viral vector as described below.

In other embodiments, the container containing the separated cells (e.g. sterile bag such as a blood bag) is used as the contacting chamber and the composition containing viral vector particles is introduced directly into the container (e.g. sterile bag such as a blood bag) containing the separated cells.

In some of any of the provided embodiments, contacting the separated cells with a lipid particle or viral vector or nucleic acid encoding a payload gene proceeds at the completion of collecting cells by separation as described in II.B, such as at the completion of leukaphoresis. In some embodiments, the container containing separated cells (e.g., a Leukopak) is connected via a fluid in-line circuit to the pheresis return line, such as an apheresis return line.

In some embodiments, the method comprises administering to a subject lipid particles (e.g., viral vectors) such that the subject is connected via catheter to the fluid in-line circuit during the completion of two or more, three or more streps of (a)-(d) of the method. In some embodiments, two or more steps of a) obtaining whole blood from a subject; b) collecting the fraction of blood containing peripheral blood mononuclear cells (PBMCs) or leukocyte components; c) contacting the PBMCs or subset thereof with a composition comprising lipid particles comprising a nucleic acid encoding a payload gene to create a transfection mixture; and d) reinfusing the contacted PBMCs or subset thereof and/or the transfection mixture to the subject, thereby administering the lipid particle to the subject are carried out in-line in a closed fluid circuit. In some embodiments, three or more steps of a) obtaining whole blood from a subject; b) collecting the fraction of blood containing peripheral blood mononuclear cells (PBMCs) or leukocyte components; c) contacting the PBMCs or subset thereof with a composition comprising lipid particles comprising a nucleic acid encoding a payload gene to create a transfection mixture; and d) reinfusing the contacted PBMCs or subset thereof and/or the transfection mixture to the subject, thereby administering the lipid particle to the subject are carried out in-line in a closed fluid circuit.

In some embodiment, the subject is connected via catheter to the fluid in-line circuit during the steps of obtaining whole blood PBMCs, collecting the cells by separation, and/or contacting the separated cells with a lipid particle. In some embodiment, the subject is connected via catheter to the fluid in-line circuit during the steps of obtaining whole blood PBMCs, collecting the cells by separation, and contacting the separated cells with a lipid particle. In some embodiments, the subject is disconnected from the fluid in-line circuit following the collecting of cells by separation and reconnected to the same fluid in-line circuit prior to contacting separated cells as described below (e.g., such as by disconnection and clamping of a catheter, and then removal of the clamp from the lead for reconnection to the circuit). In some embodiments, the catheter comprises two or three lumens. In some embodiments, the catheter is a dialysis catheter with two lumens (e.g., venous and arterial). In some embodiments, the catheter is a trialysis catheter with three lumens (e.g., venous, arterial, and therapeutic). In some embodiments, each lumen of the catheter (e.g., dialysis or trialysis catheter) comprises a port. In some embodiments, the container containing collected cells by separation, such as a Leukopak, is connected via a fluid in-line circuit to an alternative port of a catheter, such as the 3^{rd} port of a trialysis catheter. In some embodiments, the subject is disconnected from the fluid in-line circuit following the contacting separated cells as described below and reconnected to the same fluid in-line circuit prior to reinfusion as described in Section II.D (e.g., such as by disconnection and clamping of a catheter, and then removal of the clamp from the lead for reconnection to the circuit).

In some embodiments, the method comprises administering to a subject viral vectors such that the subject is connected via catheter to the fluid in-line circuit during the completion of two or more, three or more streps of (a)-(d) of the method. In some embodiment, the subject is connected via catheter to the fluid in-line circuit during the steps of obtaining whole blood PBMCs, collecting the cells by separation, and/or contacting the separated cells with a viral vector. In some embodiment, the subject is connected via catheter to the fluid in-line circuit during the steps of obtaining whole blood PBMCs, collecting the cells by separation, and contacting the separated cells with a viral vector. In some embodiments, the subject is disconnected from the fluid in-line circuit following the collecting of cells by separation and reconnected to the same fluid in-line circuit prior to contacting separated cells as described below (e.g., such as by disconnection and clamping of a catheter, and then removal of the clamp from the lead for reconnection to the circuit). In some embodiments, the catheter two or three lumens. In some embodiments, the catheter is a dialysis catheter with two lumens (e.g., venous and arterial). In some embodiments, the catheter is a trialysis catheter with three lumens (e.g., venous, arterial, and therapeutic). In some embodiments, each lumen of the catheter (e.g., dialysis or trialysis catheter) comprises a port. In some embodiments, the container containing collected cells by separation, such as a Leukopak, is connected via a fluid in-line circuit to an alternative port of a catheter, such as the 3^{rd} port of a trialysis catheter. In some embodiments, the subject is disconnected from the fluid in-line circuit following the contacting separated cells as described below and reconnected to the same fluid in-line circuit prior to reinfusion as described in Section II.D (e.g., such as by disconnection and clamping of a catheter, and then removal of the clamp from the lead for reconnection to the circuit).

In some embodiments, the subject is disconnected from the fluid in-line circuit for no more than 30 mins, 1 hour, 2 hours, 3, hours, or 4 hours before reconnecting to the same fluid in-line circuit.

C. Contacting Separated Cells with a Lipid Particle or Viral Vector or Nucleic Acid Encoding a Payload Gene

In some of any of the provided embodiments, the method comprises contacting the separated cells (e.g. leukocyte components or precursors thereof) with a lipid particle or viral vector, such as a lipid particle comprised within a composition of lipid particles or a viral vector comprised within a composition. In particular embodiments, the lipid particle or a viral vector, such as a lentiviral vector. In some of any of the provided embodiments, the method comprises contacting the separated cells (e.g. leukocyte components or precursors thereof) with a nucleic acid encoding a payload gene, such by contacting the separated cells with a composition of nucleic acids (e.g. plasmids). In some embodiments, the contacting of the leukocyte components or precursors thereof with the lipid particle or viral vector or nucleic acid(s) creates a transfection mixture.

In some of any of the provided embodiments, contacting the separated cells with a lipid particle or viral vector or nucleic acid encoding a payload gene proceeds at the completion of collecting cells by separation as described in II.B, such as at the completion of leukaphoresis.

In some of any of the provided embodiments, the contacting of the separated cells (e.g. leukocyte) is within a contacting chamber. In some embodiments, the contacting chamber is in-line with a blood processing set and/or separation chamber as described above. In some embodiments, the contacting chamber is operably connected to any of the blood processing set and/or separation chamber. In some embodiments, the contacting occurs in the collection container (e.g. bag) into which the separated cells have been collected as described above. Hence, in some cases the contacting chamber and the collection container are the same unit. In some embodiments, the separation chamber and contacting chamber are connected by a fluid circuit, optionally a closed fluid circuit. In some embodiments, the separation chamber and contacting chamber are connected via a fluid circuit that is a closed pathway between the separation and contacting chamber, optionally wherein the circuit is sterile.

In some embodiments, contacting the separated cells with a lipid particle or viral vector or a composition comprising lipid particles or viral vectors results in the transfection of at least a portion of the separated cells. In some embodiments, the number of transfected cells is 1-5%, 5-10%, 10-20%, 20-30%, 30-40%, 40-50%, or 50-60% of the total number of contacted cells, each range inclusive. In some embodiments, the number of transfected cells is at least 5%, 10%, 20%, 30%, 40%, 50%, 60%, 70%, 80%, or 90% of the total cell number of contacted cells. In some embodiments, the number of transfected cells is 5%, 10%, 20%, 30%, 40%, 50%, 60%, 70%, 80%, 90%, or 100% of the total cell number of contacted cells. In some embodiments, the total number of transfected cells is 5-10×10⁸, 10-20×10⁸, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸, each range inclusive. In some embodiments, the number of transfected cells is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of transfected cell is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of transfected cells is or is about 1×10⁸, 5×10⁸, 10×10⁸, 20×10⁸, 30×10⁸, 40×10⁸, or 50×10⁸ cells.

In some embodiments, the contacting of separated cells is initiated within 0.5-1 hours, 1-2 hours, 2-4 hours, 4-6 hours, 6-8 hours, 8-10 hours, 10-12 hours, 12-14 hours, 14-16 hours, 16-18 hours, 18-20 hours, 20-22 hours, 22-24 hours after collection of the blood fraction comprising the separated cells (e.g., after apheresis for a first blood component as described in Section II. B.). In some embodiments, the contacting of separated cells is initiated no more than 12 hours after collection of the blood fraction comprising the separated cells. In some embodiments, the contacting of separated cells is initiated at most 12 hours after collection of the blood fraction comprising the separated cells. In some embodiments, the contacting of separated cells is initiated within at least 30 minutes, 1 hour, 2 hours, 2 hours, 4 hours, 5 hours, 6 hours, 7 hours, 8 hours, 9 hours, 10 hours, 11 hours, or 12 hours after collection of the blood fraction comprising the separated cells. In some embodiments, the contacting of separated cells is initiated within 30 minutes, 1 hour, 2 hours, 2 hours, 4 hours, 5 hours, 6 hours, 7 hours, 8 hours, 9 hours, 10 hours, 11 hours, or 12 hours after collection of the blood fraction comprising the separated cells. In some embodiments, the contacting of separated cells is initiated at least 12 hours after collection of the blood fraction comprising the separated cells. In some embodiments, the contacting of separated cells is initiated within 12 hours, 13 hours, 14 hours, 15 hours, 16 hours, 17 hours, 18 hours, 19 hours, 20 hours, 21 hours, 22 hours, 23 hours or 24 hours after collection of the blood fraction comprising the separated cells. In some embodiments, the contacting of separated cells is initiated no more than 1 hour after collection of the blood fraction comprising the separated cells. In some embodiments, the contacting of separated cells is initiated within 0-5 minutes, 5-10 minutes, 10-15 minutes, 15-30 minutes. 30-45 minutes, 45-60 minutes after collection of the blood fraction comprising the separated cells. In some embodiments, the contacting of separated cells is initiated at least 0-5 minutes, 5-10 minutes, 10-15 minutes, 15-30 minutes. 30-45 minutes, 45-60 minutes after collection of the blood fraction comprising the separated cells.

In some embodiments, separated cells (e.g. leukocyte components) are contacted with a composition in a contacting chamber. In some embodiments, the method comprises contacting the separated cells (e.g. leukocyte components) with a composition comprising lipid particles or non-lipid particles. In some embodiments, such lipid or non-lipid particles carry a payload gene so that the method can be used to deliver the payload gene to a subject via lipid or non lipid based methods. In particular embodiments, the separated cells (e.g. leukocyte components) are contacted with a composition of lipid particles, such as a viral vector or viral-like particles. In some embodiments, the method comprises contacting the separated cells (e.g. leukocyte components) with a composition comprising nucleic acids (e.g., such as nucleic acids encoding a payload gene). In some embodiments, the separated cells (e.g. leukocyte components) are contacted with a composition comprising lipid particles or nucleic acids within a contacting chamber.

In some embodiments, the separated cells (e.g. leukocyte components) that have been separated from whole blood as described in Section II.B are pumped (e.g., via an in-line pump) into the inner cavity (i.e., lumen) of a contacting chamber (e.g. which in some cases can be the collection container). In some aspects, any suitable contacting chamber known in art may be used in the provided methods. In some embodiments, the contacting chamber is made from hard plastic and comprises a lumen with a set volume. In some embodiments, the contacting chamber is not made from hard plastic and comprises a lumen with a variable volume. In some embodiments, the contacting chamber is made from a flexible plastic such as polyvinyl chloride. In some embodiments, the contacting chamber is a blood bag.

In some embodiments, the contacting chamber is open along at least one wall. In some embodiments, the contacting chamber comprises at least one opening (e.g. inlet) capable of permitting the aspiration of liquid in and out of the internal cavity. In some embodiments, the contacting chamber is closed. In some embodiments, the contacting chamber is sterile.

In some embodiments, the contacting of the separated cells (e.g. leukocyte components) and the lipid particle or viral vector (e.g. viral vector or viral-like particle) or nucleic acid can generate a transfection mixture. In some embodiments, the transfection mixture includes all of the separated cells (e.g. leukocyte components) collected from the whole blood of the subject and a fixed amount or concentration of the lipid particle or viral vector (e.g. viral vector or viral-like particle) or nucleic acid. In some aspects, transfection is a process by which a non-endogenous nucleic acid is inserted into eukaryotic cells, such as by viral or plasmid vector. In some embodiments, transfection of the separated cells is via contacting the separated cells with a composition comprising lipid particles or viral vector or nucleic acid (e.g., contacting such as in the contacting chamber).

In some embodiments, the composition comprising lipid particles or viral vector or the composition comprising nucleic acids is present within the lumen of the contacting chamber. In some embodiments, the contacting chamber is pre-filled with the composition prior to the introduction of the separated cells (e.g. leukocyte components). In some embodiments, the composition comprising lipid particle or viral vector (e.g. viral vector or viral-like particle) or nucleic acids is introduced into the contacting chamber simultaneously as the separated cells (e.g. leukocyte components). In some embodiments, the composition comprising lipid particle or viral vector (e.g. viral vector or viral-like particle) or nucleic acids is introduced into the contacting chamber subsequent to the separated cells (e.g. leukocyte components). In some embodiments, the composition comprising lipid particles or viral vectors or nucleic acids is connected to the contacting chamber via an operable connection, optionally with a tube, line, valve, luer port, or spike. In some embodiments, the composition comprising lipid particles or viral vectors or nucleic acids is introduced (i.e., via an in-line pump as described above) directly into the lumen of the contacting chamber.

In some embodiments, the concentration of cells (e.g. leukocyte components, such as PBMCs) in the contacting chamber is between 1×10⁶ cells/mL and 1×10 cells/mL, between 1×10⁶ cells/mL and 1×10⁸ cells/mL, between 1×10⁶ cells/mL and 1×10⁷ cells/mL, between 1×10⁷ cells/mL and 1×10⁸ cells/mL, between 1×10⁷ cells/mL and 1×108 cells/mL or between 1×108 cells/mL and 1 x 10⁸ cells/mL. In some embodiments, the concentration of cells (e.g. leukocyte components, such as PBMCs) in the contacting chamber is at or about 1×10⁶ cells/mL, 5×10⁶ cells/mL, 1×10⁷ cells/mL, 5×10⁷ cells/mL, 1×108 cells/mL, 5×10⁸ cells/mL or 1×10⁸ cells/mL, or is any value between any of the foregoing. In some embodiments, the concentration of cells (e.g. leukocyte components, such as PBMCs) in the contacting chamber is at or about 1×10⁷ cells/mL.

In some embodiments, there is a fixed concentration of lipid particles or viral vectors within the lumen of the contacting chamber. In some embodiments, the fixed concentration of lipid particles or viral vectors is 1-5×10⁹, 5-10×10⁹, 10-20×10⁹, 20-30×10⁹, 30-40×10⁹, 40-50×10⁹, 50-60×10⁹, 60-70×10⁹, 70-80×10⁹, 80-90×10⁹,1-5×10⁹, 5-10×10⁹, 10-20×10⁹, 20-30×10⁹, 30-40×10⁹, or 40-50×10⁹ particles, each range inclusive. In some embodiments, the fixed concentration of lipid particles or viral vectors is 1-5×10¹”, 5-10×10¹”, 10-20×10¹”, 20-30×10¹”, 30-40×10¹”, 40-50×10¹”, 50-60×10¹”, 60-70×10¹”, 70-80×10¹⁰, 80-90×10¹”,1-5×10¹”, 5-10×10¹”, 10-20×10¹”, 20-30×10¹”, 30-40×10¹”, or 40-50×10¹⁰ particles, each range inclusive. In some embodiments, the fixed concentration of lipid particles or viral vectors is at least 5×10⁹, 10×10⁹, 20×10⁹, 30×10⁹, 40×10⁹, 50×10⁹, 60×10⁹, 70×10⁹, 80×10⁹, 90×10⁹,1×10¹”, 5×10¹”, 10×10¹⁰, 20×10¹”, 30×10¹”, 40×10¹⁰ or 50×10¹⁰ particles. In some embodiments, the fixed concentration of lipid particles or viral vectors is at or about 5×10⁹, 10×10⁹, 20×10⁹, 30×10⁹, 40×10⁹, 50×10⁹, 60×10⁹, 70×10⁹, 80×10⁹, 90×10⁹,1×10¹”, 5×10¹”, 10×10¹”, 20×10¹”, 30×10¹”, 40×10¹⁰ or 50×10¹⁰ particles, or any value between any of the foregoing. In some embodiments, the fixed concentration of lipid particles or viral vectors is or is at or about 1×10⁹, 10×10⁹, 20×10⁹, 30×10⁹, 40×10⁹, or 50×10⁹ particles, or any value between any of the foregoing. In some embodiments, the fixed concentration of lipid particles or viral vectors is or is at or about 1×10¹”, 10×10¹⁰, 20×10¹⁰, 30×10¹⁰, 40×10¹⁰, or 50×10¹⁰ particles, or any value between any of the foregoing.

In some embodiments, the lipid particle is a viral vector (e.g. lentiviral vector) or viral-like particle. In some embodiments, there is a fixed concentration of lipid particles within the lumen of the contacting chamber. In some embodiments, the fixed concentration of lipid particles is 1-5×109, 5-10×10⁹, 10-20×10⁹, 20-30×10⁹, 30-40×10⁹, 40-50×10⁹, 50-60×10⁹, 60-70×10⁹, 70-80×10⁹, 80-90×10⁹,1-5×10⁹, 5-10×10⁹, 10-20×10⁹, 20-30×10⁹, 30-40×10⁹, or 40-50×10⁹ infectious units (IU), each range inclusive. In some embodiments, the fixed concentration of lipid particles is 1-5×10¹⁰, 5-10×10¹⁰, 10-20×10¹⁰, 20-30×10¹⁰, 30-40×10¹⁰, 40-50×10¹⁰, 50-60×10¹⁰, 60-70×10¹⁰, 70-80×10¹⁰, 80-90×10¹⁰,1-5×10¹⁰, 5-10×10¹⁰, 10-20×10¹⁰, 20-30×10¹⁰, 30-40×10¹⁰, or 40-50×10¹⁰ infectious units (IU), each range inclusive. In some embodiments, the fixed concentration of lipid particles is at least 5×10⁹, 10×10⁹, 20×10⁹, 30×10⁹, 40×10⁹, 50×10⁹, 60×10⁹, 70×10⁹, 80×10⁹, 90×10⁹,1×10^{1O}, 5×10¹⁰, 10×10¹⁰, 20×10¹′, 30×10¹⁰, 40×10¹⁰ or 50×10¹⁰ IU. In some embodiments, the fixed concentration of lipid particles is at or about 5×10⁹, 10×10⁹, 20×10⁹, 30×10⁹, 40×10⁹, 50×10⁹, 60×10⁹, 70×10⁹, 80×10⁹, 90×10⁹,1×10^{1O}, 5×10¹⁰, 10×10¹⁰, 20×10¹⁰, 30×10¹⁰, 40×10¹⁰ or 50×10¹⁰ IU, or any value between any of the foregoing. In some embodiments, the fixed concentration of lipid particles is or is at or about 1×10¹⁰, 10×10¹⁰, 20×1010, 30×10¹⁰, 40×10¹⁰, or 50×10¹⁰ IU, or any value between any of the foregoing.

In some embodiments, the lipid particle is a viral vector (e.g. lentiviral vector) or viral-like particle. In some embodiments, there is a fixed concentration of lipid particles within the lumen of the contacting chamber. In some embodiments, the fixed concentration of lipid particles (e.g. viral vector or viral-like particle) is 1-5×10³, 5-10×10³, 10-20×10³, 20-30×10³, 30-40×10³, 40-50×10³, 50-60×10³, 60-70×10³, 70-80×10³, 80-90×10³,1-5×10⁴, 5-10×104, 10-20×10⁴, 20-30×10⁴, 30-40×10⁴, or 40-50×10⁴ viral genomic (Vg)/cell, each range inclusive. In some embodiments, the fixed concentration of lipid particles (e.g. viral vector or viral-like particle) is at least 5×10³, 10×10³, 20×10³, 30×10³, 40×10³, 50×10³, 60×10³, 70×10³, 80×10³, 90×10³,1×10⁴, 5×10⁴, 10×10⁴, 20×10⁴ cells, 30×10⁴, 40×10⁴ or 50×10⁴ Vg/cell. In some embodiments, the fixed concentration of lipid particles (e.g. viral vector or viral-like particle) is at or about 5×10³, 10×10³, 20×10³, 30×10³, 40×10³, 50×10³, 60×10³, 70×10³, 80×10³, 90×10³,1×10⁴, 5×10⁴, 10×10⁴, 20×10⁴ cells, 30×10⁴, 40×10⁴ or 50×10⁴ Vg/cell, or any value between any of the foregoing. In some embodiments, the fixed concentration of lipid particles is or is about 1×10³, 5×10³, 10×10³, 20×10³, 30×10³, 40×10³, or 50×10³ Vg/cell.

In some embodiments, there is a fixed amount of lipid particles or viral vectors (e.g. viral vector or viral-like particle) within the lumen of the contacting chamber. In some embodiments the viral vector or viral-like particle is a retroviral vector or retroviral-like particle, such as a lentiviral vector or lentiviral-like particle. In some embodiments, the fixed amount of the viral vector or virus-like particle is from about 10⁴ to about 10¹⁰ plaque forming units (pfu), inclusive. In some embodiments, the fixed amount of a viral vector or virus-like particle is from about 10′ to about 10¹⁵ pfu, inclusive In some embodiments, the fixed amount of a viral vector or virus-like particle is from about 105 to about 10¹¹ pfu. In some embodiments, the fixed amount of a viral vector or virus-like particle is from about 10⁶ to about 109 pfu. In some embodiments, the fixed amount of a viral vector or virus-like particle is from about 10¹² to about 10¹⁴pfu, inclusive. In some embodiments, the fixed amount is 1.0×10⁹ pfu, 5.0×10⁹ pfu, 1.0×10¹⁰ pfu, 5.0×10^{O} pfu, 1.0×10” pfu, 5.0×10” pfu, 1.0×10¹² pfu, 5.0×10¹² pfu, or 1.0×10” pfu, 5.0×10¹¹ pfu, 1.0×10¹⁴ pfu, 5.0×10¹⁴ pfu, or 1.0×10^{O} pfu.

In some embodiments, the viral vector that is an adenovirus vector. In some aspects, the fixed amount of adenovirus to humans can range from about 10⁷ to 10′, inclusive, plaque forming units (pfu).

In some embodiments, there is a fixed concentration of nucleic acid within the lumen of the contacting chamber. In some embodiments, the fixed concentration of nucleic acid is 1-5, 5-10, 10-20, 20-30, 30-40, 40-50, 50-60, 60-70, 70-80, 80-90, or 90-100 ng/mL, each range inclusive. In some embodiments, the fixed concentration of lipid particles is at least 1, 5, 10, 20, 30, 40, 50, 60, 70, 80, 90 or 100 ng/mL. In some embodiments, the fixed concentration of lipid particles is 1, 5, 10, 20, 30, 40, 50, 60, 70, 80, 90 or 100 ng/mL.

In some embodiments, there is a variable concentration of lipid particles within the lumen of the contacting chamber. In some embodiments, there is a variable concentration of nucleic acid within the lumen of the contacting chamber. In some embodiments, the concentration of lipid particle or nucleic acid within the contacting chamber is variable over time, and/or variable with cell density. In some embodiments, the concentration of lipid particle or nucleic acid is maintained over cell density such that more or less of the composition containing the lipid particles or nucleic acid is introduced into the contacting chamber in accordance with the total number of cells (i.e., the concentration of lipid particles or nucleic acid per cell is maintained over the contacting period).

In some embodiments, the composition comprising lipid particles or viral vectors or the composition comprising the nucleic acid is present within the lumen of the contacting chamber In some embodiments, the composition comprising lipid particles or viral vectors or nucleic acids has a volume of 100, 200, 300, 400, or 500 milliliters. In some embodiments, the composition comprising lipid particles or nucleic acids is present within the lumen of the contacting chamber and has a volume of at most 1 liter. In some embodiments, the composition comprising lipid particles or nucleic acids is present within the lumen of the contacting chamber and has a volume of at most 500 milliliters.

In some embodiments, the contacting of separated cells (e.g. fraction of blood containing leukocyte components) with the composition comprising lipid particle or viral vector within the contacting chamber is for a set limit of time. In some embodiments, the contacting of separated cells within the contacting chamber is for 15 minutes to 12 hours, such as 15 minutes to 6 hours, 15 minutes to 4 hours, 15 minutes to 2 hours, 15 minutes to 1 hour, 1 hour to 12 hours, 1 hour to 6 hours, 1 hour to 4 hours, 1 hour to 2 hours, 2 hours to 12 hours, 2 hours to 6 hours, 2 hours to 4 hours, 4 hours to 12 hours, 4 hours to 6 hours or 6 hours to 12 hours.

In some embodiments, the contacting of separated cells within the contacting chamber is for 1-2 hours, 2-4 hours, 4-6 hours, 6-8 hours, 8-10 hours, 10-12 hours, 12-14 hours, 14-16 hours, 16-18 hours, 18-20 hours, 20-22 hours, 22-24 hours, each range inclusive. In some embodiments, the contacting of separated cells is for at most 12 hours. In some embodiments, the contacting of separated cells is for at most 1 hour, 2 hours, 2 hours, 4 hours, 5 hours, 6 hours, 7 hours, 8 hours, 9 hours, 10 hours, 11 hours, or 12 hours. In some embodiments, the contacting of separated cells is for 1 hour, 2 hours, 2 hours, 4 hours, 5 hours, 6 hours, 7 hours, 8 hours, 9 hours, 10 hours, 11 hours, or 12 hours. In some embodiments, the contacting of separated cells is for at least 12 hours. In some embodiments, the contacting of separated cells is for 12 hours, 13 hours, 14 hours, 15 hours, 16 hours, 17 hours, 18 hours, 19 hours, 20 hours, 21 hours, 22 hours, 23 hours or 24 hours.

In some embodiments, the contacting of separated cells with the composition comprising the lipid particles or viral vectors or nucleic acids is for no more than 1 hour. In some embodiments, the contacting of separated cells is for 0-5 minutes, 5-10 minutes, 10-15 minutes, 15-30 minutes. 30-45 minutes, 45-60 minutes, each range inclusive. In some embodiments, the contacting of separated cells within the contacting chamber is for 30-60 minutes. In some embodiments, the contacting of separated cells is for at or about 60 minutes. In some embodiments, the contacting of separated cells is for at or about 30 minutes. In some embodiments, the contacting of separated cells is for at or about 15 minutes.

In some embodiments, the transfection mixture is mixed manually or by automatic methods during at least a portion of the contacting. In some embodiments, mixing is by physical manipulation of the contacting chamber (e.g. bag). In some embodiments, the mixing is carried out without disconnecting or disengaging the contacting chamber (e.g. bag) from the in-line system. In some embodiments, the mixing is carried out under sterile conditions.

In some embodiments, the contacting chamber is centrifugal. In some embodiments, the contacting chamber is rotatable about a rotation axis. In some embodiments, the contacting chamber is rotating for at least a portion of the contacting period. In some embodiments, the contacting chamber is rotating for 0-10%, 10-20%, 20-30%, 30-40%, 40-50%, 50-60%, 60-70%, 70-80%, 80-90%, or 90-100% of the total contacting period, each range inclusive. In some embodiments, the contacting chamber is rotating for the entire contacting period. In some embodiments, the contacting chamber is rotating for at least 5 minutes, at least 10 minutes, or at least 15 minutes, or at least 20 minutes, or at least 30 minutes, 45 minutes or more, or 60 minutes or more, or 90 minutes or more, or 120 minutes or more; or 5 minutes to 60 minutes, 10 minutes to 60 minutes, 15 minutes to 60 minutes, 15 minutes to 45 minutes, 30 minutes to 60 minutes, or 45 minutes to 60 minutes.

In some embodiments, centrifugation at high speeds, for example, at a force (relative centrifugal force (RCF)) of between 200 g and 3000 g, such as between 500 g and 2500 g, between 500 g and 2000 g, between 500 g and 1500 g, between 500 g and 1000 g, between 1000 g and 3000 g, between 1000 g and 2500 g, between 1000 g and 2000 g, between 1000 g and 1500 g, between 1500 g and 3000 g, between 1500 g and 2500 g, between 1500 g and 2000 g, between 2000 g and 3000 g, between 2000 g and 2500 g or between 2500 g and 3000 g. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, the contacting chamber includes one or more opening(s), such as one or more inlet, one or more outlet, and/or one or more inlet/outlet, which can permit intake and output of liquid fluid to and from the cavity. In some embodiments, liquid (e.g. containing a composition of lipid particles or viral vectors) may be taken into the cavity through a tubing line or other channel that is or is placed in connection with the opening (e.g. inlet), for example, by placing the line or channel in connection with and control of a pump, syringe, or other machinery, which may be controlled in an automated fashion. In some embodiments, liquid (e.g. containing a composition of contacted leukocytes containing the separated leukocytes and lipid particles or viral vectors ) may be expelled or outputted through the cavity through a tubing line or other channel that is or is placed in connection with the opening (e.g. outlet), for example, by placing the line or channel in connection with and control of a pump, syringe, or other machinery, which may be controlled in an automated fashion. In some embodiments, the chamber is pre-connected to one or more of the additional components, directly and/or indirectly. Such a chamber may be provided as part of a pre-assembled kit, e.g., a kit packaged for single, sterile, use in connection with the provided methods. In some embodiments, various components are packaged separately, for example, to allow for custom configurations in which a user connects and arranges the components for a particular embodiment of the processing methods.

The components typically include at least one tubing line, and generally a set or system of tubing lines, and at least one connector. Exemplary connectors include valves, ports, spikes, welds, seals, and hose clamps. The connectors and/or other components may be aseptic, for example, to permit the entire process to be carried out in a closed, sterile system, which can eliminate or reduce the need for clean rooms, sterile cabinets, and/or laminar flow systems.

In some embodiments, the contacting chamber is comprised in a fluid circuit, optionally a closed in-line circuit. In some embodiments, the contacting chamber can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the contacting chamber is disposable. In some embodiments, the contacting chamber is disposable. Thus, in some embodiments, the contacting chamber is part of a closed system, such as a sterile system, having various additional components such as tubing lines and connectors and caps, within which processing steps occur. Thus, in some embodiments, the provided methods and/or steps thereof are carried out in a completely closed or semi-closed environment, such as a closed or semi-closed sterile system, facilitating the processing of the lipid particle or viral vector for therapeutic administration to subjects without the need for a separate sterile environment, such as a biosafety cabinet or room. The methods in some embodiments are carried out in an automated or partially automated fashion.

In some embodiments, the composition comprising lipid particle or viral vector or nucleic acids as present in the contacting chamber is supplemented with at least one agent to enhance transfection (i.e., an adjuvant of transfection). In some embodiments, one or more transfection reagents are used. Any suitable transfection reagent known in the art may be used in the provided method, for example some commercially available transfection reagents such as Effectene and TransIT-X2 (e.g., Effectene and FuGENE 6) are specially dedicated for use with plasmid DNA, while some transfection reagents such as Lipofectamine RNAiMAX are more suited for use with small oligonucleotides. Other agents to enhance transfection may include members of the Lipofectamine and DharmaFECT families, which in some aspects are associated with higher transfection efficiencies in transfecting primary human cells (Hunt et al., 2010). In some embodiments, composition comprising lipid particle or viral vector or nucleic acids as present in the contacting chamber is supplemented with at least one agent chosen from the group comprising Lipofectamine, Lipofectamine 3000, Lipofectamine 2000, PEI-based reagents, Transporter™ 5 and PEI25, PEG, Xfect, Nanofectamin, TransIT-X2, TransIT-2020, FuGENE 6, Effectene, HiperFect, and ExGen 500.

In some embodiments, the composition comprising lipid particle or viral vector or nucleic acids as present in the contacting chamber is supplemented with a T cell activation element. In some embodiments, the T cell activation element may be either in solution or on the surface of the viral vector (e.g. lentiviral vector particles) to facilitate genetic modification (e.g. transduction) of T cells in the transfection mixture. In some embodiments, the T cell activation element activates a T cell through T cell receptor associated complex. Such an activation element can be an anti-CD3 antibody, for example an anti-CD3 scFv or an anti-CD3 scFvFc. In some embodiments, the T cell activation agent includes anti-CD3 and another polypeptide that binds to a costimulatory receptor such as CD28. In some embodiments, the T cell activation element may include anti-CD3.anti-CD28 antibodies or T cell stimulatory cytokines such as IL-2, IL15 or IL-7. In some embodiments, the T cell activation element is a reagent that is soluble. In some embodiments, the T cell activation element is membrane bound of the surface of a viral vector. In some embodiments, the T cell activation element is part of a pseudotyping element on the surface of a viral vector, in which the T cell activation element is not encoded by a polynucleotide of in the viral vector.

In some embodiments, the T cell activation element can be an anti-CD3 antibody, such as an anti-CD3 scFv or anti-CD3 scFvFc. In some embodiments, the T cell activation element may include a polypeptide capable of binding to CD28. In some embodiments, the polypeptide capable of binding to CD28 is an anti-CD28 antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an extracellular domain portion of CD80. In some embodiments, the anti-CD28 antibody or fragment thereof is a single chain anti-CD28 antibody, such as, but not limited to, an anti-CD28 scFv. In some embodiments, an activation element is fused to a heterologous signal sequence and/or a heterologous membrane attachment sequence, both of which help direct the activation element to the membrane. In some embodiments, the membrane attachment sequence is a GPI anchor. In some embodiments, the T cell activation element can be included on the surface of a viral vector, such as by pseudotyping as part of a fusogen (e.g. described in Section III.A).

In some embodiments, the T cell activation element also may include a membrane bound cytokine, such as IL-2, IL-17, IL-15 or an active fragment thereof. In some embodiments, the cytokine a heterologous signal sequence and/or a heterologous membrane attachment sequence, both of which help direct the activation element to the membrane. In some embodiments, the membrane attachment sequence is a GPI anchor. In some embodiments, the T cell activation element can be included on the surface of a viral vector, such as by pseudotyping as part of a fusogen (e.g. described in Section III.A).

Exemplary T cell activation elements and agents are described in WO20190559546 or WO2021042072.

In some embodiments, the composition comprising lipid particle or viral vector or nucleic acids as present in the contacting chamber are not supplemented with a T cell activation element. In some embodiments, the T cells of the leukocyte component are non-activated T cells.

In some embodiments, the contacting step is performed at a temperature between at or about 18° C. and 42° C. In some embodiments, the temperature is between 20° C. and 25° C., such as at or about 22° C. In some embodiments, the contacting step is performed at temperatures between 32° C. and 42° C., such as at or about 37° C. In some embodiments, the contacting step is performed at or about 5% CO2.

In some embodiments, the transfection mixture containing all separated cells collected from the whole blood fraction and the fixed amount or concentration of lipid particle or viral vector or nucleic acid(s) is not washed or subjected to further processing after the contacting. In some embodiments, the entire composition of the transfection mixture is used for reinfusion to the subject.

### D. Reinfusion of Lipid Particles or Viral Vectors to Subject

In some embodiments, the method further provides reinfusing the contacted cell component or the transfection mixture containing the lipid particle or viral vector (e.g. encoding a payload gene) to a subject. In some embodiments, the reinfusion thus administers the lipid particle or viral vector and/or payload gene to the subject. In some embodiments, the transfection mixture is directly administered to the subject. In some embodiments the transfection mixture is not further washed or processed after the contacting with the lipid particle or viral vector prior to reinfusion to the subject.

In some embodiments, the contacted cell component or the transfection mixture are contained in a transfer container for infusion to a subject. In some embodiments, the composition containing the contacted leukocyte components, such as the transfection mixture, are moved from the contacting chamber to the transfer chamber, such as via one or more operably connected tubing lines. In some embodiments, the transfer container is a bag. In some embodiments, the transfer container is a rigid container. In some embodiments, the transfer container is opaque or partially opaque.

In some embodiments, the transferred contacted leukocyte components, such as the transfection mixture, contained in the transfer container are severed or otherwise separated from the tubing sets used during the process, in which the reinfusion to the subject is offline. In some embodiments, offline reinfusion is a manual reinfusion. Thus, in some embodiments, the transfer container containing the contacted leukocyte components or precursors thereof are detached from the donor subject prior to their reinfusion to the donor subject.

In some embodiments, the transfer container remains in-line with the processing system for reinfusion of the contacted leukocyte components or precursors thereof, such as the transfection mixture, directly to the subject without detachment from the donor subject or separation from the tubing sets used during the process. The provided methods that improve efficiency of the process avoids any additional product labeling and/or traceable handling requirements because the transfection mixture for reinfusion never leaves the disposable set which remains connected to the donor subject during the entire treatment procedure.

In some embodiments, the time to reinfusion to the subject following the contacting is no more than 24 hours after obtaining the whole blood from the subject(e.g., as described in Section II. A.) In some embodiments, the time to reinfusion to the subject following the contacting of the separated cell is for a time of from 1 to 24 hours, 1 to 12 hours, 1 to 6 hours, 1 to 4 hours, 1 to 2 hours, 2 hours to 24 hours, 2 hours to 12 hours, 2 hours to 6 hours, 2 hours to 4 hours, 4 hours to 24 hours, 4 hours to 12 hours, 4 hours to 6 hours, 6 hours to 24 hours, 6 hours to 12 hours or 12 hours to 24 hours, after obtaining whole blood from the subject(e.g., as described in Section II. A.) In some embodiments, the time to reinfusion to the subject following the contacting of separated cells is for a time ofi-2 hours, 2-4 hours, 4-6 hours, 6-8 hours, 8-10 hours, 10-12 hours, 12-14 hours, 14-16 hours, 16-18 hours, 18-20 hours, 20-22 hours, 22-24 hours after obtaining whole blood from the subject(e.g., as described in Section II. A.). In some embodiments, the time to reinfusion to the subject following the contacting of separated cells is no more than 1, 2, 3, 4, 5, or 6 hours. In some embodiments, the time to reinfusion to the subject following the contacting of the separated cells is at most 1, 2, 3, 4, 5, or 6 hours. In some embodiments, the time to reinfusion to the subject following the contacting of the separated cells is at or about 1, 2, 3, 4, 5, or 6 hours, or any value between any of the foregoing. In some embodiments, the time to reinfusion to the subject following the contacting of separated cells is no more than 1, 2, or 3 days after obtaining whole blood (e.g., as described in Section II. A.).

In some embodiments, the composition comprising contacted cells is connected to the return processing unit via an operable connection, optionally with a tube, line, valve, luer port, or spike. In some embodiments, the composition comprising contacted cells is pumped (i.e., via an in-line pump as described above) directly into the lumen of the return processing unit.

In some of any of the provided embodiments, the reinfusion of the contacted cells for administration of the lipid particle or viral vector or payload gene is via a return processing unit. In some embodiments, the return processing unit returns the separated cells, the first blood component, the second blood component, and/or the third blood component to the subject.. In some embodiments, the return processing unit device has an inlet. In some embodiments, the return processing unit device also has an outlet and may optionally alternate between discharging the first blood component (e.g., leukocytes) and the second blood component (i.e. red blood cells and/or plasma) through the outlet.. In some embodiments, the second and/or third blood component may be returned to the subject in addition to the first blood component via the return line, optionally wherein the return line is operably connected to the return processing unit. In some embodiments, The first blood component is leukocytes and/or the second blood component is red blood cells, and/or the third blood component is plasma and/or platelets. In some embodiments, the return line operably connects to the venous-access device at a point between the draw line pump and the venous-access device. In some embodiments, the venous-access device is operably connected to the return processing unit.

In some embodiments, the return processing unit is comprised in a fluid circuit, optionally a closed in-line circuit.. In some embodiments, the return processing unit can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the return processing unit set is disposable. In some embodiments, the return processing unit is disposable. In some embodiments, the return processing unit is sterile.

In some embodiments, the composition comprising contacted cells present within the lumen of the return processing unit has a volume of 100-200 milliliters, 200-300 milliliters, 300-400 milliliters, or 400-500 milliliters, each range inclusive. In some embodiments, the composition comprising contacted cells present within the lumen of the contacting chamber has a volume of no more than 500 milliliters. In some embodiments, the composition comprising contacted cells present within the lumen of the contacting chamber has a volume of at least 100, 200, 300, 400, or 500 milliliters. In some embodiments, the composition comprising contacted cells present within the lumen of the contacting chamber has a volume of 100, 200, 300, 400, or 500 milliliters. In some embodiments, the composition comprising contacted cells present within the lumen of the return processing unit has a volume of no more than 1 liter.

In some embodiments, the return processing unit comprises an in-line pump for reinfusion of separated cells to the subject. In some embodiments, the total number of reinfused cells is 5-10×10⁸, 10-20×108, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the total number of reinfused cells is at least 5×10⁸, 10×10⁸, 20×10⁸, 30×10⁸, 40×10⁸, 50×10⁸, 60×10⁸, 70×10⁸, 80×10⁸, 90×10⁸, 100×108, 150×10⁸, 200×10⁸, or 300×10⁸cells. In some embodiments, the total number of reinfused cells is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the total number of reinfused cells is 1-5%, 5-10%, 10-20%, 20-30%, 30-40%, 40-50%, or 50-60% of the total number of separated cells, each range inclusive. In some embodiments, the total number of reinfused cells is at least 5%, 10%, 20%, 30%, 40%, 50%, 60%, 70%, 80%, or 90% of the total cell number of separated cells. In some embodiments, the total number of reinfused cells is 5%, 10%, 20%, 30%, 40%, 50%, 60%, 70%, 80%, 90%, or 100% of the total cell number of separated cells.

### E. Modules for Monitoring and Adjusting Administration

In some embodiments, the system for administration comprises at least one module for monitoring and/or adjusting administration of the lipid particles or viral vectors or payload gene. In some embodiments, the system for administration in in-line, optionally wherein the system is a closed system. In some embodiments, the module for monitoring and/or adjusting administration is comprised in a fluid circuit, optionally a closed in-line circuit.. In some embodiments, the module can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable.

In some embodiments, the module is operably connected to the return processing unit, optionally wherein the module is connected via a fluid and/or signal connection with the return processing unit. In some embodiments, the module is operably connected to the return processing unit and/or to an in-line pump, optionally wherein the module is connected via a fluid and/or signal connection with the return processing unit and/or to an in-line pump. In some embodiments, the module can adjust the speed and/or duration of reinfusion of the contacted cells according to the provided methods.

### Lipid Particles, Viral Vectors and Nucleic Acids for Administration

The provided methods and embodiments can be used to deliver of lipid particles or viral vectors or nucleic acids for administration to a subject. In some embodiments, the nucleic acid (e.g. polynucleotides) can be a naked nucleic acid (e.g. mRNA or DNA) or can be delivered in a carrier or vehicle for delivery. In some embodiments, a nucleic acid is contained in a vehicle, such as viral-particles, viral-like particles, or non-viral particles. In some embodiments, the nucleic acid is delivered as a naked nucleic acid. In some embodiments, the nucleic acid is an mRNA. In some embodiments, the nucleic acid is a DNA, e.g., a plasmid.

In some embodiments, vectors that package a polynucleotide encoding a payload agent may be used to deliver the payload agent according to the provided methods. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In some embodiments, the vector may be a viral vector such as a lentiviral vector, a gamma-retroviral vector, a recombinant AAV, an adenoviral vector or an oncolytic viral vector. In other aspects, non-viral vectors for example, nanoparticles and liposomes may also be used for introducing and delivery of a polynucleotide encoding the payload agent.

In some embodiments, the lipid particle is a viral vector or is derived from a viral vector. In other embodiments, the vehicle is a non-viral vector, such as a cellular particle, liposome, nanoparticle, or other synthetic particle. Non-viral vectors and methods employing the use of polymers, surfactants, and/or excipients have been employed to introduce polynucleotides and polypeptides into cells including conjugation with a targeting moiety, conjugation with a cell penetrating peptide, derivatization with a lipid and incorporation into liposomes, lipid nanoparticles, and cationic liposomes. The majority of non-viral vectors are composed of plasmid DNA complexed with lipids or polycations. Many different lipids with ability to deliver plasmid DNA to cells in vitro and in vivo have been reported (Gao, et al., Gene Therapy 2:710-722 (1995)).

In any of the provided embodiments, the lipid particle or viral vector or nucleic acid is or encodes a payload gene for delivery to a cell or a cell in a subject.

In particular embodiments, the nucleic acid encoding the payload gene is encapsulated within the lumen of a lipid particle in which the lipid particle contains a lipid bilayer, a lumen surrounded by the lipid bilayer. In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

### A. Viral Vectors

In some embodiments, the lipid particles include viral vector particles. In some embodiment the viral particles include those derived from retroviruses or lentiviruses. In some embodiments, the viral particle's bilayer of amphipathic lipids is or comprises the viral envelope. In some embodiments, the viral particle's bilayer of amphipathic lipids is or comprises lipids derived from an infected host cell.

Biological methods for introducing an exogenous agent to a host cell include the use of DNA and RNA vectors. DNA and RNA vectors can also be used to house and deliver polynucleotides and polypeptides. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the polynucleotides (e.g. encoding a payload gene) are comprised within a viral vector. In some embodiments, the polynucleotides (e.g. encoding a payload gene) comprised within a recombinant virus particles.

In some embodiments, the viral vector is a vectors derived from adenoviruses and adeno-associated virus (AAV). Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772; Pulicherla et al. Molecular Therapy, 2011, 19(6): 1070-1078; U.S. Pat. Nos. : 6,156,303; 7,198,951; U.S. Patent Publication Nos. : US2015/0159173 and US2014/0359799; and International Patent Publication NOs.: WO1998/011244, WO2005/033321 and W02014/14422.

In some embodiments, the AAV vector is of serotype 1, 2, 6, 8 or 9. In some embodiments, the AAV vector is of serotype 6.2.In some embodiments, the AAV vector includes a capsid that is a chimera between AAV2 (aa 1-128) and AAV5 (aa 129-725) with one point mutation (A581T) (AAV2.5T, Excoffon et al. Proc Natl Acad Sci. 106(10):3875-70, 2009).

In some embodiments, the AAV is a single-stranded DNA parvovirus which is capable of host genome integration during the latent phase of infectivity. For example, AAV of serotype 2 is largely endemic to the human and primate populations and frequently integrates site-specifically into human chromosome 19 q13.3. In some aspects, AAV is considered a dependent virus because it requires helper functions from either adenovirus or herpes-virus in order to replicate. In the absence of either of these helper viruses, AAV has been observed to integrate its genome into the host cell chromosome. However, these virions are not capable of propagating infection to new cells.
AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiment, suitable host cells for producing AAV derived vehicles include microorganisms, yeast cells, insect cells, and mammalian cells. In some embodiments, the term host cell includes the progeny of the original cell which has been transfected. Thus, as indicated above, a “host cell,” or “producer cell,” as used herein, generally refers to a cell which has been transfected with a vector vehicle as described herein. For example, cells from the stable human cell line, 293 (ATCC Accession No. CRL1573) are familiar to those in the art as a producer cell for AAV vectors. The 293 cell line is a human embryonic kidney cell line that has been transformed with adenovirus type-5 DNA fragments (Graham et al., J. Gen. Virol., 36:59 (1977)), and expresses the adenoviral E1a and E1b genes (Aiello et al., Virol., 94:460 (1979)). The 293 cell line is readily transfected, and thus provides a particularly useful system in which to produce AAV virions.

Producer cells as described above containing the AAV vehicles provided herein must be rendered capable of providing AAV helper functions. In some embodiments, producer cells allow AAV vectors to replicate and encapsulate polynucleotide sequences. In some embodiments, producer cells yield AAV virions. AAV helper functions are generally AAV-derived coding sequences that may be expressed to provide AAV gene products that, in turn, function for productive AAV replication. In some embodiments, AAV helper functions are used to complement necessary AAV functions that are missing from the AAV vectors. In some embodiments, AAV helper functions include at least one of the major AAV ORFs. In some embodiments, the helper functions include at least the rep coding region, or a functional homolog thereof. In some embodiments, the helper function includes at least the cap coding region, or a functional homolog thereof.

In some embodiments, the AAV helper functions are introduced into the host cell by transfecting the host cell with a mixture of AAV helper constructs either prior to, or concurrently with, the transfection of the AAV vector. In some embodiments, the AAV helper constructs are used to provide transient expression of AAV rep and/or cap genes. In some embodiments, the AAV helper constructs lack AAV packaging sequences and can neither replicate nor package themselves.

In some embodiments, an AAV genome can be cross-packaged with a heterologous virus. Cross-genera packing of the rAAV2 genome into the human bocavirus type 1 (HBoV1) capsid (rAAV2/HBoV1 hybrid vector), for example, results in a hybrid vector that is highly tropic for airway epithelium (Yan et al., 2013, Mol. Ther., 21:2181-94).

In some embodiments, the virus particles are lentivirus. In some embodiments, the lentiviral vector particle is Human Immunodeficiency Virus-1 (HIV-1).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740).

In some aspects, such viral vector particles contain viral nucleic acid, such as retroviral nucleic acid, for example lentiviral nucleic acid. In particular embodiments, the viral vector particle is replication defective. In some embodiments, the viral vector particle is a lentiviral vector.

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003. Exemplary methods for generating viral vectors including lentiviral vectors are described further below.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non- dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomycin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the payload gene. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T- based producer cell lines (e.g., Stewart et al., Hum Gene Ther. _2011, 2,2.(3):357˜369; Lee et al, Biotechnol Bioeng, 2012, 10996): 1551-1560; Throm et al., Blood. 2009, 113(21): 5104-5110).

In some aspects, the envelope proteins may be heterologous envelope protein from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Aiagoas virus (VSAV), Vesicular stomatitis Indiana virus (VSTV) and Vesicular stomatitis New Jersey virus (VSNJV) and/or stains provisionally classified in the vesiculovims genus as Grass carp rhabdovirus, BeAn 157575 virus (BeAn 157575), Boteke virus (BTKV), Calchaqui virus (CQFV), Eel virus American (EVA), Gray Lodge virus (GLOV), Jurona virus (JURY), Klamath virus (KLAVj. Kwatta virus (KWAV), La Joya virus (LJV), Malpais Spring virus (MSPV), Mount Elgon bat virus (MEB V), Ferine t virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedroviras (AcMNPV), Anagrapha falcifera nuclear polyhedrosis virus, Bombyx mori nuclear polyhedrosis virus, Choristoneura fiimiferana nucleopolyhedroviras, Orgyia pseudotsugata single capsid nuclear polyhedrosis virus, Epiphyas postvittana nucleopolyhedroviras, Hypharitria cunea nucleopolyhedroviras, Galleria mellonella nuclear polyhedrosis virus, Dhori virus, Thogoto virus, Antheraea pemyi nucleopolyhedroviras or Batken virus.

In some embodiments, the envelope protein may be a fusogen. Exemplary fusogens include paramyxovirus fusogens such as described below.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMI, FUGW, pWPXL, pWPI, pLenti CMV puro DEST, pLJMI-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725: 9,068,199: 9,023,646: 8,900,858: 8,748,169; 8,709,799; 8,420,104; 8,329,462; 8,076,106; 6,013,516; and 5,994, 136; International Patent Publication NO.: WO2012079000).

Other retroviral vectors also may be used to package a payload agent for delivery to a target cell. Retroviral vectors (RVs) allow the permanent integration of a transgene in target cells. In addition to lentiviral vectors based on complex HIV- 1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retro viral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xprl) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag- pol) polyprotein, one encoding the envelope (env) protein, and one encoding the vector mRNA comprising polynucleotide encoding the payload agent that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelope proteins include the gibbon ape leukemia vims envelope protein (GALV) or vesicular stomatitis virus G protein (VSV-G), or Simian endogenous retrovirus envelope protein, or Measles Virus H and F proteins, or Human immunodeficiency virus gp120 envelope protein, or cocal vesiculovirus envelope protein (See, e.g., U.S. application publication NO.: 2012/164118). In other aspects, envelope glycoproteins may be genetically modified to incorporate targeting/binding ligands into gamma-retroviral vectors, binding ligands including, but not limited to, peptide ligands, single chain antibodies and growth factors (Waehier et aL, Nat. Rev. Genet. 2007, 8(8):573-587). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand- receptor, avidin-biotin, and chemical conjugations, monoclonal antibodies and engineered fusogenic proteins, can direct the attachment of viral vectors to target cells for transduction (Yang et al, Biotechnol Bioeng., 2008, 101(2): 357-368; and Maetzig et al, Viruses, 2011, 3, 677-713).

Exemplary envelope proteins including fusogens retargeted with a target moiety for binding to a target cell are described below.

In some embodiments, the recombinant gamma-retroviral vectors are self-inactivating (SIN) gammaretroviral vectors. The vectors may be replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promoter of choice, and/or an enhancer element. The choice of the internal promoters may be made according to specific requirements of gene expression needed for a particular purpose.

In some embodiments, polynucleotides encoding the payload agent are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′- long terminal repeat (LTR), and/or 3′-SIN elements modified in the US- region of the 3 -LTR. These modifications may increase the titers and the ability of infection. Gamma retroviral vectors suitable for delivering the heterologous agent(s) (e.g. CAR and/or immunomodulator, such as a cytokine) may be selected from those disclosed in U.S. Pat. Nos. 8,828,718; 7,585,676; 7,351,585; U.S. application publication NO.: US2007/048285; PCT application publication NOs.: WO2010/113037; WO2014/121005; WO2015/056014; and EP Pat, NOs.: EP1757702; EP1757703).

### B. Virus-Like Particles

In some embodiments, the lipid particle is a virus-like particle. The VLPs include those derived from retroviruses or lentiviruses. While VLPs mimic native virion structure, they lack the viral genomic information necessary for independent replication within a host cell. Therefore, in some aspects, VLPs are non-infectious. In some embodiments, the VLP's bilayer of amphipathic lipids is or comprises the viral envelope. In some embodiments, the lipid particle's bilayer of amphipathic lipids is or comprises lipids derived from a cell. A VLP typically comprises at least one type of structural protein from a virus. In most cases this protein will form a proteinaceous capsid (e.g. VLPs comprising a lentivirus, adenovirus or paramyxovirus structural protein). In some cases the capsid will also be enveloped in a lipid bilayer originating from the cell from which the assembled VLP has been released (e.g. VLPs comprising a human immunodeficiency virus structural protein such as GAG). In some embodiments, the VLP is pseudotyped and/or further comprises a targeting moiety as an envelope protein within the lipid bilayer.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

Among VLPs are that are derived from virus, such as those derived from retroviruses or lentiviruses. In some embodiments, the viral particles are derived from paramyxoviruses. Thus, in some examples, the viral-like particle is derived from Nipah, Hendra, or Rubeola viruses.

Exemplary methods of producing paramyxovirus-based VLPs are disclosed in US2017/0175086.

### C. Non-Viral Vectors

In some embodiments, the nucleic acid encoding the payload gene is not comprised in a viral or virally derived vector. In some embodiments, synthetic or natural biodegradable agents may be used for delivery of a payload agent such as cationic lipids, lipid nano emulsions, nanoparticles, peptide based vectors, or polymer based vectors.

In some embodiments, the lipid particle is a non-viral vector. In some embodiments, the lipid particle comprises a naturally derived bilayer of amphipathic lipids. In some embodiments, the bilayer may be comprised of one or more lipids of the same or different type. In some embodiments, the lipids comprise phospholipids such as phosphocholines and phosphoinositols. In some embodiments, the lipids comprise DMPC, DOPC, and DSPC.

In some embodiments, the lipid particles contain a cationic lipid. Cationic lipids are amphiphilic molecules that have a cationic head group and a hydrophobic tail group connected by either stable or degradable linkages. Felgner and colleagues were the first to demonstrate the use of cationic lipids for DNA delivery in 1987 (Felgner et al. PNAS (84) 21:7413-7417, 1987). Many cationic lipids since then have been synthesized and evaluated for nucleic acid delivery, including for example GL67A.

In some embodiments, the payload agent such as a nucleic acid encoding the payload agent is incorporated in lipid nanoparticles. In some embodiments, the lipid particle is a lipid nanoparticle. In some embodiments, the formulation is a nanoparticle which may comprise at least one lipid. The lipid may be selected from, but is not limited to, DLin-DMA, DLin-K-DMA, 98N12- 5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC 3 -DMA, DLin-KC2-DMA and DODMA

Lipid nanoparticles can be used for the delivery of encapsulated or associated (e.g., complexed) therapeutic agents, including mRNA. In particular, some nanoparticle compositions are particularly useful for the delivery of nucleic acids including messenger RNA (mRNA), antisense oligonucleotide, plasmid DNA, microRNA (miRNA), miRNA inhibitors (antagomirs/antimers), messenger-RNA-interfering complementary RNA (micRNA), DNA, multivalent RNA, dicer substrate RNA, complementary DNA (cDNA), and self-amplifying RNA (saRNA). See, e.g., U.S. Pat. No. 10,723,692 B2.

LNPs particularly useful for in the present methods comprise a cationic lipid selected from DLin-DMA (1,2-dilinoleyloxy-3-dimethylaminopropane), DLin-MC3 -DM A (dilinoleylmethyl-4-dimethylaminobutyrate), DLin-KC2-DMA (2,2-dilinoleyl-4-(2- dimethylaminoethyl)-[1,3]-dioxolane), DODMA (1,2- dioleyloxy-N,N-dimethyl-3- aminopropane), SS-OP (Bis[2-(4-{2-[4-(cis-9 octadecenoyloxy)phenylacetoxy]ethyl}piperidinyl)ethyl]disulfide), and derivatives thereof. DLin-MC3-DMA and derivatives thereof are described, for example, in WO 2010144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at www. nofamerica.com/store/index.php?dispatch=products. view &product_id=962. Additional and non-limiting examples of cationic lipids include methylpyridiyl- dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2- dioleoyl-3-trimethylammonium-propane (DOTAP), Bis{2-[N-methyl-N-(a-D-tocopherolhemisuccinatepropyl)amino]ethyl}disulfide (SS-33/3AP05), Bis{2-[4-(a-D-tocopherolhemisuccinateethyl)piperidyl]ethyl}disulfide (SS33/4PE15), Bis{2-[4-(cis-9-octadecenoateethyl)-1-piperidinyl]ethyl}disulfide (SS18/4PE16), and Bis{2-[4-(cis,cis-9,12-octadecadienoateethyl)-1-piperidinyl]ethyl}disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to mRNA of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles utilized in the presently disclosed methods can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3- phosphocholine (DDPC), 1,2-Dierucoyl-sn-glycero-3-phosphate(Sodium Salt) (DEPA-NA), 1,2-Dierucoyl-sn-glycero-3-phosphocholine (DEPC), 1,2-Dierucoyl-sn-glycero-3- phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn- glycero-3-phosphate(Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn- glycero-3[Phospho-rac-(1-glycerol . . . )(Sodium Salt) (DLPG-NA), 1,2-Dilauroyl-sn-glycero- 3[Phospho-rac-(1-glycerol)(Ammonium Salt) (DLPG-NH4), 1,2-Dilauroyl-sn-glycero-3- phosphoserine(Sodium Salt) (DLPS-NA), 1,2-Dimyristoyl-sn-glycero-3-phosphate(SodiumSalt) (DMPA-NA), 1,2-Dimyristoyl-sn-glycero-3-phosphocholine (DMPC), 1,2-Dimyristoyl- sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn- glycero-3-phosphoethanolamine (DOPE), 1,2-Dioleoyl-sn-glycero-3[Phospho-rac-(1- glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2- Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero- 3[Phospho-rac-(1-glycerol)(Sodium Salt) (DPPG-NA), 1,2-Dipalmitoyl-sn-glycero- 3[Phospho-rac-(1-glycerol)(Ammonium Salt) (DPPG-NH4), 1,2-Dipalmitoyl-sn-glycero-3- phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2- Distearoyl-sn-glycero-3[Phospho-rac-(1-glycerol)(Sodium Salt) (DSPG-NA), 1,2-Distearoyl- sn-glycero-3[Phospho-rac-(1-glycerol)(Ammonium Salt) (DSPG-NH4), 1,2-Distearoyl-sn- glycero-3-phosphoserine(Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LY S OPCM YRIS TIC), 1-Palmitoyl-sn-glycero-3-phosphocholine (LYSOPCPALMITIC), 1- Stearoyl-sn-glycero-3-phosphocholine (LYSOPC STEARIC), 1-Myristoyl-2-palmitoyl-sn- glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2- oleoyl-sn-glycero-3-phosphocholine (POPC), 1-Palmitoyl-2-oleoyl-sn-glycero-3- phosphoethanolamine (POPE), 1-Palmitoyl-2-oleoyl-sn-glycero-3[Phospho-rac-(1- glycerol)](Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-oleoyl- sn-glycero-3-phosphocholine (SOPC), and 1-Stearoyl-2-palmitoyl-sn-glycero-3- phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)- dehydrocholesterol, 8(14)-dehydrocholesterol, diosgenin, DHEA sulfate, DHEA, lanosterol- d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG 1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG- PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG- PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG- PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells, and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and their method of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise an immune cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to target cells.

### D. Methods of Generating Viral-Based Particles

The provided viral-based particles include particles derived from a virus, such as viral particles or virus-like particles, including those derived from retroviruses or lentiviruses.

In some embodiments, the viral particle or virus-like particle is produced from virus family members comprising Parvoviridae (e.g. adeno-associated virus), Retroviridae (e.g. HIV), Flaviviridae (e.g. Hepatitis C virus), Paramyxoviridae (e.g. Nipah) and bacteriophages.

In some embodiments, the viral particle or virus-like particle is produced utilizing proteins (e.g., envelope proteins) from a virus within the Paramyxoviridae family. In some embodiments, the Paramyxoviridae family comprises members within the Henipavirus genus. In some embodiments, the Henipavirus is or comprises a Hendra (HeV) or a Nipah (NiV) virus. In particular embodiments, the viral particles or virus-like particles incorporate a fusogen, such as a targeted envelope protein and fusogen as described in Section IV.

In some embodiments, viral particles or virus-like particles may be produced in multiple cell culture systems including bacteria, mammalian cell lines, insect cell lines, yeast and plant cells.

In some embodiments, the assembly of a viral particle or virus-like particle is initiated by binding of the core protein to a unique encapsidation sequence within the viral genome (e.g. UTR with stem-loop structure). In some embodiments, the interaction of the core with the encapsidation sequence facilitates oligomerization.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, a viral vector comprises e.g., a virus or viral particle capable of transferring a nucleic acid into a cell, or to the transferred nucleic acid (e.g., as naked DNA). In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, the virus particle of viral-like particle, such as a retrovirus or retroviral-like particle (e.g. a lentivirus or lentiviral-like particle) is pseudotyped. In some examples, a pseudotyped virus of viral-like particle has a modification to one or more of its envelope proteins, e.g., an envelope protein is substituted with an envelope protein from another virus. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+presenting cells.

In some embodiments, retroviral envelope proteins, e.g. lentiviral envelope proteins, are pseudotyped with VSV-G. In one embodiment, source cells produce recombinant retrovirus or retrovirus-like particles, e.g., lentivirus or lentiviral-like particles, pseudotyped with the VSV-G envelope glycoprotein.

In one embodiment, source cells produce recombinant retrovirus or retrovirus-like particles, e.g., lentivirus or lentiviral-like particles, pseudotyped with the VSV-G envelope glycoprotein. In some embodiments, retroviral envelope proteins, e.g. lentiviral envelope proteins, are pseudotyped with an envelope glycoprotein G or H of a virus of the Paramyxoviridae family. In some embodiments, the virus of the Paramyxovirus family is a Henipavirus or is a Morbillivirus. In some aspects, the envelope glycoprotein a Nipah virus G (Niv-G) protein. In other aspects, the envelope glycoprotein is a Hendra virus G protein. In some embodiment, source cells produce recombinant retrovirus or retrovirus-like particles, e.g., lentivirus or lentiviral-like particles, pseudotyped with the envelope glycoprotein G or H of a virus of the Paramyxoviridae family.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV—I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease4 (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).

In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ]sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit P-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read- through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

In some embodiments, the retroviral vector systems described herein comprise viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. In some embodiments, by separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

In some embodiments, a viral vector particle which comprises a sequence that is devoid of or lacking viral RNA may be the result of removing or eliminating the viral RNA from the sequence. In one embodiment this may be achieved by using an endogenous packaging signal binding site on gag. In some embodiments, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles are used to deliver therapeutic RNA, in which case functional integrase and/or reverse transcriptase is not required. In some embodiments, the vector particles could also be used to deliver a therapeutic gene of interest, in which case pol is typically included.

In some embodiments, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

In some embodiments, an alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

When a recombinant plasmid and the retroviral LTR and packaging sequences are introduced into a producer cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a producer cell line, such as an exemplary HEK 293T cell line, by introduction of plasmids to allow generation of lentiviral particles. In some embodiments, a producer cell is transfected and/or contains a polynucleotide encoding gag and pol, and, in some cases, a polynucleotide encoding an exogenous agent. In some embodiments, the producer cell line is optionally and/or additionally transfected with and/or contains a polynucleotide encoding a rev protein. In some embodiments, the producer cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1

### E. Targeting and Retargeting of Lipid Particles

In some embodiments, the lipid particle further comprises a vector-surface targeting moiety which specifically binds to a target ligand on a target cell. It will be recognized by those skilled in the art that, the lipid particles provided herein harbor the attachment and/or fusion glycoproteins and are capable of binding to target cells and delivering the vehicle contents to the cytoplasm of the target cells. It will also be recognized by those skilled in the art that this is due to the natural viral entry mechanism that involves fusion of the viral membrane directly with the target cell plasma membrane.

It will further be recognized by those skilled in the art that many viruses such as paramyxoviruses bind to sialic acid receptors, and hence the corresponding derivative vehicles can deliver their contents generically to nearly any kind of cell that expresses sialic acid receptors. Other viruses such as Nipah virus and HIV bind to protein receptors, and hence the corresponding vehicles have a specificity that matches the natural tropisms for each virus and its surface proteins.

Furthermore, it will be recognized that technology exists to “re-target” attachment proteins, making it so that the vehicles only interact with particular cells or cell types that express a marker protein of interest (Msaouel et al., Meths Mol Biol 797: 141-162, 2012). Thus, vehicle surface glycoproteins proteins can be supplemented with or replaced by other targeting proteins, including but not necessarily limited to antibodies and antigen binding fragments thereof, receptor ligands, and other approaches that will be apparent to those skilled in the art given the benefit of the present disclosure. In some embodiments, the vector-surface targeting moiety is a polypeptide. In some embodiments, the polypeptide is a fusogen.

In some embodiments, the lipid particle comprises one or more fusogens. In some embodiments, the fusogen facilitates the fusion of the lipid particle to a cell membrane to delivery the payload gene into the cell. In some embodiments, the membrane is a plasma cell membrane. In some embodiments, the fusogen targets the lipid particle to a target cell of interest. In some embodiments, the fusogen contains a targeting moiety that provides retargeting (compared to the natural tropism of the fusogen) to the target cell of interest.

Provided herein are methods of administration of lipid particles (e.g. viral vectors) containing a fusogen disposed or embedded in the lipid bilayer Exemplary fusogens are described in subsections below. In some embodiments, the fusogen is composed of one or more Paramyxovirus envelope protein or a biologically active portion thereof. In some embodiments, the Paramyxovirus envelope protein or a biologically active portion thereof harbors the attachment and/or fusion glycoproteins and are capable of binding to target cells and delivering the vehicle contents to the cytoplasm of the target cells.

### F. Fusogens

In some embodiments, the lipid particles, such as viral vectors or viral-like particles, contain one or more fusogens. In some embodiments, the lipid particle, e.g. viral vector or viral-like particle, contains an exogenous or overexpressed fusogen. In some embodiments, the fusogen is disposed in the lipid bilayer. In some embodiments, the fusogen facilitates the fusion of the lipid particle to a membrane. In some embodiments, the membrane is a plasma cell membrane of a target cell. In some embodiments, the lipid particle, such as a viral or non-viral vector, comprising the fusogen integrates into the membrane into a lipid bilayer of a target cell. In some embodiments, the fusogen results in mixing between lipids in the lipid particle and lipids in the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the lipid particle, e.g. viral vector or viral-like particle, contain a first fusogen that is a protein fusogen and a second fusogen that is a lipid fusogen or chemical fusogen. In some embodiments, the fusogen binds a fusogen binding partner on a target cell surface. In some embodiments, the lipid particle is a viral vector or viral-like particle that is pseudotyped with the fusogen. In some examples, a virus of viral-like particle has a modification to one or more of its envelope proteins, e.g., an envelope protein is substituted with an envelope protein from another virus. In some embodiments, retroviral envelope proteins, e.g. lentiviral envelope proteins, are pseudotyped with a fusogen.

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

### 1. Mammalian Proteins

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

### 2. Viral Proteins

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of a-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of 0- sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and f sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is any of the fusogenic moieties described in WO2017/182585; WO2022/164935; WO2021/076788; Hamilton et al. bioRxiv 2022.08.24.505004; Nikolic et al. Nat Commun 9, 1029 (2018); Dobson et al. Nat. Methods. 19, 449-460 (2022); and Yu et al. bioRxiv 2021.12.13.472464, for instance any of the VSV or variant VSV glycoproteins described therein, such as VSV glycoproteins that have reduced binding to native receptors.

In some embodiments, the fusogen is a poxviridae fusogen.

In some embodiments the fusogen is a paramyxovirus fusogen. In some embodiments, the fusogen may be an envelope glycoprotein G, H HN and/or an F protein of the Paramyxoviridae family. In some embodiments the fusogen contains a Nipah virus protein F, a measles virus F protein, a Tupaia paramyxovirus F protein, a paramyxovirus F protein, a Hendra virus F protein, a Henipavirus F protein, a Morbillivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the fusogen is a F and/or HN protein from the murine parainfluenza virus type 1 (See e.g.., U.S. patent Ser. No. 10/704,061).

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a canine distemper virus F protein, a Tupaia paramyxovirus F protein, a paramyxovirus F protein, a Hendra virus F protein, a Henipavirus F protein, a Morbillivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In some embodiments, the lipid particle (e.g. viral vector) is pseudotyped with viral glycoproteins as described herein such as a NiV—F and/or NiV-G protein.

In some embodiments, the viral vector further comprises a vector-surface targeting moiety which specifically binds to a target ligand. In some embodiments, the vector-surface targeting moiety is a polypeptide. In some embodiments, the nucleic acid encoding the one of the Paramyxovirus envelope protein (e.g. G protein) is modified with a targeting moiety to specifically bind to a target molecule on a target cells. In some embodiments, the targeting moiety can be any targeting protein, including but not necessarily limited to antibodies and antigen binding fragments thereof.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g. lentiviral vector), the F protein is heterologous to the G protein, i.e. the F and G protein or biologically active portions are from different henipavirus species. For example, the G protein is from Hendra virus and the F protein is a NiV-F as described. In other aspects, the F and/or G protein can be a chimeric F and/or G protein containing regions of F and/or G proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F and/or G protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus.

a. F Proteins

In some embodiments, the lipid particles (e.g. lentiviral vectors) comprises a protein with a hydrophobic fusion peptide domain. In some embodiments, the protein with a hydrophobic fusion peptide domain may be an envelope glycoprotein F protein of the Paramyxoviridae family (i.e., a paramyxovirus F protein). In some embodiments, the envelope glycoprotein F protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mojiang virus F protein or a bat Paramyxovirus F protein or a biologically active portion thereof.

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mójiāng virus F protein or a bat Paramyxovirus F protein or a biologically active portion thereof.

F proteins of henipaviruses are encoded as F₀ precursors containing a signal peptide. Following cleavage of the signal peptide, the mature F₀ is transported to the cell surface, then endocytosed and cleaved by cathepsin L into the mature fusogenic subunits F1 and F2. For instance, with reference to NiV-F the NiV-F is encoded as F0 precursors containing a signal peptide (e.g. corresponding to amino acid residues 1-26 of the below). Following cleavage of the signal peptide, the mature F0 (SEQ ID NO:2 lacking the signal peptide, i.e. set forth in SEQ ID NO:7) is transported to the cell surface, then endocytosed and cleaved by cathepsin L (e.g. between amino acids 109-110 of NiV-F corresponding to amino acids set forth in SEQ ID NO:2) into the mature fusogenic subunits F1 (e.g. corresponding to amino acids 110-546 of NiV-F set forth in SEQ ID NO:2) and F2 (e.g. corresponding to amino acid residues 27-109 of NiV-F set forth in SEQ ID NO:2). The F1 and F2 subunits are associated by a disulfide bond and recycled back to the cell surface. The F1 subunit contains the fusion peptide domain located at the N terminus of the F1 subunit (e.g. corresponding to amino acids 110-129 of the below e.g. NiV-F set forth in SEQ ID NO:2), where it is able to insert into a cell membrane to drive fusion. Without wishing to be bound by theory, in some aspects, fusion is blocked by association of the F protein with G protein, until the G protein engages with a target molecule resulting in its disassociation from F and exposure of the fusion peptide to mediate membrane fusion.

In some embodiments, the F protein (e.g. NiV-F protein) of the lipid particle, such as lentiviral vector, exhibits fusogenic activity. In some embodiments, the F protein (e.g. NiV-F) facilitates the fusion of the lipid particle (e.g. lentiviral vector) to a membrane. In particular embodiments, the F protein or the functionally active variant or biologically active portion thereof retains fusogenic activity in conjunction with a Henipavirus G protein, such as a G protein set forth below. Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L(e.g. corresponding to the cleavage site between amino acids 109-110 of SEQ ID NO:2).

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

Table 2A provides non-limiting examples of F proteins. In some embodiments, the N-terminal hydrophobic fusion peptide domain of the F protein molecule or biologically active portion thereof is exposed on the outside of lipid bilayer.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects of the provided lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 2A
Non-limiting Examples of F Proteins

| | | | | | SEQ ID |
|---|---|---|---|---|---|
| | Nucleo- | | | | NO |
| Gen | tides | | | | (without |
| bank | of | Full | | SEQ | signal |
| ID | CDS | Gene Name | Sequence | ID | sequence) |
| AF017149 | 6618- | gb: AF017149\| | MATQEVRLKCLLCGIIVLVLSLEGLGILH | 1 | 6 |
| | 8258 | Organism: | YEKLSKIGLVKGITRKYKIKSNPLTKDIVI | | |
| | | Hendra virus\| | KMIPNVSNVSKCTGTVMENYKSRLTGILS | | |
| | | Strain | PIKGAIELYNNNTHDLVGDVKLAGVVMA | | |
| | | Name: | GIAIGIATAAQITAGVALYEAMKNADNIN | | |
| | | UNKNOWN- | KLKSSIESTNEAVVKLQETAEKTVYVLTA | | |
| | | AF017149\| | LQDYINTNLVPTIDQISCKQTELALDLALS | | |
| | | Protein | KYLSDLLFVFGPNLQDPVSNSMTIQAISQ | | |
| | | Name: | AFGGNYETLLRTLGYATEDFDDLLESDSI | | |
| | | fusion\| | AGQIVYVDLSSYYIIVRVYFPILTEIQQAY | | |
| | | Gene | VQELLPVSFNNDNSEWISIVPNFVLIRNTL | | |
| | | Symbol: | ISNIEVKYCLITKKSVICNQDYATPMTAS | | |
| | | F | VRECLTGSTDKCPRELVVSSHVPRFALSG | | |
| | | | GVLFANCISVTCQCQTTGRAISQSGEQTL | | |
| | | | LMIDNTTCTTVVLGNIIISLGKYLGSINYN | | |
| | | | SESIAVGPPVYTDKVDISSQISSMNQSLQQ | | |
| | | | SKDYIKEAQKILDTVNPSLISMLSMIILYV | | |
| | | | LSIAALCIGLITFISFVIVEKKRGNYSRLDD | | |
| | | | RQVRPVSNGDLYYIGT | | |
| Q9IH63 | | Additional | MVVILDKRCYCNLLILILMISECSVGILHY | 2 | 7 |
| | | in cluster: | EKLSKIGLVKGVTRKYKIKSNPLTKDIVIK | | |
| | | sp/ | MIPNVSNMSQCTGSVMENYKTRLNGILT | | |
| | | Q9IH63\| | PIKGALEIYKNNTHDLVGDVRLAGVIMA | | |
| | | FUS_NIPAV | GVAIGIATAAQITAGVALYEAMKNADNI | | |
| | | Fusion | NKLKSSIESTNEAVVKLQETAEKTVYVLT | | |
| | | glycoprotein | ALQDYINTNLVPTIDKISCKQTELSLDLAL | | |
| | | F0 | SKYLSDLLFVFGPNLQDPVSNSMTIQAIS | | |
| | | OS = | QAFGGNYETLLRTLGYATEDFDDLLESD | | |
| | | Nipah virus | SITGQIIYVDLSSYYIIVRVYFPILTEIQQA | | |
| | | | YIQELLPVSFNNDNSEWISIVPNFILVRNT | | |
| | | | LISNIEIGFCLITKRSVICNQDYATPMTNN | | |
| | | | MRECLTGSTEKCPRELVVSSHVPRFALSN | | |
| | | | GVLFANCISVTCQCQTTGRAISQSGEQTL | | |
| | | | LMIDNTTCPTAVLGNVIISLGKYLGSVNY | | |
| | | | NSEGIAIGPPVFTDKVDISSQISSMNQSLQ | | |
| | | | QSKDYIKEAQRLLDTVNPSLISMLSMIILY | | |
| | | | VLSIASLCIGLITFISFIIVEKKRNTYSRLED | | |
| | | | RRVRPTSSGDLYYIGT | | |
| JQ001776 | 6129- | gb: JQ001776: | MSNKRTTVLIIISYTLFYLNNAAIVGFDFD | 3 | 8 |
| | 8166 | 6129-8166\| | KLNKIGVVQGRVLNYKIKGDPMTKDLVL | | |
| | | Organism: | KFIPNIVNITECVREPLSRYNETVRRLLLPI | | |
| | | Cedar virus\| | HNMLGLYLNNTNAKMTGLMIAGVIMGG | | |
| | | Strain | IAIGIATAAQITAGFALYEAKKNTENIQKL | | |
| | | Name: | TDSIMKTQDSIDKLTDSVGTSILILNKLQT | | |
| | | CG1a\| | YINNQLVPNLELLSCRQNKIEFDLMLTKY | | |
| | | Protein | LVDLMTVIGPNINNPVNKDMTIQSLSLLF | | |
| | | Name: | DGNYDIMMSELGYTPQDFLDLIESKSITG | | |
| | | fusion | QIIYVDMENLYVVIRTYLPTLIEVPDAQIY | | |
| | | glycoprotein\| | EFNKITMSSNGGEYLSTIPNFILIRGNYMS | | |
| | | Gene | NIDVATCYMTKASVICNQDYSLPMSQNL | | |
| | | Symbol: | RSCYQGETEYCPVEAVIASHSPRFALTNG | | |
| | | F | VIFANCINTICRCQDNGKTITQNINQFVSM | | |
| | | | IDNSTCNDVMVDKFTIKVGKYMGRKDIN | | |
| | | | NINIQIGPQIIIDKVDLSNEINKMNQSLKDS | | |
| | | | IFYLREAKRILDSVNISLISPSVQLFLIIISVL | | |
| | | | SFIILLIIIVYLYCKSKHSYKYNKFIDDPDY | | |
| | | | YNDYKRERINGKASKSNNIYYVGD | | |
| NC_ | 5950- | gb: | MALNKNMFSSLFLGYLLVYATTVQSSIH | 4 | 9 |
| 025352 | 8712 | NC_025352: | YDSLSKVGVIKGLTYNYKIKGSPSTKLM | | |
| | | 5950-8712\| | VVKLIPNIDSVKNCTQKQYDEYKNLVRK | | |
| | | Organism: | ALEPVKMAIDTMLNNVKSGNNKYRFAG | | |
| | | Mojiang | AIMAGVALGVATAATVTAGIALHRSNEN | | |
| | | virus\| | AQAIANMKSAIQNTNEAVKQLQLANKQT | | |
| | | Strain | LAVIDTIRGEINNNIIPVINQLSCDTIGLSV | | |
| | | Name: | GIRLTQYYSEIITAFGPALQNPVNTRITIQA | | |
| | | Tongguan 1\| | ISSVFNGNFDELLKIMGYTSGDLYEILHSE | | |
| | | Protein | LIRGNIIDVDVDAGYIALEIEFPNLTLVPN | | |
| | | Name: | AVVQELMPISYNIDGDEWVTLVPRFVLT | | |
| | | fusion | RTTLLSNIDTSRCTITDSSVICDNDYALPM | | |
| | | protein\| | SHELIGCLQGDTSKCAREKVVSSYVPKFA | | |
| | | Gene | LSDGLVYANCLNTICRCMDTDTPISQSLG | | |
| | | Symbol: | ATVSLLDNKRCSVYQVGDVLISVGSYLG | | |
| | | F | DGEYNADNVELGPPIVIDKIDIGNQLAGI | | |
| | | | NQTLQEAEDYIEKSEEFLKGVNPSIITLGS | | |
| | | | MVVLYIFMILIAIVSVIALVLSIKLTVKGN | | |
| | | | VVRQQFTYTQHVPSMENINYVSH | | |
| NC_ | 6865- | gb: | MKKKTDNPTISKRGHNHSRGIKSRALLRE | 5 | 10 |
| 025256 | 8853 | NC025256: | TDNYSNGLIVENLVRNCHHPSKNNLNYT | | |
| | | 6865-8853\| | KTQKRDSTIPYRVEERKGHYPKIKHLIDK | | |
| | | Organism: | SYKHIKRGKRRNGHNGNIITIILLLILILKT | | |
| | | Bat | QMSEGAIHYETLSKIGLIKGITREYKVKG | | |
| | | Paramyxovirus | TPSSKDIVIKLIPNVTGLNKCTNISMENYK | | |
| | | Eid_hel/ | EQLDKILIPINNIIELYANSTKSAPGNARFA | | |
| | | GH-M74a/GHA/ | GVIIAGVALGVAAAAQITAGIALHEARQN | | |
| | | 2009\| | AERINLLKDSISATNNAVAELQEATGGIV | | |
| | | Strain | NVITGMQDYINTNLVPQIDKLQCSQIKTA | | |
| | | Name: | LDISLSQYYSEILTVFGPNLQNPVTTSMSI | | |
| | | BatPV/Eid_ | QAISQSFGGNIDLLLNLLGYTANDLLDLL | | |
| | | hel/GH- | ESKSITGQITYINLEHYFMVIRVYYPIMTTI | | |
| | | M74a/GHA/ | SNAYVQELIKISFNVDGSEWVSLVPSYILI | | |
| | | 2009\| | RNSYLSNIDISECLITKNSVICRHDFAMPM | | |
| | | Protein | SYTLKECLTGDTEKCPREAVVTSYVPRFA | | |
| | | Name: | ISGGVIYANCLSTTCQCYQTGKVIAQDGS | | |
| | | fusion | QTLMMIDNQTCSIVRIEEILISTGKYLGSQ | | |
| | | protein\| | EYNTMHVSVGNPVFTDKLDITSQISNINQ | | |
| | | Gene | SIEQSKFYLDKSKAILDKINLNLIGSVPISI | | |
| | | Symbol: | LFIIAILSLILSIITFVIVMIIVRRYNKYTPLI | | |
| | | F | NSDPSSRRSTIQDVYIIPNPGEHSIRSAARS | | |
| | | | IDRDRD | | |

In some embodiments, the F protein or the biologically active portion thereof is a wild-type Nipah virus F (NiV-F) protein or a Hendra virus F protein or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO:1, SEQ ID NO:2, SEQ ID NO:3, SEQ ID NO:4, or SEQ ID NO:5, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, or SEQ ID NO:5, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, or SEQ ID NO:5.

In particular embodiments, the F protein has the sequence of amino acids set forth in SEQ ID NO:6, SEQ ID NO:7, SEQ ID NO:8, SEQ ID NO:9, or SEQ ID NO:10, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:6, SEQ ID NO:7, SEQ ID NO:8, SEQ ID NO:9, or SEQ ID NO:1, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:6, SEQ ID NO:7, SEQ ID NO:8, SEQ ID NO:9, or SEQ ID NO:1.

Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:2).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (HeV) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the NiV-F, of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:2) to produce two chains that can be linked by disulfide bond. In some embodiments, the NiV-F, is produced or encoded as an F₀ precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the F₀ precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:2 or truncated or mutant sequence thereof, and an F2 corresponding to amino acid residues 27-109 of NiV-F set forth in SEQ ID NO:2.

In some embodiments, the mutant F protein is a biologically active portion that is truncated and lacks up to 22 contiguous amino acid residues at or near the C-terminus of the wild-type NiV-F protein, such as a wild-type NiV-F protein set forth in SEQ ID NO:2 or SEQ ID NO:7. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO:2 or SEQ ID NO:7. In some embodiments, the mutant F protein contains an F1 subunit and an F2 subunit in which (1) the F1 subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:2, and (2) the F2 subunit has the sequence corresponding to amino acid residues 27-109 of NiV-F set forth in SEQ ID NO:2.

In some embodiments, the F protein is a mutant NiV-F protein that is a biologically active portion thereof that comprises a 22 amino acid truncation at or near the C-terminus of the wild-type NiV-F protein (SEQ ID NO:2 or SEQ ID NO:7). In some embodiments, the NiV-F protein is encoded by a nucleotide sequence that encodes the sequence set forth in SEQ ID NO: 11. In some embodiments, the NiV-F protein has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11. In particular embodiments, the F protein is a mutant NiV-F protein that has the sequence of amino acids set forth in SEQ ID NO:12. In some embodiments, the NiV-F protein has a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12. In some embodiments, the F protein molecule or biologically active portion thereof comprises the sequence set forth in SEQ ID NO: 12.

In some embodiments, the mutant F protein contains an F1 subunit and an F2 subunit in which (1) the F1 subunit is set forth as amino acids 110-524 of SEQ ID NO:11, and (2) the F2 subunit is set forth as amino acids 27-109 of SEQ ID NO:11.

In some embodiments, the mutant F protein contains an F1 subunit and an F2 subunit in which (1) the F1 subunit is set forth as amino acids 84-498 of SEQ ID NO:12, and (2) the F2 subunit is set forth as amino acids 1-83 of SEQ ID NO:12. b. G/H Proteins

In some embodiments, the one or more paramyxovirus fusogen includes a paramyxovirus attachment glycoprotein (e.g. G protein). Paramyxoviral attachment proteins are type II transmembrane glycoproteins that are designated as hemagglutinin-neuraminidase (HN), hemagglutinin (H), or glycoproteins (G), depending on two characteristics; the ability to agglutinate erythrocytes (hemagglutination) and the presence or absence of neuraminidase activity (cleavage of sialic acid). Specifically, the HN attachment glycoprotein is characteristic of the Avulavirus, Respirovirus, and Rubulavirus genera, the H attachment glycoproteins are found in members of the Morbillivirus genus, while the G attachment glycoproteins are utilized by the viruses of the genus Henipavirus and the Pneumovirinae subfamily. The geometries of HN, H, or G glycoproteins possess high structural similarity, however although H and G glycoproteins are capable of recognizing protein receptors, they lack neuraminidase activity.

Paramyxoviral attachment glycoproteins contain a short N-terminal cytoplasmic tail, a transmembrane domain, and an extracellular domain containing an extracellular stalk and a globular head. The N-terminal cytoplasmic domain is within the inner lumen of the lipid bilayer and the C-terminal portion is the extracellular domain that is exposed on the outside of the lipid bilayer. The receptor binding and antigenic sites reside on the extracellular domain. Regions of the stalk in the C-terminal region have been shown to be involved in interactions with the F protein and triggering of fusion with a target cell membrane (Liu et al. 2015 J of Virology 89:1838). The F protein undergoes significant conformational change that facilitates the insertion of the fusion peptide into target membranes, bringing the two HR regions together in the formation of a six-helix bundle structure or trimer-of-hairpins during or immediately following fusion of virus and cell membranes (Bishop et al. 2008. J of Virology 82(22): 11398-11409). The cytoplasmic tails play a role in particle formation, incorporation into packaged particles, and serves as a signal peptide to modulate protein maturation and surface transport (Sawatsky et al. 2016. J of Virology 97:1066-1076).

In some embodiments, any of the provided lipid particles (lentiviral vectors) that contains a paramyxovirus attachment glycoprotein (e.g. G protein, such as NiV-G) or a biologically active portion thereof may also contain an F protein, such as a NiV-F protein, such as a full-length NiV-F protein or a biologically active portion thereof or a variant thereof.

In some embodiments, the envelope protein contains a henipavirus envelope attachment glycoprotein G (G protein) or a biologically active portion thereof. In some embodiments, the G protein may be retargeted by linkage to a targeting moiety, such as a binding molecule (e.g. antibody or antigen-binding fragment, e.g. sdAb or scFv) that binds to a target cell. In some embodiments, the G protein and the NiV-F protein provided herein together exhibit fusogenic activity to a target cell, such as to deliver an exogenous agent or nucleic acid exogenous agent to the target cell.

The attachment G proteins are type II transmembrane glycoproteins containing an N-terminal cytoplasmic tail (e.g. corresponding to amino acids 1-49 of SEQ ID NO:14), a transmembrane domain (e.g. corresponding to amino acids 50-70 of SEQ ID NO:14), and an extracellular domain containing an extracellular stalk (e.g. corresponding to amino acids 71-187 of SEQ ID NO:14), and a globular head (corresponding to amino acids 188-602 of SEQ ID NO:14). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 14) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described, a lipid particle can contain heterologous G and F proteins from different species. In particular embodiments, the F protein or the functionally active variant or biologically active portion thereof retains fusogenic activity in conjunction with a G protein as provided, such as any set forth below. Fusogenic activity includes the activity of the F protein in conjunction with a G protein to promote or facilitate fusion of two membrane lumens, such as the lumen of the lipid particle provided herein (e.g. having embedded in its lipid bilayer, such as exposed on its surface, a G protein and a F protein), and a cytoplasm of a target cell, e.g. a cell that contains a surface receptor or molecule that is recognized or bound by the G protein.

Exemplary Henipavirus protein G sequences are provided in Table 3

TABLE 3
Henipavirus protein G sequence clusters. Column 1, Genbank ID includes the Genbank
ID of the whole genome sequence of the virus that is the centroid sequence of the
cluster. Column 2, nucleotides of CDS provides the nucleotides corresponding to the
CDS of the gene in the whole genome. Column 3, Full Gene Name, provides the full
name of the gene including Genbank ID, virus species, strain, and protein name.
Column 4, Sequence, provides the amino acid sequence of the gene. Column 5,
#Sequences/Cluster, provides the number of sequences that cluster with this centroid
sequence. Column 6 provides the SEQ ID numbers for the described sequences.

| | Nucleo- | | | SEQ |
|---|---|---|---|---|
| Genbank | tides | Full | | ID |
| ID | of CDS | sequence ID | Sequence | NO |
| AF017149 | 8913- | gb: AF017149\| | MMADSKLVSLNNNLSGKIKDQGKVIKNY | 13 |
| | 10727 | Organism: | YGTMDIKKINDGLLDSKILGAFNTVIALL | |
| | | Hendra virus\| | GSIIIIVMNIMIIQNYTRTTDNQALIKESLQ | |
| | | Strain | SVQQQIKALTDKIGTEIGPKVSLIDTSSTIT | |
| | | Name: | IPANIGLLGSKISQSTSSINENVNDKCKFTL | |
| | | UNKNOWN- | PPLKIHECNISCPNPLPFREYRPISQGVSDL | |
| | | AF017149\| | VGLPNQICLQKTTSTILKPRLISYTLPINTR | |
| | | Protein | EGVCITDPLLAVDNGFFAYSHLEKIGSCT | |
| | | Name: | RGIAKQRIIGVGEVLDRGDKVPSMFMTN | |
| | | glycoprotein\|Gene | VWTPPNPSTIHHCSSTYHEDFYYTLCAVS | |
| | | Symbol: G | HVGDPILNSTSWTESLSLIRLAVRPKSDSG | |
| | | | DYNQKYIAITKVERGKYDKVMPYGPSGI | |
| | | | KQGDTLYFPAVGFLPRTEFQYNDSNCPII | |
| | | | HCKYSKAENCRLSMGVNSKSHYILRSGL | |
| | | | LKYNLSLGGDIILQFIEIADNRLTIGSPSKI | |
| | | | YNSLGQPVFYQASYSWDTMIKLGDVDTV | |
| | | | DPLRVQWRNNSVISRPGQSQCPRFNVCPE | |
| | | | VCWEGTYNDAFLIDRLNWVSAGVYLNS | |
| | | | NQTAENPVFAVFKDNEILYQVPLAEDDT | |
| | | | NAQKTITDCFLLENVIWCISLVEIYDTGDS | |
| | | | VIRPKLFAVKIPAQCSES | |
| AF212302 | 8943- | gb: AF212302\| | MPAENKKVRFENTTSDKGKIPSKVIKSYY | 14 |
| | 10751 | Organism: | GTMDIKKINEGLLDSKILSAFNTVIALLGS | |
| | | Nipah virus\|Strain | IVIIVMNIMIIQNYTRSTDNQAVIKDALQG | |
| | | Name: | IQQQIKGLADKIGTEIGPKVSLIDTSSTITIP | |
| | | UNKNOWN- | ANIGLLGSKISQSTASINENVNEKCKFTLP | |
| | | AF212302\| | PLKIHECNISCPNPLPFREYRPQTEGVSNL | |
| | | Protein | VGLPNNICLQKTSNQILKPKLISYTLPVVG | |
| | | Name: | QSGTCITDPLLAMDEGYFAYSHLERIGSC | |
| | | attachment | SRGVSKQRIIGVGEVLDRGDEVPSLFMTN | |
| | | glycoprotein\| | VWTPPNPNTVYHCSAVYNNEFYYVLCA | |
| | | Gene Symbol: G | VSTVGDPILNSTYWSGSLMMTRLAVKPK | |
| | | | SNGGGYNQHQLALRSIEKGRYDKVMPY | |
| | | | GPSGIKQGDTLYFPAVGFLVRTEFKYNDS | |
| | | | NCPITKCQYSKPENCRLSMGIRPNSHYILR | |
| | | | SGLLKYNLSDGENPKVVFIEISDQRLSIGS | |
| | | | PSKIYDSLGQPVFYQASFSWDTMIKFGDV | |
| | | | LTVNPLVVNWRNNTVISRPGQSQCPRFNT | |
| | | | CPEICWEGVYNDAFLIDRINWISAGVFLD | |
| | | | SNQTAENPVFTVFKDNEILYRAQLASEDT | |
| | | | NAQKTITNCFLLKNKIWCISLVEIYDTGD | |
| | | | NVIRPKLFAVKIPEQCT | |
| JQ001776 | 8170- | gb: JQ001776: | MLSQLQKNYLDNSNQQGDKMNNPDKKL | 15 |
| | 10275 | 8170-10275\| | SVNFNPLELDKGQKDLNKSYYVKNKNY | |
| | | Organism: | NVSNLLNESLHDIKFCIYCIFSLLIIITIINIIT | |
| | | Cedar virus\| | ISIVITRLKVHEENNGMESPNLQSIQDSLSS | |
| | | Strain | LTNMINTEITPRIGILVTATSVTLSSSINYV | |
| | | Name: CG1a\| | GTKTNQLVNELKDYITKSCGFKVPELKLH | |
| | | Protein | ECNISCADPKISKSAMYSTNAYAELAGPP | |
| | | Name: | KIFCKSVSKDPDFRLKQIDYVIPVQQDRSI | |
| | | attachment | CMNNPLLDISDGFFTYIHYEGINSCKKSDS | |
| | | glycoprotein\| | FKVLLSHGEIVDRGDYRPSLYLLSSHYHP | |
| | | Gene Symbol: G | YSMQVINCVPVTCNQSSFVFCHISNNTKT | |
| | | | LDNSDYSSDEYYITYFNGIDRPKTKKIPIN | |
| | | | NMTADNRYIHFTFSGGGGVCLGEEFIIPV | |
| | | | TTVINTDVFTHDYCESFNCSVQTGKSLKE | |
| | | | ICSESLRSPTNSSRYNLNGIMIISQNNMTD | |
| | | | FKIQLNGITYNKLSFGSPGRLSKTLGQVL | |
| | | | YYQSSMSWDTYLKAGFVEKWKPFTPNW | |
| | | | MNNTVISRPNQGNCPRYHKCPEICYGGT | |
| | | | YNDIAPLDLGKDMYVSVILDSDQLAENPE | |
| | | | ITVFNSTTILYKERVSKDELNTRSTTTSCF | |
| | | | LFLDEPWCISVLETNRFNGKSIRPEIYSYKI | |
| | | | PKYC | |
| NC_ | 9117- | gb: NC_025256: | MPQKTVEFINMNSPLERGVSTLSDKKTLN | 16 |
| 025256 | 11015 | 9117-11015\| | QSKITKQGYFGLGSHSERNWKKQKNQND | |
| | | Organism: | HYMTVSTMILEILVVLGIMENLIVLTMVY | |
| | | Bat | YQNDNINQRMAELTSNITVLNLNLNQLT | |
| | | Paramyxovirus | NKIQREIIPRITLIDTATTITIPSAITYILATL | |
| | | Eid_hel/GH- | TTRISELLPSINQKCEFKTPTLVLNDCRINC | |
| | | M74a/GHA/2009\| | TPPLNPSDGVKMSSLATNLVAHGPSPCRN | |
| | | Strain | FSSVPTIYYYRIPGLYNRTALDERCILNPR | |
| | | Name: BatPV/ | LTISSTKFAYVHSEYDKNCTRGFKYYELM | |
| | | Eid_hel/GH- | TFGEILEGPEKEPRMFSRSFYSPTNAVNY | |
| | | M74a/GHA/ | HSCTPIVTVNEGYFLCLECTSSDPLYKAN | |
| | | 2009\|Protein | LSNSTFHLVILRHNKDEKIVSMPSFNLSTD | |
| | | Name: | QEYVQIIPAEGGGTAESGNLYFPCIGRLLH | |
| | | glycoprotein\|Gene | KRVTHPLCKKSNCSRTDDESCLKSYYNQ | |
| | | Symbol: G | GSPQHQVVNCLIRIRNAQRDNPTWDVITV | |
| | | | DLTNTYPGSRSRIFGSFSKPMLYQSSVSW | |
| | | | HTLLQVAEITDLDKYQLDWLDTPYISRPG | |
| | | | GSECPFGNYCPTVCWEGTYNDVYSLTPN | |
| | | | NDLFVTVYLKSEQVAENPYFAIFSRDQIL | |
| | | | KEFPLDAWISSARTTTISCFMFNNEIWCIA | |
| | | | ALEITRLNDDIIRPIYYSFWLPTDCRTPYP | |
| | | | HTGKMTRVPLRSTYNY | |
| NC_ | 8716- | gb: NC_025352: | MATNRDNTITSAEVSQEDKVKKYYGVET | 17 |
| 025352 | 11257 | 8716-11257\| | AEKVADSISGNKVFILMNTLLILTGAIITIT | |
| | | Organism: | LNITNLTAAKSQQNMLKIIQDDVNAKLE | |
| | | Mojiang virus\| | MFVNLDQLVKGEIKPKVSLINTAVSVSIP | |
| | | Strain | GQISNLQTKFLQKYVYLEESITKQCTCNP | |
| | | Name: | LSGIFPTSGPTYPPTDKPDDDTTDDDKVD | |
| | | Tongguan 1\|Protein | TTIKPIEYPKPDGCNRTGDHFTMEPGANF | |
| | | Name: | YTVPNLGPASSNSDECYTNPSFSIGSSIYM | |
| | | attachment | FSQEIRKTDCTAGEILSIQIVLGRIVDKGQ | |
| | | glycoprotein\| | QGPQASPLLVWAVPNPKIINSCAVAAGDE | |
| | | Gene Symbol: G | MGWVLCSVTLTAASGEPIPHMFDGFWLY | |
| | | | KLEPDTEVVSYRITGYAYLLDKQYDSVFI | |
| | | | GKGGGIQKGNDLYFQMYGLSRNRQSFKA | |
| | | | LCEHGSCLGTGGGGYQVLCDRAVMSFGS | |
| | | | EESLITNAYLKVNDLASGKPVIIGQTFPPS | |
| | | | DSYKGSNGRMYTIGDKYGLYLAPSSWNR | |
| | | | YLRFGITPDISVRSTTWLKSQDPIMKILST | |
| | | | CTNTDRDMCPEICNTRGYQDIFPLSEDSE | |
| | | | YYTYIGITPNNGGTKNFVAVRDSDGHIAS | |
| | | | IDILQNYYSITSATISCFMYKDEIWCIAITE | |
| | | | GKKQKDNPQRIYAHSYKIRQMCYNMKS | |
| | | | ATVTVGNAKNITIRRY | |

In some embodiments, the G protein has a sequence set forth in any of SEQ ID NOS: 14, 13, 15, 16 or 17 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 14, 13, 15, 16 or 17.

In particular embodiments, the G protein or functionally active variant or biologically active portion is a protein that retains fusogenic activity in conjunction with a Henipavirus F protein, such as a NiV-F protein described herein. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F).

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 14, 13, 15, 16 or 17.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOS: 14, 13, 15, 16 or 17. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:14, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:14.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 10 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 15 contiguous amino acid residues at or near the N-terminus of the wild-type NiV-G protein, such as compared to wild-type NiV-G set forth in SEQ ID NO: 14. n some embodiments, the mutant NiV-G protein is truncated and lacks up to 20 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 25 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 30 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 35 contiguous amino acid residues at or near the N-terminus of the wild-type NiV-G protein, such as compared to wild-type NiV-G set forth in SEQ ID NO: 14. In some embodiments, the mutant NiV-G protein (also called variant NiV-G) contains an N-terminal methionine.

In some embodiments, the mutant NiV-G protein is truncated and lacks up to amino acid 34 at or near the N-terminus of the wild-type NiV-G protein, such as compared to wild-type NiV-G set forth in SEQ ID NO: 14. In some embodiments, the mutant NiV-G protein (also called variant NiV-G) contains an N-terminal methionine. In some embodiments, the mutant NiV-G protein lacks amino acids 2-34 as compared to wild-type NiV-G set forth in SEQ ID NO: 14.

In some embodiments, the G protein or the functionally active variant or biologically active portion thereof binds to Ephrin B2 or Ephrin B3. In some embodiments, the G protein is a mutant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 14, 13, 15, 16 or 17., such as at least 50%, at least 60%, at least 70%, at least 80% or at least 90% of the binding of the wild-type G protein.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such as reduce the binding to at least one of Ephrin B2 or Ephrin B3. In some embodiments, the mutations described herein interfere with natural receptor recognition.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:14. In some embodiments, the G protein is a mutant G protein containing one or more amino acid substitutions selected from the group consisting of E501A, W504A, Q530A and E533A with reference to numbering set forth in SEQ ID NO:14. In some embodiments, the G protein is a mutant G protein that contains one or more amino acid substitutions elected from the group consisting of E501A, W504A, Q530A and E533A with reference to SEQ ID NO:14 and is a biologically active portion thereof containing an N-terminal truncation.

In particular embodiments, the G protein has the sequence of amino acids set forth in SEQ ID NO: 19, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 19 and retains fusogenic activity in conjunction with a NiV-F protein as described.

Reference to retaining fusogenic activity includes activity of a lipid particle (e.g. lentiviral vector) containing a variant NiV-F protein as described or biologically active portion or functionally active variant of the F protein (in conjunction with a G protein, such as a NiV-G protein as described) that is between at or about 10% and at or about 150% or more of the level or degree of binding of a reference lipid particle (e.g. lentiviral vector) that is similar, such as contains the same variant NiV-F, but that contains the corresponding wild-type G protein, such as set forth in SEQ ID NO: 14. For instance, a lipid particle (e.g. lentiviral vector) that retains fusogenic activity has at least or at least about 10% of the level or degree of fusogenic activity of the reference lipid particle that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

c. Baboon Endogenous Retrovirus

In some embodiments, the fusogen is a Baboon Endogenous Retrovirus (BaEV) envelope glycoprotein. Exemplary BaEV envelope glycoproteins and variants thereof are described in PCT/US2022/031459; U.S. Pat. No. 9,249,426; Aguila et al. Journal of Virology 2003 77(2):1281-1291; Bernadin et al. Blood Advances 2019 3(3):461-475; Colamartino et al. Frontiers in Immunology 2019 10:2873; Girard-Gagnepain et al. Blood 2014 124(8):1221-1231; and Levy et al. Journal of Thrombosis and Haemostasis 2016 14:2478-2492.

Wild-type BaEV envelope glycoproteins are retroviral envelope proteins containing a C-terminal cytoplasmic tail (e.g., corresponding to amino acids 512-545 of SEQ ID NO:293), a transmembrane domain (e.g., corresponding to amino acids 489-511 of SEQ ID NO:293), and an extracellular domain (e.g., corresponding to amino acids 1-488 of SEQ ID NO:318). Maturation of the precursor protein in the Golgi, which requires the minimal sequence [KR]-X-[KR]-R (wherein X is any amino acid), results in two subunits, the surface unit protein or gp70, and the transmembrane protein p20E. The surface unit protein or gp70 (e.g., corresponding to amino acids 1-358 of SEQ ID NO:293) and the transmembrane protein p20E (e.g., corresponding to amino acids 359-545 of SEQ ID NO:293) remain associated in a labile interaction that may include a disulfide bond. In wild-type BaEV envelope glycoproteins, fusogenicity is controlled by a short, 17 amino acid sequence termed a fusion inhibitory R peptide (e.g., set forth in SEQ ID NO:293), which is localized on the C-terminal of the cytoplasmic tail domain. The fusion inhibitory R peptide harbors the tyrosine endocytosis signal YXXL, and its cleavage by the viral protease is thought to potentiate fusogenic activation through molecular rearrangements in the membrane-spanning domain and the extracellular region of the envelope glycoprotein (Salamango et al (2015) Journal of virology 89(24):12492-12500). In wild-type BaEV envelope glycoproteins, the gp70 mediates receptor binding to the ASCT-2 and ASCT-1 receptors on host cells. In some embodiments, the glycoprotein 70 (g70) subunit or a biologically active portion thereof binds the ASCT-2 and ASCT-1 receptors. In wild-type BaEV envelope glycoproteins, the p20E acts as a class I viral fusion protein. The interaction of the gp70 subunit with a host cell membrane triggers refolding of the p20E and is believed to activate the fusogenic potential by unmasking the fusion peptide.

In some embodiments, the fusogen is a truncated BaEV envelope glycoprotein. Exemplary BaEV envelope glycoproteins and truncates thereof are described in PCT/US2022/031459. In some embodiments, the truncated BaEV envelope glycoprotein comprises a cytoplasmic tail with a partial fusion inhibitory R peptide relative to a wild-type BaEV envelope glycoprotein, wherein the R peptide contains a contiguous portion of the inhibitory R peptide but lacks the full length R peptide of wild-type BaEV envelope glycoprotein. In some embodiments, the truncated BaEV envelope glycoprotein has a cytoplasmic tail that is composed of a partial inhibitory R peptide with at least one, at least two, or at least three contiguous amino-terminal amino acids of the inhibitory R peptide but less than the full-length R peptide relative to wild-type BaEV envelope glycoprotein. In some embodiments, the truncated BaEV envelope glycoprotein has a cytoplasmic tail that has a partial inhibitory R peptide composed of 1 to 16 contiguous amino-terminal amino acids of the inhibitory R peptide of the wild-type BaEV envelope glycoprotein, such as is composed of 1, 2, 3, 4, 5, 6, 7, 8, 9, 10, 11, 12, 12, 14, 15 or 16 amino-terminal amino acids of the inhibitory R peptide of the wild-type BaEV envelope glycoprotein. In some embodiments, the truncated BaEV envelope glycoprotein is set forth in any of SEQ ID NO:295-301.

In some embodiments, the fusogen is a modified BaEV envelope glycoprotein. In some embodiments, the cytoplasmic tail domain of the BaEV envelope glycoprotein is devoid of the fusion inhibitory R peptide. The expression “fusion inhibitory R peptide” refers to the C-terminal portion of the cytoplasmic tail domain of the envelope glycoprotein which harbors a tyrosine endocytosis signal—YXXL—and which is cleaved by viral protease during virion maturation, thus enhancing membrane fusion of the envelope glycoprotein. The fusion inhibitory R peptide of the BaEV envelope glycoprotein is typically located between amino acids 547 and 564 of the wild-type BaEV envelope glycoprotein. In some embodiments, the modified BaEV envelope glycoprotein is set forth in SEQ ID NO: 302 (BaEVRLess).

In some embodiments, the cytoplasmic tail domain of the BaEV envelope glycoprotein is replaced by the cytoplasmic tail domain of a murine leukemia virus (MLV) envelope glycoprotein. The Murine Leukemia Virus envelope glycoprotein is notably described in Ott et al. (1990) J. Virol. 64:757-766. In some embodiments, the Murine Leukemia Virus envelope glycoprotein is that of strain 4070A. The term “MLV envelope glycoprotein” refers to the wild-type form of the MLV envelope glycoprotein or to a mutant of said wild-type MLV envelope glycoprotein which is at least 80%, preferably at least 85%, still preferably at least 90%, more preferably at least 95%, still more preferably at least 99% identical to said wild-type MLV envelope glycoprotein, provided that said mutant glycoprotein retains the capacity of the wild-type envelope glycoprotein of interacting with viral core proteins, in particular with lentiviral core proteins. Typically, the cytoplasmic tail domain of the MLV envelope glycoprotein is located between amino acids 622 and 654 of the wild-type MLV envelope glycoprotein. In some embodiments, the modified BaEV envelope glycoprotein is set forth in SEQ ID NO: 303 (BaEVTR).

3. Re-Targeted Fusogens (e.g. Re-Targeted G Proteins)

In some embodiments, the fusogen (e.g. F or G protein) is a targeted envelope protein that contains a vector-surface targeting moiety. In some embodiments, the vector-surface targeting moiety binds a target ligand, such as a target molecule expressed on the cells (also referred to as a cell surface molecule or cell surface marker). The terms targeting agent or binding domain may also be used interchangeably with the term targeting moiety, and each are able to direct binding of the fusogen to a target ligand, such as a cell surface molecule. In some embodiments, the target ligand can be expressed on a target cell of interest, such as a target cell present as a leukocyte component. In some aspects, a fusogen can be retargeted to display altered tropism. In some embodiments, the binding confers re-targeted binding compared to the binding of a wild-type surface glycoprotein protein in which a new or different binding activity is conferred.

In some embodiments, a G protein (such as NiV-G) is further attached or linked to a binding domain that binds to a target molecule, such as a cell surface marker. For instance, provided in some aspects is a targeted lipid particle (e.g. targeted lentiviral vector) that includes a re-targeted G protein containing any of the provided G proteins attached to a binding domain, in which the re-targeted G protein is exposed on the surface of the targeted lipid particle (e.g. targeted lentiviral vector). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. In particular embodiments, the binding confers re-targeted binding compared to the binding of a wild-type G protein in which a new or different binding activity is conferred.

In some embodiments, the targeted envelope protein contains a G protein provided herein.

In some embodiments the G protein is any as described above, including NiV-G proteins with cytoplasmic domain modifications, truncated NiV-G cytoplasmic tails, or modified NiV-G cytoplasmic tails.

In some embodiments, the binding domain can be any agent that binds to a cell surface molecule on a target cells. In some embodiments, protein fusogens may be re-targeted by covalently conjugating a targeting-moiety to the fusion protein. In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002).

In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, the targeting agent is an antibody or antigen binding fragment thereof.

In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi- specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the targeting moiety is a binding domain that can be an antibody or an antibody portion or fragment. In some embodiments, the binding domain is a single domain antibody (sdAb). In some embodiments, the binding domain is a single chain variable fragment (scFv). In some examples, the binding domain can be linked directly or indirectly to the G protein (e.g. NiV-G or a biologically active portion). In particular embodiments, the binding domain is linked to the C-terminus (C-terminal amino acid) of the G protein or the biologically active portion thereof. The linkage can be via a peptide linker, such as a flexible peptide linker.

The binding domain may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target..

The binding domain may comprise a humanized antibody molecule, intact IgA, IgG, IgE or IgM antibody; bi- or multi- specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies@); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence (s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein. In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary target cells include cells present in a blood sample from a subject. In some embodiments, the cells include a leukocyte component. In some embodiments, the target cells include polymorphonuclear cells (also known as PMN, PML, PMNL, or granulocytes), In some embodiments, the target cells include lymphocytes, monocytes, macrophages, dendritic cells, natural killer cells, T cells (e.g. CD4 or CD8 T cells including cytotoxic T lymphocytes) or B cells. In some embodiments, the target cells include hematopoietic stem cells (HSCs). ,

In some embodiments, the target cell is a hematopoietic lineage cell. Reference to a “hematopoietic cell” includes blood cells, both from the myeloid and the lymphoid lineage. In particular, the term “hematopoietic cell” includes both undifferentiated or poorly differentiated cells, such as hematopoietic stem cells and progenitor cells, and differentiated cells such as T lymphocytes, B lymphocytes, or dendritic cells. In some embodiments, the hematopoietic cells are hematopoietic stem cells (HSCs), CD34+progenitor cells, in particular peripheral blood CD34+cells, very early progenitor CD34+cells, B-cell CD19+progenitors, myeloid progenitor CD13+cells, T lymphocytes, B lymphocytes, monocytes, dendritic cells, cancer B cells in particular B-cell chronic lymphocytic leukemia (BCLL) cells and marginal zone lymphoma (MZL) B cells, or thymocytes.

As known from the skilled person, many hematopoietic cells are produced from bone marrow hematopoietic stem cells.

In some embodiments, a hematopoietic cell is a hematopoietic stem cell (HSC), which are cells able to replenish all blood cell types and to self-renew. Hematopoietic stem cells may be in particular defined as cells that keep the levels of myeloid, T cells, and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207).

In some embodiments, the hematopoietic cell is a “CD34+progenitor cell,” which is a heterogeneous cell population that includes a subpopulation of HSCs, pluripotent stem cells and cells in the early stages of lineage commitment. CD34+progenitor cells continuously migrate to and from the bone marrow in normal adult animals. They can differentiate to produce all hematopoietic cell lineages found in the circulation. In some embodiments, the hematopoietic cell is a very early progenitor CD34+cell which is a subgroup of CD34+progenitor cells enriched from HSCs.

In some embodiments, the hematopoietic cell is a “peripheral blood CD34+cell”, which is a CD34+cell present in the blood.

In some embodiments, the hematopoietic cell is a B cell CD19+progenitor, which is a population of B-lineage cells that express cell surface CD10, CD34, and CD19.

In some embodiments, the hematopoietic cell is a myeloid progenitor CD13+cells, which is a population of myeloid lineage cells that express cell surface CD34 and CD13, and in some cases, also CD33.

In some embodiments, the target cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells. In some embodiments, the target cell is selected from monocytes, macrophages, neutrophils, basophils, eosinophils, erythrocytes, megakaryocytes and platelets. In some embodiments, the target cell is selected from T cells, B cells, natural killer (NK) cells and innate lymphoid cells.

In some embodiments the target cell is an effector cell, e.g., a cell of the immune system that expresses one or more Fc receptors and mediates one or more effector functions. In some embodiments, a target cell may include one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including humans, mice, rats, rabbits, and monkeys.

In some embodiment, the hematopoietic cell is a T cell. In some embodiments, the T cell is a naive T cell. In some embodiments, the T cell is a memory T cell.

In some embodiments, the hematopoietic cell is a B cell. In some embodiments, the target cell is a resting B cell, such as a naive or a memory B cell. In some embodiments, the target cell is a cancer B cell, such as a B-cell chronic lymphocytic leukemia (BCLL) cell or a marginal zone lymphoma (MZL) B cell.

In some embodiments, the target cell is a thymocyte. In some embodiments, the target cell is a natural killer (NK) cell. In some embodiments, the thymocyte expresses CD4 or CD8. In some embodiments, the thymocyte does not express CD4 or CD8. In some embodiments, the natural killer (NK) cell is a cell that expresses CD56.

In some embodiments, the target cell is a CD3+ T cell, a CD4+ T cell, a CD8+ T cell.

In some embodiments, the target cell is an antigen presenting cell, an MHC class II+cell, a professional antigen presenting cell, an atypical antigen presenting cell, a macrophage, a dendritic cell, a myeloid dendritic cell, a plasmacytoid dendritic cell, a CD11c+cell, a CD1lb+cell, or a B cell.

In some embodiments, the binding domain (e.g. sdAb) variable domain binds a cell surface molecule or antigen. In some embodiments, the cell surface molecule is ASGR1, ASGR2, TM4SF5, CD3, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R.

a. CD3

The viral vectors disclosed herein include one or more CD3 binding agents. For example, a CD3 binding agent may be fused to or incorporated in a retargeted attachment protein. In another embodiment, a CD3 binding agent may be incorporated into the lipid particle (e.g., viral vector) envelope via fusion with a transmembrane domain.

Exemplary CD3 binding agents include antibodies and fragments thereof (e.g., scFv, VHH) that bind to CD3. Such antibodies may be derived from any species, and may be for example, mouse, rabbit, human, humanized, or camelid antibodies.

Exemplary antibodies include OKT3, CRIS-7, I2C, blinatumomab, catumaxomab, muromonab-CD3, A-319, AFM11, AMG 199, AMG 211, AMG 424, AMG 427, AMG 562, AMG 564, APV0436, CC-93269, ERY974, GBR1302, GEM333, GEM2PSCA, GNC-035, HPN424, IGM-2323, JNJ-63709178, JNJ-63898081, JNJ-75348780, JNJ-78306358, M701, M802, MGD007, MOR209/ES414, PF-06671008, REGN5459, RO7283420, SAR442257, SAR443216, TNB-383B, TNB-486, TNB-585, Y150, acapatamab, cevostamab, cibisatamab, duvortuxizumab, eluvixtamab, emerfetamab, etevritamab, glofitamab, gresonitamab, obrindatamab, pavurutamab, plamotamab, solitomab, tarlatamab, tepoditamab, tidutamab, vibecotamab, vixtimotamab, alnuctamab, dafsolimab setaritox, pacanalotamab, pasotuxizumab, runimotamab, nivatrotamab, elranatamab, ertumaxomab, flotetuzumab, odronextamab, talquetamab, teclistamab, visilizumab, epcoritamab, otelixizumab, 3F8BiAb, CCW702, DKTK CC-1, EMB-06, GEN1044, GEN1047, GTB-3550, HPN217, IMC-C103C, NVG-111, REGN4018, REGN4336, REGN5458, A-2019, A-337, ABP-100, AFM15, AFM21, AMG 701, APV0425, CLN-049, Dow2, EM801, Ektomab, FBTA05, GBR1342, GBR1372, GSK3537142, HBM7020, HLX31, IGM-2644, MG1122, MGD015, ND003, ND007, PF-07062119, RO7293583, STA551, TT19, ZW38; and anti-CD3 antibodies disclosed in U.S. Pat. Nos. 4,361,549, 7,728,114, 9,657,102, 9,587,021, and 11007267; US Patent Application Nos. US20120269826, US20180057597, and US20180112000; and PCT Application Nos. WO2005118635, WO2011050106, WO2012162067, WO2014047231, WO2016116626, WO2016180721, and WO2016204966. Other exemplary binding agents include designed ankyrin repeat proteins (DARPins) and binding agents based on fibronectin type III (Fn3) scaffolds.

In some embodiments, the CD3 binding agent comprises a heavy chain variable (VH) region comprising a CDR-H1, a CDRH-2, and a CDR-H3 comprising the amino acid sequence set forth in SEQ ID NO:304, 305, and 306 respectively; and a light chain variable region comprising a CDR-L1, a CDR-L2, and a CDR-L3 comprising the amino acid sequence set forth in SEQ ID NO:307, 308, and 309, respectively. In some embodiments, the CD3 binding agent comprises a VH region comprising an amino acid sequence having at least about 90% sequence identity to the amino acid sequence set forth in SEQ ID NO:310, and a VL region comprising an amino acid sequence having at least about 90% sequence identity to the amino acid sequence set forth in SEQ ID NO:311. In some embodiments, the CD3 binding agent comprises a VH region comprising the amino acid sequence set forth in SEQ ID NO:310, and a VL region comprising the amino acid sequence set forth in SEQ ID NO:311. In some embodiments, the CD3 binding agent is an scFv. In some embodiments, the CD3 binding agent comprises the amino acid sequence set forth in SEQ ID NO:312. In some embodiments, the CD3 binding agent is OKT3.

In some embodiments, the CD3 binding agent is activating (e.g., the CD3 binding agent activates T cells). In some embodiments, the CD3 binding agent is non-activating (e.g., it does not activate T cells).

In some embodiments, a CD3 binding agent comprises a humanized antibody molecule, intact IgA, IgG, IgE or IgM antibody; bi- or multi- specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD3 binding agent is a peptide. In some embodiments, the CD3 binding agent is an antibody, such as a single-chain variable fragment (scFv). In some embodiments, the CD3 binding agent is an antibody, such as a single domain antibody. In some embodiments, the antibody can be human or humanized. In some embodiments, the CD3 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD3 binding agent is attached to the C-terminus of the G protein (e.g., fusogen) or biologically active portion thereof. In some embodiments, the N-terminus of the CD3 binding agent is exposed on the exterior surface of the lipid bilayer.

In some embodiments, the CD3 binding agent is the only surface displayed non-viral sequence of the viral vector. In some embodiments, the CD3 binding agent is the only membrane bound non-viral sequence of the viral vector. In some embodiments, the viral vector does not contain a molecule that engages or stimulates T cells other than the CD3 binding agent. In some embodiments, the viral vector contains a non-activating CD3 binding agent.

In some embodiments, viral vectors may display CD3 binding agents that are not conjugated to protein fusogens in order to redirect the fusion activity towards a cell that is bound by the targeting moiety, or to affect homing.

b. CD7

The viral vectors disclosed herein include one or more CD7 binding agents. For example, a CD7 binding agent may be fused to or incorporated in a protein fusogen or attachment protein. In another embodiment, a CD7 binding agent may be incorporated into the lipid particle (e.g., viral vector) envelope via fusion with a transmembrane domain.

Exemplary CD7 binding agents include antibodies and fragments thereof (e.g., scFv, VHH) that bind to CD7. Such antibodies may be derived from any species, and may be for example, mouse, rabbit, human, humanized, or camelid antibodies. Exemplary antibodies include grsinilimab, SPV-T3a and those disclosed in WO2015/184941; U.S. Ser. No. 10/106,609; WO2017/213979; WO2018/098306; WO2019086534; U.S. Ser. No. 11/447,548; WO2019/102234; WO2022/136887; WO2022/136888; WO2020/212710; WO2021/160267; WO2022/095803; WO2022/151851. Further exemplary anti-CD7 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, protein fusogens or attachment proteins may be re-targeted by mutating amino acid residues in a fusion protein or a targeting protein (e.g. retargeted attachment protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens (e.g., attachment proteins) may be re-targeted by covalently conjugating a CD7 binding agent to the fusion protein or attachment protein (e.g. retargeted attachment protein). In some embodiments, the fusogen and CD7 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD7 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a CD7 binding agent comprises a humanized antibody molecule, intact IgA, IgG, IgE or IgM antibody; bi- or multi- specific antibody (e.g., Zybodies®, etc.); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD7 binding agent is a peptide. In some embodiments, the CD7 binding agent is an antibody, such as a single-chain variable fragment (scFv). In some embodiments, the CD7 binding agent is an antibody, such as a single domain antibody. In some embodiments, the CD7 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD7 binding agent is attached to the C-terminus of the G protein (e.g., fusogen) or biologically active portion thereof. In some embodiments, the N-terminus of the CD7 binding agent is exposed on the exterior surface of the lipid bilayer.

In some embodiments, the CD7 binding agent is the only surface displayed non-viral sequence of the viral vector. In some embodiments, the CD7 binding agent is the only membrane bound non-viral sequence of the viral vector. In some embodiments, the viral vector does not contain a molecule that engages or stimulates T cells other than the CD7 binding agent.

In some embodiments, viral vectors may display CD7 binding agents that are not conjugated to protein fusogens in order to redirect the fusion activity towards a cell that is bound by the targeting moiety, or to affect homing.

c. CD4

The viral vectors disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain.

In some of any of the provided embodiments, the CD4 binding agent is an anti-CD4 antibody or an antigen-binding fragment. In some of any of the provided embodiments, the anti-CD4 antibody or antigen-binding fragment is mouse, rabbit, human, or humanized. In some embodiments, the antigen-binding fragment is a single chain variable fragment (scFv). In some embodiments, the antigen-binding fragment is an anti-CD4 scFv.

In some embodiments, the anti-CD4 scFv comprises a CDR-H1, a CDR-H2, and a CDR-H3 comprising the amino acid sequence set forth in SEQ ID NO: 262, 263, and 264, respectively. In some embodiments, the anti-CD4 scFv comprises a CDR-L1, a CDR-L2, and a CDR-L3 comprising the amino acid sequence set forth in SEQ ID NO: 265, 266, and 267, respectively. In some embodiments, the anti-CD4 scFv comprises a CDR-H1, a CDR-H2, and a CDR-H3 comprising the amino acid sequence set forth in SEQ ID NO: 262, 263, and 264, respectively; and a CDR-L1, a CDR-L2, and a CDR-L3 comprising the amino acid sequence set forth in SEQ ID NO: 265, 266, and 267, respectively. In some embodiments, the anti-CD4 scFv comprises a CDR-H1, a CDR-H2, and a CDR-H3 comprising the amino acid sequence set forth in SEQ ID NO: 251, 252, and 253, respectively. In some embodiments, the anti-CD4 scFv comprises a CDR-L1, a CDR-L2, and a CDR-L3 comprising the amino acid sequence set forth in SEQ ID NO: 254, 255, and 267, respectively. In some embodiments, the anti-CD4 scFv comprises a CDR-H1, a CDR-H2, and a CDR-H3 comprising the amino acid sequence set forth in SEQ ID NO: 251, 252, and 253, respectively; and a CDR-L1, a CDR-L2, and a CDR-L3 comprising the amino acid sequence set forth in SEQ ID NO: 254, 255, and 267, respectively. In some embodiments, the anti-CD4 scFv comprises a CDR-H1, a CDR-H2, and a CDR-H3 comprising the amino acid sequence set forth in SEQ ID NO: 256, 257, and 253, respectively. In some embodiments, the anti-CD4 scFv comprises a CDR-L1, a CDR-L2, and a CDR-L3 comprising the amino acid sequence set forth in SEQ ID NO: 254, 255, and 267, respectively. In some embodiments, the anti-CD4 scFv comprises a CDR-H1, a CDR-H2, and a CDR-H3 comprising the amino acid sequence set forth in SEQ ID NO: 256, 257, and 253, respectively; and a CDR-L1, a CDR-L2, and a CDR-L3 comprising the amino acid sequence set forth in SEQ ID NO: 254, 255, and 267, respectively. In some embodiments, the anti-CD4 scFv comprises a heavy chain variable region (VH) comprising the amino acid sequence set forth in SEQ ID NO:268. In some embodiments, the anti-CD4 scFv comprises a light chain variable region (VL) comprising the amino acid sequence set forth in SEQ ID NO:269. In some embodiments, the anti-CD4 scFv comprises a heavy chain variable region (VH) comprising the amino acid sequence set forth in SEQ ID NO:268; and a light chain variable region (VL) comprising the amino acid sequence set forth in SEQ ID NO:269. In some embodiments, the VH and VL are joined by a linker. In some embodiments, the linker comprises the amino acid sequence set forth in SEQ ID NO:51. In some embodiments, the anti-CD4 scFv comprises the amino acid sequence set forth in SEQ ID NO:270.

In some embodiments, the anti-CD4 scFv comprises a CDR-H1, a CDR-H2, and a CDR-H3 comprising the amino acid sequence set forth in SEQ ID NO: 271, 272, and 273, respectively. In some embodiments, the anti-CD4 scFv comprises a CDR-L1, a CDR-L2, and a CDR-L3 comprising the amino acid sequence set forth in SEQ ID NO: 274, 275, and 276, respectively. In some embodiments, the anti-CD4 scFv comprises a CDR-H1, a CDR-H2, and a CDR-H3 comprising the amino acid sequence set forth in SEQ ID NO: 271, 272, and 273, respectively; and a CDR-L1, a CDR-L2, and a CDR-L3 comprising the amino acid sequence set forth in SEQ ID NO: 274, 275, and 276, respectively. In some embodiments, the anti-CD4 scFv comprises a CDR-H1, a CDR-H2, and a CDR-H3 comprising the amino acid sequence set forth in SEQ ID NO: 258, 190, and 191, respectively. In some embodiments, the anti-CD4 scFv comprises a CDR-L1, a CDR-L2, and a CDR-L3 comprising the amino acid sequence set forth in SEQ ID NO: 261, 262, and 276, respectively. In some embodiments, the anti-CD4 scFv comprises a CDR-H1, a CDR-H2, and a CDR-H3 comprising the amino acid sequence set forth in SEQ ID NO: 258, 190, and 191, respectively; and a CDR-L1, a CDR-L2, and a CDR-L3 comprising the amino acid sequence set forth in SEQ ID NO: 261, 262, and 276, respectively. In some embodiments, the anti-CD4 scFv comprises a CDR-H1, a CDR-H2, and a CDR-H3 comprising the amino acid sequence set forth in SEQ ID NO: 263, 264, and 191, respectively. In some embodiments, the anti-CD4 scFv comprises a CDR-L1, a CDR-L2, and a CDR-L3 comprising the amino acid sequence set forth in SEQ ID NO: 261, 262, and 276, respectively. In some embodiments, the anti-CD4 scFv comprises a CDR-H1, a CDR-H2, and a CDR-H3 comprising the amino acid sequence set forth in SEQ ID NO: 263, 264, and 191, respectively; and a CDR-L1, a CDR-L2, and a CDR-L3 comprising the amino acid sequence set forth in SEQ ID NO: 261, 262, and 276, respectively. In some embodiments, the anti-CD4 scFv comprises a heavy chain variable region (VH) comprising the amino acid sequence set forth in SEQ ID NO:208. In some embodiments, the anti-CD4 scFv comprises a light chain variable region (VL) comprising the amino acid sequence set forth in SEQ ID NO:278. In some embodiments, the anti-CD4 scFv comprises a heavy chain variable region (VH) comprising the amino acid sequence set forth in SEQ ID NO:208; and a light chain variable region (VL) comprising the amino acid sequence set forth in SEQ ID NO:278. In some embodiments, the VH and VL are joined by a linker. In some embodiments, the linker comprises the amino acid sequence set forth in SEQ ID NO:51. In some embodiments, the anti-CD4 scFv comprises the amino acid sequence set forth in SEQ ID NO:279.

In some embodiments, the anti-CD4 scFv comprises a CDR-H1, a CDR-H2, and a CDR-H3 comprising the amino acid sequence set forth in SEQ ID NO: 280, 281, and 282, respectively. In some embodiments, the anti-CD4 scFv comprises a CDR-L1, a CDR-L2, and a CDR-L3 comprising the amino acid sequence set forth in SEQ ID NO: 145, 284, and 285, respectively. In some embodiments, the anti-CD4 scFv comprises a CDR-H1, a CDR-H2, and a CDR-H3 comprising the amino acid sequence set forth in SEQ ID NO: 280, 281, and 282, respectively; and a CDR-L1, a CDR-L2, and a CDR-L3 comprising the amino acid sequence set forth in SEQ ID NO: 145, 284, and 285, respectively. In some embodiments, the anti-CD4 scFv comprises a CDR-H1, a CDR-H2, and a CDR-H3 comprising the amino acid sequence set forth in SEQ ID NO: 290, 291, 292, respectively. In some embodiments, the anti-CD4 scFv comprises a CDR-L1, a CDR-L2, and a CDR-L3 comprising the amino acid sequence set forth in SEQ ID NO: 268, 269, and 285, respectively. In some embodiments, the anti-CD4 scFv comprises a CDR-H1, a CDR-H2, and a CDR-H3 comprising the amino acid sequence set forth in SEQ ID NO: 265, 266, 267, respectively; and a CDR-L1, a CDR-L2, and a CDR-L3 comprising the amino acid sequence set forth in SEQ ID NO: 268, 269, and 285, respectively. In some embodiments, the anti-CD4 scFv comprises a CDR-H1, a CDR-H2, and a CDR-H3 comprising the amino acid sequence set forth in SEQ ID NO: 270, 271, 272, respectively. In some embodiments, the anti-CD4 scFv comprises a CDR-L1, a CDR-L2, and a CDR-L3 comprising the amino acid sequence set forth in SEQ ID NO: 268, 269, and 285, respectively. In some embodiments, the anti-CD4 scFv comprises a CDR-H1, a CDR-H2, and a CDR-H3 comprising the amino acid sequence set forth in SEQ ID NO: 270, 271, 267, respectively; and a CDR-L1, a CDR-L2, and a CDR-L3 comprising the amino acid sequence set forth in SEQ ID NO: 268, 269, and 285, respectively. In some embodiments, the anti-CD4 scFv comprises a heavy chain variable region (VH) comprising the amino acid sequence set forth in SEQ ID NO:286. In some embodiments, the anti-CD4 scFv comprises a light chain variable region (VL) comprising the amino acid sequence set forth in SEQ ID NO:287. In some embodiments, the anti-CD4 scFv comprises a heavy chain variable region (VH) comprising the amino acid sequence set forth in SEQ ID NO:286; and a light chain variable region (VL) comprising the amino acid sequence set forth in SEQ ID NO:287. In some embodiments, the VH and VL are joined by a linker. In some embodiments, the linker comprises the amino acid sequence set forth in SEQ ID NO:55. In some embodiments, the anti-CD4 scFv comprises the amino acid sequence set forth in SEQ ID NO:288.

In some embodiments, the anti-CD4 scFv comprises a CDR-H1, a CDR-H2, and a CDR-H3 comprising the amino acid sequence set forth in SEQ ID NO: 289, 290, and 291, respectively. In some embodiments, the anti-CD4 scFv comprises a CDR-L1, a CDR-L2, and a CDR-L3 comprising the amino acid sequence set forth in SEQ ID NO: 292, 224, and 225, respectively. In some embodiments, the anti-CD4 scFv comprises a CDR-H1, a CDR-H2, and a CDR-H3 comprising the amino acid sequence set forth in SEQ ID NO: 289, 290, and 291, respectively; and a CDR-L1, a CDR-L2, and a CDR-L3 comprising the amino acid sequence set forth in SEQ ID NO: 292, 224, and 225, respectively. In some embodiments, the anti-CD4 scFv comprises a CDR-H1, a CDR-H2, and a CDR-H3 comprising the amino acid sequence set forth in SEQ ID NO: 272, 273, 274, respectively. In some embodiments, the anti-CD4 scFv comprises a CDR-L1, a CDR-L2, and a CDR-L3 comprising the amino acid sequence set forth in SEQ ID NO: 275, 276, and 225, respectively. In some embodiments, the anti-CD4 scFv comprises a CDR-H1, a CDR-H2, and a CDR-H3 comprising the amino acid sequence set forth in SEQ ID NO: 272, 273, 274, respectively; and a CDR-L1, a CDR-L2, and a CDR-L3 comprising the amino acid sequence set forth in SEQ ID NO: 275, 276, and 225, respectively. In some embodiments, the anti-CD4 scFv comprises a CDR-H1, a CDR-H2, and a CDR-H3 comprising the amino acid sequence set forth in SEQ ID NO: 208, 278, 274, respectively. In some embodiments, the anti-CD4 scFv comprises a CDR-L1, a CDR-L2, and a CDR-L3 comprising the amino acid sequence set forth in SEQ ID NO: 275, 276, and 225, respectively. In some embodiments, the anti-CD4 scFv comprises a CDR-H1, a CDR-H2, and a CDR-H3 comprising the amino acid sequence set forth in SEQ ID NO: 208, 278, 274, respectively; and a CDR-L1, a CDR-L2, and a CDR-L3 comprising the amino acid sequence set forth in SEQ ID NO: 275, 276, and 225, respectively. In some embodiments, the anti-CD4 scFv comprises a heavy chain variable region (VH) comprising the amino acid sequence set forth in SEQ ID NO:226. In some embodiments, the anti-CD4 scFv comprises a light chain variable region (VL) comprising the amino acid sequence set forth in SEQ ID NO:227. In some embodiments, the anti-CD4 scFv comprises a heavy chain variable region (VH) comprising the amino acid sequence set forth in SEQ ID NO:226; and a light chain variable region (VL) comprising the amino acid sequence set forth in SEQ ID NO:227. In some embodiments, the VH and VL are joined by a linker. In some embodiments, the linker comprises the amino acid sequence set forth in SEQ ID NO:55. In some embodiments, the anti-CD4 scFv comprises the amino acid sequence set forth in SEQ ID NO:228.

In some embodiments, the anti-CD4 scFv comprises a CDR-H1, a CDR-H2, and a CDR-H3 comprising the amino acid sequence set forth in SEQ ID NO: 229, 230, and 231, respectively. In some embodiments, the anti-CD4 scFv comprises a CDR-L1, a CDR-L2, and a CDR-L3 comprising the amino acid sequence set forth in SEQ ID NO: 232, 284, and 233, respectively. In some embodiments, the anti-CD4 scFv comprises a CDR-H1, a CDR-H2, and a CDR-H3 comprising the amino acid sequence set forth in SEQ ID NO: 229, 230, and 231, respectively; and a CDR-L1, a CDR-L2, and a CDR-L3 comprising the amino acid sequence set forth in SEQ ID NO: 232, 284, and 233, respectively. In some embodiments, the anti-CD4 scFv comprises a CDR-H1, a CDR-H2, and a CDR-H3 comprising the amino acid sequence set forth in SEQ ID NO: 279, 280, and 281, respectively. In some embodiments, the anti-CD4 scFv comprises a CDR-L1, a CDR-L2, and a CDR-L3 comprising the amino acid sequence set forth in SEQ ID NO: 282, 283, and 233, respectively. In some embodiments, the anti-CD4 scFv comprises a CDR-H1, a CDR-H2, and a CDR-H3 comprising the amino acid sequence set forth in SEQ ID NO: 279, 280, and 281, respectively; and a CDR-L1, a CDR-L2, and a CDR-L3 comprising the amino acid sequence set forth in SEQ ID NO: 282, 283, and 233, respectively. In some embodiments, the anti-CD4 scFv comprises a CDR-H1, a CDR-H2, and a CDR-H3 comprising the amino acid sequence set forth in SEQ ID NO: 284, 285, and 281, respectively. In some embodiments, the anti-CD4 scFv comprises a CDR-L1, a CDR-L2, and a CDR-L3 comprising the amino acid sequence set forth in SEQ ID NO: 282, 283, and 233, respectively. In some embodiments, the anti-CD4 scFv comprises a CDR-H1, a CDR-H2, and a CDR-H3 comprising the amino acid sequence set forth in SEQ ID NO: 284, 285, and 281, respectively; and a CDR-L1, a CDR-L2, and a CDR-L3 comprising the amino acid sequence set forth in SEQ ID NO: 282, 283, and 233, respectively. In some embodiments, the anti-CD4 scFv comprises a heavy chain variable region (VH) comprising the amino acid sequence set forth in SEQ ID NO:234. In some embodiments, the anti-CD4 scFv comprises a light chain variable region (VL) comprising the amino acid sequence set forth in SEQ ID NO:235. In some embodiments, the anti-CD4 scFv comprises a heavy chain variable region (VH) comprising the amino acid sequence set forth in SEQ ID NO:234; and a light chain variable region (VL) comprising the amino acid sequence set forth in SEQ ID NO:235. In some embodiments, the VH and VL are joined by a linker. In some embodiments, the linker comprises the amino acid sequence set forth in SEQ ID NO:55. In some embodiments, the anti-CD4 scFv comprises the amino acid sequence set forth in SEQ ID NO:236.

In some embodiments, the anti-CD4 scFv comprises a CDR-H1, a CDR-H2, and a CDR-H3 comprising the amino acid sequence set forth in SEQ ID NO: 237, 238, and 239, respectively. In some embodiments, the anti-CD4 scFv comprises a CDR-L1, a CDR-L2, and a CDR-L3 comprising the amino acid sequence set forth in SEQ ID NO: 240, 241, and 242, respectively. In some embodiments, the anti-CD4 scFv comprises a CDR-H1, a CDR-H2, and a CDR-H3 comprising the amino acid sequence set forth in SEQ ID NO: 237, 238, and 239, respectively; and a CDR-L1, a CDR-L2, and a CDR-L3 comprising the amino acid sequence set forth in SEQ ID NO: 240, 241, and 242, respectively. In some embodiments, the anti-CD4 scFv comprises a CDR-H1, a CDR-H2, and a CDR-H3 comprising the amino acid sequence set forth in SEQ ID NO: 286, 287, and 288, respectively. In some embodiments, the anti-CD4 scFv comprises a CDR-L1, a CDR-L2, and a CDR-L3 comprising the amino acid sequence set forth in SEQ ID NO: 289, 290, and 242, respectively. In some embodiments, the anti-CD4 scFv comprises a CDR-H1, a CDR-H2, and a CDR-H3 comprising the amino acid sequence set forth in SEQ ID NO: 286, 287, and 288, respectively; and a CDR-L1, a CDR-L2, and a CDR-L3 comprising the amino acid sequence set forth in SEQ ID NO: 289, 290, and 242, respectively. In some embodiments, the anti-CD4 scFv comprises a CDR-H1, a CDR-H2, and a CDR-H3 comprising the amino acid sequence set forth in SEQ ID NO: 291, 292, and 288, respectively. In some embodiments, the anti-CD4 scFv comprises a CDR-L1, a CDR-L2, and a CDR-L3 comprising the amino acid sequence set forth in SEQ ID NO: 289, 290, and 242, respectively. In some embodiments, the anti-CD4 scFv comprises a CDR-H1, a CDR-H2, and a CDR-H3 comprising the amino acid sequence set forth in SEQ ID NO: 291, 292, and 293, respectively; and a CDR-L1, a CDR-L2, and a CDR-L3 comprising the amino acid sequence set forth in SEQ ID NO: 289, 290, and 242, respectively. In some embodiments, the anti-CD4 scFv comprises a heavy chain variable region (VH) comprising the amino acid sequence set forth in SEQ ID NO:243. In some embodiments, the anti-CD4 scFv comprises a light chain variable region (VL) comprising the amino acid sequence set forth in SEQ ID NO:244. In some embodiments, the anti-CD4 scFv comprises a heavy chain variable region (VH) comprising the amino acid sequence set forth in SEQ ID NO:243; and a light chain variable region (VL) comprising the amino acid sequence set forth in SEQ ID NO:244. In some embodiments, the VH and VL are joined by a linker. In some embodiments, the linker comprises the amino acid sequence set forth in SEQ ID NO:55. In some embodiments, the anti-CD4 scFv comprises the amino acid sequence set forth in SEQ ID NO:245.

In some embodiments, the anti-CD4 antibody or antigen-binding fragment is a single domain antibody. In some embodiments, the anti-CD4 antibody or antigen-binding fragment is a camelid (e.g. llama, alpaca, camel) anti-CD4 antibody or antigen-binding fragment (e.g. VHH). In some embodiments, the anti-CD4 antibody or antigen-binding fragment is an anti-CD4 VHH. In some embodiments, the anti-CD4 VHH comprises a CDR-H1, a CDR-H2, and a CDR-H3 comprising the amino acid sequence set forth in SEQ ID NO: 258, 190, and 191, respectively. In some embodiments, the anti-CD4 VHH comprises a CDR-H1, a CDR-H2, and a CDR-H3 comprising the amino acid sequence set forth in SEQ ID NO: 246, 247, and 248, respectively. In some embodiments, the anti-CD4 VHH comprises a CDR-H1, a CDR-H2, and a CDR-H3 comprising the amino acid sequence set forth in SEQ ID NO: 249, 250, and 248, respectively. In some embodiments, the anti-CD4 VHH comprises the amino acid sequence set forth in SEQ ID NO:261.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963,U.S. Pat. Nos. 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UGO (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or targeting protein (e.g. the hemagglutinin protein). In some embodiments, the fusogen and CD4 binding agent are covalently conjugated by expression of a chimeric protein comprising the fusogen linked to the CD4 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi- specific antibody (e.g., Zybodies®, etc.); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the viral vector. In some embodiments, the CD4 binding agent is the only membrane bound non-viral sequence of the viral vector. In some embodiments, the viral vector does not contain a molecule that engages or stimulates T cells other than the CD4 binding agent.

In some embodiments, viral vectors may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients and/or subjects, and thus has high specificity.

d. CD8

The viral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain.

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Ser. No. 10/730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SKi, RPA-T8 (GeneTex); UCHT4 (Absolute Antibody); BW135/80 (Miltenyi); G42-8 (BD Biosciences); C8/1779R, mAb 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, the CD8 binding agent is an scFv that contains a VH and VL set forth from any as below, in which the VH and VL are separated by linker. In some embodiments, the CD8 binding agent is a VHH having the sequence set forth below. In some embodiments, the CD8 binding agent is linked to the C-terminus of a truncated NiV-G set forth in SEQ ID NO: 19 to provide a re-targeted NiV-G. In some embodiments, the retargeted NiV-G is pseudotyped on a lentiviral vector with the a NiV-F (e.g. set forth in SEQ ID NO:12). In some embodiments, the lentiviral vector further contains a payload gene encoding an anti-CD19 CAR. In some embodiments, the anti-CD19 CAR contains an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36. a CD3zeta signaling domain set forth in SEQ ID NO: 38.

CD8_1
VH (SEQ ID NO.: 120):
QVQLVQSGAEVKKPGASVKVSCKASGGTFSSYAISWVRQAPGQGLEWMG
IIDPSDGNTNYAQNFQGRVTMTRDTSTSTVYMELSSLRSEDTAVYYCAK
ERAAAGYYYYMDVWGQGTTVTVSS
VL (SEQ ID NO.: 121):
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIKR
CD8_2
VH (SEQ ID NO.: 122):
QVQLVQSGAEVKKPGASVKVSCKASGYTFTDYYIQWVRQAPGQGLEWMG
WINPNSGGTSYAQKFQGRVTMTRDTSTSTVYMELSSLRSEDTAVYYCAK
EGDYYYGMDAWGQGTMVTVSS
VL (SEQ ID NO.: 123):
DIVMTQSPLSLPVTPGEPASISCRSSQSLLHSNGYNYLDWYLQKPGQSP
QLLIYLGSNRASGVPDRFSGSGSGTDFTLKISRVEAEDVGVYYCMQGLQ
TPHTFGQGTKVEIKR
CD8_3
VH (SEQ ID NO.: 124):
QVQLVQSGAEVKKPGASVKVSCKASGYTFTSYYMHWVRQAPGQGLEWMG
GFDPEDGETIYAQKFQGRVTMTRDTSTSTVYMELSSLRSEDTAVYYCAR
DQGWGMDVWGQGTTVTVSS
VL (SEQ ID NO.: 125):
DIQMTQSPSSLSASVGDRVTITCRASQSISSYLNWYQQKPGKAPKLLIY
AASSLQSGVPSRFSGSGSGTDFTLTISSLQPEDFATYYCQQTYSTPYTF
GQGTKLEIKR
CD8_4
VH (SEQ ID NO.: 126):
QVQLVQSGAEVKKPGASVKVSCKASGYTFTNHYMHWVRQAPGQGLEWMG
WMNPNSGNTGYAQKFQGRVTMTRDTSTSTVYMELSSLRSEDTAVYYCAS
SESGSDLDYWGQGTLVTVSS
VL (SEQ ID NO.: 127):
DIQMTQSPSSLSASVGDRVTITCRASQTIGNYVNWYQQKPGKAPKLLIY
GASNLHTGVPSRFSGSGSGTDFTLTISSLQPEDFATYYCQQTYSAPLTF
GGGTKVEIKR

In some embodiments, the CD8 binding agent is VHH set forth as:

VHH (SEQ ID NO.: 128):
QVQLVESGGGLVQAGGSLRLSCAASGRTFSGYVMGWFRQAPGKQRKFVA
AISRGGLSTSYADSVKGRFTISRDNAKNTVFLQMNTLKPEDTAVYYCAA
DRSDLYEITAASNIDSWGQGTLVTVSS

In some embodiments, protein fusogens or viral envelope proteins may be re-targeted by mutating amino acid residues in a fusion protein or a targeting protein (e.g. the hemagglutinin protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD8 binding agent to the fusion protein or targeting protein (e.g. the hemagglutinin protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a CD8 binding agent comprises a humanized antibody molecule, intact IgA, IgG, IgE or IgM antibody; bi- or multi- specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the viral vector. In some embodiments, the CD8 binding agent is the only membrane bound non-viral sequence of the viral vector. In some embodiments, the viral vector does not contain a molecule that engages or stimulates T cells other than the CD8 binding agent.

In some embodiments, viral vectors may display CD8 binding agents that are not conjugated to protein fusogens in order to redirect the fusion activity towards a cell that is bound by the targeting moiety, or to affect homing.

e. HSC

In some embodiments, the target cell is a CD34+progenitor cells. In some embodiments, the target cell molecule is expressed on at least a subset of CD34+progenitor cells.

In some embodiments, the cell surface molecule is expressed on HSCs. In some embodiments, the cell surface molecule is expressed on MPPs. In some embodiments, the cell surface molecule is expressed on MLPs. In some embodiments, the cell surface molecule is expressed on ETPs. In some embodiments, the cell surface molecule is expressed on MEPs. In some embodiments, the cell surface molecule is expressed on CMPs. In some embodiments, the cell surface molecule is expressed on GMPs. In some embodiments, the cell surface molecule is expressed on any combination of the foregoing CD34+progenitor subpopulations. In some embodiments, the cell surface molecule is expressed on HSCs and MPPs. In some embodiments, the cell surface molecule is expressed on myeloid progenitors. In some embodiments, the cell surface molecule is expressed on lymphoid progenitors. In some embodiments, the cell surface molecule is expressed on myeloid progenitors. In some embodiments, the cell surface molecule is expressed on HSCs, MPPs, MEPs, CMPs, and GMPs.

In some embodiments, the cell surface molecule is ASCT2. In some embodiments, the target cell is ASCT2+.

In some embodiments, the cell surface molecule is CD105. In some embodiments, the target cell is CD105+.

In some embodiments, the cell surface molecule is CD110. In some embodiments, the target cell is CD110+.

In some embodiments, the cell surface molecule is CD117. In some embodiments, the target cell is CD117+.

In some embodiments, the cell surface molecule is CD133. In some embodiments, the target cell is CD133+.

In some embodiments, the cell surface molecule is CD146. In some embodiments, the target cell is CD146+.

In some embodiments, the cell surface molecule is CD164. In some embodiments, the target cell is CD164+.

In some embodiments, the cell surface molecule is CD34. In some embodiments, the target cell is CD34+.

In some embodiments, the cell surface molecule is CD46. In some embodiments, the target cell is CD46+.

In some embodiments, the cell surface molecule is CD49f. In some embodiments, the target cell is CD49f+.

In some embodiments, the ta cell surface target molecule is CD90. In some embodiments, the target cell is CD90+.

In some embodiments, the cell surface molecule is EPCR. In some embodiments, the target cell is EPCR+.

In some embodiments, the cell surface molecule is ITGA3. In some embodiments, the target cell is ITGA3+.

In some embodiments, the target molecule is CD133. In some embodiments, the target cell is CD133+. In some embodiments, the targeting agent is an anti-CD133 antibody. Exemplary anti-CD133 antibodies include CART133, AC133, 293C3-SDIE, CMab-43, RWO3, 293C3H9 (293C3), and W6B3H10 (W6B3); and anti-CD133 antibodies disclosed in US Patent Nos. U.S. Pat. Nos. 8,722,858, 9,249,225, 9,624,303, U.S. Ser. No. 10/106,623, U.S. Ser. No. 10/711,068, U.S. Ser. No. 11/098,109, U.S. Ser. No. 11/214,628, U.S. Ser. No. 11/352,435, and U.S. Ser. No. 11/220,551; US Patent Application Nos. US20130224202; PCT Application Nos. W0200901840, WO2011089211, WO2011149493, WO2014128185, WO2015121383, WO2016154623, WO2018045880, WO2018072025, and WO2022022718; and Canadian Patent Application No. CA2962157.

In some embodiments, the target molecule is CD105. In some embodiments, the target cell is CD105+. In some embodiments, the targeting agent is an anti-CD105 antibody. Exemplary anti-CD105 antibodies include carotuximab, TRC105, huRH105, and TCR205; and anti-CD105 antibodies disclosed in US Patent Nos. U.S. Pat. Nos. 8,221,753, 8,609,094, 9,150,652, 95,181,212, 9,926,375, 9,944,714, U.S. Ser. No. 10/155,820, and U.S. Ser. No. 10/336,831; US Patent Application Nos. US20100098692, US20100196398, US20170007714, and US20220233591; PCT Application Nos. WO2010039873, WO2011041441, WO2016077451, WO2018067819, WO2010032059, WO2012149412, WO2015118031, WO2021118955, and WO2021118957; and Korean Patent No. KR101398707B1.

In some embodiments, the target molecule is EPCR. In some embodiments, the target cell is EPCR+. In some embodiments, the targeting agent is an anti-EPCR antibody. Exemplary anti-EPCR antibodies include JRK1494, JRK1535; and anti-EPCR antibodies disclosed in US Patent Application Nos. US20210355231 and US20220127374; and PCT Application Nos. WO2020051277 and WO2020161478.

In some embodiments, the target molecule is CD34. In some embodiments, the target cell is CD34+. In some embodiments, the targeting agent is an anti-CD34 antibody. Exemplary anti-CD34 antibodies include h4C8, 9C5; and anti-CD34 antibodies disclosed in US Patent Nos. U.S. Pat. Nos. 8,399,249, 8,927,696, and U.S. Ser. No. 10/106,623; US Patent Application Nos. US20090221003, US20130143238, US20100311955, US20130172533, US20170320966, US20170298148, US20180169177, US20190135945; and PCT Application Nos. WO2009079922 and WO2015121383.

In some embodiments, the target molecule is ASCT2. In some embodiments, the target cell is ASCT2+. In some embodiments, the targeting agent is an anti-ASCT2 antibody. Exemplary anti-ASCT2 antibodies include idactamab, MEDI7247, KM4008, KM4012, KM4018; and anti-ASCT2 antibodies disclosed in US Patent Nos. U.S. Pat. Nos. 8,268,592, 8,501,180, 8,945,870, 8,673,592, and U.S. Ser. No. 10/829,554; US Patent Application Nos. US20180273617, US20190367605, US20210024629; and PCT Application Nos. WO2017083451, WO2018089393.

In some embodiments, the target molecule is CD90. In some embodiments, the target cell is CD90+. In some embodiments, the targeting agent is an anti-CD90 antibody. Exemplary anti-CD90 antibodies include EPR3133, CL1028, CL1040, AF-9, JF10-09, 5E10, 7E1B11; and anti-CD90 antibodies disclosed in US Patent Application No. US20210054068; and PCT Application No. WO2017214050.

In some embodiments, the target molecule is CD164. In some embodiments, the target cell is CD164+. In some embodiments, the targeting agent is an anti-CD164 antibody. Exemplary anti-CD164 antibodies include 67D2, H-4, 32G1, EML2058, 5C5, N6B6, 4B4, and 15-11-14; and anti-CD164 antibodies disclosed in PCT Application No. WO2006002438; and German Patent Nos. DE19727813C1 and DE19727815C1.

In some embodiments, the target molecule is CD49f. In some embodiments, the target cell is CD49f+. In some embodiments, the targeting agent is an anti-CD49f antibody. Exemplary anti-CD49f antibodies include CL6957, GoH3, SR45-00, and MP4F10; and anti-CD49f antibodies disclosed in US Patent Nos. U.S. Pat. No. 5,538,725, U.S. Ser. No. 10/030,071; US Patent Application Nos. US20110301227, US20160194400, US20160280789; and PCT Application Nos. WO2015034052 and WO2018127655.

In some embodiments, the target molecule is CD146. In some embodiments, the target cell is CD146+. In some embodiments, the targeting agent is an anti-CD146 antibody. Exemplary anti-CD146 antibodies include imaprelimab, PRX003, ABX-MA1, huAA98, M2H-1, M2J-1, and JM1-24-3; and anti-CD146 antibodies disclosed in US Patent Nos. U.S. Pat. Nos. 6,924,360, 7,067,131, US709844, U.S. Pat. Nos. 9,447,190, 9,782,500, U.S. Ser. No. 10/407,506, U.S. Ser. No. 10/414,825, U.S. Ser. No. 10/407,507, U.S. Ser. No. 10/584,177, U.S. Ser. No. 10/905,771, U.S. Ser. No. 11/427,648; US Patent Application Nos. US20030147809, US20040115205, US20060246077, US20140314744, US20150239980, US20140227292, US20160206764, US20190192573, US20170002089, US20150259419, US20170037144, US20170129954, US20170101470, US20180271994, US20180105602, US20200010563, US20200165336, US20200216560, US20200262929, US20200100838, US20220041748, US20220041749; and PCT Application Nos. WO2003057006, WO2003057837, WO2003057838, WO2012170071, WO2014000338, WO2015044218, WO2015136469, WO2015136470, WO2017046776, WO2017046774, WO2017149513, WO2017153953, WO2017208210, WO2018033630, WO2018220467, WO2018223140, WO2019068842, WO2019133639, WO2019137309, WO2020132190, WO2020132232, and WO2022082073.

Further exemplary targeting agents and corresponding target molecules are described in the table below. The targeting agent can be any described in the referenced associated documents that bind to the associated target molecule.

| | | Associated | |
|---|---|---|---|
| Target Molecule | Targeting Agent | Documents | Company/Institution |
| CD117 (c-Kit) | N/A | WO2022187050 | Biolegend |
| CD117 (c-Kit) | Briquilimab (also known | US20130288303 | Amgen/Jasper Therapeutics |
| | as JSP191 or AMG191) | US8791249 | |
| | | US20110223165 | |
| | | US8436150 | |
| | | WO2007127317 | |
| | | US7915391 | |
| CD117 (c-Kit) | CDX-0158 (also known | US20200385483 | Celldex |
| | as KTN0158) | US20220010027 | |
| | | US10774146 | |
| | | US20190153094 | |
| | | US20190106510 | |
| | | US10781267 | |
| | | US20190100598 | |
| | | US10793639 | |
| | | US20170158778 | |
| | | US10184007 | |
| | | US20170073422 | |
| | | US10189907 | |
| | | US20160311924 | |
| | | US9605081 | |
| | | WO2015179737 | |
| | | US20170121408 | |
| | | US10239943 | |
| | | WO2015112822 | |
| | | WO2015050959 | |
| | | WO2014018625 | |
| | | US20140065168 | |
| | | US9334332 | |
| | | WO2012103165 | |
| | | US20140056905 | |
| | | US9540443 | |
| | | WO2011119948 | |
| | | US20130011406 | |
| CD117 (c-Kit) | CDX-0159 | WO2022159737 | Celldex |
| CD117 (c-Kit) | | US20220127358 | Forty Seven |
| | | US20210122822 | |
| | | US11041022 | |
| | | WO2020242895 | |
| | | US20200165337 | |
| | | WO2020112687 | |
| | | US11208482 | |
| CD117 (c-Kit) | | WO2021099418 | IDD-Tech |
| CD117 (c-Kit) | | WO2012154861 | ImClone |
| | | US8552157 | |
| CD117 (c-Kit) | | WO2015067667 | INSERM |
| | | US20160264680 | |
| | | US9932410 | |
| CD117 (c-Kit) | LOP628 (also known as | US20220305134 | Novartis |
| | LMJ729) | US20200405875 | |
| | | US20200129632 | |
| | | WO2019244082 | |
| | | US20210228731 | |
| | | WO2018116178 | |
| | | US20190321483 | |
| | | US11357864 | |
| | | US20180125996 | |
| | | US20170035905 | |
| | | US10117953 | |
| | | WO2016020791 | |
| | | US20180193475 | |
| | | US10786578 | |
| | | US20150320880 | |
| | | US20170021033 | |
| | | US20140271688 | |
| | | WO2014150937 | |
| | | US20160030594 | |
| | | US9498543 | |
| | | US9789203 | |
| CD117 (c-Kit) | MGTA-117 (also known | US20220177575 | Magenta Thera |
| | as CD117-ADC or | US20220177578 | |
| | MGTA-C100) | US20220177577 | |
| | | US20220175944 | |
| | | US20220226493 | |
| | | US20210283267 | |
| | | US20210317228 | |
| | | US20200255523 | |
| | | WO2020219964 | |
| | | US20200376135 | |
| | | WO2020219748 | |
| | | WO2020219770 | |
| | | WO2020219774 | |
| | | WO2020219778 | |
| | | WO2020219775 | |
| | | WO2019234694 | |
| | | WO2019084067 | |
| | | WO2019084064 | |
| | | WO2019084057 | |
| | | WO2019084053 | |
| | | US20190153114 | |
| | | US10882915 | |
| | | US10899843 | |
| | | US20190134217 | |
| CD117 (c-Kit) | NN2101 (also known | WO2020076105 | Ajou University |
| | as 2G4) | WO2021107566 | |
| | | US20210355212 | |
| CD117 (c-Kit) | N/A (internal name | WO2016033201 | Stanford University |
| | SR1) | US20170224737 | |
| | | US10406179 | |
| CD117 (c-Kit) | N/A | US20200283521 | Granular |
| | | US11377493 | Therapeutics/Ultrahuman |
| | | US20200010548 | Four |
| | | US10611838 | |
| | | WO2019155067 | |
| CD117 (c-Kit) | N/A | WO2021188590 | Magenta Thera |
| (CD117/CD3 | | | |
| bispecific) | | | |
| CD117 (c-Kit) | N/A | WO2021044008 | University of Zurich |
| (CD117/CD3 | | | |
| bispecific) | | | |
| CD110 (TPO | N/A | WO2011060076 | Amgen |
| Receptor/c-Mpl) | | US20120269814 | |
| CD110 (TPO | N/A (also known as | US20120015436 | Chugai Pharmaceutical |
| Receptor/c-Mpl) | TA136) | US8586039 | |
| | | US20090297501 | |
| | | US9493569 | |
| | | WO2006106903 | |
| | | US7691588 | |
| | | US8034903 | |
| CD110 (TPO | N/A | WO2022114603 | Daegu Gyeongbuk Institute |
| Receptor/c-Mpl) | | | of Science and Technology |
| CD110 (TPO | N/A | US20130053547 | Kyowa Hakko Kirin Co. |
| Receptor/c-Mpl) | | US8999329 | |
| | | US20120053326 | |
| | | US8236314 | |
| | | WO2009041734 | |
| | | US20100004429 | |
| | | US8048421 | |
| ITGA3 (Integrin alpha | N/A | US20190309072 | Cytomx Therapeutics |
| 3, VLA3A, CD49c) | | WO2016179335 | |
| | | US20160355592 | |
| | | US10233244 | |
| ITGA3 (Integrin alpha | N/A (also known as OV- | Ke et al. Cancer Sci. | Academia Sinica |
| 3, VLA3A, CD49c) | Ab 30-7) | 2020; 111: | |
| | | 3478-3492. | |
| CD46 (MCP, | N/A | WO2021257542 | BioAtla |
| Membrane cofactor | | | |
| protein) | | | |
| CD46 (MCP, | FOR46 (also known as | WO2022150512 | Fortis Therapeutics Inc/ |
| Membrane cofactor | YS5FL) | WO2022150514 | University of California |
| protein) | | WO2022150517 | |
| | | WO2022032020 | |
| | | US20200199245 | |
| | | US20200040094 | |
| | | US11208493 | |
| | | WO2018089807 | |
| | | US20190276553 | |
| | | US11434301 | |
| | | US20170240643 | |
| | | US10400045 | |
| | | WO2016040683 | |
| | | US20170362330 | |
| | | US10533056 | |
| | | US20140271685 | |
| | | US9567402 | |
| CD46 (MCP, | N/A | WO2003032814 | MacroGenics Inc |
| Membrane cofactor | | | |
| protein) | | | |
| CD46 (MCP, | N/A | US20170081419 | StemCentRx |
| Membrane cofactor | | US10017565 | |
| protein) | | WO2012031273 | |
| | | US9458231 | |
| | | US20130302355 | |

In some embodiments, a protein fusogen derived from a virus or organism that do not infect humans does not have a natural fusion targets in patients and/or subject, and thus has high specificity.

In some embodiments, the G protein or functionally active variant or biologically active portion thereof is linked directly to the binding domain and/or variable domain thereof. In some embodiments, the targeted envelope protein is a fusion protein that has the following structure: (N′-single domain antibody-C′)-(C′-G protein-N′).

In some embodiments, the G protein or functionally active variant or biologically active portion thereof is linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C‘-G protein-N’).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:20), GGGGGS (SEQ ID NO:21) or combinations thereof. In some embodiments, the polypeptide linker has the sequence (GGS)n, wherein n is 1 to 10. In some embodiments, the polypeptide linker has the sequence (GGGGS)n, (SEQ ID NO:22) wherein n is 1 to 10. In some embodiments, the polypeptide linker has the sequence (GGGGGS)n (SEQ ID NO:23), wherein n is 1 to 6.

### G. Payload Gene

In some embodiments, the lipid particle comprising a nucleic acid encoding a payload gene. For example, the lipid particle may comprise a nucleic acid that is or encodes an RNA to enhance expression of an endogenous protein, or a siRNA or miRNA that inhibits protein expression of an endogenous protein. For example, the endogenous protein may modulate structure or function in the target cells. In some embodiments, the lipid particle may comprise a nucleic acid that is or encodes an engineered protein that modulates structure or function in the target cells. In some embodiments, the lipid particle may comprise a nucleic acid that is or encodes a transcriptional activator that modulate structure or function in the target cells.

In some embodiments, the lipid described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide.

In some embodiments, the lipid particle contains a nucleic acid that encodes a payload gene (also referred to as a “heterologous, recombinant, exogenous, or therapeutic gene.”).

In some embodiments, the payload gene encodes a protein that comprises a cytosolic protein, e.g., a protein that is produced in the recipient cell and localizes to the recipient cell cytoplasm. In some embodiments, payload gene encodes a protein that comprises a secreted protein, e.g., a protein that is produced and secreted by the recipient cell. In some embodiments, the payload gene encodes a protein that is a nuclear protein, e.g., a protein that is produced in the recipient cell and is imported to the nucleus of the recipient cell. In some embodiments, the payload gene encodes a protein that comprises an organellar protein (e.g., a mitochondrial protein), e.g., a protein that is produced in the recipient cell and is imported into an organelle (e.g., a mitochondrial) of the recipient cell.

In some embodiments, the payload gene encodes a protein that comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the payload gene encodes a protein that is a nuclease for use in gene editing methods. In some embodiments, the nuclease is a zinc-finger nucleases (ZFNs), transcription-activator like effector nucleases (TALENs), or a CRISPR-associated protein- nuclease (Cas). In some embodiments, the Cas is Cas9 from Streptococcus pyogenes. In some embodiments, the Cas is a Cas12a (also known as cpf1) from a Prevotella or Francisella bacteria, or the Cas is a Cas12b from a Bacillus, optionally Bacillus hisashii. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020)

In some embodiments, the provided the lipid particle contains a payload gene that encodes a protein that is a nuclease protein. In some embodiments, the provided the lipid particle contains a protein that is a nuclease protein and the nuclease protein is directly delivered to a target cell Methods of delivering a nuclease protein include those as described, for example, in Cai et al. Elife, 2014, 3:e01911 and International patent publication No. WO2017068077. For instance, the provided lipid particle comprises one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. paramyxovirus lipid particles). For instance, a chimeric Cas9-protein fusion with the structural GAG protein can be packaged inside a paramyxovirus lipid particle. In some embodiments, the fusion protein is a cleavable fusion protein between (i) a viral structural protein (e.g. GAG) and (ii) a nuclease protein (e.g. Cas protein, such as Cas 9).

In some embodiments, the lipid particle is a particle which further comprises an encapsulated polypeptide or polynucleotide encoding a payload gene, a therapeutic gene, an exogenous gene, and/or a recombinant gene, such as any recombinant gene, particularly a therapeutic gene.

In some embodiments, the payload gene comprises a nucleic acid (i.e., a heterologous, recombinant, exogenous, or therapeutic gene) that encodes a cytosolic protein, e.g., a protein that is produced in the recipient cell and localizes to the recipient cell cytoplasm. In some embodiments, the payload gene comprises a nucleic acid that encodes a secreted protein, e.g., a protein that is produced and secreted by the recipient cell. In some embodiments, the payload gene comprises a nucleic acid that encodes a nuclear protein, e.g., a protein that is produced in the recipient cell and is imported to the nucleus of the recipient cell. In some embodiments, the payload gene comprises a nucleic acid that encodes an organellar protein (e.g., a mitochondrial protein), e.g., a protein that is produced in the recipient cell and is imported into an organelle (e.g., a mitochondrial) of the recipient cell.

In some embodiments, the payload gene comprises a nucleic acid (i.e., a heterologous, recombinant, exogenous, or therapeutic gene) that encodes a membrane protein. In some embodiments, the membrane protein comprises a nucleic acid that encodes a chimeric antigen receptor (CAR), a T cell receptor, an integrin, an ion channel, a pore forming protein, a Toll-Like Receptor, an interleukin receptor, a cell adhesion protein, or a transport protein. In some embodiments, delivery of the nuclease is by a provided vector encoding the nuclease (e.g. Cas).

In some embodiments, the payload gene is a globin gene. In some embodiments, the payload gene is ADA, IL2RG, JAK3, IL7R, HBB, F8, F9, WAS, CYBA, CYBB, NCF1, NCF2, NCF4, UROS, TCIRG1, CLCN7, MPL, ITGA2B, ITGB3, ITGB2, PKLR, SLC25, A38, RAGI, RAG2, FANCA, FANCC, FANCG, ABCD1, MAN2B1, AGA, LYST, CTNS, LAMP2, GLA, CTSA, GBA, GAA, IDS, IDUA, ISSD, ARSB, GALNS, GLB1, NEU1, GNPTA, SUMFI, SMPD1, NPC1, NPC2, CTSK, GNS, HGSNAT, NAGLU, SGSH, NAGA, GUSB, PSAP, LAL. In some embodiment, the payload gene can be a gene for delivery to a hematopoietic stem cell (HSC). Exemplary payload genes are described in WO2020102485, which is incorporated by reference.

For example, the payload gene can be, but is not limited to antisense ras, antisense myc, antisense raf, antisense erb, antisense src, antisense fins, antisense jun, antisense trk, antisense ret, antisense gsp, antisense hst, antisense bcl, antisense abl, Rb, CFTR, pi 6, p21, p27, p57, p73, C-CAM, APC, CTS-I, zacl, scFV ras, DCC, NF—I, NF-2, WT-I, MEN-I, MEN-II, BRCAl, VHL, MMACl, FCC, MCC, BRCA2, IL-I, IL-2, IL-3, IL- 4, IL-5, IL-6, IL-7, IL-8, IL-9, IL-10, IL-Il IL-12, GM-CSF, G-CSF, thymidine kinase, mda7, fus-1, interferon a, interferon J, interferon y, ADP, p53, ABLI, BLCl, BLC6, CBFAl, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIMI, PML, RET, SRC, TALI, TCL3, YES, MADH4, RBl, TP53, WT1, TNF, BDNF, CNTF, NGF, IGF, GMF, aFGF, bFGF, NT3, NT5, ApoAI, ApoAIV, ApoE, RaplA, cytosine deaminase, Fab, ScFv, BRCA2, zacl, ATM, HIC-I, DPC-4, FHIT, PTEN, INGI, NOEYl, NOEY2, OVCAl, MADR2, 53BP2, IRF-I, Rb, zacl, DBCCR-I, rks-3, COX-I, TFPI, PGS, Dp, E2F, ras, myc, neu, raf, erb, fins, trk, ret, gsp, hst, abl, ElA, p300, VEGF, FGF, thrombospondin, BAI-I, GDAIF, or MCC. In further embodiments of the present invention, the payload gene is a gene encoding an ACP desaturase, an ACP hydroxylase, an ADP- glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyrne, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. In other embodiments of the present invention, the payload gene is a gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, gmcose-6-phosphatase, low-density-lipoprotein receptor, porphobilinogen deaminase, factor VIII, factor IX, cystathione a-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase. galactose- 1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase. Alternatively, the recombinant gene may encode growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

### 1. Chimeric Antigen Receptors

In certain embodiments, the payload gene may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 4 below.

TABLE 4
Exemplary sequences of signal peptides

| SEQ ID | | |
|---|---|---|
| NO: | Sequence | Description |
| 24 | MALPVTALLLPLALLLHAARP | CD8α signal peptide |
| 25 | METDTLLLWVLLLWVPGSTG | IgK signal peptide |
| 26 | MLLLVTSLLLCELPHPAFLLIP | GMCSFR-α (CSF2RA) signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The VH and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRa, IL-13Ra, Mesothelin, MUCI, MUC16, and RORI (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 5 below.

TABLE 5
Exemplary sequences of hinge domains

| SEQ ID | | |
|---|---|---|
| NO: | Sequence | Description |
| 27 | TTTPAPRPPTPAPTIASQPLSLRPEACRPAAGGAVH | CD8α hinge domain |
| | TRGLDFACD | |
| 28 | IEVMYPPPYLDNEKSNGTIIHVKGKHLCPSPLFPGP | CD28 hinge domain |
| | SKP | |
| 29 | AAAIEVMYPPPYLDNEKSNGTIIHVKGKHLCPSPLF | CD28 hinge domain |
| | PGPSKP | |
| 30 | ESKYGPPCPPCP | IgG4 hinge domain |
| 31 | ESKYGPPCPSCP | IgG4 hinge domain |
| 32 | ESKYGPPCPPCPAPEFLGGPSVFLFPPKPKDTLMISR | IgG4 hinge-CH2-CH3 |
| | TPEVTCVVVDVSQEDPEVQFNWYVDGVEVHNAK | domain |
| | TKPREEQFNSTYRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCSV | |
| | MHEALHNHYTQKSLSLSLGK | |

In certain embodiments, the transmembrane domain of the CAR may comprise a transmembrane region of the alpha, beta, or zeta chain of a T cell receptor, CD28, CD3E, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8a, CD80, 4-1BB/CD137, CD2, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B3, or a functional variant thereof, including the human versions of each of these sequences. Table 6 provides the amino acid sequences of a few exemplary transmembrane domains.

TABLE 6
Exemplary sequences of transmembrane domains

| SEQ ID | | |
|---|---|---|
| NO: | Sequence | Description |
| 33 | IYIWAPLAGTCGVLLLSLVITLYC | CD8α transmembrane domain |
| 34 | FWVLVVVGGVLACYSLLVTVAFIIFWV | CD28 transmembrane domain |
| 35 | MFWVLVVVGGVLACYSLLVTVAFIIFWV | CD28 transmembrane domain |

In certain embodiments, the intracellular signaling domain and/or intracellular costimulatory domain of the CAR may comprise one or more signaling domains selected from B7-1/CD80, 1B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSFi3C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSFi4, Lymphotoxin-alpha/TNFO, OX40/TNFRSF4, OX40 Ligand/TNFSF4, RELT/TNFRSF19L, TACI/TNFRSF13B, TL1A/TNFSFi5, TNFa, TNF RII/TNFRSFIB, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3(, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3(domain, an ITAM, a CD28 domain, 4-1BB domain, or a functional variant thereof. Table 7 provides the amino acid sequences of a few exemplary intracellular costimulatory and/or signaling domains. In certain embodiments, as in the case of tisagenlecleucel as described below, the CD3ζ signaling domain of SEQ ID NO:38 may have a mutation, e.g., a glutamine (Q) to lysine (K) mutation, at amino acid position 14 (see SEQ ID NO:39).

TABLE 7
Exemplary sequences of intracellular costimulatory and/or signaling domains

| SEQ ID | | |
|---|---|---|
| NO: | Sequence | Description |
| 36 | KRGRKKLLYIFKQPFMRPVQTTQEEDGCSCR | 4-1BB costimulatory domain |
| | FPEEEEGGCEL | |
| 37 | RSKRSRLLHSDYMNMTPRRPGPTRKHYQPY | CD28 costimulatory domain |
| | APPRDFAAYRS | |
| 38 | RVKFSRSADAPAYQQGQNQLYNELNLGRRE | CD3ζ signaling domain |
| | EYDVLDKRRGRDPEMGGKPRRKNPQEGLYN | |
| | ELQKDKMAEAYSEIGMKGERRRGKGHDGLY | |
| | QGLSTATKDTYDALHMQALPPR | |
| 39 | RVKFSRSADAPAYKQGQNQLYNELNLGRRE | CD3ζ signaling domain (with Q |
| | EYDVLDKRRGRDPEMGGKPRRKNPQEGLYN | to K mutation at position 14) |
| | ELQKDKMAEAYSEIGMKGERRRGKGHDGLY | |
| | QGLSTATKDTYDALHMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

a. CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8a signal peptide. In some embodiments, the CD8a signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 8 below. In some embodiments, the CD19-specific scFv comprises or consists of an amino acid sequence set forth in SEQ ID NO:40, 41, or 46, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:40, 41, or 46. In some embodiments, the CD19-specific scFv may comprise one or more CDRs having amino acid sequences set forth in SEQ ID NOs: 42-44 and 48-50. In some embodiments, the CD19-specific scFv may comprise a light chain with one or more CDRs having amino acid sequences set forth in SEQ ID NOs: 42-44. In some embodiments, the CD19-specific scFv may comprise a heavy chain with one or more CDRs having amino acid sequences set forth in SEQ ID NOs: 48-50. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:45. In some embodiments, the Whitlow linker may be replaced by a different linker, for example, a 3xG₄S linker having an amino acid sequence set forth in SEQ ID NO:51, which gives rise to a different FMC63-derived scFv having an amino acid sequence set forth in SEQ ID NO:50. In certain of these embodiments, the CD19-specific scFv comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50.

TABLE 8
Exemplary sequences of anti-CD19 scFv and components

| SEQ ID NO: | Amino Acid Sequence | Description |
|---|---|---|
| 40 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 41 | DIQMTQTTSSLSASLGDRVTISCRASQDI | Anti-CD19 FMC63 scFv |
| | SKYLNWYQQKPDGTVKLLIYHTSRLHS | light chain variable region |
| | GVPSRFSGSGSGTDYSLTISNLEQEDIAT | |
| | YFCQQGNTLPYTFGGGTKLEIT | |
| 42 | QDISKY | Anti-CD19 FMC63 scFv |
| | | light chain CDR1 |
| 43 | HTS | Anti-CD19 FMC63 scFv |
| | | light chain CDR2 |
| 44 | QQGNTLPYT | Anti-CD19 FMC63 scFv |
| | | light chain CDR3 |
| 45 | GSTSGSGKPGSGEGSTKG | Whitlow linker |
| 46 | EVKLQESGPGLVAPSQSLSVTCTVSGVS | Anti-CD19 FMC63 scFv |
| | LPDYGVSWIRQPPRKGLEWLGVIWGSE | heavy chain variable region |
| | TTYYNSALKSRLTIIKDNSKSQVFLKMN | |
| | SLQTDDTAIYYCAKHYYYGGSYAMDY | |
| | WGQGTSVTVSS | |
| 47 | GVSLPDYG | Anti-CD19 FMC63 scFv |
| | | heavy chain CDR1 |
| 48 | IWGSETT | Anti-CD19 FMC63 scFv |
| | | heavy chain CDR2 |
| 49 | AKHYYYGGSYAMDY | Anti-CD19 FMC63 scFv |
| | | heavy chain CDR3 |
| 50 | DIQMTQTTSSLSASLGDRVTISCRASQDI | Anti-CD19 FMC63 scFv |
| | SKYLNWYQQKPDGTVKLLIYHTSRLHS | entire sequence, with 3xG4S |
| | GVPSRFSGSGSGTDYSLTISNLEQEDIAT | linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 51 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28. In some embodiments, the CD28 hinge domain comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:31 or SEQ ID NO:30, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:31 or SEQ ID NO:30. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD19 CAR comprises a CD8α transmembrane domain, for example, a human CD8a transmembrane domain. In some embodiments, the CD8α transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:34 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:34. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the CD8α hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the IgG4 hinge domain of SEQ ID NO:30 or SEQ ID NO:31, the CD28 transmembrane domain of SEQ ID NO:34, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the CD28 hinge domain of SEQ ID NO:28, the CD28 transmembrane domain of SEQ ID NO:34, the CD28 costimulatory domain of SEQ ID NO:37, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:52 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:52 (see Table 10). The encoded CD19 CAR has a corresponding amino acid sequence set forth in SEQ ID NO:53 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53, with the following components: CD8α signal peptide, FMC63 scFv (V_{L}—Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3xG₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 10, with annotations of the sequences provided in Table 10.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}—Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 9, with annotations of the sequences provided in Table 11.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}—Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 9, with annotations of the sequences provided in Table 12.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 54, 56, or 58, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 54, 56, or 58. The encoded CD19 CAR has a corresponding amino acid sequence set forth in SEQ ID NO: 55, 57, or 59, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 55, 57, or 59, respectively.

TABLE 9
Exemplary sequences of CD19 CARs

| SEQ ID NO: | Sequence | Description |
|---|---|---|
| 52 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattcgcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaccggcgcccaccatcgcgtcgcagc | |
| | ccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctcgc | |
| 53 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 54 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaccggcgcccaccatcgcgtcgcagcccctgtccctgc | |
| | gcccagaggcgtgccggccagcggcggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 55 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 56 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcagcggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagcgaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggcggggcaagggccacgacggcctgtatc | |
| | agggcctgtccaccgccaccaaggatacctacgacgccctgcacatgca | |
| | ggccctgcccccaagg | |
| 57 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 58 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 59 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 10
Annotation of tisagenlecleucel CD19 CAR sequences

| | Nucleotide Sequence | Amino Acid Sequence |
|---|---|---|
| Feature | Position | Position |
| CD8α signal peptide | 1-63 | 1-21 |
| FMC63 scFv | 64-789 | 22-263 |
| (V_{L}-3xG4S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 11
Annotation of lisocabtagene maraleucel CD19 CAR sequences

| | Nucleotide Sequence | Amino Acid Sequence |
|---|---|---|
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 12
Annotation of axicabtagene ciloleucel CD19 CAR sequences

| | Nucleotide Sequence | Amino Acid Sequence |
|---|---|---|
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 54, 56, or 58, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 54, 56, or 58. The encoded CD19 CAR has a corresponding amino acid sequence set forth in SEQ ID NO: 55, 57, or 59, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 55, 57, or 59, respectively. b. CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu 16 connected by a linker. See Wu et al., Protein Engineering. 14(12):1025-1033 (2001). In some embodiments, the linker is a 3xG₄S linker. In other embodiments, the linker is a Whitlow linker as described herein. In some embodiments, the amino acid sequences of different portions of the entire Leu6-derived scFv (also referred to as Leu16 scFv) and its different portions are provided in Table 13 below. In some embodiments, the CD20-specific scFv comprises or consists of an amino acid sequence set forth in SEQ ID NO:60, 61, or 65, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:60, 61, or 65 In some embodiments, the CD20-specific scFv may comprise one or more CDRs having amino acid sequences set forth in SEQ ID NOs: 62-64, 66, 67, and 68. In some embodiments, the CD20-specific scFv may comprise a light chain with one or more CDRs having amino acid sequences set forth in SEQ ID NOs: 62-64. In some embodiments, the CD20-specific scFv may comprise a heavy chain with one or more CDRs having amino acid sequences set forth in SEQ ID NOs: 66, 67, and 68. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 13
Exemplary sequences of anti-CD20 scFv and components

| SEQ ID NO: | Amino Acid Sequence | Description |
|---|---|---|
| 60 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 61 | DIVLTQSPAILSASPGEKVTMTCRASSS | Anti-CD20 Leu16 scFv |
| | VNYMDWYQKKPGSSPKPWIYATSNLA | light chain variable region |
| | SGVPARFSGSGSGTSYSLTISRVEAEDA | |
| | ATYYCQQWSFNPPTFGGGTKLEIK | |
| 62 | RASSSVNYMD | Anti-CD20 Leu16 scFv |
| | | light chain CDR1 |
| 63 | ATSNLAS | Anti-CD20 Leu16 scFv |
| | | light chain CDR2 |
| 64 | QQWSFNPPT | Anti-CD20 Leu16 scFv |
| | | light chain CDR3 |
| 65 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 66 | SYNMH | Anti-CD20 Leu16 scFv |
| | | heavy chain CDR1 |
| 67 | AIYPGNGDTSYNQKFKG | Anti-CD20 Leu16 scFv |
| | | heavy chain CDR2 |
| 68 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:30 or SEQ ID NO:31, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:30 or SEQ ID NO:31. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD8α hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD28 hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the IgG4 hinge domain of SEQ ID NO:30 or SEQ ID NO:31, the CD8α transmembrane domain of SEQ ID NO:33, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD8α hinge domain of SEQ ID NO:27, the CD28 transmembrane domain of SEQ ID NO:29, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD28 hinge domain of SEQ ID NO:29, the CD28 transmembrane domain of SEQ ID NO:35, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the IgG4 hinge domain of SEQ ID NO:30 or SEQ ID NO:31, the CD28 transmembrane domain of SEQ ID NO:34, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

c. CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:85 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:85. In some embodiments, the signal peptide comprises an IgK signal peptide. In some embodiments, the IgK signal peptide comprises or consists of an amino acid sequence set forth in SEQ ID NO:86 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:87 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:87.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3xG₄S linker. In other embodiments, the Whitlow linker may be used instead. In some embodiments, the amino acid sequences of the entire m971-derived scFv (also referred to as m971 scFv) and its different portions are provided in Table 15 below. In some embodiments, the CD22-specific scFv comprises or consists of an amino acid sequence set forth in SEQ ID NO:69, 70, or 74, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:69, 70, or 74. In some embodiments, the CD22-specific scFv may comprise one or more CDRs having amino acid sequences set forth in SEQ ID NOs: 71-73 and 75-77. In some embodiments, the CD22-specific scFv may comprise a heavy chain with one or more CDRs having amino acid sequences set forth in SEQ ID NOs: 71-73. In some embodiments, the CD22-specific scFv may comprise a light chain with one or more CDRs having amino acid sequences set forth in SEQ ID NOs: 75-77. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the VH and the V_{L} of m971-L7 connected by a 3xG₄S linker. In other embodiments, the Whitlow linker may be used instead. In some embodiments, the amino acid sequences of the entire m971-L7-derived scFv (also referred to as m971-L7 scFv) and its different portions are provided in Table 14 below. In some embodiments, the CD22-specific scFv comprises or consists of an amino acid sequence set forth in SEQ ID NO:78, 79, or 83, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:78, 79, or 83. In some embodiments, the CD22-specific scFv may comprise one or more CDRs having amino acid sequences set forth in SEQ ID NOs: 80-82, or 84-86. In some embodiments, the CD22-specific scFv may comprise a heavy chain with one or more CDRs having amino acid sequences set forth in SEQ ID NOs: 80-82. In some embodiments, the CD22-specific scFv may comprise a light chain with one or more CDRs having amino acid sequences set forth in SEQ ID NOs: 84-86. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 14
Exemplary sequences of anti-CD22 scFv and components

| SEQ ID NO: | Amino Acid Sequence | Description |
|---|---|---|
| 69 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG₄S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 70 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 71 | GDSVSSNSAA | Anti-CD22 m971 scFv |
| | | heavy chain CDR1 |
| 72 | TYYRSKWYN | Anti-CD22 m971 scFv |
| | | heavy chain CDR2 |
| 73 | AREVTGDLEDAFDI | Anti-CD22 m971 scFv |
| | | heavy chain CDR3 |
| 74 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 75 | QTIWSY | Anti-CD22 m971 scFv |
| | | light chain CDR1 |
| 76 | AAS | Anti-CD22 m971 scFv |
| | | light chain CDR2 |
| 77 | QQSYSIPQT | Anti-CD22 m971 scFv |
| | | light chain CDR3 |
| 78 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG₄S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 79 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 80 | GDSVSSNSVA | Anti-CD22 m971-L7 scFv |
| | | heavy chain CDR1 |
| 81 | TYYRSTWYN | Anti-CD22 m971-L7 scFv |
| | | heavy chain CDR2 |
| 82 | AREVTGDLEDAFDI | Anti-CD22 m971-L7 scFv |
| | | heavy chain CDR3 |
| 83 | DIQMIQSPSSLSASVGDRVTITCRASQTI | Anti-CD22 m971-L7 scFv |
| | WSYLNWYRQRPGEAPNLLIYAASSLQS | light chain variable |
| | GVPSRFSGRGSGTDFTLTISSLQAEDFAT | region |
| | YYCQQSYSIPQTFGQGTKLEIK | |
| 84 | QTIWSY | Anti-CD22 m971-L7 scFv |
| | | light chain CDR1 |
| 85 | AAS | Anti-CD22 m971-L7 scFv |
| | | light chain CDR2 |
| 86 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO: 88 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:88. In some embodiments, the hinge domain comprises a CD28 hinge domain, for example, a human CD28 hinge domain. In some embodiments, the CD28 hinge domain comprises or consists of an amino acid sequence set forth in SEQ ID NO:89 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:89. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:91 or SEQ ID NO:92, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:91 or SEQ ID NO:92. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:93.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:94 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:94. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:95 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:95.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:97 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:97. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:98 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:98.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:99 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:99.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD8α hinge domain of SEQ ID NO:88, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD28 hinge domain of SEQ ID NO:89, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:78, the IgG4 hinge domain of SEQ ID NO:91 or SEQ ID NO:92, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:69 or SEQ ID NO:78, the CD8α hinge domain of SEQ ID NO:8, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:69 or SEQ ID NO:78, the CD28 hinge domain of SEQ ID NO:89, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:69 or SEQ ID NO:78, the IgG4 hinge domain of SEQ ID NO:91 or SEQ ID NO:92, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

d. BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:85 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:85. In some embodiments, the signal peptide comprises an IgK signal peptide. In some embodiments, the IgK signal peptide comprises or consists of an amino acid sequence set forth in SEQ ID NO:86 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:87 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:87.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 16 below. In some embodiments, the BCMA-specific extracellular binding domain comprises or consists of an amino acid sequence set forth in SEQ ID NO:87, 88, or 92, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:87, 88, or 92. In some embodiments, the BCMA-specific extracellular binding domain may comprise one or more CDRs having amino acid sequences set forth in SEQ ID NOs: 89-91 and 93-95. In some embodiments, the BCMA-specific extracellular binding domain may comprise a light chain with one or more CDRs having amino acid sequences set forth in SEQ ID NOs: 89-91. In some embodiments, the BCMA-specific extracellular binding domain may comprise a heavy chain with one or more CDRs having amino acid sequences set forth in SEQ ID NOs: 93-95. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 15 below. In some embodiments, the BCMA-specific extracellular binding domain comprises or consists of an amino acid sequence set forth in SEQ ID NO:96, 97, or 101, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:96, 97, or 101. In some embodiments, the BCMA-specific extracellular binding domain may comprise one or more CDRs having amino acid sequences set forth in SEQ ID NOs: 98-100 and 103-104. In some embodiments, the BCMA-specific extracellular binding domain may comprise a light chain with one or more CDRs having amino acid sequences set forth in SEQ ID NOs: 98-100. In some embodiments, the BCMA-specific extracellular binding domain may comprise a heavy chain with one or more CDRs having amino acid sequences set forth in SEQ ID NOs: 102-104. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 15 below. In some embodiments, the BCMA-specific extracellular binding domain comprises or consists of an amino acid sequence set forth in SEQ ID NO:105 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:105. In some embodiments, the BCMA-specific extracellular binding domain may comprise one or more CDRs having amino acid sequences set forth in SEQ ID NOs: 106-108. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 15 below. In some embodiments, the BCMA-specific extracellular binding domain comprises or consists of an amino acid sequence set forth in SEQ ID NO:168, 169, or 173, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 109, 110, or 114. In some embodiments, the BCMA-specific extracellular binding domain may comprise one or more CDRs having amino acid sequences set forth in SEQ ID NOs: 111-113 and 115-117. In some embodiments, the BCMA-specific extracellular binding domain may comprise a light chain with one or more CDRs having amino acid sequences set forth in SEQ ID NOs: 111-113. In some embodiments, the BCMA-specific extracellular binding domain may comprise a heavy chain with one or more CDRs having amino acid sequences set forth in SEQ ID NOs: 115-117. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 15
Exemplary sequences of anti-BCMA binder and components

| SEQ ID NO: | Amino Acid Sequence | Description |
|---|---|---|
| 87 | DIVLTQSPASLAMSLGKRATISCRASES | Anti-BCMA C11D5.3 |
| | VSVIGAHLIHWYQQKPGQPPKLLIYLAS | scFv entire sequence, with |
| | NLETGVPARFSGSGSGTDFTLTIDPVEE | Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 88 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 89 | RASESVSVIGAHLIH | Anti-BCMA C11D5.3 |
| | | scFv light chain CDR1 |
| 90 | LASNLET | Anti-BCMA C11D5.3 |
| | | scFv light chain CDR2 |
| 91 | LQSRIFPRT | Anti-BCMA C11D5.3 |
| | | scFv light chain CDR3 |
| 92 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 93 | DYSIN | Anti-BCMA C11D5.3 |
| | | scFv heavy chain CDR1 |
| 94 | WINTETREPAYAYDFRG | Anti-BCMA C11D5.3 |
| | | scFv heavy chain CDR2 |
| 95 | DYSYAMDY | Anti-BCMA C11D5.3 |
| | | scFv heavy chain CDR3 |
| 96 | DIVLTQSPPSLAMSLGKRATISCRASESV | Anti-BCMA C12A3.2 |
| | TILGSHLIYWYQQKPGQPPTLLIQLASN | scFv entire sequence, with |
| | VQTGVPARFSGSGSRTDFTLTIDPVEED | Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 97 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 98 | RASESVTILGSHLIY | Anti-BCMA C12A3.2 |
| | | scFv light chain CDR1 |
| 99 | LASNVQT | Anti-BCMA C12A3.2 |
| | | scFv light chain CDR2 |
| 100 | LQSRTIPRT | Anti-BCMA C12A3.2 |
| | | scFv light chain CDR3 |
| 101 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 102 | HYSMN | Anti-BCMA C12A3.2 |
| | | scFv heavy chain CDR1 |
| 103 | RINTESGVPIYADDFKG | Anti-BCMA C12A3.2 |
| | | scFv heavy chain CDR2 |
| 104 | DYLYSLDF | Anti-BCMA C12A3.2 |
| | | scFv heavy chain CDR3 |
| 105 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 106 | GFTFSSYA | Anti-BCMA FHVH33 |
| | | CDR1 |
| 107 | ISGSGDYI | Anti-BCMA FHVH33 |
| | | CDR2 |
| 108 | AKEGTGANSSLADY | Anti-BCMA FHVH33 |
| | | CDR3 |
| 109 | DIQMTQSPSSLSASVGDRVTITCRASQSI | Anti-BCMA CT103A |
| | SSYLNWYQQKPGKAPKLLIYAASSLQS | scFv entire sequence, with |
| | GVPSRFSGSGSGTDFTLTISSLQPEDFAT | Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 110 | DIQMTQSPSSLSASVGDRVTITCRASQSI | Anti-BCMA CT103A |
| | SSYLNWYQQKPGKAPKLLIYAASSLQS | scFv light chain variable |
| | GVPSRFSGSGSGTDFTLTISSLQPEDFAT | region |
| | YYCQQKYDLLTFGGGTKVEIK | |
| 111 | QSISSY | Anti-BCMA CT103A |
| | | scFv light chain CDR1 |
| 112 | AAS | Anti-BCMA CT103A |
| | | scFv light chain CDR2 |
| 113 | QQKYDLLT | Anti-BCMA CT103A |
| | | scFv light chain CDR3 |
| 114 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 115 | GGSISSSSYY | Anti-BCMA CT103A |
| | | scFv heavy chain CDR1 |
| 116 | ISYSGST | Anti-BCMA CT103A |
| | | scFv heavy chain CDR2 |
| 117 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:88 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:88. In some embodiments, the hinge domain comprises a CD28 hinge domain, for example, a human CD28 hinge domain. In some embodiments, the CD28 hinge domain comprises or consists of an amino acid sequence set forth in SEQ ID NO:89 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:89. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:91 or SEQ ID NO:92, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:91 or SEQ ID NO:92. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:93.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:94 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:94. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:95 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:95.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:97 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:97. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:98 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:98.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:99 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:99.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:88, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:88, the CD8α transmembrane domain of SEQ ID NO:94, the CD28 costimulatory domain of SEQ ID NO:98, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:118 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:118 (see Table 16). The encoded BCMA CAR has a corresponding amino acid sequence set forth in SEQ ID NO:119 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 119, with the following components: CD8α signal peptide, CT103A scFv (V_{L}—Whitlow linker-V_{H}), CD8α hinge domain, CD8α transmembrane domain, 4-11B1 costimulatory domain, and CD3 signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb212t). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-11B1 costimulatory domain, and CD3ζ signaling domain.

TABLE 16
Exemplary sequences of BCMA CARs

| SEQ ID NO: | Sequence | Description |
|---|---|---|
| 118 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaatcgccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggcggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 119 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### 2. Gene Editing Enzymes

In some embodiments, the payload gene is or comprises a genome editing technology. In some embodiments, the payload gene is or comprises a heterologous protein that is associated with a genome editing technology. Any of a variety of agents associated with gene editing technologies can be included as the payload gene and/or heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the payload gene and/or heterologous protein mediates single-strand breaks (SSB). In some embodiments, the payload gene and/or heterologous protein mediates double-strand breaks (DSB), including in connection with non-homologous end-joining (NHEJ) or homology-directed repair (HDR). In some embodiments, the payload gene and/or heterologous protein does not mediate SSB. In some embodiments, the payload gene and/or heterologous protein does not mediate DSB. In some embodiments, the payload gene and/or heterologous protein can be used for DNA base editing or prime-editing. In some embodiments, the payload gene and/or heterologous protein can be used for Programmable Addition via Site-specific Targeting Elements (PASTE).

In some embodiments, the payload gene is a nuclease for use in gene editing methods. In some embodiments, the nuclease is a zinc-finger nucleases (ZFNs), transcription-activator like effector nucleases (TALENs), or a CRISPR-associated protein- nuclease (Cas). In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is Cas9 from Streptococcus pyogenes. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). In some embodiments, the Cas is a Cas12a (also known as Cpf1) from a Prevotella or Francisella bacteria, or the Cas is a Cas12b from a Bacillus, optionally Bacillus hisashii. In some embodiments, the Cas is a Cas12a (also known as cpf1) from a Prevotella, Francisella novicida, Acidaminococcus sp., Lachnospiraceae bacterium, or Francisella bacteria. In some embodiments, the nuclease is MAD7 or CasX. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020). The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety.

In some embodiments, delivery of the nuclease is by a provided vector encoding the nuclease (e.g. Cas).

In some embodiments, the provided viral vector particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided viral vector particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe- Petersen et al., 2013, Proc Natl Acad Sci USA, Vol. 110: 19860-19865; Gong et al., 2005, Nat. Struct Mol Biol, Vol. 12: 304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol.337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein. In some embodiments, the Cas protein comprises one or more mutations such that the Cas protein is converted into a nickase that is able to cleave only one strand of a double stranded DNA molecule (e.g., a SSB). In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx1O, Csxl1, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A.

In some embodiments, the Cas protein is selected from the group consisting of Cas3, Cas9, Cas10, Cas12, and Cas13. In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T, S4091, M6941, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., one or more payload gene and/or heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., one or more payload gene and/or heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., one or more payload gene and/or heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., one or more payload gene and/or heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, the provided lipid particles can be for use in a method to deliver an payload gene which involves introducing, into a cell, one or more agent(s) (e.g., one or more payload gene and/or heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., one or more payload gene and/or heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the one or more payload gene and/or heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the PAM sequence on the sense strand and the PAM sequence on the antisense strand comprise the same nucleic acid sequence, which can be any PAM sequence disclosed herein. In some embodiments, the PAM sequence on the sense strand and the PAM sequence on the antisense strand each comprise a different nucleic acid sequence, each of which can be any of the PAM sequences disclosed herein.

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg SH et al., Nature 2014 (doi: 10.1038/naturel30l1). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041,20150056705,20150031134,20150020223,20140357530,20140335620,20140310830, 20140273234,20140273232,20140273231,20140256046,20140248702,20140242700,20140242699, 20140242664,20140234972,20140227787,20140189896,20140186958,20140186919,20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C·G to T·A and adenine base editors (e.g., ABE7.10) that convert target A·T to G·C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the payload gene and/or heterologous protein is or encodes a base editor (e.g., a nucleobase editor). In some embodiments, the payload gene and/or heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the payload gene and/or heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and- replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the heterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the payload gene and/or heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the payload gene and/or heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, the payload gene and/or heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g. Cas).

In some embodiments, the provided lipid particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided lipid particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g. Cas protein, such as Cas9). In some embodiments, the fusion protein is a cleavable fusion protein between (i) a viral matrix (MA) protein and (ii) a nuclease protein (e.g. Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, a dCas9 version of the CRISPR/Cas9 system can be used to target protein domains for transcriptional regulation, epigenetic modification, and microscopic visualization of specific genome loci.

In some embodiments, the provided virus particles (e.g. lentiviral particles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing viral particles. In some embodiments, the provided virus particles (e.g. lentiviral particles) further comprises, or is further complexed with a targeting nucleic acid.

### Cytokine Receptor Agonists

In some embodiments, the methods provided herein include administering to a subject a cytokine receptor agonist. In some embodiments the cytokine receptor agonist is an agent which binds to a cytokine receptor on a T cell, such as any agent which interacts with a cytokine receptor and/or a cytokine that interacts with T cells. In some embodiments, the cytokine receptor is an IL-2 receptor, such as an intermediate affinity IL-2 receptor (IL-2Ry). In some embodiments, the cytokine receptor is an IL-7 receptor. In some embodiments, the cytokine receptor is an IL-15 receptor. In some embodiments, the cytokine receptor is an IL-21 receptor.

In some embodiments, the cytokine receptor agonist may include a cytokine or mutein thereof, cytokine mimetic, an antibody or antigen binding fragment thereof that is directed to a cytokine or cytokine receptor, or any such cytokine receptor agonist that is modified and/or conjugated. In some embodiments, the cytokine receptor agonist is a recombinant protein, a chemically synthesized protein, or a conjugate. In some embodiments, the cytokine receptor agonist is a cytokine or cytokine mutein, such as IL-2, IL-15, IL-21, IL-7, and/or a combination of any of the foregoing. In some embodiments, the cytokine receptor agonist is a cytokine mimetic such as a peptide that exhibits agonist activity of a T cell stimulating cytokine receptor. In some embodiments, the cytokine receptor agonist is an antibody or antigen binding fragment thereof that binds a cytokine receptor on a T cell, such as an antibody which binds with a cytokine receptor. In some embodiments, the cytokine receptor agonist is an antibody or antigen binding fragment thereof that binds a T cell stimulating cytokine, such as an antibody which binds with a cytokine peptide and, in some cases, promotes its interaction with a cytokine receptor. In some embodiments, the cytokine receptor agonist is modified, such as any modification which extends the half-life of the cytokine receptor agonist (e.g., a half-life extending moiety). In some embodiments, the cytokine receptor agonist is a conjugate, such as a conjugate with a polymer (e.g., water soluble polymer).

In some embodiments, the cytokine receptor agonist may be any as disclosed in U.S. Ser. No. 10/610,571, WO2021119534, WO2021263026, WO2020163532, US20180068055, AU2021225133, WO2022010928, CN111647068, US20210340208, US20210246183, WO2021258213, US20210188969, WO2020247388, TW202132332, TW202132330, WO2021133476, WO2021188374, WO2021081193, JP2021066748, WO2019165453, AU2021258008, IL283020, WO2020206395, WO2021054867, WO2017112528, CN105597092, CN102145178, CN101912599, US20210244825, WO2021102063, WO2022020637, US20210324027, WO3032046404, WO2021158623, WO2021158619, U.S. Pat. Nos. 8,153,114, 7,585,947, the entire contents of which are hereby incorporated by reference.

Exemplary cytokine receptor agonists are described in the following subjections. A non-limiting list of cytokine receptor agonists are shown in Table 17.

TABLE 17
Exemplary Cytokine Receptor Agonists

| Cytokine | Name |
|---|---|
| IL-2/IL-15 | NL-201 |
| IL-2 | SAR444245 |
| IL-2 | STK-012 |
| IL-2 | BPT-143 |
| IL-2 | AU-007 |
| IL-7 | IL-7 |
| IL-15 | IL-15 Synthorin |
| IL-21 | PIO-001 |
| IL-2 | Bempegaldesleukin |
| | (NKTR-214) |
| IL-2 | SHR-1916 |
| IL-2 | ARX102 |
| IL-2 | 8MW-2311 |
| IL-15 | NKTR-255 |
| IL-2 | Exenokine-2 |
| IL-2 | MDNA11 |
| IL-2 | IL-2 mAb |
| IL-7 | GX-17 / NT-17 |
| IL-15 | SHR-1501 |
| IL-15 | ASKG-215 |
| IL-15 | BCD-225 |
| IL-21 | Exenokine-21 |
| IL-7 | IL-7 Peptide |
| IL-15 | MK-1169 |
| IL-2 | Hu-Mikß1 |
| IL-2 | JS08-1 |
| IL-7 | CYT-107 |
| IL-15 | AM0015 |
| IL-15 | KW-007 |

In some embodiments, the cytokine receptor agonist is administered by the in-line method of administration in accord with the provided methods. In such examples, the transfection mixture may contain the cytokine receptor agonist, in which the methods of reinfusing the transfection mixture to the subject further administers the cytokine receptor agonist to the subject by the in-line method of administration. In some embodiments, the collected PBMCs or subset are contacted with the cytokine receptor agonist to produce the transfection mixture comprising the cytokine receptor agonist, wherein the contacting with the cytokine receptor agonist is carried out prior to the reinfusing of the transfection mixture or the contacted PBMCs or subset thereof to the subject. In some embodiments, the contacting with the cytokine receptor agonist is carried out prior to the contacting with the composition comprising lipid particles or lentiviral vector. In some embodiments, the contacting with the cytokine receptor agonist is carried out concurrently with the contacting with the composition comprising lipid particles or lentiviral vector. In some embodiments, the contacting with the cytokine receptor agonist is carried out after the contacting with the composition comprising lipid particles or lentiviral vector. In some embodiments, the contacting with the lipid particles or the lentiviral vector is performed under sterile conditions. In some embodiments, the contacting is in a closed system. In some embodiments, the contacting with the cytokine receptor agonist is performed in-line in the closed fluid circuit.

In some embodiments, the amount of the cytokine receptor agonist contacted with the lipid particles or lentiviral vectors is from or from about 0.05 mg to 10 mg. In some embodiments, the amount of the cytokine receptor agonist present in the transfection mixture is from or from about 0.05 mg to 10 mg. In some embodiments, the amount is from or from about 0.05 mg to 7.5 mg, from or from about 0.05 mg to 5 mg, from or from about 0.05 mg to 2.5 mg, from or from about 0.05 mg to 1 mg, from or from about 0.05 mg to 0.5 mg, from or from about 0.05 mg to 0.25 mg, from or from about 0.05 mg to 0.1 mg, from or from about 0.05 mg to 0.075 mg, from or from about 0.075 mg to 10 mg, from or from about 0.075 mg to 7.5 mg, from or from about 0.075 mg to 5 mg, from or from about 0.075 mg to 2.5 mg, from or from about 0.075 mg to 1 mg, from or from about 0.075 mg to 0.5 mg, from or from about 0.075 mg to 0.25 mg, from or from about 0.075 mg to 0.1 mg, from or from about 0.1 mg to 10 mg, from or from about 0.1 mg to 7.5 mg, from or from about 0.1 mg to 5 mg, from or from about 0.1 mg to 2.5 mg, from or from about 0.1 mg to 1 mg, from or from about 0.1 mg to 0.5 mg, from or from about 0.1 mg to 0.25 mg, from or from about 0.25 mg to 10 mg, from or from about 0.25 mg to 7.5 mg, from or from about 0.25 mg to 5 mg, from or from about 0.25 mg to 2.5 mg, from or from about 0.25 mg to 1 mg, from or from about 0.25 mg to 0.5 mg, from or from about 0.5 mg to 10 mg, from or from about 0.5 mg to 7.5 mg, from or from about 0.5 mg to 5 mg, from or from about 0.05 mg to 2.5 mg, from or from about 0.05 mg to 1 mg, from or from about 1 mg to 10 mg, from or from about 1 mg to 7.5 mg, from or from about 1 mg to 5 mg, from or from about 1 mg to 2.5 mg, from or from about 2.5 mg to 10 mg, from or from about 2.5 mg to 7.5 mg, from or from about 2.5 mg to 5 mg, from or from about 5 mg to 10 mg, from or from about 5 mg to 7.5 mg, or from or from about 7.5 mg to 10 mg.

In some embodiments, the contacting of the cytokine receptor agonist with the lipid particles or the lentiviral vector is in a volume that is between 100 mL and 1000 mL, inclusive. In some embodiments, the cytokine receptor agonist is present as part of the transfection mixture. In some embodiments, the cytokine receptor agonist is added to the composition of PBMCs or subset thereof and the lipid particles or lentiviral vector to produce a transfection mixture containing the PBMCs or subset thereof, the lipid particles or lentiviral vector and the cytokine receptor agonist. In some embodiments, the volume of the transfection mixture is between 100 mL and 1000 mL, inclusive. In some of any embodiments, the volume of the transfection mixture is between 100 mL and 750 mL, 100 mL and 500 mL, or 100 mL and 400 mL, inclusive. In some embodiments, the volume is between 100 mL and 400 mL, inclusive.

In some of embodiments, one or more doses of the cytokine receptor agonist is administered to the subject separate from the in-line administration of the lentiviral vector. In some embodiments, the provided methods further include administering one or more doses of the cytokine receptor agonist to the subject after the in-line administration of the lipid particle or lentiviral vector. In some embodiments, the first dose of the cytokine receptor agonist is administered after the in-line administration of the lipid particle or the lentiviral vector. In some embodiments, the first dose of the cytokine receptor agonist is administered no more than one month, no more than 21 days, no more than 14 days or no more than 7 days after the in-line administration of the lipid particle or the lentiviral vector. In some embodiments, the provided methods further include administering one or more doses of the cytokine receptor agonist to the subject prior to the in-line administration of the lipid particle or lentiviral vector. In some embodiments, a first dose of the cytokine receptor agonist is administered prior to the in-line administration of the lipid particle or the lentiviral vector. In some embodiments, the first dose of the cytokine receptor agonist is administered within one month, within one week or within three days of the in-line administration of the lipid particle or the lentiviral vector. In some embodiments, the first dose of the cytokine receptor agonist is administered on the same day as the in-line administration of the lipid particle or the lentiviral vector.

In some embodiments, each of the one or more doses of the cytokine receptor agonist is from at or about 0.001 mg/kg to at or about 0.1 mg/kg. In some embodiments, each of the one or more doses of the cytokine receptor is from at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg. In some embodiments, each of the one or more doses of the cytokine receptor agonist is from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

In some embodiments, the cytokine receptor agonist is administered daily. In some embodiments, the cytokine receptor agonist is administered once a week (Q1W). In some embodiments, the cytokine receptor agonist is administered once every two weeks (Q2W). In some embodiments, the cytokine receptor agonist is administered once every three weeks (Q3W). In some embodiments, the cytokine receptor agonist is administered once every four weeks (Q4W). In some embodiments, the cytokine receptor agonist is administered one time.

In some embodiments, the cytokine receptor agonist is administered for one week, two weeks, three weeks, four weeks, five weeks, six weeks, seven weeks or eight weeks. In some embodiments, the cytokine receptor agonist is administered for four weeks. In some embodiments, the cytokine receptor agonist is administered for five weeks. In some embodiments, the cytokine receptor agonist is administered for six weeks. In some embodiments, the cytokine receptor is administered for seven weeks.

In some embodiments, the cytokine receptor agonist may be provided as a pharmaceutical composition. In some embodiments, the pharmaceutical composition contains the cytokine receptor agonist and a pharmaceutically acceptable carrier. In some embodiments, pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). In some embodiments, the carrier may be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity may be maintained, for example, by the use of a coating such as lecithin, by the maintenance of the required particle size in the case of dispersion and by the use of surfactants. In some embodiments, prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. I In one embodiment, the pharmaceutically acceptable carrier is not DMSO alone.

In some embodiments, formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. In some embodiments, the pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. In some embodiments, pharmaceutical preparations may also be combined where desired with other active agents, e.g., other analgesic agents. In some embodiments, the pharmaceutical compositions may include an additional ingredient that include but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. In some embodiments, “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

In some embodiments, the pharmaceutical compositions containing a cytokine receptor agonist may be suitably developed for intravenous, intratumoral oral, rectal, vaginal, parenteral, topical, pulmonary, intranasal, buccal, ophthalmic, or another route of administration. In some embodiments, the cytokine receptor agonist is administered subcutaneously. In some embodiments, the cytokine receptor agonist is administered intravenously. In some embodiments, the cytokine receptor agonist is administered intramuscularly.

In some embodiments, administering the cytokine receptor agonist in combination with the provided in-line methods increases the percentage of T cells in the subject transfected with the lipid particles or lentiviral vector compared to a similar method but in which the subject is not administered a cytokine receptor agonist. For instance, in some embodiments the methods include infusion or administration of a lentiviral vector in combination with a cytokine receptor agonist and the methods increase the percentage of T cells in the subject transduced with the lentiviral vector compared to a similar method but in which the subject is not administered a cytokine receptor agonist. In some embodiments, the percentage of T cells in the subject comprising the transgene, such as the payload gene (e.g. CAR), is increased compared to a similar method but in which the subject is not administered a cytokine receptor agonist. In some embodiments of the above improvements, the increase is by greater than at or about 1.5-fold, greater than at or about 2-fold, greater than at or about 3-fold, greater than at or about 5-fold, or greater than at or about 10-fold or more.

In some embodiments, administering the cytokine receptor agonist in combination with the provided in-line methods increases the persistence of T cells transduced with the lentiviral vector compared to a similar method but in which the subject is not administered a cytokine receptor agonist. In some embodiments, the persistence of T cells comprising the transgene, such as the payload gene (e.g. CAR) is increased compared to a similar method but in which the subject is not administered a cytokine receptor agonist. In some embodiments, the increase in persistence is observed at or about 12 months after administration of the lentiviral vector to the subject.

In some embodiments, a skilled artisan is familiar with methods to assess the exposure, number, concentration, persistence and proliferation of the T cells in the subject or T cells expressing the transgene, such as payload gene (e.g. CAR). In some embodiments, the concentration or number of T cells, e.g. CAR⁺ T cells, in the plasma following administration can be measured using any method known in the art suitable for assessing concentrations of cells or particular cells expressing a transgene, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, the presence and/or amount of cells expressing the engineered receptor (e.g., CAR-expressing cells administered for T cell based therapy) in the subject following the administration by the provided methods is detected. In some aspects, nucleic acid-based methods, such as quantitative PCR (qPCR), are used to assess the quantity of cells expressing the engineered receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid expressing the transgene, such as encoding the engineered receptor, e.g., CAR, per microgram of DNA, or as the number of transgene-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying the transgene, such as nucleic acids encoding the engineered receptor (e.g. CAR), and/or other components or elements of the lipid particle, such as lentiviral vector, including regulatory elements, e.g., promoters, transcriptional and/or post-transcriptional regulatory elements or response elements, or markers, e.g., surrogate markers. In some embodiments, the primers can be specific for regulatory elements, such as the woodchuck hepatitis virus post-transcriptional regulatory element (WPRE). In some examples, the presence and/or amount of cells expressing the transgene, such as engineered receptor (e.g. CAR) is expressed as copies of the nucleic acid sequence (e.g., transgene sequence) per mass of DNA (e.g., copies/pg of DNA); AUC of the curve of copies/pg of DNA over time, maximum or peak copies/pg of DNA following treatment, or copies/pg of DNA. In some embodiments, the presence and/or amount of cells can be determined at any time after the administration or infusion by the provided methods, such as at day 1, 2, 3, 4, 5, 6, 7, 8, 9, 10, 11, 12, 13, 14, 15, 16, 17, 18, 19, 20 or 21, or week 1, 2, 3, 4, 5, 6, 7, 8, 9, 10, 11, or 12 or more post-treatment or initiation thereof.

### 1. Cytokines and Cytokine Muteins

In some embodiments, the cytokine receptor agonist is a cytokine or a functionally active variant thereof. In some embodiments, the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21) or a functionally active variant thereof or a biologically active portion thereof. In some embodiments, the cytokine receptor agonist is a mutein (also called mutant or variant) of any of the above T cell stimulating cytokine muteins.

In some embodiments the cytokine receptor agonist is a cytokine receptor agonist that is or comprises a functionally active variant or biologically active portion of a cytokine. In some embodiments, the cytokine receptor agonist sequence is the wild-type sequence of a cytokine or a biologically active portion thereof. In some embodiments, the functionally active variant or the biologically active portion thereof is a mutein of a wild-type IL-2, or biologically active portion thereof. In some embodiments, the functionally active variant or the biologically active portion thereof is a mutein of a wild-type IL-7 or biologically active portion thereof. In some embodiments, the functionally active variant or the biologically active portion thereof is a mutein of a wild-type IL-15 or biologically active portion thereof. In some embodiments, the functionally active variant or the biologically active portion thereof is a mutein of a wild-type IL-21 or biologically active portion thereof.

The cytokine or cytokine mutein can be derived from non-recombinant methods and from recombinant methods, and the disclosure is not limited in this regard. In addition, the cytokine or cytokine mutein can be derived from human sources, animal sources (including insects), fungi sources (including yeasts), and plant sources. For instance, the cytokine or cytokine mutein can be obtained according to the procedures described by Namen et al. (1998) J. Exp. Med. 167:988-1002 and in Heufler, C., et al. (1993) J. Exp. Med., 178:1109-1114. The cytokine or cytokine mutein can also be derived from recombinant methods. See, for example, Ouellette et al. (2003) Protein Expression and Purification 30:156-166. Alternatively, the cytokine or cytokine mutein can be purchased commercially. Illustrative suppliers include, for example, eBioscience, Inc. (San Diego, CA); Sigma- Aldrich (St. Louis, MO); Proteintech (Rosemont, IL), Prospec (East Brunswick, NJ), ThermoFisher Scientific (Waltham, MA), among various other commercial sources.

A recombinant cytokine or cytokine mutein can be expressed in any of a number of suitable expression systems, and the disclosure is not limited in this regard. The cytokine or cytokine mutein can be expressed, for example, in bacterial (e.g., E. coli, see, for example, Fischer et al. (1995) Biotechnol. Appl. Biotechnol. 21_(3):295-311; Ouellette, T., et al, Protein Expression and Purification. 2003 August; 30(2): 156-66; Zaremba-Czogalla M., et al, Protein expression and Purification. 2015; 110:65-71), mammalian (see, for example, U.S. Pat. No. 4,965,195, Kronman et al. (1992) Gene 121:295-304. and Toghraie, F. S., et al, Reports of Biochemistry and Molecular Biology, 2017 October; 6(1): 66-73), yeast (e.g., Pichia pastoris, see, for example, Morel et al. (1997) Biochem. J. 328(1): 121-129; Luo, Y., et al, Protein Expr Purif. 2009 January; 63(1): 1-4), plant (see, for example, Mor et al. (2001) Biotechnol. Bioeng. 75(3):259-266), and insect cell (see, for example, Mirzaei, et al, (2008) Appl. Biochem. Biotechnol. 151(1):93-103) expression systems. The expression can occur via exogenous expression (when the host cell naturally contains the desired genetic coding) or via endogenous expression. Recombinant human cytokine expressed in E. coli is available from a variety of commercial sources including but not limited to, for example, Sigma- Aldrich (St. Louis, MO), Prospec (East Brunswick NJ), Thermo Fisher Scientific, Inc. (Waltham, MA), and R&D Systems (Minneapolis, MN). Recombinant human cytokine expressed in CHO-cells may be purchased from, e.g., Reprokine Ltd. (Valley Cottage, NY). For example, recombinant IL-7 expressed in human 293 cells (HEK293; IL-7(Asp26-His77, referred to as” tag-free”) is available from Acro Biosystems (Newark, DE); human IL-7 expressed in human 293 cells is available from Proteintech Group, Inc. (Rosemont, IL).

In some embodiments the cytokine receptor agonist is a cytokine mutein that is a functionally active variant or biologically active portion of a wild-type cytokine that contains one or more amino acid mutations, such as one or more amino acid insertions, deletions, substitutions or truncations. In some embodiments, the mutations described herein relate to amino acid insertions, deletions, substitutions or truncations of amino acids compared to a reference cytokine sequence. In some embodiments, the reference cytokine sequence is the wild-type sequence of a cytokine, a functionally active variant or a biologically active portion thereof. In some embodiments, the functionally active variant or the biologically active portion thereof is a mutant of a wild-type IL-2, IL-15, IL-7, or IL-21 or biologically active portion thereof.. In some embodiments, the wild-type cytokine is human IL-2 that has or comprises the sequence set forth in SEQ ID NO:129. In some embodiments, the wild-type cytokine is human IL-7 that has or comprises the sequence set forth in SEQ ID NO:187. In some embodiments, the wild-type cytokine is human IL-15 that has or comprises the sequence set forth in SEQ ID NO:159. In some embodiments, the wild-type cytokine is human IL-21 that has or comprises the sequence set forth in SEQ ID NO:188.

In some embodiments, the cytokine receptor agonist is a mutant cytokine that is a biologically active portion that is an N-terminally and/or C-terminally truncated fragment of a wild-type cytokine. In some embodiments, the cytokine receptor agonist is an IL-2 mutein that is a biologically active portion that is an N-terminally and/or C-terminally truncated fragment of a wild-type IL-2. In some embodiments, the cytokine receptor agonist is an IL-7 mutein that is a biologically active portion that is an N-terminally and/or C-terminally truncated fragment of a wild-type IL-7. In some embodiments, the cytokine receptor agonist is an IL-15 mutein that is a biologically active portion that is an N-terminally and/or C-terminally truncated fragment of a wild-type IL-15. In some embodiments, the cytokine receptor agonist is an IL-21 mutein that is a biologically active portion that is an N-terminally and/or C-terminally truncated fragment of a wild-type IL-21.

In some embodiments, the cytokine receptor agonist is wild-type IL-2 or IL-2 mutein. In some embodiments, the cytokine receptor agonist has the sequence set forth in SEQ ID NO:129 or a sequence that has at least 85%, at least 90%, at least 95%, or at least 97% sequence identity to SEQ ID NO:129. In some embodiments, the cytokine receptor agonist has the sequence set forth in SEQ ID NO:130 or a sequence that has at least 85%, at least 90%, at least 95%, or at least 97% sequence identity to SEQ ID NO:130. In some embodiments, the cytokine receptor agonist has the sequence set forth in SEQ ID NO:131 or a sequence that has at least 85%, at least 90%, at least 95%, or at least 97% sequence identity to SEQ ID NO:131.

In some embodiments, the cytokine receptor agonist is wild-type IL-7 or IL-7 mutein. In some embodiments, the cytokine receptor agonist has the sequence set forth in SEQ ID NO:156 or a sequence that has at least 85%, at least 90%, at least 95%, or at least 97% sequence identity to SEQ ID NO: 156. In some embodiments, the cytokine receptor agonist has the sequence set forth in SEQ ID NO:187 or a sequence that has at least 85%, at least 90%, at least 95%, or at least 97% sequence identity to SEQ ID NO: 187. In some embodiments, the cytokine receptor agonist has the sequence set forth in SEQ ID NO:179 or a sequence that has at least 85%, at least 90%, at least 95%, or at least 97% sequence identity to SEQ ID NO: 179. In some embodiments, the cytokine receptor agonist has the sequence set forth in SEQ ID NO:180 or a sequence that has at least 85%, at least 90%, at least 95%, or at least 97% sequence identity to SEQ ID NO: 180. In some embodiments, the cytokine receptor agonist has the sequence set forth in SEQ ID NO:181 or a sequence that has at least 85%, at least 90%, at least 95%, or at least 97% sequence identity to SEQ ID NO: 181. In some embodiments, the cytokine receptor agonist has the sequence set forth in SEQ ID NO:182 or a sequence that has at least 85%, at least 90%, at least 95%, or at least 97% sequence identity to SEQ ID NO: 182. In some embodiments, the cytokine receptor agonist has the sequence set forth in SEQ ID NO:183 or a sequence that has at least 85%, at least 90%, at least 95%, or at least 97% sequence identity to SEQ ID NO: 183. In some embodiments, the cytokine receptor agonist has the sequence set forth in SEQ ID NO:184 or a sequence that has at least 85%, at least 90%, at least 95%, or at least 97% sequence identity to SEQ ID NO: 184. In some embodiments, the cytokine receptor agonist has the sequence set forth in SEQ ID NO:185 or a sequence that has at least 85%, at least 90%, at least 95%, or at least 97% sequence identity to SEQ ID NO: 185. In some embodiments, the cytokine receptor agonist has the sequence set forth in SEQ ID NO:186 or a sequence that has at least 85%, at least 90%, at least 95%, or at least 97% sequence identity to SEQ ID NO: 186.

In some embodiments, the cytokine receptor agonist is wild-type IL-15 or IL-15 mutein. In some embodiments, the cytokine receptor agonist has the sequence set forth in SEQ ID NO:157 or a sequence that has at least 85%, at least 90%, at least 95%, or at least 97% sequence identity to SEQ ID NO:157. In some embodiments, the cytokine receptor agonist has the sequence set forth in SEQ ID NO:158 or a sequence that has at least 85%, at least 90%, at least 95%, or at least 97% sequence identity to SEQ ID NO:158. In some embodiments, the cytokine receptor agonist has the sequence set forth in SEQ ID NO:159 or a sequence that has at least 85%, at least 90%, at least 95%, or at least 97% sequence identity to SEQ ID NO:159.

In some embodiments, the cytokine receptor agonist is wild-type IL-21 or IL-21 mutein. In some embodiments, the cytokine receptor agonist has the sequence set forth in SEQ ID NO:176 or a sequence that has at least 85%, at least 90%, at least 95%, or at least 97% sequence identity to SEQ ID NO:176. In some embodiments, the cytokine receptor agonist has the sequence set forth in SEQ ID NO:177 or a sequence that has at least 85%, at least 90%, at least 95%, or at least 97% sequence identity to SEQ ID NO:177. In some embodiments, the cytokine receptor agonist has the sequence set forth in SEQ ID NO:178 or a sequence that has at least 85%, at least 90%, at least 95%, or at least 97% sequence identity to SEQ ID NO:178.

e. IL-2

IL-2 is a pleiotropic type-1 cytokine whose structure comprises a 15.5 kDa four α-helix bundle. The precursor of IL-2 is 153 amino acid residues in length, with the first 20 amino acids forming a signal peptide and residues 21-153 forming the mature form. IL-2 is produced primarily by CD4+T cells post antigen stimulation and by CD8+cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2RP (also known as CD122), and IL-2Rγ(also known as CD132). Interaction of IL-2 with the IL-2Rα forms the “low-affinity” IL-2 receptor complex with a Kd of about 10-8 M. Interaction of IL-2 with IL-2RP and IL-2Rγ forms the “intermediate-affinity” IL-2 receptor complex with a Kd of about 10-9 M. Interaction of IL-2 with all three subunits, IL-2Ra, IL-2RP, and IL-2Ry, forms the “high-affinity” IL-2 receptor complex with a Kd of about >10-11 M.

In some aspects, IL-2 signaling via the “high-affinity” IL-2RaXy complex modulates the activation and proliferation of regulatory T cells. Regulatory T cells, or CD4+CD25+Foxp3+regulatory T (Treg) cells, mediate maintenance of immune homeostasis by suppression of effector cells such as CD4+ T cells, CD8+ T cells, B cells, NK cells, and NKT cells. In some instances, Treg cells are generated from the thymus (tTreg cells) or are induced from naive T cells in the periphery (pTreg cells). In some cases, Treg cells are considered as the mediator of peripheral tolerance. Indeed, in one study, transfer of CD25-depleted peripheral CD4+ T cells produced a variety of autoimmune diseases in nude mice, whereas cotransfer of CD4+CD25+ T cells suppressed the development of autoimmunity (Sakaguchi, et al., “Immunologic self-tolerance maintained by activated T cells expressing IL-2 receptor alpha-chains (CD25),” J. Immunol. 155(3): 1151-1164 (1995)). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8+effector T (Teff) cells, NK cells, and NKT cells. CD8+Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8+Teff cells, target cancerous cells and pathogen-infected cells.

In some aspects, IL-2 signaling is utilized to modulate T cell responses and subsequently for treatment of a cancer. For example, IL-2 is administered in a high-dose form to induce expansion of Teff cell populations for treatment of a cancer. However, high-dose IL2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome VLS).

Therefore, in some aspects, selective stimulation of engineered cells to maximize their therapeutic effectiveness is a challenge with adoptive cell therapy currently, and typical means to provide for the continued maintenance of activated engineered T cell products is the systemic administration of cytokines, including IL-2. Importantly, the systemic administration of IL-2 is associated with non-specific stimulatory effects beyond the population of engineered cells and is in some aspects associated with significant toxicity in human subjects. IL-2 is known to have a short lifespan in vivo, which requires that any IL-2 be dosed frequently to maintain activated T cells.

In some embodiments, the cytokine receptor agonist is IL-2. In some embodiments, the cytokine receptor agonist is human recombinant IL-2.

In some embodiments, the cytokine receptor agonist has a sequence set forth in SEQ ID NO: 129 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO:129

In some embodiments, the cytokine receptor agonist has a sequence set forth in SEQ ID NO: 129 or is a functionally active variant or biologically active portion thereof that has a sequence that is at least at or about 80%, at least at or about 90%, at least at or about 95%, or at least at or about 99% identical to SEQ ID NO: 129. In some embodiments, the cytokine receptor agonist comprises IL-2 or a functionally active fragment thereof comprises the amino acid sequence as set forth in SEQ ID NO: 129.

In some embodiments, the cytokine receptor agonist has a sequence set forth in SEQ ID NO: 130 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO:130

In some embodiments, the cytokine receptor agonist has a sequence set forth in SEQ ID NO: 130 or is a functionally active variant or biologically active portion thereof that has a sequence that is at least at or about 80%, at least at or about 90%, at least at or about 95%, or at least at or about 99% identical to SEQ ID NO: 130. In some embodiments, the cytokine receptor agonist comprises IL-2 or a functionally active fragment thereof comprises the amino acid sequence as set forth in SEQ ID NO: 130.

In some embodiments, the cytokine receptor agonist has a sequence set forth in SEQ ID NO: 131 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO:131.

In some embodiments, the cytokine receptor agonist has a sequence set forth in SEQ ID NO: 131 or is a functionally active variant or biologically active portion thereof that has a sequence that is at least at or about 80%, at least at or about 90%, at least at or about 95%, or at least at or about 99% identical to SEQ ID NO: 131. In some embodiments, the cytokine receptor agonist comprises IL-2 or a functionally active fragment thereof comprises the amino acid sequence as set forth in SEQ ID NO: 131.

In some embodiments, the functionally active fragment comprises IL-2 region 10-133, 20-133, 30-133, 10-130, 20-130, 30-130, 10-125, 20-125, 30-125, 1-130, or 1-125, wherein the residue positions are in reference to the positions in any of SEQ ID NOs: 129-131. In some embodiments, the modified IL-2 polypeptide comprises about 80%, 85%, 90%, 95%, 96%, 97%, 98%, or 99% sequence identity to SEQ ID NO: 129. In some embodiments, the modified IL-2 polypeptide comprises about 80%, 85%, 90%, 95%, 96%, 97%, 98%, or 99% sequence identity to SEQ ID NO: 130. In some embodiments, the modified IL-2 polypeptide comprises about 80%, 85%, 90%, 95%, 96%, 97%, 98%, or 99% sequence identity to SEQ ID NO: 131.

In some embodiments, the cytokine receptor agonist is an IL-2 that is a biologically active portion that is truncated and lacks up to 49 contiguous amino acid residues at or near the N-terminus of the wild-type cytokine, such as a wild-type cytokine set forth in any one of SEQ ID NO:129, 159, 187 or 188. In some embodiments, the mutant cytokine receptor agonist is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type cytokine.

In some embodiments, the cytokine receptor agonist comprises a T cell stimulating cytokine that is IL-2 or a functionally active fragment thereof. In some embodiments, the IL-2 is an IL-2 variant that is modified.

IL-2 variants (muteins) may have improved therapeutic effects compared to wild-type IL-2. IL-2 muteins with decreased binding affinity to IL-2 Rβ, such as BAY 50-4798 (containing an N88R mutation of IL-2) and Selectikine (harboring a D20T mutation of IL-2) have been generated (Shanafelt et al, Nat. Biotechnol. 19: 1197-1202 (2000); Laurent et al, J. Transl. Med. 11,5 (2013). IL-2 muteins with decreased affinity to IL-2 Rα (such as ‘no-α mutein’ GA501, and GA504 have been generated (Carmenate et al, J. Immunol. 190: 6230-38 (2013); Klein et al, Cancer Res. 73, 486 (2013)). Agonistic IL-2 muteins H9 and D10 (also known as IL-2 superkines) associate with IL-2 Rβwith 200-fold increased affinity and efficiently bound dimeric IL-2Rs without the need for IL-2 Rα; such binding resulted in increased STATS phosphorylation and cell proliferation in vitro and in vivo (Levin et al, Nature 484: 529-33 (2012)). An H9 mutant, H9-RETR, was engineered by introducing four mutations into H9 (L18R, Q22E, Q126T, and S130R) and retained IL-2 Rβbinding but demonstrated significantly decreased affinity to γc (Mitra et al, Immunity 42: 815-25). IL-2 muteins are also described in WO2014/100014, WO2015164815, WO205/118016, WO2016/030350, U.S. Pat. Nos. 8,759,486, and 9,266,938.

Previous studies of IL-2 variants have shown that IL-2 variants with mutations in at least four of the positions 38, 42, 45, 62, 68 have reduced stimulatory effects on regulatory T cells (WO 2012062228); IL-2 variants containing mutations at positions 72, 42, 45, have reduced or eliminated binding to high binding IL-2 receptors, but retain binding to intermediate binding IL-2 receptors (CN 201280017730.1); variants with mutations at positions 91, 126, which mutations allow IL-2 to bind to CD25(IL2R a) but do not activate IL-2R on regulatory T cells (U.S. Pat. No. 8,906,356); IL-2R comprising at least one E15, H16, Q22, D84, N88 or E95 mutation for use in the treatment of graft-versus-host disease in a subject (U.S. Pat. No. 9,732,134); IL-2 variants comprising at least R38W have the ability to decrease vascular permeability and treat solid tumors (U.S. Pat. Nos. 7,371,371; 7,514,073; 8,124,066; 7,803,361); fusion protein of IL-2 variant and Fc for treating diseases, wherein the IL-2 has N88R mutation (WO 2016014428); a chimeric polypeptide comprising a cytokine linked to an immune cell surface protein targeting ligand, wherein the cytokine may be a variant of IL-2 (WO2017136818) or the like.

In some embodiments, the cytokine receptor agonist comprises IL-2 or a functionally active fragment thereof that is modified to comprise least one unnatural amino acid at a position on the polypeptide that reduces binding between the modified IL-2 polypeptide and interleukin 2 receptor α (IL-2Rα), but does not significantly impair binding with interleukin 2 βγ receptor (IL-2Rβγ) signaling complex to form an IL-2/IL-2Rβγ complex. In some embodiments, the reduced binding to IL-2Rα is compared to a binding between a wild-type IL-2 polypeptide and IL-2Rα. In some embodiments, the decrease in binding affinity is about 10%, 20%, 30%, 40%, 50%, 60%, 70%, 80%, 90%, 95%, or 99% decrease in binding affinity to IL-2Rα relative to a wild-type IL-2 polypeptide. In some embodiments, the decrease in binding affinity is about 1-fold, 2-fold, 3-fold, 4-fold, 5-fold, 6-fold, 7-fold, 8-fold, 9-fold, 10-fold, or more relative to IL-2Rα relative to a wild-type IL-2 polypeptide.

In some embodiments, the modified IL-2 polypeptide with the decrease in binding affinity to IL-2Rα is capable of expanding CD4+helper cell, CD8+effector naive and memory cell, Natural Killer (NK) cell, Natural killer T (NKT) cell populations, or a combination thereof. In some embodiments, activation of CD4+helper cell, CD8+effector naive and memory cell, Natural Killer (NK) cell, or Natural killer T (NKT) cell population via the IL-2Rβγcomplex by the modified IL-2 polypeptide is not significantly different than activation of said cell population by a wild-type IL-2 polypeptide, and wherein the potency of the modified IL-2 polypeptide is at least 1-fold higher than a potency of the wild-type IL-2 polypeptide. In some embodiments, the modified IL-2 polypeptide expands CD4+Tregulatory (Treg) cells by less than 20%, 15%, 10%, 5%, 1%, or 0.1% when said activator is in contact with said cell population. In some embodiments, the modified IL-2 polypeptide does not expand Treg cells in said cell population. In some embodiments, activation of CD4+helper cell, CD8+effector naive and memory cell, Natural Killer (NK) cell, or Natural killer T (NKT) cell population via the IL-2Rβγcomplex by the modified IL-2 polypeptide retains significant potency of activation of said cell population relative to a wild-type IL-2 polypeptide. In some embodiments, the receptor signaling potency of the modified IL-2 polypeptide to the IL-2Rβγcomplex is higher than a receptor signaling potency of the wild-type IL-2 polypeptide to the IL-2Rβγcomplex. In some embodiments, the receptor signaling potency of the modified IL-2 polypeptide the IL-2Rβγcomplex is lower than a receptor signaling potency of the wild-type IL-2 polypeptide the IL-2Rβγcomplex. In some embodiments, the modified IL-2 polypeptide exhibits a first receptor signaling potency to IL-2Rβγ and a second receptor signaling potency to IL-2Rαβγ, and wherein the first receptor signaling potency is at least 1-fold, 2-fold, 3-fold, 4-fold, 5-fold, 6-fold, 7-fold, 8-fold, 9-fold, 10-fold, 20-fold, 30-fold, 50-fold, 100-fold, 500-fold, or higher than the second receptor signaling potency. In some embodiments, the first receptor signaling potency of the modified IL-2 polypeptide is higher than a receptor signaling potency of the wild-type IL-2 polypeptide to the IL-2Rβγ, and the second receptor signaling potency of the modified IL-2 polypeptide is lower than a receptor signaling potency of the wild-type IL-2 polypeptide to the IL-2Rαβγ. In some embodiments, the first receptor signaling potency of the modified IL-2 polypeptide is at least 1-fold lower than a receptor signaling potency of the wild-type IL-2 polypeptide.

In some embodiments, the modified IL-2 provides a first EC50 value for activating IL-20y signaling complex and a second EC50 value for activating IL-2αβγ signaling complex, and wherein a difference between the first EC50 and the second EC50 value is less than 10-fold. In some embodiments, the difference is less than 5-fold, less than 4-fold, less than 3-fold, less than 2-fold, or less than 1-fold. In some embodiments, the difference in receptor signaling potency is less than 5-fold, less than 4-fold, less than 3-fold, less than 2-fold, or less than 1-fold.

IL-2 orthologs with diminished affinity for the intermediate affinity (CD122/CD132) IL-2 receptor complex or high-affinity (CD25/CD 122/CD 132) IL-2 receptor complex are also useful to selectively target the activity of ortholog IL-2. IL-2 orthologs with significantly diminished affinity for the native wild-type hCD122 extracellular domain (BCD) but retain binding to the BCD of CD25 may also be used as competitive antagonists of wild-type IL-2 by interfering with the high-affinity IL-2 receptor complex formation and consequently may be employed in the treatment of autoimmune diseases or graft-versus-host (GVH) disease. Sockolosky, et al. (Science (2018) 359: 1037-1042) and Garcia, et al. (United States Patent Application Publication US2018/0228841 A1 published Aug. 16, 2018) describe an orthogonal IL2/CD122 ligand/receptor system to facilitate selective stimulation of cells engineered to express the orthogonal CD 122 receptor. IL2 muteins that are cognate ligands for the orthogonal receptor are also described. The contact of engineered T cells that express the orthogonal CD122 with a corresponding orthogonal ligand for such orthogonal CD 122 (“IL2 orthologs”) enables specific activation of such engineered T cells. In particular this orthogonal IL2 receptor ligand complex provides for selective expansion of cells engineered to express the orthogonal receptor in a mixed population of cells, in particular a mixed population of T cells.

In some embodiments, the cytokine receptor agonist is or comprises IL-2 orthogonal ligands (“IL-2 orthologs”) that specifically and selectively bind to the extracellular domain (BCD), or a transmembrane polypeptide comprising of CD122 polypeptide.

In some embodiments, the mutation is at one or more of the amino acid residues in positions 29-44, or positions 41-44, or positions 35-44 of IL-2. In some embodiments, one or more amino acid mutations at positions 11, 26, 27, 45, 70, 72, 78, 82, 132 in addition to one or more amino acid residue mutations at positions 29-44; one or more amino acid mutations at positions 11, 26, 27, 29, 30, 45, 70, 72, 78, 82, 132 in addition to one or more amino acid residue mutations at positions 41-44; or one or more amino acid mutations at positions 11, 26, 27, 29, 30, 45, 70, 72, 78, 82, 132 in addition to one or more amino acid residue mutations at positions 35-44.

In some embodiments, the variants are asparagine (N) at position 26 and/or asparagine (N) at position 30 and/or glutamine (Q) at position 11 and/or leucine (L) at position 132 and/or leucine (L) at position 70 and/or proline (P) at position 82 and/or glycine (G) at position 27 and/or phenylalanine (F) at position 78 and/or asparagine (N) at position 29 and/or asparagine (N) at position 30 and/or tyrosine (Y) at position 31 and/or lysine (K) at position 32 and/or asparagine (N) at position 33 and/or proline (P) at position 34 and/or lysine (K) at position 35 and/or leucine (L) at position 36 and/or threonine (T) at position 37 and/or arginine (R) at position 38 and/or methionine (M) at position 39 and/or methionine (M) at position 35 of the wild-type human interleukin 2(SEQ ID NO:2) sequence Or leucine (L) at position 40 and/or threonine (T) at position 41 and/or phenylalanine (F) at position 42 and/or lysine (K) at position 43 and/or phenylalanine (F) at position 44 and/or tyrosine (Y) at position 45 and/or asparagine (N) at position 71 to other amino acids. The numbering of the sites is from position 1 of the mature human IL-2 protein set forth in SEQ ID NO.: 129.

In some embodiments, IL-2 variants are provided that comprise mutations at positions 29-44 (mutations at positions 29-44 to QSMHIDATL, or mutations at positions 41-44 to DATL, or mutations at positions 35-44 to MHIDATL) that have reduced binding to IL-R a and substantially unchanged binding to IL-R β/γ. In some embodiments, IL-2 variants are provided that comprise mutations at positions 29-44 (including mutations at positions 29-44 to QSMHIDATL, or mutations at positions 41-44 to DATL, or mutations at positions 35-44 to MHIDATL), while comprising one or more of N26Q, N29S, N30S, Q11C/L132C, L70C/P82C, G27C/F78C, that have reduced binding to IL-R α and substantially unchanged binding to IL-R β/γ with increased stability.

In some embodiments, the cytokine receptor agonist comprises IL-2 or a functionally active fragment thereof is modified to comprise least one unnatural amino acid. In some embodiments, the position of the at least one unnatural amino acid is selected from K35, T37, R38, T41, F42, K43, F44, Y45, E60, E61, E62, K64, P65, E68, V69, N71, L72, M104, C105, and Y107, wherein the residue positions correspond to the positions 35, 37, 38, 41, 42, 43, 44, 45, 61, 62, 64, 65, 68, 69, 71, 72, 104, 105, and 107 as set forth in SEQ ID NO: 129. In some embodiments, the position of the at least one unnatural amino acid is selected from T37, R38, T41, F42, K43, F44, Y45, E61, E62, P65, E68, and L72, wherein the residue positions correspond to the positions 37, 38, 41, 42, 43, 44, 45, 61, 62, 65, 68, and 72 as set forth in SEQ ID NO: 129. In some embodiments, the position of the at least one unnatural amino acid is selected from K35, K64, V69, N71, M104, C105, and Y107, wherein the residue positions correspond to the positions 35, 64, 69, 71, 104, 105, and 107 as set forth in SEQ ID NO: 129. In some embodiments, the position of the at least one unnatural amino acid is selected from T37, R38, T41, Y45, E61, E68, and L72, wherein the residue positions correspond to the positions 37, 38, 41, 45, 61, 68, and 72 as set forth in SEQ ID NO: 129. In some embodiments, the position of the at least one unnatural amino acid is selected from F42, K43, F44, E62, and P65, wherein the residue positions correspond to the positions 42, 43, 44, 62, and 65 as set forth in SEQ ID NO: 129.

In some embodiments, the at least one unnatural amino acid is a lysine analogue; such as a lysine analogue comprising an aromatic side chain; comprising an azido group; or comprising an aldehyde or ketone group. In some embodiments, the at least one unnatural amino acid does not comprise an aromatic side chain. In some embodiments, the at least one unnatural amino acid comprises N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, 0-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine.

In some embodiments, the cytokine receptor agonist comprises IL-2 or a functionally active fragment thereof and is modified by one or more substitutions. In some embodiments, the one or more substitutions is selected from K34, F41, F43, K42, E61, P64, R37, T40, E67, Y44, V68, and L71, wherein the position is in reference to the positions in SEQ ID NO: 131. In some embodiments, the one or more substitutions is selected from F41, E61, and P64, wherein the position is in reference to the positions in SEQ ID NO: 131. In some embodiments, the one or more substitutions is K34, wherein the position is in reference to the positions in SEQ ID NO: 131. In some embodiments, the one or more substitutions is F41, wherein the position is in reference to the positions in SEQ ID NO: 131. In some embodiments, the one or more substitutions is F43, wherein the position is in reference to the positions in SEQ ID NO: 131. In some embodiments, the one or more substitutions is K42, wherein the position is in reference to the positions in SEQ ID NO: 131. In some embodiments, the one or more substitutions is E61, wherein the position is in reference to the positions in SEQ ID NO: 131. In some embodiments, the one or more substitutions is P64, wherein the position is in reference to the positions in SEQ ID NO: 131. In some embodiments, the one or more substitutions is R37, wherein the position is in reference to the positions in SEQ ID NO: 131. In some embodiments, the one or more substitutions is T40, wherein the position is in reference to the positions in SEQ ID NO: 131. In some embodiments, the one or more substitutions is E67, wherein the position is in reference to the positions in SEQ ID NO: 131. In some embodiments, the one or more substitutions is Y44, wherein the position is in reference to the positions in SEQ ID NO: 131. In some embodiments, the one or more substitutions is V68, wherein the position is in reference to the positions in SEQ ID NO: 131. In some embodiments, the one or more substitutions is L71, wherein the position is in reference to the positions in SEQ ID NO: 131.

For any given cytokine receptor agonist that is or comprises a moiety of IL-2, it is possible to determine whether that cytokine receptor agonist has IL-2 activity. Various methods for determining the in vitro IL-2 activity are described in the art. An exemplary approach is the CTTL-2 cell proliferation assay described in the experimental below. An exemplary approach is described in Moreau et al. (1995) Mol. Immunol. 32:1047-1056). Briefly, in a non-specific binding assay, a proposed IL-2 moiety is allowed to preincubate for one hour at 40 C in the presence of a cell line bearing a receptor of IL-2. Thereafter, 12s-labelled IL-2 is allowed to incubate in the system for three hours at 4° C. Data is expressed as % inhibitory capacity of the proposed IL-2 moiety activity versus wild type IL-2. Other methodologies known in the art can also be used to assess IL-2 function, including electrometry, spectrophotometry, chromatography, and radiometric methodologies. f. Il-7

Interleukin-7 (“IL-7”) is a non-hematopoietic cell-derived cytokine that promotes survival and/or proliferation of T cells, long term memory T cells, B-cells, and other immune cells. In some aspects, IL-7 is required for T cell development as well as for the maintaining and restoring homeostasis of mature T cells. IL-7 also maintains survival of naive and memory T cell homeostasis in the periphery. IL-7 is secreted by stromal cells in the thymic and bone marrow environment (Mazzucchelli, R., Durum, S.K., Nat Rev Immunol. 2007; 7:144-154); IL-7 is produced by fibroblastic reticular cells in T cell zones of lymph nodes (Link, A., et al., Nat Immunol. 2007; 8:1255-1265). IL-7 signals through binding to its heterodimeric receptor, which is a cell surface protein consisting of two chains: the IL-7Ra- chain (IL-7Ra, CD 127), and the common cytokine receptor g-chain (yc. CD 132) for IL-2, IL- 4, IL-9, IL-15 and IL-21 (Kroemer et al. (1996) Protein Eng. 9(12): 1135-1142). The g-chain appears to expand the T cell repertoire to allow more diverse targeting of tumor antigens. Importantly, the relatively high levels of IL-7 that can occur following administration of IL-7 can cause a profound and sustained reduction in the expression of IL-7 receptors. See, for example, Ghazawi et al. (2013) Immunology and Cell Biology 9L 149-158. Further polymer conjugates of interleukin-7 and other interleukin-7 variants have been previously described, such as for example in International Publication No. WO 2016/145388.

Based on its amino acid primary sequence (no glycans), interleukin-7 has a molecular weight of approximately 17.5 kilodaltons. Native (endogenous) human interleukin-7 contains three potential N-glycosylation sites (Asn71, Asn92, and Asnll6) and one potential O-glycosylation site (ThrllO), wherein the molecular weight of human native IL-7 is 25 kDa (Zaremba-Czogalla M., et al, Protein expression and purification. 2015; 110:65-71). A “glycosylation site” designates any amino acid residue or region in a polypeptide which is subject to glycosylation, i.e., the attachment of a carbohydrate structure. Such sites are typically N-glycosylation sites (i.e., any amino acid residue or region in a polypeptide which allows the attachment of a carbohydrate structure through N-linkage) and/or O-glycosylation sites (i.e., any amino acid residue or region in a polypeptide which allows the attachment of a carbohydrate structure through O-linkage). Consensus sequences for glycosylation sites are known per se in the art. As an illustration, a consensus N-glycosylation site typically has the following structure: Asn-X-Ser/Thr, where X is any amino acid except Proline. As will be disclosed below, such glycosylation sites may be either naturally present within an IL-7 polypeptide sequence and/or artificially added or created within said sequence.

In some embodiments, the IL-7 is hyperglycosylated. In some embodiments, “hyperglycosylated IL-7” designates an IL-7 polypeptide having at least three occupied glycosylation sites, i.e., having at least three glycosylated amino acid residues. In some embodiments, provided herein is hyperglycosylated IL-7 in which the IL-7 polypeptide has at least three occupied glycosylation sites, i.e., having at least three glycosylated amino acid residues. In some embodiments, the IL-7 that is hyperglycosylated has a glycosylated residue at least, at three N-glycosylation sites(s) and, optionally, at one O-glycosylation site. In some embodiments, the IL-7 is not an un- or mono-glycosylated IL-7 polypeptide.

As described above, Human IL-7 amino acid sequence contains three putative N-linked glycosylation sites, located at Asn residues at positions 70, 91 and 116 with respect to numbering as set forth in SEQ ID NO: 180. Transient recombinant expression of hIL-7 (human IL-7) in COS cells allowed the visualization of r-huIL-7 (recombinant human IL-7) as three protein bands of apparent molecular weight of about 20, 24 and 28 kDa (Cosman et al.; Lymphokine Receptor Interactions; 1989; 179:229-236). Stable recombinant expression of hIL-7 in BHK cells was also reported (Armitage et al.; The Journal of Immunology; 1990; 144:938-941). Furthermore, it has been observed that the IL-7 sequence may also contains one O-glycosylation site, namely Threonine (Thr) residue at position 110 (SEQ ID NO: 180). In some embodiments, the hyperglycosylated IL-7 polypeptide is an IL-7 polypeptide having the above three N-glycosylation sites occupied, optionally wherein the IL-7 polypeptide is associated or not to one occupied O-glycosylation site.

In some embodiments, a hyperglycosylated IL-7 polypeptide may comprise additional artificially added or created glycosylation sites. Accordingly, a hyperglycosylated IL-7 polypeptide is an IL-7 polypeptide having at least three N-glycosylation sites and one O-glycosylation site occupied, said sites being either naturally-occurring and/or artificially added/created.

In some embodiments, the IL-7 polypeptide has a modified amino acid sequence, wherein said sequence comprises at least one artificially created glycosylation site. In some embodiments, IL-7 polypeptides of this invention comprise 1, 2, 3 or 4 artificially created glycosylation sites, more preferably 1, 2 or 3; even more preferably 1 or 2.

In some embodiments, the artificially created glycosylation sites are N-linked glycosylation sites. Consensus N-glycosylation sites typically have the following structure: Asn-X-Ser/Thr, where X is any amino acid except Proline.

The glycosylation sites may be created or added chemically from assembled synthetic oligonucleotides or using several techniques including mutagenesis methods at various positions within IL-7 primary amino acid sequence, and following techniques known per se in the art. Because the modified IL-7 polypeptide shall retain the ability to bind an IL-7 receptor, the glycosylation site(s) is (are) in some embodiments created within region(s) or domain(s) of the IL-7 polypeptide sequence which do(es) not alter the ability of IL-7 to bind an IL-7 receptor. In some embodiments, the binding affinity is not altered or may be reduced without impacting the in vivo effects resulting from interaction with the receptor.

In some embodiments, the site(s) is (are) introduced outside of the alpha helices of the polypeptide, optionally with the exception of immediate proximity of the glycine residues. In some embodiments, they are introduced in the most flexible region, avoiding regions that are more rigid and important for tertiary structure of the polypeptide. In some embodiments, the creation of a glycosylation site does not affect any Cystein residue involved in a disulfide bridge (e.g., Cys 2, 34, 47, 92, 129 and 141 with respect to numbering as set forth in SEQ ID NO: 180), nor any critical residue involved in the interaction of IL-7 polypeptide with its cognate receptor (e.g., Ser 19, Leu 23 and 77, Tyr 12, Val 15, GIn 22, Lys 81 and Glu 84 with respect to numbering as set forth in SEQ ID NO: 180), nor any conserved residue involved in the activity of the polypeptide (e.g., Arg 133, Gln 136, Glu 137, Lys 139 and 144, Thr 140 and Asn 143 with respect to numbering as set forth in SEQ ID NO: 180). In some embodiments, the glycosylation sites are created by mutation, deletion or addition of one or several amino acid residues within the primary sequence of a reference IL-7 polypeptide, to create a typical consensus glycosylation site.

In some embodiments, the IL-7 polypeptide comprises the sequence of a human IL-7 polypeptide comprising one or several amino acid modifications selected from Lys28Asn-Ile30Ser-Ile30Thr-Ile30Asn-Ser32Thr-Leu35Ser-Leu35Thr-Glu38Ser-Glu38Thr-Phe39Ser-Phe39Thr-Phe42Ser-Phe42Thr-Glu52Ser-Glu52Thr-Val82Asn-Glu84Thr-Glu84Ser-Lys97Asn-Arg99Thr-Arg99Ser-Ala102Asn-Leu104Thr-Leu104Ser-Leu104Asn-Glu106Thr-Glu106Ser-Leu128Ser-Leu128Thr-Ile145Asn-Met147Thr-Met147Ser-Met147Asn-Thrl49Ser.

In some embodiments, the IL-7 polypeptide comprises the sequence of a human IL-7 polypeptide comprising one or several amino acid modifications selected from Phe39Ser-Phe39Thr-Phe42Ser-Phe42Thr-Leu104Asn-Glu106Thr-Glu106Ser-Leul28Ser-Leul28Thr-Metl47Asn and a combination thereof, or a distinctive fragment thereof.

TABLE 18
Exemplary modifications to IL-7 with
respect to numbering as set forth in SEQ ID NO: 180

Lys28Asn; Ile30Ser
Lys28Asn; Ile30Thr
Ile30Asn; Ser32Thr
Leu35Ser
Leu35Thr
Glu38Ser
Glu38Thr
Phe39Ser
Phe39Thr
Phe42Ser
Phe42Thr
Glu52Ser
Glu52Thr
Val82Asn; Glu84Ser
Val82Asn; Glu84Thr
Lys97Asn; Arg99Ser
Lys97Asn; Arg99Thr
Ala102Asn; Leu104Ser
Ala102Asn; Leu104Ser
Leu104Asn; Glu106Ser
Leu104Asn; Glu106Ser
Leu128Ser
Leu128Ser
Ile145Asn; Met147Ser
Ile145Asn; Met147Thr
Met147Asn; Thr149Ser

In some embodiments, the modified IL-7 has any one of the modifications in the table above. In some embodiments, the above modifications can be combined to create several additional glycosylation sites within an IL-7 polypeptide. In this regard, In some embodiments the IL-7 is any biologically active IL-7 polypeptide having the primary sequence of interleukin-7 modified by the addition of at least from one to four additional (N-linked) glycosylation sites. In some embodiments, the IL-7 is a biologically active IL-7 polypeptide comprising the primary sequence of interleukin-7 comprising one or two additional (N-linked) glycosylation sites.

In some embodiments, the hyperglycosylated IL-7 shows an improved stability, and an in vivo extended half-life and mean residence time (MRT) in mammalian hosts. The term “improved stability”, “extended half-life” and “mean residence time (MRT)” is to be understood in comparison to non-glycosylated forms (i.e., non-glycosylated IL-7). Preferably the increase of half-life is at least about 3x, preferably at least about 5 to 20x. MRT means the average of the residence time of each IL-7 containing cytokine receptor agonist in the blood of a subject after initial dosing. In some embodiments, the increase of MRT is at least about 2x, or optionally at least about 4 to 10x compared to the MRT of non-glycosylated forms.

For instance, the plasmatic half-life of the hyperglycosylated form was shown to be in the range of 30 to 40 hours, whereas the plasmatic half-life of the non-glycosylated form is usually 5 to 8 hours (when both forms are administered in the same conditions, i.e. in one injection, subcutaneously). The mean residence time (MRT) was around 40 hours versus around 10 hours with the non glycosylated form. This as described in U.S. Pat. No. 7,708,985, the contents of which are hereby incorporated in their entirety.

The structure and number of oligosaccharide units attached to a particular glycosylation site in a hyperglycosylated IL-7 polypeptide is, in some embodiments, variable. In some embodiments, these units may be, for instance, N-acetyl glucosamine, N-acetyl galactosamine, mannose, galactose, glucose, fucose, xylose, glucuronic acid, iduronic acid and/or sialic acids.

In some embodiments, the hyperglycosylated IL-7 polypeptide comprises (or is enriched in) N-linked and/or O-linked carbohydrate chain(s) selected from:
a) a mammalian type sugar chain, preferably of the type expressed by CHO cells;
b) a sugar chain comprising a complex N-carbohydrate chain (e.g., a triantenary or biantenary structure), more preferably containing high mannose and acetylglucosamine molecules and high terminal sialic acid residues;
c) a sugar chain comprising an O-carbohydrate chain without and preferably with a terminal sialic acid residue;
d) a sugar chain sialylated by alpha-2,6-sialyltransferase or alpha-2,3-sialyltransferase; and/or
e) a sialylated sugar chain displaying between 3 to 30 sialyl-N-acetylgalactosamine, preferably 7 to 23.

In some embodiments, the carbohydrate chain(s) comprise a triantenary or biantenary structure with partial or complete terminal sialylation. In some embodiments, the carbohydrate chains comprise triantenary structures and tri or bi-sialylation, and/or a diantenary structure with disialylation. Examples of such carbohydrates are disclosed in U.S. Pat. No. 7,708,985, the contents of which are hereby incorporated in their entirety.

In some embodiments, the hyperglycosylated interleukin-7 polypeptide has an average isoelectric point inferior to 6.5 and an average apparent molecular weight superior to 27 kDa, between 28 KDa and 65 KDa (theoretical for a 7N+1O glycosylation), preferably between 28 KDa and 35 KDa (as shown for a 3N+1O glycosylation), by gel electrophoresis (confirmed by Western blot) which is translated to 25 kDa by mass spectrometry analysis.

In some embodiments, the hyperglycosylated IL-7 polypeptide is produced by a mammalian glycosylation mutant that stably expresses a2,6 sialyltransferase and presents a deficiency in CMP-NeuSAc Hydrolase activity, preferably a CHO glycosylation mutant. Such glycosylation typically includes N-acetyl glucosamine, N-acetyl galactosamine, mannose, galactose, glucose, fucose, xylose, glucuronic acid, iduronic acid and/or sialic acids.

When produced in CHO cells, the recombinant IL-7 may comprise monosaccharides (such as galactose, mannose, fucose,), N-acetylglucosamine, N- acetylgalactosamine, and sialic acids attached to amino acids (asparagine or threonine) or lipids of the protein, wherein the glycans can be attached in linear or branching chains. An IL-7 moiety that is CHO-cell derived and used to prepare the subject cytokine receptor agonist will preferably have good lot-to-lot consistency with regard to its glycosylation profile (that is, glycoforms comprising the recombinant protein) and degree of sialylation. Glycoforms comprised in CHO-derived human recombinant IL-7 may range from low to high sialylated glycoforms, with an average number of sialic acids ranging from about 2.0 to about 15, or from about 2.0 to about 14, and in some embodiments, with an average number of sialic acids ranging from about 4.0 to about 8.0. In some embodiments, recombinant IL-7 that is CHO-derived possesses a low mannose content, for example, in a range of about 3 to about 12 mannose carbohydrates per IL-7 protein molecule, preferably from about 6 to about 10 mannose carbohydrates per IL-7 protein molecule.

Exemplary IL-7 moieties are described herein, in the literature, and in, for example, U.S. Pat. Nos. 7,589,179; 7,585,947; 8,034,327; 7,708,985; 10,208,099; 8,153,114; Wong et al. (2013) Oncolmmunology 2(11), e26442:1-3; Romano et al. (1998) Protein Engineering li(1):31-40; and Vudattu e/a/. (2009) Genes and Immunity 10:132-140.

In some embodiments, the cytokine receptor agonist is IL-7. In some embodiments, the cytokine receptor agonist is human recombinant IL-7.

In some embodiments, the cytokine receptor agonist has a sequence set forth in SEQ ID NO: 156 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO:156

In some embodiments, the cytokine receptor agonist has a sequence set forth in SEQ ID NO: 156 or is a functionally active variant or biologically active portion thereof that has a sequence that is at least at or about 80%, at least at or about 90%, at least at or about 95%, or at least at or about 99% identical to SEQ ID NO: 156. In some embodiments, the cytokine receptor agonist comprises IL-7 or a functionally active fragment thereof comprises the amino acid sequence as set forth in SEQ ID NO: 156.

In some embodiments, the cytokine receptor agonist comprises a T cell stimulating cytokine that is IL-7 or a functionally active fragment thereof. In some embodiments, the IL-7 is an IL-7 variant that is modified. Variants thereof include, more preferably, natural allelic variants resulting from natural polymorphism, including SNPs, splicing variants, etc. In a specific embodiment, the term IL-7 polypeptide is meant to designate a polypeptide having the sequence of SEQ ID NO: 156 or natural variants thereof.

In some embodiments, the cytokine receptor agonist has a sequence set forth in SEQ ID NO: 179 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 179

In some embodiments, the cytokine receptor agonist has a sequence set forth in SEQ ID NO: 179 or is a functionally active variant or biologically active portion thereof that has a sequence that is at least at or about 80%, at least at or about 90%, at least at or about 95%, or at least at or about 99% identical to SEQ ID NO: 179. In some embodiments, the cytokine receptor agonist comprises IL-7 or a functionally active fragment thereof comprises the amino acid sequence as set forth in SEQ ID NO: 179.

In some embodiments, the cytokine receptor agonist has a sequence set forth in SEQ ID NO: 180 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 180.

In some embodiments, the cytokine receptor agonist has a sequence set forth in SEQ ID NO: 180 or is a functionally active variant or biologically active portion thereof that has a sequence that is at least at or about 80%, at least at or about 90%, at least at or about 95%, or at least at or about 99% identical to SEQ ID NO: 180. In some embodiments, the cytokine receptor agonist comprises IL-7 or a functionally active fragment thereof comprises the amino acid sequence as set forth in SEQ ID NO: 180.

In some embodiments, the cytokine receptor agonist has a sequence set forth in SEQ ID NO: 181 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 181.

In some embodiments, the cytokine receptor agonist has a sequence set forth in SEQ ID NO: 181 or is a functionally active variant or biologically active portion thereof that has a sequence that is at least at or about 80%, at least at or about 90%, at least at or about 95%, or at least at or about 99% identical to SEQ ID NO: 181. In some embodiments, the cytokine receptor agonist comprises IL-7 or a functionally active fragment thereof comprises the amino acid sequence as set forth in SEQ ID NO: 181.

In some embodiments, the cytokine receptor agonist has a sequence set forth in SEQ ID NO: 182 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 182.

In some embodiments, the cytokine receptor agonist has a sequence set forth in SEQ ID NO: 182 or is a functionally active variant or biologically active portion thereof that has a sequence that is at least at or about 80%, at least at or about 90%, at least at or about 95%, or at least at or about 99% identical to SEQ ID NO: 182. In some embodiments, the cytokine receptor agonist comprises IL-7 or a functionally active fragment thereof comprises the amino acid sequence as set forth in SEQ ID NO: 182.

In some embodiments, the cytokine receptor agonist has a sequence set forth in SEQ ID NO: 183 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 183.

In some embodiments, the cytokine receptor agonist has a sequence set forth in SEQ ID NO: 183 or is a functionally active variant or biologically active portion thereof that has a sequence that is at least at or about 80%, at least at or about 90%, at least at or about 95%, or at least at or about 99% identical to SEQ ID NO: 183. In some embodiments, the cytokine receptor agonist comprises IL-7 or a functionally active fragment thereof comprises the amino acid sequence as set forth in SEQ ID NO: 183.

In some embodiments, the cytokine receptor agonist has a sequence set forth in SEQ ID NO: 184 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 184.

In some embodiments, the cytokine receptor agonist has a sequence set forth in SEQ ID NO: 209 or is a functionally active variant or biologically active portion thereof that has a sequence that is at least at or about 80%, at least at or about 90%, at least at or about 95%, or at least at or about 99% identical to SEQ ID NO: 184. In some embodiments, the cytokine receptor agonist comprises IL-7 or a functionally active fragment thereof comprises the amino acid sequence as set forth in SEQ ID NO: 184.

In some embodiments, the cytokine receptor agonist has a sequence set forth in SEQ ID NO: 185 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 185.

In some embodiments, the cytokine receptor agonist has a sequence set forth in SEQ ID NO: 185 or is a functionally active variant or biologically active portion thereof that has a sequence that is at least at or about 80%, at least at or about 90%, at least at or about 95%, or at least at or about 99% identical to SEQ ID NO: 185. In some embodiments, the cytokine receptor agonist comprises IL-7 or a functionally active fragment thereof comprises the amino acid sequence as set forth in SEQ ID NO: 185.

In some embodiments, the cytokine receptor agonist has a sequence set forth in SEQ ID NO: 186 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 186.

In some embodiments, the cytokine receptor agonist has a sequence set forth in SEQ ID NO: 211 or is a functionally active variant or biologically active portion thereof that has a sequence that is at least at or about 80%, at least at or about 90%, at least at or about 95%, or at least at or about 99% identical to SEQ ID NO: 186. In some embodiments, the cytokine receptor agonist comprises IL-7 or a functionally active fragment thereof comprises the amino acid sequence as set forth in SEQ ID NO: 186.

g. IL-15

Interleukin- 15 (IL-15) is a pleiotropic cytokine whose structure is a 14-15 kDa glycoprotein. IL-15 transcription, translation and secretion are regulated through multiple complex mechanisms. IL-15 and IL-15 receptor a (IL-15R a, CD215) proteins are co-expressed predominantly by activated monocytes and dendritic cells (DCs). The transcription of the heterodimer IL-15/IL-15Rα occurs following the interaction of monocytes/DCs with type 1 or type 2 interferons (IFN) or CD40 ligation or agents that act through Toll-like receptors (TLR) that activate NF-kB. Further, IL-15/IL-15Rα protein expression is predominantly controlled at the levels of translation and secretion. IL-15 signals through a heterotrimeric receptor comprising a unique a chain (IL-15R a), a shared b subunit (IL-15R b, CD132) with IL-2 (CD122) and a common g subunit (CD132; IL-15R g) shared with several cytokines. IL-15Ra has high affinity for IL-15 with a Kd about 10 11 M.

IL-15 binds to a specific receptor complex on T-cells and NK cells. IL-15 and IL-15Ra are co-expressed on activated dendritic cells and on monocytes, and IL-15 functions in a complex with IL-15Ra (Bergamaschi, et al., J Biol Chem 283:4189-99 (2008)). IL-15/IL-15a bind as a heterodimer to two chains on T-cells and NK cells—IL-2RP (also referred to as IL- 15RP; CD122) and yc (also referred to as IL-2RG; CD132; γ-c; common γ-chain) molecules. The β and yc chains are shared between IL-2 and IL-15 and are essential for the signaling of these cytokines (Giri et al., EMBO J. 13:2822-30 (1994) and Giri et al., EMBO J. 14:3654-3663 (1995)).

Consistent with the sharing of the IL-2/IL-15Pyc receptor complex, IL-15 has been shown to mediate many functions similar to those of IL-2 in vitro. They share many biological activities and exhibit similar contributions to the survival of T lymphocytes (see Waldmann, et al., Annu Rev Immunol 17: 19-49 (1999)). It is believed that the biological differences between IL-2 and IL-15 are likely due to, for example, their different production sites, their strength of association with membrane receptor proteins, termed IL-2a and IL-15Rα, respectively, and the regulation of these extra receptor molecules. IL-2 and IL-15 play a role in regulating the number of CD8+memory cells.

In some aspects, IL-15 signaling is utilized to modulate T cell responses and subsequently for treatment of cancer. In some embodiments, IL-15 signaling is utilized to simulate proliferation of activated CD4 CD8, CD4+CD8+, CD4+, and CD8+ T cells and their differentiation in defined effector T-cell subsets. In some embodiments, IL-15 signaling is utilized to simulate the generation and proliferation of natural killer (NK) cells. In some embodiments, IL-15 signaling is utilized to promote maintenance and survival of memory CD8 T cells, naive CD8 T cells, and NK cells. In some embodiments, IL-15 signaling is utilized to induce formation of memory CD8 T cells. In some embodiments, IL-15 signaling is utilized for priming NK cell target-specific activation. In some embodiments, IL-15 signaling does not result in Treg expansion.

Mature human IL-15 is a 114 amino acid monomeric polypeptide. Two transcripts have been reported, one with a 48 amino acid signal peptide (Long Signal Peptide; LSP) (SEQ ID NO: 157), and the other with a 21 amino acid signal peptide (Short Signal Peptide; SSP) (SEQ ID NO:158), both of which produce the same mature protein (SEQ ID NO:159). Mature human IL-15 is described as comprising four helices (A-D), also referred to as inter-helices junctions, linked by three distinct amino acid segments (A/B Loop; B/C Turn; and C/D Loop).

In some embodiments, the cytokine receptor agonist is IL-15. In some embodiments, the cytokine receptor agonist has a sequence set forth in SEQ ID NO: 157 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 157.

In some embodiments, the cytokine receptor agonist has a sequence set forth in SEQ ID NO: 157 or is a functionally active variant or biologically active portion thereof that has a sequence that is at least at or about 80%, at least at or about 90%, at least at or about 95%, or at least at or about 99% identical to SEQ ID NO: 157. In some embodiments, the cytokine receptor agonist comprises IL-15 or a functionally active fragment thereof comprises the amino acid sequence as set forth in SEQ ID NO: 157.

In some embodiments, the cytokine receptor agonist has a sequence set forth in SEQ ID NO: 158 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 158.

In some embodiments, the cytokine receptor agonist has a sequence set forth in SEQ ID NO: 158 or is a functionally active variant or biologically active portion thereof that has a sequence that is at least at or about 80%, at least at or about 90%, at least at or about 95%, or at least at or about 99% identical to SEQ ID NO: 158. In some embodiments, the cytokine receptor agonist comprises IL-15 or a functionally active fragment thereof comprises the amino acid sequence as set forth in SEQ ID NO: 158.

In some embodiments, the IL-15 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 41 amino acids to about 50 amino acids, from about 51 amino acids to about 60 amino acids, from about 61 amino acids to about 70 amino acids, from about 71 amino acids to about 80 amino acids, from about 81 amino acids to about 90 amino acids, from about 91 amino acids to about 100 amino acids, from about 101 amino acids to about 105 amino acids, from about 106 amino acids to about 110 amino acids, or from about 111, 112, or 113 amino acids up to the full-length peptide or polypeptide as set forth in SEQ ID NO: 159.

IL-15 variants can be prepared with various objectives in mind, including increasing serum half-life, reducing an immune response against IL-15, facilitating purification or preparation, decreasing degradation, improving therapeutic efficacy, and lessening the severity or occurrence of side effects during therapeutic use. The amino acid sequence variants are usually predetermined variants not found in nature, although some may be post-translational variants, e.g., glycosylated variants. Any variant of IL-15 can be used provided it retains a suitable level of IL-15 activity. IL-15 activities are described elsewhere herein (e.g., regulation of T cell and natural killer (NK) cell activation and proliferation). Examples of such variants are disclosed in WO2017112528, the contents of which are hereby incorporated in their entirety.

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2)R, K; 3) F, Y, H, W, R; 4) G, A, T, S; 5) Q, N; and 6) D, E. Guidance for substitutions, insertions, or deletions may be based on alignments of amino acid sequences of different variant proteins or proteins from different species. Thus, in addition to any naturally-occurring IL-15 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution. If should be noted that one or more unnatural amino acids may be introduced into IL-15 as a means of fostering site-specific conjugation.

In some embodiments, the cytokine receptor agonist is an IL-15 mutein (e.g., variant) comprising at least one amino acid substitution from a wildtype IL-15 as set forth in any of SEQ ID NOs 157, 158, or 159. In some embodiments, the at least one amino acid substitution is a conservative substitution. In some embodiments, the conservative substitution comprises a substitution of tyrosine and/or cysteine. In some embodiments, the at least one amino acid substitution is at one of the following positions: 1, 3, 13-15, 17-29, 33, 34, 36-39, 41, 45, 48, 49, 51-58, 60, 67, 71-84, 86, 87, 89-98, 101, 102, 104, 113, or 114 of SEQ ID NO: 159. In some embodiments, the at least one amino acid substitution is a substitution of tyrosine at one of the following positions: 1, 3, 13-15, 17-29, 33, 34, 36-39, 41, 45, 48, 49, 51-58, 60, 67, 71-84, 86, 87, 89-98, 101, 102, 104, 113, or 114 of SEQ ID NO: 159. In some embodiments, the at least one amino acid substitution is a substitution of cysteine at one of the following positions: 1, 3, 13-15, 17-29, 33, 34, 36-39, 41, 45, 48, 49, 51-58, 60, 67, 71-84, 86, 87, 89-98, 101, 102, 104, 113, or 114 of SEQ ID NO: 159. In some embodiments, the at least one amino acid substitution comprises substitution of an N—X—S glycosylation motif. In some embodiments, the at least one amino acid substitution comprises substitution of an N—X—S glycosylation motif at one of the following positions: 1, 3, 13-15, 17-29, 33, 34, 36-39, 41, 45, 48, 49, 51-58, 60, 67, 71-84, 86, 87, 89-98, 101, 102, 104, 113, or 114 of SEQ ID NO: 159. In some embodiments, the at least one amino acid substitution comprises substitution of an N-X-T glycosylation motif. In some embodiments, the at least one amino acid substitution comprises substitution of an N-X-T glycosylation motif at one of the following positions: 1, 3, 13-15, 17-29, 33, 34, 36-39, 41, 45, 48, 49, 51-58, 60, 67, 71-84, 86, 87, 89-98, 101, 102, 104, 113, or 114 of SEQ ID NO: 159.

Provided herein are biologically active fragments (e.g., subsequences) of mature IL-15 containing contiguous amino acid residues derived from the mature IL-15. The length of contiguous amino acid residues of a peptide or a polypeptide subsequence varies depending on the specific naturally-occurring amino acid sequence from which the subsequence is derived. In general, peptides and polypeptides may be from about 20 amino acids to about 40 amino acids, from about 41 amino acids to about 50 amino acids, from about 51 amino acids to about 60 amino acids, from about 61 amino acids to about 70 amino acids, from about 71 amino acids to about 80 amino acids, from about 81 amino acids to about 90 amino acids, from about 91 amino acids to about 100 amino acids, from about 101 amino acids to about 105 amino acids, from about 106 amino acids to about 110 amino acids, or from about 111, 112, or 113 amino acids up to the full-length peptide or polypeptide. Additionally, IL-15 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

A cysteine residue or a cysteine analog can be introduced into an IL-15 polypeptide to provide for linkage to another peptide via a disulfide linkage or to provide for cyclization of the IL-15 polypeptide. Methods of introducing a cysteine or cysteine analog are known in the art (see, e.g., U.S. Pat. No. 8,067,532). Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and -(CH2)n-CO- or -(CH2)n-C6H4-CO-) with functional groups which allow for the introduction of a bridge. Some examples are disulfides, disulfide mimetics such as the -(CH2)n- carba bridge, thioacetal, thioether bridges (cystathionine or lanthionine) and bridges containing esters and ethers. In these examples, n can be any integer, but is frequently less than ten.

h. IL-21

Interleukin-21 (IL-21) is a pleiotropic cytokine that regulates the activity of both innate and specific immunity. Croce et ah, J. Immunol. Res. 2015, 696578. An IL-21 stimulates T and natural killer (NK) cell proliferation and function and regulates B cell survival and differentiation and the function of dendritic cells. Id. An interleukin-21 receptor (IL-21R) has been shown to be expressed in diverse hematopoietic malignancies, including chronic lymphocytic leukemia (CLL), follicular lymphoma, diffuse large B-cell lymphoma (DLBCL), and mantle cell lymphoma. Conlon et ah, J. Interferon Cytokine Res. 2019, 39, 6-21. Several preclinical studies showed that IL-21 has antitumor activity in different tumor models, through mechanism involving the activation of NK and T or B cell responses. Croce et ah, J. Immunol. Res. 2015, 696578. However, just like aldesleukin, the recombinant IL-2 used in a clinical trial for treating metastatic melanoma has a half-life of only about 1 to 4 hours in human following an intravenous infusion. Davis etal., Clin. Cancer Res. 2007, 13, 3630-36.

IL-21 has been shown to be a potent modulator of cytotoxic T cells and NK cells. IL-21 has been shown to co-stimulate the expansion of NK cells, and it has been demonstrated to enhance the effector functions of these cells. T cell responses include enhancement of primary antigen response as modulation of memory T cell functions. A role for IL-21 in modulating the differentiation programming of human T cells was first reported by Li et al (Journal of immunology 175 (4): 2261-9 (2005)), where it was shown to enrich for a population of central memory-type CTL with a unique CD28+CD127hi CD45RO+phenotype with IL-2 producing capacity. Tumor-reactive antigen-specific CTL generated by priming in the presence of IL-21 led to a stable, ‘helper-independent’ phenotype.

In some embodiments, the cytokine receptor agonist is IL-21. In some embodiments, the cytokine receptor agonist has a sequence set forth in SEQ ID NO: 176 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 176.

In some embodiments, the cytokine receptor agonist has a sequence set forth in SEQ ID NO: 176 or is a functionally active variant or biologically active portion thereof that has a sequence that is at least at or about 80%, at least at or about 90%, at least at or about 95%, or at least at or about 99% identical to SEQ ID NO: 176. In some embodiments, the cytokine receptor agonist comprises IL-21 or a functionally active fragment thereof comprises the amino acid sequence as set forth in SEQ ID NO: 176.

In some embodiments, the cytokine receptor agonist has a sequence set forth in SEQ ID NO: 177 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 177.

In some embodiments, the cytokine receptor agonist has a sequence set forth in SEQ ID NO: 177 or is a functionally active variant or biologically active portion thereof that has a sequence that is at least at or about 80%, at least at or about 90%, at least at or about 95%, or at least at or about 99% identical to SEQ ID NO: 177. In some embodiments, the cytokine receptor agonist comprises IL-21 or a functionally active fragment thereof comprises the amino acid sequence as set forth in SEQ ID NO: 177.

In some embodiments, the cytokine receptor agonist has a sequence set forth in SEQ ID NO: 178 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to SEQ ID NO: 178.

In some embodiments, the cytokine receptor agonist has a sequence set forth in SEQ ID NO: 178or is a functionally active variant or biologically active portion thereof that has a sequence that is at least at or about 80%, at least at or about 90%, at least at or about 95%, or at least at or about 99% identical to SEQ ID NO: 178. In some embodiments, the cytokine receptor agonist comprises IL-21 or a functionally active fragment thereof comprises the amino acid sequence as set forth in SEQ ID NO: 178.

### 2. Cytokine Mimetics

In some embodiments, the cytokine receptor agonist is a cytokine mimetic or agonist thereof. In some embodiments, the cytokine mimetic is a biologically active ligand or compound that binds to a complementary biologically active cytokine receptor or cytokine receptor subunit and activates the cytokine receptor to cause a biological response mediated by the receptor, or to enhance a preexisting biological activity mediated by the receptor. In some embodiments, the cytokine or cytokine receptor is selected from the group consisting of interleukin-2 (IL-2), an interleukin-15 (IL-15), an interleukin-7 (IL-7), an interleukin-21 (IL-21) or a combination of any of the foregoing.

In some embodiments, the cytokine mimetic is a ligand which binds the IL-2 receptor or a subunit thereof. In some embodiments, the cytokine mimetic is an IL-2Rb ligand that is capable of binding to the IL-2Rb subunit of a mammalian IL-2 receptor, such as a human IL-2 receptor, with an affinity (IC50) less than 100 μM. Suitable IL-2Rb ligands are disclosed, for example, in U.S. Application Publication No. 2020/0040034 A1 and in U.S. Provisional Application No. 62/930,758 filed on Nov. 5, 2019, each of which is incorporated by reference in its entirety. In some embodiments, the cytokine mimetic is an IL-2Rgc ligand capable of binding to the IL-2Rgc subunit of a mammalian IL-2 receptor, such as a human IL-2 receptor, with an affinity (IC50) less than 100 μM. IL-2Rgc ligands are disclosed in U.S. Application Publication No.2020/0040036 A1 and in U.S. Provisional Application No. 62/930,758 filed on Nov. 5, 2019, each of which is incorporated by reference in its entirety. Further ligands are disclosed in WO2020217388, which is similarly incorporated by reference in its entirety.

In some embodiments, the cytokine mimetic is an IL-2Ra ligand capable of binding to the IL-2Ra subunit of a mammalian IL-2 receptor, such as a human IL-2 receptor, with an affinity (IC50) less than 100 μM. In some embodiments, the cytokine mimetic is an IL-2Rb ligand capable of binding to the IL-2Rb subunit of the human IL-2 receptor with an affinity less than 100 μM.

In some embodiments, the cytokine mimetic is a ligand which binds the IL-7 receptor or a subunit thereof. In some embodiments, the cytokine mimetic is an IL-7 receptor agonist effective to bind to IL-7 receptor a (CD127). In some embodiments, the IL-7 cytokine mimetic binds to IL-7 receptor a with a lower binding affinity than the unmodified parent IL-7 molecule (for example, when determined by surface plasmon resonance). In one or more additional embodiments, the IL-7 receptor agonist (i.e., IL-7 cytokine mimetic) has a binding affinity to IL-7 receptor a that is decreased by about 5 -fold to about 150-fold, or from about 8-fold to about 125-fold, or from about 10-fold to about 100- fold, when compared to the unmodified parent IL-7 molecule (for example, when determined by surface plasmon resonance). In some embodiments, the IL-7 receptor agonist (i.e., IL-7 cytokine mimetic) exhibits an IL-7 receptor off-rate that is substantially the same as that of the unmodified parent IL-7 molecule. In some embodiments, the IL-7 receptor agonist (i.e., IL-7 cytokine mimetic) exhibits an IL-7 receptor off-rate that is within about +1.7 times the value of the IL-7 receptor off-rate of the unmodified parent IL-7 molecule. IL-7R ligands are disclosed in WO2022020637, which is incorporated by reference in its entirety.

### 3. Antibodies

In some embodiments, the cytokine receptor agonist is an antibody or antigen binding fragment thereof. In some embodiments, the antibody of antigen binding fragment thereof is one that binds a cytokine or cytokine receptor. In some embodiments, the cytokine or cytokine receptor is selected from the group consisting of interleukin-2 (IL-2), an interleukin-15 (IL-15), an interleukin-7 (IL-7), an interleukin-21 (IL-21) or a combination of any of the foregoing. In some embodiments, an antibody of the disclosure further comprises a heavy chain constant region, wherein the heavy chain constant region is a modified or unmodified IgG, IgM, IgA, IgD, IgE, a fragment thereof, or combinations thereof.

a. IL-2

In various embodiments, the cytokine receptor agonist is an antibody that inhibits IL-2 signaling through IL-2 Rαβγ and through IL-2 Rβγ, and/or the antibody inhibits IL-2 signaling through IL-2 Rαβγto a greater extent than through IL-2 Rβγ. In some embodiments, the antibody binds IL-2 and inhibits binding of IL-2 with an IL-2 receptor alpha (IL-2 Rα) subunit. In various embodiments, the antibody inhibits IL-2 signaling through IL-2 Rαβγ to a greater extent than through IL-2 Rβγ. In some embodiments, the antibody does not completely block binding of human IL-2 to cells expressing human or mouse IL-2 Rβ or IL-2 Rβγcomplex. In a related aspect, the antibody binds at a site allosteric to binding of IL-2 to IL-2 Rα or IL-2 Rβ and γc chains.

In certain embodiments, an antibody described herein inhibits binding of IL-2 with an IL-2 receptor alpha (IL-2 R-a) subunit. In a related embodiment, an antibody described herein does not completely block binding of IL-2 to an IL-2 Rβγcomplex. In certain embodiments, an antibody described herein binds at a site allosteric to binding of IL-2 to IL-2 Rα, IL-2RP, IL-2Rγ and/or IL-2 Rβγ. In certain embodiments, an antibody described herein binds to IL-2 and induces a conformational change that impacts binding preference to IL-2Rα, IL-2Rβ, and/or γc.

In some embodiments, the antibody is a negative modulator antibody, optionally wherein the antibody is capable of weakening the binding affinity between IL-2 and IL-2 receptor a (IL-2 Rα) by at least about 2-fold, optionally up to 1000-fold. In other embodiments, an antibody described herein is capable of weakening the binding affinity between IL-2 and IL-2 Rα by at least 2-1000 fold, 10-100 fold, 2-fold, 5-fold, 10-fold, 25-fold, 50-fold, 100-fold, 200-fold, 300-fold, 400-fold, 500-fold, 600-fold, 700-fold, 800-fold, 900-fold or 1000-fold. In various embodiments, the antibody complexed with IL-2 binds to cells expressing IL-2 Rβ and γc with an EC50 of about 5 nM or less. In some embodiments, the antibody binds to cells expressing IL-2 Rβ(but not γc) with an EC50 of about 200 nM or less. In some embodiments, the antibody complexed with IL-2 binds to cells expressing IL-2 Rβand γc with an EC50 in a range of 0.1-100 nM, 0.1-10 nM, 1-5 nM. In various embodiments, the antibody complexed with IL-2 binds to cells expressing IL-2 Rβand γc with an EC50 of 1, 2, 3, 4 or 5 nM. In some embodiments, the antibody complexed with IL-2 binds to cells expressing IL-2 Rβ(but not γc) with an EC50 in a range of 10-500 nM, 10-300 nM, 10-200 nM. In various embodiments, the antibody complexed with IL-2 binds to cells expressing IL-2 Rβand γc with an EC50 of 10, 50, 100, 150 or 200 nM. In related embodiments, the antibody complexed with IL-2 binds to cells expressing IL-2 Rβand γc 9- to 40-fold more than cells expressing IL-2 Rβ(but not γc).

In various embodiments, the antibody inhibits IL-2 stimulation of proliferation in cells expressing IL-2 Rα, IL-2 Rβ, and γc to a greater extent than in cells expressing IL-2 Rβand γc, but not IL-2 Rα. In various embodiments, the antibody inhibits IL-2 stimulation of proliferation of NK cells that express IL-2 Rα, IL-2 Rβ, and γc, by greater than 20-fold. In various embodiments, the antibody inhibits IL-2 stimulation of proliferation of BaF3 cells that express IL-2 Rβand γc, but not IL-2 Rα, by less than 12-fold.

In some embodiments, the antibody is selected from the group consisting of XPA.92.019, XPA.92.041, XPA.92.042 or XPA.92.099. In some embodiments, the disclosure provides an antibody that binds human interleukin-2 (IL-2) comprising: (a) a heavy chain CDR1; (b) a heavy chain CDR2. In some embodiments, at least two of the heavy chain CDR1, CDR2 or CDR3 amino acid sequences are set forth in any one of SEQ ID NOs: 17-28. In a related embodiment, three of the heavy chain CDR1, CDR2 and CDR3 amino acid sequences are set forth in any one of SEQ ID NOs: 98-109. In some embodiments, an antibody contemplated herein further comprises any one of the light chain CDR amino acid sequences set forth in any one of SEQ ID NOs: 144-155.

b. IL-21

In some embodiments, the cytokine receptor agonist is an antibody or antigen binding fragment thereof that binds IL-21. In some embodiments, the antigen binding fragment binds to or specifically binds to IL-21 and enhances IL-21 activity, wherein the antigen binding fragment comprises one or more sequences at least about 80%, at least 85%, at least 90%, at least 92%, at least 95%, at least 97%, at least 99% identical to a sequence selected from the group consisting of SEQ ID NO: 160-175. In some embodiments, the antigen binding fragment binds to or specifically binds to IL-21 and enhances IL-21 activity, wherein the antigen binding fragment comprises one or more sequences selected from the group consisting of SEQ ID NO: 160-175.

In some examples of the present invention, a protein is capable of enhancing IL-21 activity. Various assays are known in the art for assessing the ability of a protein to enhance signaling of a ligand through a receptor. In one example, the antigen binding site enhances proliferation of cells (e.g., BaF3 cells) expressing IL-21R and gp130 (e.g., cells modified to express the both proteins) which are cultured in the presence of IL-21. Cells (e.g., about 1×104 cell) are cultured in the presence of IL-21 (e.g., between about 0.5 ng/mL to about 5 ng/mL (such as 0.5 ng/mL or 5 ng/mL) for hIL-21 and the presence or absence of a test antigen binding site. Methods for assessing cell proliferation are known in the art and include, for example, MTT reduction and/or thymidine incorporation. An antigen binding site that enhances the level of proliferation compared to the level observed in the absence of the antigen binding site is considered to enhance or agonize IL-21 signaling. A similar assay to that described in the foregoing paragraph can be performed with B9 cells or T10 cells (Dams-Kozlowska et al., BMC Biotechnol, 12: 8, 2012; and Yokote et al., J AOAC, 83: 1053-1057, 2000). In the case of an assay making use of T10 cells, proliferation can be measured by colorimetrically detecting reduction of the tetrazolium compound, 4- [3-(4-iodophenyl)-2-(4-nitrophenyl)-2H-5-tetrazolio]-1,3-benzene disulfonate (WST-1). Other methods for assessing enhancement of IL-21 signaling are contemplated by the present disclosure.

### 4. Half Life Extension

In some embodiments, the cytokine receptor agonist further comprises a moiety to extend half-life in vivo. In some embodiments, the half-life extending moiety is any one selected from the group consisting of an Fc region of immunoglobulin or a part thereof, albumin, an albumin-binding polypeptide, Pro/Ala/Ser (PAS), C-terminal peptide (CTP) of 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof. In some embodiments, the half-life extending moiety is a XTEN peptide, a glycine-rich homoamino acid polymer (HAP), a PAS polypeptide, an elastin-like polypeptide (ELP), a CTP peptide, or a gelatin-like protein (GLK) polymer.

In some embodiments, the cytokine receptor agonist is a cytokine or cytokine mutein, a cytokine mimetic, or an antibody or antigen binding fragment thereof that is modified, such as a modification to extend half-life as compared to the unmodified cytokine receptor agonist.

In some embodiments, the cytokine receptor agonist is functionally associated to a human serum albumin (“HSA”) or a portion of a HSA, as a fusion protein. Such fusion molecules have potentially increased stability and prolonged half-life in vivo.

The half-life of IL-21 can be measured, for example, by pharmacokinetic studies, e.g., according to the method described by Kim et al, Eur J of Immunol 24:542, 1994. According to this method radiolabeled IL-21 is injected intravenously into mice and its plasma concentration is periodically measured as a function of time, for example at 3 minutes to 72 hours after the injection. The clearance curve thus obtained should be biphasic, that is, an alpha phase and beta phase. For the determination of the in vivo half-life of the protein, the clearance rate in beta-phase is calculated and compared with that of the wild type or unmodified protein.

c. Fc Domains

In one or more embodiments of the invention, a conjugate is provided, the conjugate comprising a residue of a cytokine receptor agonist covalently attached to an Fc domain which prolongs the half-life of the cytokine receptor agonist. In particular, the Fc domain of the cytokine receptor agonist conjugate may be one in which the antibody-dependent cellular cytotoxicity (ADCC) or complement-dependent cytotoxicity (CDC) weakened due to the modification in the binding affinity with the Fc receptor and/or a complement. The modified Fc domain may be selected from the group consisting of IgG1, IgG2, IgG3, IgG4, IgA1, IgA2, IgD, IgE and a combination thereof. Specifically, the Fc region may include a hinge region, a CH2 domain, and a CH3 domain from the N-terminal to the C-terminal. In particular, the hinge region may include the human IgD hinge region; the CH2 domain may include a part of the amino acid residues of the human IgD and a part of the amino acid residues of the human IgG4 CH2 domain; and the CH3 domain may include a part of the amino acid residues of the human IgG4 CH3 domain.

The Fc domain may be in the form of having native sugar chains, increased sugar chains, or decreased sugar chains compared to the native form, or may be in a deglycosylated form. The Fc sugar chains may be modified by conventional methods such as a chemical method, an enzymatic method, and a genetic engineering method using a microorganism. The removal of sugar chains from an Fc fragment results in a sham decrease in binding affinity to the Clq part of the first complement component Cl, and a decrease or loss of ADCC or CDC, thereby not inducing any unnecessary immune responses in vivo. As used herein, the term “deglycosylation” refers to an Fc domain in which sugars are removed enzymatically from an Fc fragment. Additionally, the term “aglycosylation” means that an Fc fragment is produced in an unglycosylated form by a prokaryote, and preferably in E. coli.

d. Water Soluble Polymers

In one or more embodiments of the invention, a conjugate is provided, the conjugate comprising a residue of a cytokine receptor agonist covalently attached to a water-soluble polymer. In one or more embodiments of the invention, wherein the residue of the cytokine receptor agonist is covalently attached to the water-soluble polymer via a releasable linkage.

In some embodiments, a conjugate is provided, the conjugate comprising a residue of a cytokine moiety covalently attached to a water-soluble polymer, wherein the cytokine moiety is a precursor cytokine moiety. In some embodiments, a conjugate is provided, the conjugate comprising a residue of an IL-2 moiety covalently attached to a water-soluble polymer, wherein the IL-2 moiety is a precursor IL-2 moiety.

Provided herein is a cytokine receptor agonist that is a conjugate comprising a cytokine, cytokine mimetic, or antibody moiety attached to a water-soluble polymer. With respect to the water-soluble polymer, the water-soluble polymer is nonpeptidic, nontoxic, non-naturally occurring and biocompatible. With respect to biocompatibility, a substance is considered biocompatible if the beneficial effects associated with use of the substance alone or with another substance (e.g., an active cytokine receptor agonist such as an IL-2 moiety) in connection with living tissues (e.g., administration to a patient and/or subject) outweighs any deleterious effects as evaluated by a clinician, e.g., a physician. With respect to non-immunogenicity, a substance is considered non-immunogenic if the intended use of the substance in vivo does not produce an undesired immune response (e.g., the formation of antibodies) or, if an immune response is produced, that such a response is not deemed clinically significant or important as evaluated by a clinician. It is particularly preferred that the nonpeptidic water-soluble polymer is biocompatible and non-immunogenic.

In some embodiments, the water-soluble polymer comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), or a combination thereof. In some embodiments, the water-soluble polymer comprises a PEG molecule. In some embodiments, the PEG molecule is a linear PEG. In some embodiments, the PEG molecule is a branched PEG. In some embodiments, the water-soluble polymer comprises a polysaccharide. In some embodiments, the polysaccharide comprises dextran, polysialic acid (PSA), hyaluronic acid (HA), amylose, heparin, heparan sulfate (HS), dextrin, or hydroxyethyl-starch (HES).

In some embodiments, “PEG,” “polyethylene glycol” and “poly(ethylene glycol)” as used herein, are interchangeable and encompass any nonpeptidic water-soluble poly(ethylene oxide). In some embodiments, PEGs comprises the following structure “-(OCH2CH 2)n-” where (n) is 2 to 4000. As used herein, PEG also includes “-CH2 CH2-O(CH 2CH2 O),-CH 2 CH 2-” and “-(OCH2 CH2)nO-,” depending upon whether or not the terminal oxygens have been displaced, e.g., during a synthetic transformation. In some embodiments, “PEG” includes structures having various terminal or “end capping” groups and so forth. In some embodiments, “PEG” also means a polymer that contains a majority, that is to say, greater than 50%, of -OCH 2 CH2- repeating subunits. In some aspects, PEG can take any number of a variety of molecular weights, as well as structures or geometries such as “branched,” “linear,” “forked,” “multifunctional,” and the like. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer.

The terms “end-capped” and “terminally capped” are interchangeably used herein to refer to a terminal or endpoint of a polymer having an end-capping moiety. In some aspects, the end-capping moiety comprises a hydroxy or C1 20 alkoxy group, more preferably a C o alkoxy group, and still more preferably a C1 5 alkoxy group. Thus, examples of end-capping moieties include alkoxy (e.g., methoxy, ethoxy and benzyloxy), as well as aryl, heteroaryl, cyclo, heterocyclo, and the like. In some embodiments, the end-capping moiety may include one or more atoms of the terminal monomer in the polymer.

Typically, activated water-soluble polymers (e.g., polymeric reagents such as PEG) are activated with a suitable activating group appropriate for coupling to a desired site on the cytokine, cytokine mimetic, or antibody moiety (e.g., IL-2 moiety). Thus, a polymeric reagent will possess a reactive group for reaction with the cytokine, cytokine mimetic, or antibody moiety. Representative polymeric reagents and methods for conjugating these polymers to an active moiety are known in the art and further described in Zalipsky, S., et al., “Use of Functionalized Poly(Ethylene Glycols)for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182. Exemplary activating groups suitable for coupling to a cytokine, cytokine mimetic, or antibody moiety include hydroxyl, maleimide, ester, acetal, ketal, amine, carboxyl, aldehyde, aldehyde hydrate, ketone, vinyl ketone, thione, thiol, vinyl sulfone, hydrazine, among others. Chemistries currently exist for pegylation of, for example, a polypeptide's N- terminus, lysine residues, cysteine residues, histidine residues, arginine residues, aspartic acid residues, glutamic acid residues, serine residues, threonine residues, tyrosine residues, and C- terminus.

In some embodiments, the polymeric reagent used to prepare the conjugates described herein is prepared without the use of phosgene. Such an approach stands in contrast to, for example, the disclosure set forth in U.S. Pat. No. 4,902,502, which specifically describes forming a chloroformate and subsequent used to form a PEG active ester, which is then reacted with IL-2. Use of phosgene leads to the formation of hydrogen chloride, which can lead to chain cleavage in the polymer, thereby increasing impurities, which may not be able to be removed using conventional techniques. Thus, without wishing to be bound by theory, cytokine moiety conjugates prepared from polymeric reagents formed without the use of phosgene provides higher quality compositions that are substantially absent polymer chain degradation products.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values are known in the art can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the invention are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

In some embodiments, the PEG group has an average molecular weight selected from about 5 kDa, 10 kDa, 20 kDa and 30 kDa. In some embodiments, the PEG group has an average molecular weight of about 5 kDa. In some embodiments, the PEG group has an average molecular weight of about 10 kDa. In some embodiments, in the PEG group has an average molecular weight of about 15 kDa. In some embodiments, the PEG group has an average molecular weight of about 20 kDa. In some embodiments, the PEG group has an average molecular weight of about 25 kDa. In some embodiments, the PEG group has an average molecular weight of about 30 kDa. In some embodiments, the PEG group has an average molecular weight of about 35 kDa. In some embodiments, the PEG group has an average molecular weight of about 40 kDa. In some embodiments, the PEG group has an average molecular weight of about 45 kDa. In some embodiments, the PEG group has an average molecular weight of about 50 kDa. In some embodiments, the PEG group has an average molecular weight of about 60 kDa.

In some embodiments, a water soluble polymer that is or comprises PEGs will typically comprise a number of (OCH 2 CH2) monomers [or (CH 2CH 20) monomers, depending on how the PEG is defined]. The number of repeating units is identified by the subscript “n” in “(OCH 2CH 2)n.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

In addition to the above-described forms of PEG, the water soluble polymer can also be prepared with one or more weak or degradable linkages in the polymer, including any of the above-described polymers. For example, PEG can be prepared with ester linkages in the polymer that are subject to hydrolysis. This hydrolysis results in cleavage of the polymer into fragments of lower molecular weight.

Other hydrolytically degradable linkages, useful as a degradable linkage within a polymer backbone and/or as a degradable linkage to a cytokine moiety, include: carbonate linkages; imine linkages resulting, for example, from reaction of an amine and an aldehyde (see, e.g., Ouchi et al. (1997) Polymer Preprints38(1):582-3); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; orthoester linkages that are, for example, formed by reaction between a formate and an alcohol; amide linkages formed by an amine group, e.g., at an end of a polymer such as PEG, and a carboxyl group of another PEG chain; urethane linkages formed from reaction of, e.g., a PEG with a terminal isocyanate group and a PEG alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′hydroxyl group of an oligonucleotide.

The pegylated agonists contemplated herein may comprise at least one PEG molecule covalently attached through a linker to at least one amino acid residue of the agonist (e.g., N-terminal or C-terminal pegylation). In some embodiments, two or more different sites on the agonist may be pegylated by introducing more than one mutation and then modifying each of them. In further embodiments, the N-terminus may be pegylated in combination with the introduction of one or more mutations, and the pegylation thereof, elsewhere within the cytokine receptor agonist. In still further embodiments, the C-terminus may be pegylated in combination with the introduction of one or more mutations, and the pegylation thereof, elsewhere within the agonist. In additional embodiments, the cytokine receptor agonist may comprise pegylation at the N-terminus and the C-terminus. In further embodiments, the cytokine receptor agonist N-terminus may be pegylated in combination with the introduction of one or more mutations, and the pegylation thereof, elsewhere within the cytokine receptor agonist. The PEG component may be any PEG tolerated by the peptides.

The water-soluble polymer associated with the cytokine receptor agonist can also be “releasable.” That is, the water-soluble polymer releases (either through hydrolysis, enzymatic processes, catalytic processes or otherwise), thereby resulting in the unconjugated cytokine receptor agonist moiety (e.g., a cytokine moiety). In some instances, releasable polymers detach from the moiety in vivo without leaving any fragment of the water-soluble polymer. In other instances, releasable polymers detach from the moiety in vivo leaving a relatively small fragment (e.g., a succinate tag) from the water-soluble polymer. An exemplary cleavable polymer includes one that attaches to the cytokine receptor agonist moiety via a carbonate linkage.

In some aspects, the cytokine receptor agonist is administered as a cytokine prodrug, meaning that the linkage between the polymer and the cytokine moiety is releasable to allow release of the parent moiety. Exemplary releasable linkages include carboxylate ester, phosphate ester, thiol ester, anhydrides, acetals, ketals, acyloxyalkyl ether, imines, orthoesters, peptides and oligonucleotides. Such linkages can be readily prepared by appropriate modification of either the cytokine moiety (e.g., the carboxyl group C terminus of the protein, or a side chain hydroxyl group of an amino acid such as serine or threonine contained within the protein, or a similar functionality within the carbohydrate) and/or the polymeric reagent using coupling methods commonly employed in the art.

### Methods of Use

In some embodiments, the lipid particles and payload genes provided herein or pharmaceutical compositions containing same can be administered to a subject, e.g. a mammal, e.g. a human. In some embodiments, the lipid particles and payload genes are administered by the provided system of ex vivo dosing and administration. In some embodiments, the methods and uses involve dosing the therapy in-line in a closed fluid circuit attached or operably connected to the subject being treated. In some embodiments, the fluid pathway from the whole blood sample from the subject to the reinfusion of the transfection mixture containing the lipid particles (e.g. viral vector) and PBMCs or subset, such as a leukapheresis or apheresis cell composition, is closed so that the entire process occurs while the system is connected to the subject or patient.

In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle, such as a targeted lipid particle, contains nucleic acid sequences encoding the payload agent (also interchangeably called an exogenous agent or in some cases “cargo”) for treating the disease or condition in the subject. Thus, in some embodiments, the disease or condition that is treated is any that may be treatable by the encoded payload agent. For instance, in some embodiments the payload agent encodes a chimeric antigen receptor (CAR) that specifically binds to an antigen, and the disease or condition to be treated can be any in which expression of the antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein.

For example, the exogenous agent is one that targets or is specific for a protein of a neoplastic cells and the lipid particle is administered to a subject for treating a tumor or cancer in the subject. In another example, the exogenous agent is an inflammatory mediator or immune molecule, such as a cytokine, and lipid particle is administered to a subject for treating any condition in which it is desired to modulate (e.g. increase) the immune response, such as a cancer or infectious disease. In some embodiments, the lipid particle is administered in an effective amount or dose to effect treatment of the disease, condition or disorder.

Provided herein are uses of any of the provided lipid particles in such methods and treatments, and in the preparation of a medicament in order to carry out such therapeutic methods. In some embodiments, the methods are carried out by administering the lipid particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the lipid particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the payload agent is a chimeric antigen receptor, including any as described in Section III.G and/or that specifically binds an antigen described in Section III.G. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, and RORI (associated with solid tumors).

In some embodiments, the disease or condition is a B cell malignancy and the antigen targeted by the CAR is expressed by cells associated with the B cell malignancy. In some embodiments, the antigen is CD19. In some embodiments, the antigen is CD20. In some embodiments, the antigen is CD22. In some embodiments, the antigen is BCMA. In some embodiments, the B cell malignancy is a Large B-cell Lymphoma (LBCL). In some embodiments, the disease or condition has relapsed in or the subject is refractory to treatment of the disease or condition. For instance, in some embodiments, the disease or condition is relapsed and/or refractory Large B-cell Lymphoma (LBCL). In some embodiments, LBCL include Non-Hodgkin's lymphoma, including diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma. In some embodiments, the subject has received or is receiving prior to the ex vivo dosing provided herein prior therapies, such as two or more lines of systemic therapy for treating the disease or condition. In some embodiments, the subject has relapsed and/or is refractory to the prior therapies. In some embodiments, the prior therapies include two or more prior therapies from a chemotherapy containing regimen, such as with anthracycline, or an anti-CD20 mAb (unless CD20 negative), or after autologous stem cell transplant (ASCT). In some embodiments, the subject has one or more measurable PET-positive lesion, such as measured per Lugano classification. In some embodiments, the subject as an ECOG performance status of 0 or 1. In some embodiments, the subject has adequate organ function.

In some embodiments, the disease or condition is a multiple myeloma and the antigen targeted by the CAR is expressed by cells associated with the multiple myeloma. In some embodiments, the antigen is BCMA. In some embodiments, the subject has or is suspected of having a multiple myeloma that is associated with expression of B cell maturation antigen (BCMA). In some embodiments, the multiple myeloma is a relapsed and/or refractory multiple myeloma.

In some aspects, response rates in subjects, such as subjects with LBCL, are based on the Lugano criteria. (Cheson et al., (2014) JCO., 32(27):3059-3067; Johnson et al., (2015) Radiology 2:323-338; Cheson, B.D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake≥mediastinum but≤liver; 4, uptake moderately >liver; 5, uptake markedly higher than liver and/or new lesions; X, new areas of uptake unlikely to be related to lymphoma.

In some embodiments, response is based on lack of detectable minimal residual disease (MRD negativity) which means that no disease is detected. Methods for assessing MRD include, but are not limited to flow cytometry, polymerase chain reaction (PCR) and next-generation sequencing. In some embodiments, a sample of bone marrow cells and/or peripheral blood cells is assessed for disease. For instance, certain mutations or genetic abnormalities can be assessed that are known to be associated with the cancer. A skilled artisan is familiar with methods to assess MRD.

In some cases, the pharmacokinetics of cells expressing the payload agent (e.g. CAR) are determined to assess the bioavailability of the engineered cells in vivo. Methods for determining the pharmacokinetics of engineered cells in vivo may include drawing peripheral blood from subjects that have received the ex vivo dosing and determining the number of engineered cells in the blood based on detection of the engineered payload agent (CAR) expressed by the cells. For example, an anti-idiotypic antibody against the CAR may be used for detection of the CAR-expressing cells.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In some embodiments, the administration is by infusion such as by intravenous infusion.

In some embodiments, the provided embodiments do not involve a lymphodepletion therapy prior to the ex vivo administration of the lipid particle (e.g. viral vector). Thus, the provided methods do not involve administration of lymphopleting regimens, such as those including cyclophosphamide and/or fludarabine and/or bendamustine, or other lymphodepleting regimens or protocols, prior to receiving administration of the lipid particles. It is understood that the exclusion of lymphodepleting therapies in accord to the provided methods does not exclude that the subject may have previous in time (e.g. months to years earlier) may have received a lymphodepleting therapy. Rather, the provided embodiments include those in which the subject has not in accord with the present dosing methods received a lymphodepleting therapy, such as within 60 days or 30 days, prior to the ex vivo administration of the lipid particles (e.g. viral vector) of the present methods.

In some embodiments, the lipid particle composition comprising an exogenous agent or cargo, may be used to deliver such exogenous agent or cargo to a cell tissue or subject. In some embodiments, delivery of a cargo by administration of a lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the lipid particle composition described herein is delivered for ex-vivo administration to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered for ex vivo administration to a tissue that is in an injured state (e.g., from trauma, disease, hypoxia, ischemia or other damage).

In some embodiments, the composition is delivered for ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of lipid particles are from the same subject that is administered a lipid particle composition. In other embodiments, they are different. In some embodiments, the source of lipid particles and recipient tissue may be autologous (from the same subject) or heterologous (from different subjects). In some embodiments, the donor tissue for lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the lipid particle particles is co-administered with an inhibitor of sypressyn, e.g., a siRNA or inhibitory antibody.

### A. Mobilization

In some embodiments, prior to performing the therapy or method, the subject is administered an agent to mobilize peripheral blood hematopoietic cells, including hematopoietic stem cells.

In some embodiments, also provided herein are methods of mobilizing hematopoietic cells, such as hematopoietic stem cells (HSCs), in connection with the provided methods of delivering a lipid particle (e.g. lentiviral vector), including a lipid particle containing or encoding an exogenous agent and/or payload gene, to a target stem cell. In some embodiments, the method of delivery include administering a mobilization agent, e.g., a mobilization regimen, to the subject and administering the lipid particle (e.g. lentiviral vector) to the subject in accord with the provided methods. In some embodiments, the mobilization agent is administered to the subject prior to obtaining the whole blood sample from the subject. In some embodiments, the mobilization agent includes a mobilization regimen that cause therapeutically inaccessible hematopoietic cells to become therapeutically accessible. In some embodiments, a mobilization agent increases the number of hematopoietic cells in peripheral blood, thus allowing for a more accessible source of hematopoietic cells for targeting by the lipid particles (e.g. lentiviral vectors) in accord with the methods as described.

In some embodiments, the hematopoietic cells are CD34+ and may include CD34+progenitor cells. In some embodiments, the hematopoietic cells are HSCs. In some aspects, the mobilization agent is a stem cell mobilization agent.

As used herein, “mobilizing” and “mobilizing hematopoietic cells” are used interchangeably to refer to the act of inducing the migration of hematopoietic cells, such as CD34+cells, including progenitor cells and/or hematopoietic stem cells, from a first location (e.g., stem cell niche, e.g., bone marrow) into a second location (e.g., tissue (e.g., peripheral blood) or organ (e.g., spleen). In some embodiments, the process of mobilizing hematopoietic cells involves the recruitment of stem cells from their tissue or organ of residence to peripheral blood following treatment with a mobilization agent, such as using a mobilization agent known to a skilled artisan, including any cytokine and chemotherapeutic drugs known in the art for this purpose (e.g., G-CSF). In some aspects, this process mimics the enhancement of the physiological release of stem cells from tissues or organs in response to stress signals during injury and inflammation. In some embodiments, the mobilization agent or agents act as agonists or antagonists that prevent the attachment of hematopoietic cells to cells or tissues of their microenvironment. In some embodiments, the mobilization agent or agents induce the release of proteases that cleave the adhesion molecules or support structures between hematopoietic cells and their sites of attachment. In some embodiments, the mobilization agent is capable of mobilizing any hematopoietic cell, such as stem cells and/or progenitor cells, in which heparan sulfate proteoglycans are responsible for maintaining adhesion of the cells in their cell niche. In an aspect, a method of mobilizing hematopoietic cells in a subject comprises administering to a subject an effective amount of an agent that inhibits the level or activity of heparan sulfate proteoglycans, thereby mobilizing hematopoietic cells in the subject.

In some embodiments, a stem cell mobilization agent increases the circulation of hematopoietic cells and/or mobilizes hematopoietic cells sequestered in bone marrow to exit bone marrow into compartments where they are accessible, e.g., accessible for transduction by the lipid particle and/or viral vector. For example, administration to a subject of a mobilization therapy can increase the circulation of hematopoietic cells and/or mobilize hematopoietic cells sequestered in bone marrow to exit bone marrow into compartments where they are accessible, such as the peripheral blood.

In some embodiments, the mobilization agent is administered prior to taking or obtaining whole blood from the subject. In some embodiments, the mobilization agent is administered to the subject within 7 days prior to collecting or obtaining whole blood from the subject, such as within 6 days, 5 days, 4 days, 3 days, 2 days or 1 day prior to obtaining whole blood. In some embodiments, the mobilization agent is administered twice a day, once a day or two or three times within a week. In some embodiments, the mobilization agent is administered once daily for consecutive days prior to obtaining the whole blood from the subject. In some embodiments, at least one dose of the mobilization agent is administered to the subject on the same day as the provided methods of obtaining whole blood and PBMCs contacting target cells with a lipid particle (e.g. lentiviral vector). In some embodiments, at least one dose of the mobilization agent is administered to the subject within 12 hours prior to obtaining whole blood from the subject, such as within 10 hours, 8 hours, 6 hours, 5 hours, 4 hours, 3 hours, 2 hours or 1 hour prior to obtaining whole blood from the subject.

In some embodiments, after administering the mobilization agent, the method includes obtaining whole blood from the subject for collecting or harvesting the peripheral blood cells, including mobilized hematopoietic cells, from the donor in accord with the provided methods. In some embodiments, harvesting the mobilized hematopoietic cells is by apheresis. In some embodiments, administration of the at least one mobilization agent to the subject is performed on the same day as the apheresis procedure. In some embodiments, the apheresis procedure is performed within an hour of administration of the at least one mobilization agent.

Exemplary mobilization agents include, without limitation, stem cell factor (SCF), small molecule VLA-4 inhibitor BI05192, BOP (N-(benzenesulfonyl)-L-prolyl-L-O-(1-pyrrolidinylcarbonyl)tyrosine), heparin, granulocyte colony-stimulating factor (G-CSF), and plerixafor (also known as AMD3100). In some embodiments, the mobilization agent of the method is selected from the group consisting of granulocyte-macrophage colony-stimulating factor (GM-CSF), Fms-related tyrosine kinase 3 (flt-3) ligand, stromal cell-derived factor 1 (SDF-1), agonists of the chemokine (C-C motif) receptor 1 (CCR1), such as chemokine (C-C motif) ligand 3 (CCL3, also known as macrophage inflammatory protein-1α (Mip-1α)), agonists of the chemokine (C—X-C motif) receptor 1 (CXCR1) and CXCR2, such as chemokine (C—X-C motif) ligand (CXCL1), CXCL2 (also known as growth-related oncogene protein-β (Gro-β)), and CXCL8 (also known as interleukin-8 (IL-8)), agonists of CXCR4, such as CTCE-002, ATI-2341, and Met-SDF-1, Very Late Antigen (VLA)-4 inhibitor, TG-0054, AMD3465, and any combination thereof.

In some embodiments, the mobilization agent is stem cell factor (SCF), small molecule VLA-4 inhibitor BI05192, BOP (N-(benzenesulfonyl)-L-prolyl-L-O-(1-pyrrolidinylcarbonyl)tyrosine), heparin, granulocyte colony-stimulating factor (G-CSF), or plerixafor (AMD3100). In some embodiments, the mobilization agent includes the combination of G-CSF and plerixafor (AMD3100). In some of any embodiments, G-CSF is filgrastim (e.g. Neupogen® or Zarzio®). In some embodiments, the G-CSF is a pegylated G-CSF, such as pegfilgrastim (e.g. Neulasta®).

Any of various known methods for mobilizing hematopoietic cells using a mobilization agent can be used, including but not limited to, any as described in PCT publication No. WO2021211450; U.S. publication Nos. US20200268850 and US20170106021; and U.S. Pat. Nos. 7,939,057 and 10,907,177.

In some embodiments, the mobilization regimen includes administration of at least one mobilizing agent. In various embodiments, at least one mobilizing agent is administered to a subject (i) daily on the four days prior to obtaining whole blood from the subject; (ii) on the day of obtaining whole blood from the subject and/or on the day of administering the contacted PBMCs to the subject for administering the lipid particle (e.g. lentiviral vector). In some embodiments, at least one mobilizing agent is administered to a subject (i) on the day prior to obtaining the whole blood from the subject and (ii) on the day of obtaining whole blood from the subject and/or on the day of administering the contacted PBMCs to the subject.

In some embodiments, the stem cell mobilization regimen includes administration of one or both of G- CSF and plerixafor/AMD3100. In various embodiments G-CSF is administered to a subject (i) daily on the four days prior to obtaining whole blood from the subject; and (ii) on the day of obtaining whole blood from the subject and/or on the day of administering the contacted PBMCs to the subject for administering the lipid particle (e.g. lentiviral vector). In various embodiments plerixafor/AMD3100 is administered to a subject (i) on the day prior to obtaining whole blood from the subject and (ii) on the day of obtaining whole blood from the subject and/or on the day of administering the contacted PBMCs to the subject.

In some embodiments, the at least one mobilizing agent is administered once daily at a dose that is, or is at least, 0.1, 1.0, 10, 20, 30, 40, 50, 75, 100, 150, or 200 μg/kg. In various embodiments, a daily dose of the at least one mobilizing agent has a range having a lower bound of 0.1 pg/kg/day, 1.0 pg/kg/day, 10 μg/kg/day, 20 μg/kg/day, 30 μg/kg/day, 40 μg/kg/day, 50 μg/kg/day, or 75 μg/kg/day and an upper bound of 100 μg/kg/day, 150 μg/kg/day, or 200 μg/kg/day. In various embodiments the at least one mobilizing agent is administered once daily at a dose that is, or is at least, 1 mg/kg, 2 mg/kg, 3 mg/kg, 4 mg/kg, 5 mg/kg, 7.5 mg/kg, 10 mg/kg, 15 mg/kg, or 20 mg/kg. In various embodiments, a daily dose of the at least one mobilizing agent has a range having a lower bound of 1 mg/kg/day, 2 mg/kg/day, 3 mg/kg/day, 4 mg/kg/day, 5 mg/kg/day, or 7.5 mg/kg/day and an upper bound of 10 mg/kg/day, 15 mg/kg/day, or 20 mg/kg/day.

In various embodiments G-CSF is administered once daily at a dose that is, or is at least, 10 μg/kg, 20 μg/kg, 30 μg/kg, 40 μg/kg, 50 μg/kg, 75 μg/kg, 100 μg/kg, 150 μg/kg, or 200 μg/kg. In various embodiments, a daily dose of G-CSF has a range having a lower bound of 10 μg/kg/day, 20 μg/kg/day, 30 μg/kg/day, 40 μg/kg/day, 50 μg/kg/day, or 75 μg/kg/day and an upper bound of 100 μg/kg/day, 150 μg/kg/day, or 200 μg/kg/day. In various embodiments plerixafor/AMD3100 is administered once daily at a dose that is, or is at least, 1 mg/kg, 2 mg/kg, 3 mg/kg, 4 mg/kg, 5 mg/kg, 7.5 mg/kg, 10 mg/kg, 15 mg/kg, or 20 mg/kg. In various embodiments, a daily dose of G-CSF has a range having a lower bound of 1 mg/kg/day, 2 mg/kg/day, 3 mg/kg/day, 4 mg/kg/day, 5 mg/kg/day, or 7.5 mg/kg/day and an upper bound of 10 mg/kg/day, 15 mg/kg/day, or 20 mg/kg/day.

In some embodiments, G-CSF may be administered daily as a dose of 0.5-16 μg/kg (e.g., 5-16 μg/kg or 10-16 μg/kg) for 1-10 days (e.g., 1-7 days, or particularly, 1-3 days). In another example, G-CSF may be administered to a healthy donor at a dosage of 10-16 μg/kg daily for up to seven days. Three or four days of treatment may be sufficient when the peripheral blood collections are combined with apheresis starting on, e.g., day 4. In another example, G-CSF may be administered at a dosage of 10 g/kg daily for four days with apheresis starting on, e.g., day 4. The most commonly used dosage of G-CSF in healthy donors is 10 μg/kg body weight daily with leukapheresis starting on day 5 onward until collection of an adequate number of stem cells (e.g., collections can be taken once or twice daily for 1 to 4 days, such as 1 or 2 days). G-CSF may be administered subcutaneously or intravenously. Methods of G-CSF administration and dosage are described in Juttner et al. (Blood 89:2233-2258, 1997), Kroschinsky et al. (Haematologica 90:1556-1671, 2005), U.S. Pat. No. 6,162,427, 2005/0186182, WO 2010051335, and WO 2005014023, all of which are incorporated herein by reference in their entireties. If cyclophosphamide is administered for mobilization, G-CSF is usually started 2-5 days after completion of cyclophosphamide infusion. Methods of administering combined mobilization agents of G-CSF and one or more chemotherapeutic agents are described in Andre et al. (Transfusion 43:50-57, 2003), Ataergin et al. (Am. J. Hematol. 83:644-648, 2008), and Demirer et al. (Br. J. Haematol. 116:468-474, 2002), all of which are incorporated herein by reference in their entireties.

Plerixafor may be administered at a dosage of 1-300 μg/kg. At 240 μg/kg, the number of mobilized stem cells peak at around 4-10 hours after plerixafor administration. In some examples, plerixafor may be administered once or twice daily at a dose of about 1-300 μg/kg (e.g., 100-300 μg/kg or 200-300 μg/kg) for 1-10 days (e.g., 1-5 days).

The combination of plerixafor and G-CSF for stem cell mobilization was approved by the FDA in 2008 for use in patients and/or subjects with non-Hodgkin's lymphoma and multiple myeloma. This combination therapy may also be used to mobilize stem cells. A typical combination therapy may include, e.g., the administration of G-CSF at ˜0.5-16 μg/kg (e.g., 10 μg/kg) daily with plerixafor at 1-300 μg/kg (e.g., 240 μg/kg) given a few days (e.g., 1-3 days) after G-CSF administration. Both agents may be given together for about 2-10 days (e.g., 4 days) or until adequate hematopoietic cells are collected. Plerixafor, either alone or in combination with G-CSF, may be administered subcutaneously or intravenously.

In some embodiments, the method of mobilizing hematopoietic cells and/or progenitor cells in a subject includes harvesting the peripheral blood cells mobilized in the subject. In some embodiments, the method of mobilizing hematopoietic cells and/or progenitor cells in a subject includes harvesting the peripheral blood cells via apheresis. In some embodiments, the hematopoietic cell mobilization and apheresis are performed on the same day. In some embodiments, a single session of apheresis collects enough peripheral blood cells for a cell dose of between about 2×10⁶/kg and 10×10⁶/kg of the recipient's body weight. In some embodiments, the method of mobilizing hematopoietic cells in a subject includes administering to the subject at least one mobilization agent. In some embodiments, the method of mobilizing hematopoietic cells includes administering to the subject at least one mobilization agent comprising (i) at least one heparan sulfate inhibitor and (ii) at least one of a CXCR2 agonist and a CXCR4 antagonist.

In some embodiments, administration of the at least one mobilization agent mobilizes an amount of circulating peripheral blood cells in the subject to harvest a cell dose of between about 1×10⁶/kg body weight and 10×10⁶/kg body weight in a single apheresis session. In some embodiments, administration of the at least one mobilization agent mobilizes an amount of circulating peripheral blood cells in the subject to harvest a cell dose of between about 2×10⁶/kg body weight and 8×10⁶/kg body weight in a single apheresis session. In some embodiments, administration of the at least one mobilization agent mobilizes an amount of circulating peripheral blood cells in the subject to harvest a cell dose of between about 3×10⁶/kg body weight and 6×10⁶/kg body weight in a single apheresis session.

In some embodiments, harvesting the peripheral blood cells comprises apheresis and the hematopoietic cell mobilization and apheresis are performed on the same day. In some embodiments, a single session of apheresis collects enough CD34+peripheral blood cells for a cell dose of between about 2×10⁶/kg and 10×10⁶/kg of the recipient's body weight.

In some embodiments, the mobilized hematopoietic cells comprise KLS-CD150+CD48-cells. In some embodiments, the mobilized hematopoietic cells comprise CD34- CD133+cells. In some embodiments, the mobilized hematopoietic cells and/or progenitor cells comprise common myeloid progenitor cells. In some embodiments, the mobilized hematopoietic cells and/or progenitor cells comprise granulocyte/monocyte progenitor cells. In some embodiments, the mobilized hematopoietic cells and/or progenitor cells comprise megakaryocyte/erythroid progenitor cells. In some embodiments, the mobilized hematopoietic cells and/or progenitor cells comprise committed lymphoid progenitor cells. In some embodiments, the mobilized hematopoietic cells and/or progenitor cells comprise a combination of common myeloid progenitor cells, granulocyte/monocyte progenitor cells, megakaryocyte/erythroid progenitor cells. In some embodiments, the hematopoietic progenitor cells comprise CD150- CD48-CD244+cells. In some embodiments, the hematopoietic progenitor cells comprise CD150-CD48+CD244+cells. In some embodiments, the hematopoietic progenitor cells comprise Lin- SCA-1-c-Kit+CD34+CD16/32mid cells. In some embodiments, the hematopoietic progenitor cells comprise lin-SCA-1- c-kit+CD34- CD16/32low cells. In some embodiments, the mobilized hematopoietic cells comprise CD34+peripheral blood stem cells.

### Exemplary Embodiments

### Among the Provided Embodiments are:

1. An method for administration of a lipid particle to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lipid particles to create a transfection mixture; and d) reinfusing the contacted PBMCs or subset and/or the transfection mixture to the subject, thereby administering the lipid particle to the subject.

2. An in-line method for administration of a lipid particle to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lipid particles to create a transfection mixture; and d) reinfusing the contacted PBMCs or subset and/or the transfection mixture to the subject, thereby administering the lipid particle to the subject, wherein steps (a)-(d) are performed in-line in a closed fluid circuit.

3. The method of embodiment 1 or 2, wherein the lipid particle comprises a nucleic acid encoding a payload gene.

4. A method for administration of a payload gene to a subject, the method comprising: a) obtaining whole blood from a subject; b) collecting the fraction of blood containing peripheral blood mononuclear cells (PBMCs) or subset thereof; c) contacting the PBMCs or subset with a composition comprising lipid particles comprising a nucleic acid encoding a payload gene to create a transfection mixture; and d) reinfusing the contacted PBMCs or subset and/or the transfection mixture to the subject, thereby administering the payload gene to the subject.

5. An in-line method for administration of a payload gene to a subject, the method comprising: a) obtaining whole blood from a subject; b) collecting the fraction of blood containing peripheral blood mononuclear cells (PBMCs) or subset thereof; c) contacting the PBMCs or subset with a composition comprising lipid particles comprising a nucleic acid encoding a payload gene to create a transfection mixture; and d) reinfusing the contacted PBMCs or subset and/or the transfection mixture to the subject, thereby administering the payload gene to the subject, wherein steps (a)-(d) are performed in-line in a closed fluid circuit.

6. A method for administration of a lipid particle to a subject, the method comprising: a) obtaining whole blood from a subject; b) collecting the fraction of blood containing peripheral blood mononuclear cells (PBMCs) subset thereof; c) contacting the PBMCs or subset thereof with a composition comprising lipid particles to create a transfection mixture; and d) reinfusing the contacted PBMCs or leukocyte components and/or the transfection mixture to the subject, thereby administering the lipid particle to the subject, wherein the method is characterized by one or more of:
(i) wherein the method is characterized by the collecting in (b) not comprising a selection step (e.g. immunoaffinity selection) for target cells;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, collected PBMCs or subset thereof, and transfection mixture are not subjected to cryopreservation or freezing; and/or
(v) steps (a)-(d) are carried out for a time that is no more than 24 hours.

7. The method of embodiment 6, wherein the lipid particle comprises a nucleic acid encoding a payload gene.

8. A method for delivering a payload gene to a subject, the method comprising: a) obtaining whole blood from a subject; b) collecting the fraction of blood containing peripheral blood mononuclear cells (PBMCs) or leukocyte components; c) contacting the PBMCs or subset thereof with a composition comprising lipid particles comprising a nucleic acid encoding a payload gene to create a transfection mixture; and d) reinfusing the contacted PBMCs or subset thereof and/or the transfection mixture to the subject, thereby administering the lipid particle to the subject, wherein the method is characterized by one or more of:
(i) wherein the method is characterized by the collecting in (b) not comprising a selection step (e.g. immunoaffinity selection) for target cells.;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

9. A method for administration of a lentiviral vector encoding a chimeric antigen receptor (CAR) to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising a lentiviral vector comprising a nucleic acid encoding a CAR to create a transfection mixture; and d) reinfusing the contacted PBMCs or subset and/or the transfection mixture to the subject, thereby administering the lentiviral particle to the subject.

10. An in-line method for administration of a lentiviral vector encoding a chimeric antigen receptor (CAR) to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising a lentiviral vector comprising a nucleic acid encoding a CAR to create a transfection mixture; and d) reinfusing the contacted PBMCs or subset and/or the transfection mixture to the subject, thereby administering the lentiviral particle to the subject, wherein steps (a)-(d) are performed in-line in a closed fluid circuit.

11. A method for delivering a lentiviral vector encoding a chimeric antigen receptor (CAR) to a subject, the method comprising: a) obtaining whole blood from a subject; b) collecting the fraction of blood containing peripheral blood mononuclear cells (PBMCs) or leukocyte components; c) contacting the PBMCs or subset thereof with a composition comprising a lentiviral vector comprising a nucleic acid encoding a CAR to create a transfection mixture; and d) reinfusing the contacted PBMCs or subset thereof and/or the transfection mixture to the subject, thereby administering the lentiviral particle to the subject, wherein the method is characterized by one or more of:
(i) wherein the method is characterized by the collecting in (b) not comprising a selection step (e.g. immunoaffinity selection) for target cells;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

12. The method of embodiments 9-11, wherein the lentiviral vector is pseudotyped for targeting to a T cell.

13. The method of embodiment 12, wherein the T cell is a CD3+ T cell, a CD4+ T cell or a CD8+ T cell.

14. The method of embodiment 12 or embodiment 13, wherein the T cell is a CD8+ T cell.

15. The method of any of embodiments 9-14, wherein the CAR is an anti-CD19 CAR, an anti-CD22 CAR or an anti-CD22 CAR.

16. The method of any of embodiments 9-15, wherein the CAR is an anti-CD19 CAR.

17. The method of embodiment 15 or embodiment 16, wherein the anti-CD19 CAR comprises an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-lbb signaling domain set forth in SEQ ID NO:36, and a CD3zeta signaling domain set forth in SEQ ID NO: 38. 18. The method of any of embodiments 6-17, wherein the method is carried out in a single in-line procedure to maintain a closed or functionally closed fluid circuit.

19. The method of any of embodiments 1, 3, 4, 6, 7, 8, 9 11-18 wherein two or more of steps (a)-(d) are carried out in-line in a closed fluid circuit.

20. The method of any of embodiments 1, 3, 4, 6, 7, 8, 9, 11-19, wherein three or more of steps (a)-(d) are carried out in-line in a closed fluid circuit.

21. The method of embodiment 19 or embodiment 20, wherein between at least two steps, the method includes separating the subject from the in-line closed fluid circuit and then reconnecting the subject prior to the next step.

22. The method of any of embodiments 19-21, wherein steps (a)-(c) are carried out in-line in a closed fluid circuit, and wherein the method comprises separating the subject from the closed fluid circuit after step (c) and reconnecting the subject to the closed fluid circuit before step (d).

23. The method of any of embodiments 1, 3, 4, 6, 7, 8, 9, 11-20, wherein all of steps (a)-(d) are carried out in-line in a closed fluid circuit.

24. The method of any of embodiments 6-23, wherein the method is characterized by at least two of (i)-(v).

25. The method of any of embodiments 6-23, wherein the method is characterized by at least three of (i)-(v).

26. The method of any of embodiments 6-23, wherein the method is characterized by at least four of (i)-(v).

27. The method of any of embodiments 6-23, wherein the method is characterized by (i)-(v).

28. The method of any of embodiments 1-27, wherein the method is characterized by the collecting in (b) not comprising a selection step (e.g. immunoaffinity selection) for target cells.

29. The method of embodiment 28, wherein the target cells are T cells and the method does not include a selection step for T cells positive for a T cell marker (e.g. CD3, CD4 or CD8).

30. The method of embodiment 28, wherein the target cells are CD34+cells and the method does not include a selection step for cells positive for CD34.

31. The method of any of embodiments 1-26 and 28-30, wherein the method is characterized by the contacting in step (c) being initiated within 24 hours after collecting the fraction of blood containing PBMCs or subset thereof.

32. The method of any of embodiments 1-31, wherein the contacting in step (c) is initiated immediately after collecting the fraction of blood containing PBMCs or subset thereof following transfer to a contacting chamber.

33. The method of any of embodiments 1-32, wherein the contacting in step (c) is initiated 0 to 12 hours, 0 to 6 hours, 0 to 4 hours, 0 to 2 hours or 0 to 1 hour, or 0 to 30 minutes after collecting the fraction of blood containing PBMCs or subset thereof.

34. The method of any of embodiments 1-33, wherein the contacting in step (c) is initiated within at or about 12 hours, within at or about 6 hours, within at or about 2 hours, within at or about 1 hour, within at or about 30 minutes or within at or about 15 minutes after collecting the fraction of blood containing PBMCs or subset thereof.

35. The method of any of embodiments 1-26, and 28-34, wherein the method is characterized by the contacting in step (c) being no more than 24 hours prior to the reinfusing in step (d).

36. The method of any of embodiments 1-35, wherein the contacting in step (c) is for 15 minutes to 24 hours, 15 minutes to 12 hours, 15 minutes to 6 hours, 15 minutes to 4 hours, 15 minutes to 2 hours, 15 minutes to 1 hour, 15 minutes to 30 minutes, 30 minutes to 24 hours, 30 minutes to 12 hours, 30 minutes to 6 hours, 30 minutes to 4 hours, 30 minutes to 2 hours, 30 minutes to 1 hour, 1 hour to 24 hours, 1 hour to 12 hours, 1 hour to 6 hours, 1 hour to 4 hours, 1 hour to 2 hours, 2 hours to 24 hours, 2 hours to 12 hours, 2 hours to 6 hours, 2 hours to 4 hours, 4 hours to 24 hours, 4 hours to 12 hours, 4 hours to 6 hours, 6 hours to 24 hours, 6 hours to 12 hours or 12 hours to 24 hours.

37. The method of any of embodiments 1-36, wherein the contacting in step (c) is for at or about 15 minutes, at or about 30 minutes, at or about 1 hour, or at or about 2 hours, or any value between any of the foregoing.

38. The method of any of embodiments 1-37, wherein at least a portion of the contacting in (c) is carried out under centrifugation.

39. The method of any of embodiments 1-26 and 28-38, wherein the method is characterized by the whole blood, PBMCs or subset thereof, and transfection mixture having not been subjected to cryopreservation or freezing.

40. The method of any of embodiments 1-37, wherein the fraction of blood, PBMCs or subset thereof, and transfection mixture are not formulated with a cryoprotectant (e.g. DMSO).

41. The method of any of embodiments 1-40, wherein the transfection mixture is directly reinfused to the subject, optionally without any further processing or washing steps.

42. The method of any of embodiments 1-26 and 28-41, wherein the method is characterized by steps (a)-(d) being carried out for a time that is no more than 24 hours.

43. The method of any of embodiments 1-42, wherein the steps (a)-(d) are carried out for a time that is between 1 hour and 24 hours, between 1 hour and 12 hours, between 1 hour and 6 hours, between 1 hour and 4 hours, between 1 hour and 2 hours, between 2 hours and 24 hours, between 2 hours and 12 hours, between 2 hours and 6 hours, between 2 hours and 4 hours, between 2 hours and 24 hours, between 2 hours and 12 hours, between 2 hours and 6 hours, between 2 hours and 4 hours, between 4 hours and 24 hours, between 4 hours and 12 hours, between 4 hours and 6 hours, between 6 hours and 24 hours, between 6 hours and 12 hours, or between 12 hours and 24 hours.

44. The method of any of embodiments 1-43, wherein the steps (a)-(d) are carried out for a time that is between 2 hours and 6 hours.

45. The method of any of embodiments 1-44, wherein the steps (a)-(d) are carried out for a time that is between 2 hours and 4 hours or between 3 hours and 4 hours.

46. The method of any of embodiments 2, 5, 10, 12-45, wherein the closed fluid circuit comprises one or more of a blood processing set for obtaining the whole blood from the subject, a separation chamber for the separating the PBMCS or subset from the blood to collect the PBMCs or subset, a contacting container for the contacting the collected PBMCs or subset thereof with the composition comprising lipid particles or lentiviral vector, and a transfer container containing the contacted PBMCs or subset thereof and/or the transfection mixture for reinfusion to the subject.

47. The method of any of embodiments 2, 5, 10, 12-45, wherein the closed fluid circuit comprises a blood processing set for obtaining the whole blood from the subject, a separation chamber for the separating the PBMCs or subset from the blood to collect the PBMCs or subset, a contacting container for the contacting the collected PBMCs or subset thereof with the composition comprising lipid particles or lentiviral vector, and a transfer container containing the contacted PBMCs or subset thereof and/or the transfection mixture for reinfusion to the subject.

48. The method of embodiment 46 or embodiment 47, wherein the blood processing set, the separation chamber, the contacting chamber and/or the transfer container are operably connected via at least one connector set comprising at least one tubing line and optionally one or more connectors.

49. The method of any of embodiments 46-48, wherein the closed fluid circuit further comprises a collection container operably connected to the separation chamber to collect the PBMCs or subset, optionally wherein the collection container is a bag, more optionally a sterile bag.

50. The method of embodiment 46-49, wherein the contacting chamber and the transfer container are the same container, optionally wherein the container is a bag, more optionally a sterile bag.

51. The method of any of embodiments 46-50, wherein the collecting container, the contacting chamber, and the transfer container are the same container, wherein the container is a bag, more optionally a sterile bag.

52. The method of any of embodiments 2, 5, 10, 12-45, wherein the closed fluid circuit comprises one or more of a blood processing set for obtaining the whole blood from the subject, a separation chamber for the separating the PBMCs or subset from the blood to collect the PBMCs or subset, and a container, wherein the container is configured as a collection container for collecting the PBMCs or subset from the separation chamber, a contacting chamber for contacting with the lipid particles or lentiviral vector to create a transfection mixture, and a transfer container for reinfusing the transfer mixture to the subject.

53. The method of embodiment 52, wherein the container is a bag, optionally a sterile bag.

54. The method of embodiment 52, wherein the blood processing set, the separation chamber, and the container are operably connected via at least one connector set comprising at least one tubing line and optionally one or more connectors.

55. The method of any of embodiments 46-54, wherein the container or the collecting container is operably connected to a source container comprising the composition comprising lipid particles or lentiviral vector, optionally wherein the operable connection is via at least one connector set comprising at least one tubing line and optionally one or more connectors.

56. The method of any of embodiments 46-55, wherein the container or the transfer container is operably connected to a return processing unit for reinfusion of contacted PBMCs or the transfection mixture to the subject.

57. The method of any of embodiments 46-56, wherein the operably connection is via at least one connector set comprising at least one tubing line and optionally one or more connectors.

58. The method of embodiment 48, wherein the transfer container is separably connected form the closed fluid circuit for reinfusion.

59. The method of embodiment 48, wherein the transfer container is not disengaged from the closed fluid circuit during reinfusion to the subject.

60. The method of embodiment 48 or embodiment 59, wherein the transfer container is part of a return processing unit comprised by the closed fluid circuit, said return processing unit configured to reinfuse the PBMCs or subset thereof or the transfection mixture to the subject.

61. The method of any of embodiments 2, 5, 10, 12-60, wherein the closed fluid circuit is characterized by:
(a) a blood processing set for obtaining the whole blood from the subject,
(b) a separation chamber for the separating the PBMCs or subset from the blood to collect the PBMCs or subset;
(c) a collecting container for collecting the PBMCs or subset from the separation chamber,
(d) a source container comprising the composition comprising lipid particles or lentiviral vector, wherein the collecting container is configured as a contacting container and is operably connected to the source container for contacting of the collected PBMCs or subset with a composition comprising lipid particles or lentiviral vector to create the transfection mixture; and
(e) a return processing unit configured to reinfuse to the subject the transfection mixture, wherein the collecting container containing the transfection mixture is configured as a transfer container and is operably connected to the return processing unit for reinfusion to the subject,
wherein (a)-(e) are operably connected in-line in a closed fluid circuit.

62. The method of embodiment 61, wherein the container is a bag, optionally a sterile bag.

63. The method of any of embodiments 48-59, wherein the one or more connectors is selected from the group consisting of valves, luer ports and spikes.

64. The method of embodiment any of embodiments 41-44, wherein the connector set is disposable.

65. The method of any of embodiments 48-64, wherein the connector set is sterile.

66. The method of any of embodiments 2, 5, 10, 12-65, wherein the closed fluid circuit is sterile.

67. The method of any of embodiments 46-66, wherein the transfer container is operably connected to the closed fluid circuit and/or donor subject, optionally via one or more tubing lines, during the reinfusion to the subject.

68. The method of any of embodiments 46-67, wherein during at least a portion of the contacting in (c) mixing the transfection mixture comprising the PBMCs or subset and the composition comprising lipid particles or lentiviral vector.

69. The method of embodiment 68, wherein the mixing is by physical manipulation.

70. The method of embodiment 68, wherein the mixing is by centrifugation.

71. The method of any of embodiments 46-67, 68 and 70, wherein the contacting chamber comprises a centrifuge.

72. The method of any of embodiments 1-71, wherein the collected fraction of blood contains PBMCs or subset thereof separated from other blood components.

73. The method of any of embodiments 1-72, wherein collecting the fraction of blood is by apheresis.

74. The method of embodiment 73, wherein the apheresis device comprises membrane apheresis or centrifugal apheresis.

75. The method of any of embodiments 1-74, wherein the collected fraction comprises leukocytes or precursors thereof.

76. The method of embodiment 75, wherein the precursors thereof comprise hematopoietic stem cells or a CD34+progenitor cell.

77. The method of any of embodiments 1-74, wherein collecting the fraction of blood is by leukapheresis.

78. The method of any of embodiment 1-77, wherein the collected fraction of blood contains leukocytes.

79. The method of any of embodiments 1-72, 77 and 78, wherein the collected fraction is a leukapheresis composition obtained from whole blood by leukapheresis.

80. The method of any of embodiments 1-79, wherein the transfection mixture is reinfused to the subject.

81. The method of any of embodiments 1-80, wherein the transfection mixture comprises an anticoagulant.

82. The method of embodiment 81, wherein the anticoagulant is a citrate.

83. The method of any of embodiment 1-82, wherein the viability of cells of the collected fraction is greater than 95%, e.g. between 95% and 100%, between 96% and 100%, between 97% and

100%, between 98% and 100% or between 99% and 100%.

84. The method of any of embodiment 1-82, wherein the viability of cells of the contacted PBMCs or subset thereof or of cells in the transfection mixture is greater than 95%, e.g. between 95% and 100%, between 96% and 100%, between 97% and 100%, between 98% and 100% or between 99% and 100%,

85. The method of any of embodiments 1-84, wherein the lipid particle is a viral vector or viral-like particle.

86. The method of embodiment 85, wherein the viral vector or viral-like particle is a retroviral vector or retroviral-like particle.

87. The method of embodiment 85 or embodiment 86, wherein the viral vector or viral-like particle is a lentiviral vector or lentiviral-like particle.

88. The method of any of embodiments 1-87, wherein the lipid particle comprises a fusogen embedded in the lipid bilayer.

89. The method of embodiment 88, wherein the fusogen is a viral fusogen selected from a Class I viral membrane fusion protein, a Class II viral membrane protein, a Class II viral membrane fusion protein, a viral membrane glycoprotein, or a viral envelope protein.

90. The method of embodiment 88 or embodiment 89, wherein the fusogen is endogenous to the virus.

91. The method of embodiment 88 or embodiment 89, wherein the fusogen is a pseudotyped fusogen.

92. The method of any of embodiments 88-91, wherein the fusogen is a viral envelope protein.

93. The method of any of embodiments 88-92, wherein the fusogen is a vesicular stomatitis virus envelope glycoprotein (VSV-G).

94. The method of any of embodiments 88-92, wherein the fusogen is a baboon endogenous virus (BaEV) envelope glycoprotein.

95. The method of any of embodiments 88-92, wherein the fusogen is a Cocal virus envelope glycoprotein.

96. The method of any of embodiments 88-92, wherein the fusogen is an Alphavirus fusion protein (e.g. Sindbis virus) or a functional variant thereof.

97. The method of any of embodiments 88-92, wherein the fusogen is a Paramyxoviridae fusion protein (e.g., a Morbillivirus or a Henipavirus) or a functional variant thereof.

98. The method of any of embodiments 88-92 and 97, wherein the fusogen is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.

99. The method of any of embodiments 88-92, 97 and 98, wherein the fusogen is a Henipavirus fusion protein (e.g., Nipah virus, Hendra virus, Cedar virus, Kumasi virus, Mójiāng virus) or a functional variant thereof.

100. The method of any of embodiments 88-92 and 97-99, wherein the fusogen is a Nipah virus fusion protein or a functional variant thereof.

101. The method of any of embodiments 88-92 and 97-100, wherein the fusogen comprises a paramyxovirus F protein, or a biologically active portion thereof.

102. The method of any of embodiments 88-92 and 97-101, wherein the fusogen comprises a paramyxovirus G, paramyxovirus H, and/or paramyxovirus HN protein, or a biologically active portion thereof.

103. The method of embodiment 102, wherein the paramyxovirus G, paramyxovirus H, and/or paramyxovirus HN protein further comprises a targeting moiety.

104. The method of any of embodiments 1-103, wherein the lipid particle or lentiviral vector comprises a nucleic acid encoding a payload gene.

105. The method of embodiment 104, wherein the nucleic acid encoding a payload gene encodes a chimeric antigen receptor (CAR).

106. The method of any of embodiments 103-105, wherein the targeting moiety comprises a binding agent.

107. The method of embodiment 106, wherein the binding agent is targeted against CD3, CD4, CD8, CD34, CD90, CD19, CD20, CD22, CD16, or CD56.

108. The method of any of embodiments 88-92 and 97-107, wherein the fusogen comprises an F protein molecule or a biologically active portion thereof from a Paramyxovirus and a glycoprotein G (G protein) or a biologically active portion thereof from a Paramyxovirus.

109. The method of embodiment 108, wherein the Paramyxovirus is a henipavirus.

110. The method of embodiment 108 or embodiment 109, wherein the Paramyxovirus is Nipah virus.

111. The method of any of embodiments 88-92 and 97-110, wherein the fusogen comprises a Nipah virus F glycoprotein (NiV-F) or a biologically active portion thereof and a Nipah virus G glycoprotein (NiV-G) or a biologically active portion thereof.

112. The method of embodiment 108 or embodiment 109, wherein the Paramyxovirus is Hendra virus.

113. The method of any of embodiments 108-111, wherein the G protein or the biologically active portion thereof is a mutant NiV-G protein or biologically active portion thereof that exhibits reduced binding to Ephrin B2 or Ephrin B3.

114. The method of embodiment 113, wherein the mutant NiV-G protein comprises one or more amino acid substitutions corresponding to amino acid substitutions selected from the group consisting of E501A, W504A, Q530A and E533A with reference to numbering set forth in SEQ ID NO:14.

115. The method of any of embodiments 108-111, 113 and 114, wherein the G protein or biologically active portion is a biologically active portion of wild-type NiV-G that has a deletion of up to 40 amino acids at or near the N-terminus, optionally not including the initial methionine.

116. The method of any of embodiments 108-111, and 113-115, wherein the G protein is a biologically active portion that is a truncated NiV-G that has a deletion of amino acids 2-34 at or near the N-terminus of wild-type NiV-G set forth in SEQ ID NO:14.

117. The method of any of embodiments 108-111, and 113-116, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19. 118. The method of any of embodiments 108-111, and 113-117, wherein the F protein or the biologically active portion thereof is a NiV-F protein or a biologically active portion thereof.

119. The method of embodiment 118, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

120. The method of any of embodiments 108-111, and 113-119, wherein the F protein or the biologically active portion is a truncated NiV-F that lacks amino acids 525-546 of SEQ ID NO:2.

121. The method of any of embodiments 108-111, and 113-120, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

122. The method of any of embodiments 108-111, and 113-121, wherein the Niv-G protein comprises the amino acid sequence set forth in SEQ ID NO: 19, and the Niv-F protein comprises the amino acid sequence set forth in SEQ ID NO:12.

123. The method of any of embodiments 88-122, wherein the fusogen is a re-targeted fusogen that binds to a target cell.

124. The method of embodiment 123, wherein the fusogen comprises a targeting moiety that binds to the target cell.

125. The method of any of embodiments 4-124, wherein the target cell is a T cell, a B cell, an NK cell, a macrophage, a monocyte, a dendritic cell, a hematopoietic stem cell or a CD34+progenitor cell.

126. The method of any of embodiments 4-125, wherein the target cell is a T cell.

127. The method of embodiment 124 or embodiment 126, wherein the targeting moiety binds to CD4 or CD8.

128. The method of any of embodiments 124-127, wherein the targeting moiety is a Design ankyrin repeat proteins (DARPin), a single domain antibody (sdAb), a single chain variable fragment (scFv), or an antigen-binding fibronectin type III (Fn3) scaffold.

129. A method for administration of a lentiviral vector to a subject, the method comprising:
a) obtaining whole blood from a subject;
b) collecting the fraction of blood containing peripheral blood mononuclear cells (PBMCs) or subset thereof;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the contacted PBMCs or subset and/or the transfection mixture to the subject, thereby administering the lentiviral vector to the subject.

130. An in-line method for administration of a lentiviral vector to a subject, the method comprising:
a) obtaining whole blood from a subject;
b) collecting the fraction of blood containing peripheral blood mononuclear cells (PBMCs) or subset thereof;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the contacted PBMCs or subset and/or the transfection mixture to the subject, thereby administering the lentiviral vector to the subject, wherein steps (a)-(d) are performed in-line in a closed fluid circuit.

131. A method for administrating a lentiviral vector to a subject, the method comprising:
a) obtaining whole blood from a subject;
b) collecting the fraction of blood containing peripheral blood mononuclear cells (PBMCs) or leukocyte components;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the contacted PBMCs or subset thereof and/or the transfection mixture to the subject, thereby administering the lentiviral particle to the subject, wherein the method is characterized by one or more of:
(i) wherein the method is characterized by the collecting in (b) not comprising a selection step (e.g. immunoaffinity selection) for target cells.;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

132. The method of any of embodiments 129-131, wherein the CAR binds to or recognizes a protein or antigen expressed by or on cells associated with a disease or condition.

133. The method of embodiment 132, wherein the disease or condition is a cancer.

134. A method for treating cancer in a subject, the method comprising:
a) obtaining whole blood from a subject;
b) collecting the fraction of blood containing peripheral blood mononuclear cells (PBMCs) or subset thereof;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the contacted PBMCs or subset and/or the transfection mixture to the subject, thereby administering the lentiviral vector to the subject.

135. An in-line method for treating cancer in a subject, the method comprising:
a) obtaining whole blood from a subject;
b) collecting the fraction of blood containing peripheral blood mononuclear cells (PBMCs) or subset thereof;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the contacted PBMCs or subset and/or the transfection mixture to the subject, thereby administering the lentiviral vector to the subject, wherein steps (a)-(d) are performed in-line in a closed fluid circuit.

136. A method for treating a cancer in a subject, the method comprising:
a) obtaining whole blood from a subject;
b) collecting the fraction of blood containing peripheral blood mononuclear cells (PBMCs) or leukocyte components;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the contacted PBMCs or subset thereof and/or the transfection mixture to the subject, thereby administering the lentiviral particle to the subject, wherein the method is characterized by one or more of:
(i) wherein the method is characterized by the collecting in (b) not comprising a selection step (e.g. immunoaffinity selection) for target cells.;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

137. The method of any of embodiments 124-136, wherein the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker.

138. The method of any of embodiments 124-136, wherein the CD8 binding agent is a VHH having the sequence set forth in SEQ ID NO: 128.

139. The method of embodiment 137 or embodiment 138, wherein the CD8 binding agent is linked to the C-terminus of a truncated NiV-G set forth in SEQ ID NO: 19 for retargeting of the lipid particle or lentiviral vector to CD8+ T cells.

140. The method of embodiment 139, wherein the lipid particle or lentiviral vector comprising the retargeted NiV-G is pseudotyped with the a NiV-F of a biologically active portion thereof, optionally wherein the NiV-F or biologically active portion is set forth in SEQ ID NO:12. 141. The method of any of embodiments 85-140, wherein the composition comprising the lipid particle is a viral vector and the composition comprising the lipid particle or the composition comprising the lentiviral vector comprises from 1×10⁸ to 1×10¹¹ infectious units (IU), 1×108 to 1×10¹⁰ IU, 1×10⁸ to 1×109 IU, 1×109 to 1×10¹¹ IU, 1×109 to 1×10¹⁰ IU, 1×10¹⁰ to 1×10¹¹ IU.

142. The method of any of embodiments 1-141, wherein the volume of the composition comprising lipid particles or lentiviral vector is between 100 mL and 400 mL, inclusive.

143. The method of any of embodiments 85-142, wherein the collected PBMCs or subset thereof comprises from 1×108 to 1×10¹⁰ nucleated cells, 1×108 to 5×109 nucleated cells, 1×108 to 2 ×10₉ nucleated cells, 1×10⁸ to 1×10⁹ nucleated cells, 1×10⁸ to 5×10⁸ nucleated cells, 5×10⁸ to 1×10¹⁰ nucleated cells, 5×10⁸ to 5×10⁹ nucleated cells, 5×10⁸ to 2×10⁹ nucleated cells, 5×10⁸ to 1×10⁹ nucleated cells, 1×10⁹ to 1×10¹⁰ nucleated cells, 1×10⁸ to 5×10⁹ nucleated cells, 1×10⁸ to 2×10⁹ nucleated cells, 2×10⁹ to 1×10¹⁰ nucleated cells, 2×10⁸ to 5×10⁹ nucleated cells, or 5×10⁹ to 1×10¹⁰ nucleated cells.

144. The method of any of embodiments 1-143, wherein the volume of the collected PBMCs or subset thereof is between 100 mL and 400 mL, inclusive.

145. The method of any of embodiments 1-144, wherein the concentration of the PBMCs or subset during the contacting is from 1×10⁶ cells/mL to 1×108 cells/mL, from 1×10⁶ cells/mL to 5×10⁷ cells/mL, from 1×10⁶ cells/mL to 1×10⁷ cells/mL, from 1×10⁶ cells/mL to 5×10⁶ cells/mL, from 5×10⁶ cells/mL to 1×108 cells/mL, from 5×10⁶ cells/mL to 5×10⁷ cells/mL, from 5×10⁶ cells/mL to 1×10⁷ cells/mL, from 1×10⁷ cells/mL to 1×108 cells/mL, from 1×10⁷ cells/mL to 5×10⁷ cells/mL, from 5×10⁷ cells/mL to 1×108 cells/mL

146. The method of any of embodiments 1-145, wherein the method does not include a lymphodepleting regimen prior to obtaining the whole blood from the subject.

147. The method of any of embodiments 1-146, wherein the method does not include a lymphodepleting regimen prior to reinfusing the contacted PBMCs or subset or the transfection mixture to the subject, optionally wherein the subject has not been subjected to a lymphodepleting regimen within

30 days prior, optionally within one week, prior to reinfusing the contacting PBMCs or subset of the transfection mixture to the subject.

148. The method of any of embodiments 3-147, wherein the payload agent is or encodes a therapeutic agent and/or wherein the payload agent is a nucleic acid comprising a gene for correcting a genetic deficiency.

149. The method of any of embodiments 3-148, wherein the payload agent encodes a membrane protein.

150. The method of embodiment 149, wherein the membrane protein is an antigen receptor for targeting cells expressed by or associated with a disease or condition.

151. The method of embodiment 149 or embodiment 150, wherein the membrane protein is a chimeric antigen receptor (CAR).

152. The method of any of embodiments 105-151, wherein the CAR is an anti-CD19 CAR, an anti-CD22 CAR or an anti-CD22 CAR.

153. The method of any of embodiments 105-152, wherein the CAR is an anti-CD19 CAR.

154. The method of embodiment 152 or embodiment 153, wherein the anti-CD19 CAR comprises an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36, and a CD3zeta signaling domain set forth in SEQ ID NO: 38.

155. The method of any of embodiments 1-154, further comprising administering a cytokine receptor agonist to the subject.

156. The method of embodiment 155, wherein the cytokine receptor agonist is a recombinant protein, a chemically synthesized protein or a conjugate.

157. The method of embodiment 155 or embodiment 156, wherein the cytokine receptor agonist binds to a cytokine receptor on a T cell.

158. The method of embodiment 157, wherein the cytokine receptor is selected from the group consisting of an IL-2 receptor (IL-2R), an IL-15 receptor (IL-15R), an IL-7 receptor (IL-7R), or an IL-21 receptor (IL-21R).

159. The method of any of embodiments 155-158, wherein the cytokine receptor agonist comprises a T cell stimulating cytokine or a T cell stimulating cytokine mutein, a T cell stimulating cytokine mimetic, an antibody or antigen-binding fragment that binds a cytokine receptor on a T cell, or an antibody or antigen-binding fragment that binds a T cell stimulating cytokine.

160. The method of any of embodiments 155-159, wherein the cytokine receptor agonist is a conjugate comprising (1) a T cell stimulating cytokine, a T cell stimulating cytokine mutein or a T cell stimulating cytokine mimetic and (2) a water soluble polymer.

161. The method of any of embodiments 155-160, wherein the cytokine receptor agonist is a fusion protein comprising (1) a T cell stimulating cytokine, a T cell stimulating cytokine mutein or a T cell stimulating cytokine mimetic and (2) a half-life extending moiety.

162. The method of any of embodiments 159-161, wherein the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21), or a T cell stimulating cytokine mutein of any of the foregoing.

163. The method of any of embodiments 159-162, wherein the T cell stimulating cytokine mutein comprises at least one amino acid modification relative to a wild-type T cell stimulating cytokine.

164. The method of any of embodiments 159-163, wherein the T cell stimulating cytokine mutein is an IL-2 cytokine mutein comprising one or more amino acid modifications relative to wild-type human IL-2.

165. The method of embodiment 164, wherein the IL-2 mutein exhibits increased affinity for the IL-2 Rβ, relative to wild-type human IL-2.

166. The method of embodiment 164 or embodiment 165, wherein the IL-2 mutein exhibits increased IL-2 activity for the intermediate affinity IL-2 receptor composed of IL-2Rbeta and IL-2Rgamma (IL-2R β/γ), relative to wild-type human IL-2.

167. The method any of embodiment 164, wherein the IL-2 mutein exhibits reduced binding to IL-2Ralpha, relative to wild-type human IL-2.

168. The method of any of embodiments 164-167, wherein the IL-2 mutein exhibits reduced IL-2 activity for the high-affinity IL-2 receptor composed of IL-2Ralpha, IL-2Rbeta and IL-2Rgamma (IL-2R α/β/γ).

169. The method of any of embodiments 159-162, wherein the T cell stimulating cytokine or cytokine mutein is IL-15 or an IL-15 cytokine mutein and the IL-15 or IL-15 cytokine mutein is bound to IL-15Rα or a portion thereof comprising the sushi domain.

170. The method of any of embodiments 159-162 and 169, wherein the T cell stimulating cytokine mutein is a IL-15 cytokine mutein comprising one more amino acid modifications relative to human IL-15.

171. The method of embodiment 169 or embodiment 170, wherein the IL-15 mutein exhibits reduced binding to IL-15Rα.

172. The method of any of embodiments 159-162, wherein the cytokine receptor agonist comprises a T cell stimulating cytokine mimetic and the mimetic is a IL-2Rα ligand, a IL-2Rβligand, a IL-2Rγ ligand, a common γc receptor (Rγc) ligand, and/or IL-7Rα ligand.

173. The method of any of embodiments 160 and 162-172, wherein one, two, three, four, five or six water-soluble polymers are attached to the T cell stimulating cytokine.

174. The method of any of embodiments 160 and 162-173, wherein the water soluble polymer is a polymer selected from the group consisting of poly(alkylene oxide), poly(vinyl pyrrolidone), poly(vinyl alcohol), polyoxazoline, and poly(acryloylmorpholine).

175. The method of any of embodiments 160 and 162-174, wherein the water-soluble polymer has a weight-average molecular weight in a range of from about 500 Daltons to about 100,000 Daltons.

176. The method of any of embodiments 160 and 162-175, wherein the water-soluble polymer is a poly(alkylene oxide).

177. The method of embodiment 176, wherein the poly(alkylene oxide) is a poly(ethylene glycol).

178. The method of embodiment 177, wherein the cytokine receptor agonist is a human IL-2 or IL-2 mutein covalently attached to one or more poly(ethylene glycol) polymers.

179. The method of embodiment 177, wherein the cytokine receptor agonist is a human IL-15 or IL-15 mutein covalently attached to one or more poly(ethylene glycol) polymers.

180. The method of embodiment 177, wherein the cytokine receptor agonist is a human IL-7 or IL-7 mutein covalently attached to one or more poly(ethylene glycol) polymers.

181. The method of embodiment 177, wherein the cytokine receptor agonist is a human IL-21 or IL-21 mutein covalently attached to one or more poly(ethylene glycol) polymers.

182. The method of any of embodiments 161-172, wherein the half-life extending moiety is an Fc region of an immunoglobulin, human serum albumin, an albumin binding moiety, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof.

183. The method of any of embodiments 161-172 and 182, wherein the half-life extending moiety is an albumin binding moiety.

184. The method of any of embodiment 183, wherein the cytokine receptor agonist is a fusion protein comprising a human IL-2 or IL-2 mutein fused an albumin binding moiety.

185. The method of embodiment 183, wherein the cytokine receptor agonist is a fusion protein comprising a human IL-15 or IL-15 mutein fused to an albumin binding moiety.

186. The method of embodiment 183, wherein the cytokine receptor agonist is a fusion protein comprising a human IL-7 or IL-7 mutein fused to an albumin binding moiety.

187. The method of embodiment 183, wherein the cytokine receptor agonist is a fusion protein comprising a human IL-21 or IL-21 mutein fused to an albumin binding moiety.

188. The method of any of embodiments 182-187, wherein the albumin binding moiety is a single domain antibody (sdAb) that specifically binds to albumin.

189. The method of any of embodiments 161-172 and 182, wherein the half-life extending moiety is an Fc region of an immunoglobulin.

190. The method of any of embodiment 189, wherein the cytokine receptor agonist is a fusion protein comprising a human IL-2 or IL-2 mutein fused an Fc region of an immunoglobulin.

191. The method of embodiment 189, wherein the cytokine receptor agonist is a fusion protein comprising a human IL-15 or IL-15 mutein fused to an Fc region of an immunoglobulin.

192. The method of embodiment 189, wherein the cytokine receptor agonist is a fusion protein comprising a human IL-7 or IL-7 mutein fused to an Fc region of an immunoglobulin.

193. The method of embodiment 189, wherein the cytokine receptor agonist is a fusion protein comprising a human IL-21 or IL-21 mutein fused to an Fc region of an immunoglobulin.

194. The method of any of embodiments 182 and 189-193, wherein the Fc of an immunoglobulin is an Fc of human IgG1.

195. The method of any of embodiments 182 and 189-193, wherein the Fc of an immunoglobulin is an Fc of human IgG4.

196. The method of any of embodiments 162, 163, 173-177, 180, 182, 183, 186, 189, 192, 194 and 195, wherein the T cell stimulating cytokine or mutein is an IL-7 or IL-7 mutein that is glycosylated.

197. The method of embodiment 196, wherein the T cell stimulating cytokine or mutein is hyperglycosylated, relative to wild-type human IL-7.

198. The method of embodiment 196 or embodiment 197, wherein the T cell stimulating cytokine or mutein is produced from Chinese Hamster Ovary (CHO) cells.

199. The method of any of embodiments 162, 163, 173-177, 180, 182, 183, 186, 189, 192, 194 and 195, wherein the T cell stimulating cytokine or mutein is an IL-7 conformer, wherein said conformer comprises the following three disulfide bridges: Cys: 1-4 (Cys2-Cys92); 2-5 (Cys34-Cys129) and 3-6 (Cys47-Cys141).

200. The method of any of embodiments 155-159, wherein the cytokine receptor agonist is an antibody or antigen-binding fragment that binds a T cell stimulating cytokine and the T cell stimulating cytokine is human IL-2.

201. The method of embodiment 200, wherein the antibody or antigen binding fragment inhibits binding of IL-2 with an IL-2 receptor alpha (IL-2 Rα) subunit, inhibits IL-2 signaling through IL-2 Rαβγ and through IL-2 Rβγ and/or inhibits IL-2 signaling through IL-2 Rαβγ to a greater extent than through IL-2 Rβγ.

202. The method of any of embodiments 155-159, wherein the cytokine receptor agonist is an antibody or antigen-binding fragment that binds a T cell stimulating agent and the T cell stimulating agent is human IL-21.

203. The method of embodiment 202, wherein the antibody or antigen binding fragment enhances human IL-21 activity through the IL-21 receptor.

204. The method of any of embodiments 155-203, wherein the cytokine receptor agonist is selected from the group consisting of NL-201, SAR444245 (IL-2 Synthorin™), STK-012, BPT-143, AU-007, IL-15 Synthorin™, PIO-001, bempegaldesleukin (NKTR-214), SHR-1916, ARK102, 8MW-2311, NKTR-255, Exenokine-2, MDNA-11, GX-17/NT-17, SHR-1501, ASKG-215, BCD-225, Exenokine-21, MK-1169, Hu-Miko1, JS08-1, CYT-107, AM0015 and KW-007.

205. The method of any of embodiments 155-204, wherein the cytokine receptor agonist is administered by the in-line method of administration.

206. The method of any of embodiments 155-205, wherein the transfection mixture further comprises the cytokine receptor agonist, and wherein reinfusing the transfection mixture to the subject further administers the cytokine receptor agonist to the subject by the in-line method of administration.

207. The method of embodiment 206, wherein the collected PBMCs or subset are contacted with the cytokine receptor agonist to produce the transfection mixture comprising the cytokine receptor agonist, wherein the contacting with the cytokine receptor agonist is carried out prior to the reinfusing of step (d).

208. The method of embodiment 207, wherein the contacting with the cytokine receptor agonist is carried out prior to, concurrently with or after the contacting with the composition comprising lipid particles or lentiviral vector.

209. The method of embodiment 207 or embodiment 208, wherein the contacting with the cytokine receptor agonist is performed in-line in the closed fluid circuit.

210. The method of any of embodiments 206-209, wherein the amount of the cytokine receptor agonist is from or from about 0.05 mg to 10 mg, from or from about 0.05 mg to 7.5 mg, from or from about 0.05 mg to 5 mg, from or from about 0.05 mg to 2.5 mg, from or from about 0.05 mg to 1 mg, from or from about 0.05 mg to 0.5 mg, from or from about 0.05 mg to 0.25 mg, from or from about 0.05 mg to 0.1 mg, from or from about 0.05 mg to 0.075 mg, from or from about 0.075 mg to 10 mg, from or from about 0.075 mg to 7.5 mg, from or from about 0.075 mg to 5 mg, from or from about 0.075 mg to 2.5 mg, from or from about 0.075 mg to 1 mg, from or from about 0.075 mg to 0.5 mg, from or from about 0.075 mg to 0.25 mg, from or from about 0.075 mg to 0.1 mg, from or from about 0.1 mg to 10 mg, from or from about 0.1 mg to 7.5 mg, from or from about 0.1 mg to 5 mg, from or from about 0.1 mg to 2.5 mg, from or from about 0.1 mg to 1 mg, from or from about 0.1 mg to 0.5 mg, from or from about 0.1 mg to 0.25 mg, from or from about 0.25 mg to 10 mg, from or from about 0.25 mg to 7.5 mg, from or from about 0.25 mg to 5 mg, from or from about 0.25 mg to 2.5 mg, from or from about 0.25 mg to 1 mg, from or from about 0.25 mg to 0.5 mg, from or from about 0.5 mg to 10 mg, from or from about 0.5 mg to 7.5 mg, from or from about 0.5 mg to 5 mg, from or from about 0.05 mg to 2.5 mg, from or from about 0.05 mg to 1 mg, from or from about 1 mg to 10 mg, from or from about 1 mg to 7.5 mg, from or from about 1 mg to 5 mg, from or from about 1 mg to 2.5 mg, from or from about 2.5 mg to 10 mg, from or from about 2.5 mg to 7.5 mg, from or from about 2.5 mg to 5 mg, from or from about 5 mg to 10 mg, from or from about 5 mg to 7.5 mg, or from or from about 7.5 mg to 10 mg.

211. The method of any of embodiments 1-210, wherein the volume of the transfection mixture is between 100 mL and 1000 mL, inclusive, optionally between 100 mL and 400 mL, inclusive.

212. The method of any of embodiments 205-211, further comprising:
administering one or more doses of the cytokine receptor agonist to the subject after the in-line administration of the lipid particle or lentiviral vector; and/or
administering one or more doses of the cytokine receptor agonist to the subject prior to the in-line administration of the lipid particle or lentiviral vector.

213. The method of embodiment 155-211, wherein one or more doses of the cytokine receptor agonist is administered to the subject separate from the in-line administration of the lentiviral vector.

214. The method of embodiment 212 or embodiment 213, wherein each of the one or more doses of the cytokine receptor agonist is from at or about 0.001 mg/kg to at or about 0.1 mg/kg, at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg.

215. The method of any of embodiments 212-214, wherein each of the one or more doses of the cytokine receptor agonist is from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

216. The method of any of embodiments 155-204 and 212-215, wherein the cytokine receptor agonist is administered daily, once a week (Q1W), once every two weeks (Q2W), once every three weeks (Q3W) or once every four weeks (Q4W).

217. The method of any of embodiments 155-215, wherein the cytokine receptor agonist is administered one time.

218. The method of any of embodiments 155-204 and 212-216, wherein the cytokine receptor agonist is administered for one week, two weeks, three weeks, four weeks, five weeks, six weeks, seven weeks or eight weeks.

219. The method of any of embodiments 155-204 and 212-218, wherein the cytokine receptor agonist is administered subcutaneously.

220. The method of any of embodiments 155-204 and 212-218, wherein the cytokine receptor agonist is administered intravenously.

221. The method of any of embodiments 155-204 and 212-218, wherein the cytokine receptor agonist is administered intramuscularly.

222. The method of any of embodiments 155-204 and 212-221, wherein a first dose of the cytokine receptor agonist is administered prior to the in-line administration of the lipid particle or the lentiviral vector.

223. The method of embodiment 222, wherein the first dose of the cytokine receptor agonist is administered within one month, within one week or within three days of the in-line administration of the lipid particle or the lentiviral vector.

224. The method of any of embodiments 155-204 and 212-221, wherein the first dose of the cytokine receptor agonist is administered on the same day as the in-line administration of the lipid particle or the lentiviral vector.

225. The method of any of embodiments 155-204 and 212-221, wherein the first dose of the cytokine receptor agonist is administered after the in-line administration of the lipid particle or the lentiviral vector.

226. The method of embodiment 225, wherein the first dose of the cytokine receptor agonist is administered no more than one month, no more than 21 days, no more than 14 days or no more than 7 days after the in-line administration of the lipid particle or the lentiviral vector.

227. A system for infusion of lipid particles into a subject, the system comprising:
(a) an incoming processing unit for obtaining whole blood from the circulatory system of a subject;
(b) a separation chamber for collecting peripheral blood mononuclear cells (PBMCs) or subset thereof from the blood fraction; and
(c) a contacting chamber container for transfection of the PBMCs or subset thereof with a composition comprising lipid particles to create a transfection mixture; and
(d) a transfer container for reinfusing the contacted PBMCs or subset thereof or the transfection mixture to the same subject.

228. A system for delivering a payload agent into a subject, the system comprising:
(a) an incoming processing unit for obtaining whole blood from the circulatory system of a subject;
(b) a separation chamber for collecting peripheral blood mononuclear cells (PBMCs) or subset thereof from the blood fraction; and
(c) a contacting chamber container for transfection of the PBMCs or subset thereof with a composition comprising lipid particles to create a transfection mixture; and
(d) a transfer container for reinfusing the contacted PBMCs or subset thereof or the transfection mixture to the same subject.

229. The system of embodiment 227 or embodiment 228, wherein the lipid particle is a viral vector.

230. The system of embodiment 229, wherein the viral vector is a lentiviral vector.

231. The system of any of embodiments 227-230, wherein the blood processing set, the separation chamber, the contacting chamber and/or the transfer container are operably connected via at least one connector set comprising at least one tubing line and optionally one or more connectors.

232. The system of any of embodiments 227-231, wherein the separation chamber is operably connected to a collection container that collects the PBMCs or subset.

232. The system of any of embodiments 227-231, wherein the contacting chamber and the transfer container are configured as part of the same container or are the same container.

234. The system of any of embodiments 227-231, wherein the collecting container, the contacting chamber and the transfer container are configured as part of the same container or are the same container.

235. The system of any of embodiments 227-234, wherein the container is a bag, optionally sterile bag.

236. The system of any of embodiments 227-235, wherein the system is a closed fluid circuit to operate in-line.

237. The system of embodiment 236, wherein the transfer container is configured to be separably connected from the closed fluid circuit for reinfusion.

238. The system of embodiment 236, wherein the transfer container is configured not to be disengaged from the closed fluid circuit during reinfusion to the subject.

239. The system of any of embodiments 227-235 and 238, wherein the transfer container is part of a return processing unit comprised by the system, optionally the closed fluid circuit, said return processing unit configured to reinfuse the PBMCs or subset thereof or the transfection mixture to the subject.

240. The system of any of embodiments 231-239 wherein the operable connection is via at least one connector selected from the group consisting of valves, luer ports and spikes.

241. The system of embodiment 240, wherein the connector set is disposable.

242. The system of any of embodiments 231-241, wherein the connector set is sterile.

243. The system of any of embodiments 227-242, wherein the transfer container is configured to be operably connected to the closed fluid circuit and/or donor subject, optionally via one or more tubing lines, during the reinfusion to the subject.

244. The system of any of embodiments 227-243, wherein the separation chamber is an apheresis device.

245. The system of any of embodiments 227-243, wherein the separation chamber is a leukapheresis device.

246. The system of any of embodiments 227-245, wherein the contacting chamber comprises a centrifuge.

247. The system of any of embodiments 227-246, wherein the contacting chamber is configured to be operably connected, optionally via a sterile connector set, to a container comprising a composition comprising the lipid particle.

248. The system of embodiment 247, wherein the composition comprising the lipid particles comprises a cytokine receptor agonist.

249. The system of any of embodiments 227-247, wherein the contacting container is configured to be operably connected, optionally via a sterile connector set, to a container comprising a cytokine receptor agonist.

250. The system of any of embodiments 227-247, wherein the transfer container is configured to be operably connected, optionally via a sterile connector set, to a container comprising a composition comprising a cytokine receptor agonist.

251. A combination comprising the system of any of embodiments 227-250 and a container comprising a composition comprising lipid particles.

252. The combination of embodiment 251, wherein the lipid particle is a viral vector.

253. The combination of embodiment 252, wherein the viral vector is a lentiviral vector.

254. The combination of any of embodiments 251-253, wherein the viral vector comprises a fusogen embedded in the lipid bilayer.

255. The combination of embodiment 254, wherein the fusogen is a viral fusogen selected from a Class I viral membrane fusion protein, a Class II viral membrane protein, a Class II viral membrane fusion protein, a viral membrane glycoprotein, or a viral envelope protein.

256. The combination of embodiment 254 or embodiment 255, wherein the fusogen is endogenous to the virus.

257. The combination of embodiment 254 or embodiment 255, wherein the fusogen is a pseudotyped fusogen.

258. The combination of any of embodiments 254-257, wherein the fusogen is a viral envelope protein.

259. The combination of any of embodiments 254-258, wherein the fusogen is a vesicular stomatitis virus envelope glycoprotein (VSV-G).

260. The combination of any of embodiments 254-258, wherein the fusogen is a baboon endogenous virus (BaEV) envelope glycoprotein.

261. The combination of any of embodiments 254-258, wherein the fusogen is a Cocal virus envelope glycoprotein.

262. The combination of any of embodiments 254-258, wherein the fusogen is an Alphavirus fusion protein (e.g. Sindbis virus) or a functional variant thereof.

263. The combination of any of embodiments 254-258, wherein the fusogen is a Paramyxoviridae fusion protein (e.g., a Morbillivirus or a Henipavirus) or a functional variant thereof.

264. The combination of any of embodiments 254-258 and 263, wherein the fusogen is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.

265. The combination of any of embodiments 254-258, 263 and 264, wherein the fusogen is a Henipavirus fusion protein (e.g., Nipah virus, Hendra virus, Cedar virus, Kumasi virus, Mójiāng virus) or a functional variant thereof.

266. The combination of any of embodiments 254-258 and 263-265, wherein the fusogen is a Nipah virus fusion protein or a functional variant thereof.

267. The combination of any of embodiments 254-258 and 263-266, wherein the fusogen comprises a paramyxovirus F protein, or a biologically active portion thereof.

268. The combination of any of embodiments 254-258 and 263-267, wherein the fusogen comprises a paramyxovirus G, paramyxovirus H, and/or paramyxovirus HN protein, or a biologically active portion thereof.

269. The combination of any of embodiments 251-268, wherein the lipid particle or lentiviral vector comprises a nucleic acid encoding a payload gene.

270. The combination of embodiment 269, wherein the nucleic acid encoding a payload gene encodes a chimeric antigen receptor (CAR).

271. The combination of any of embodiments 254-258 and 263-270, wherein the fusogen comprises an F protein molecule or a biologically active portion thereof from a Paramyxovirus and a glycoprotein G (G protein) or a biologically active portion thereof from a Paramyxovirus.

272. The combination of embodiment 271, wherein the Paramyxovirus is a henipavirus.

273. The combination of embodiment 271 or embodiment 272, wherein the Paramyxovirus is Nipah virus.

274. The combination of any of embodiments 254-258 and 263-273, wherein the fusogen comprises a Nipah virus F glycoprotein (NiV-F) or a biologically active portion thereof and a Nipah virus G glycoprotein (NiV-G) or a biologically active portion thereof.

275. The combination of embodiment 271 or embodiment 272, wherein the Paramyxovirus is Hendra virus.

276. The combination of any of embodiments 271-274, wherein the G protein or the biologically active portion thereof is a mutant NiV-G protein or biologically active portion thereof that exhibits reduced binding to Ephrin B2 or Ephrin B3.

277. The combination of embodiment 276, wherein the mutant NiV-G protein comprises one or more amino acid substitutions corresponding to amino acid substitutions selected from the group consisting of E501A, W504A, Q530A and E533A with reference to numbering set forth in SEQ ID NO:14.

278. The combination of any of embodiments 271-274, 276 and 277, wherein the G protein or biologically active portion is a biologically active portion of wild-type NiV-G that has a deletion of up to 40 amino acids at or near the N-terminus, optionally not including the initial methionine.

279. The combination of any of embodiments 271-274 and 276-278, wherein the G protein is a biologically active portion that is a truncated NiV-G that has a deletion of amino acids 2-34 at or near the N-terminus of wild-type NiV-G set forth in SEQ ID NO:14.

280. The combination of any of embodiments 271-274 and 276-279, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

281. The combination of any of embodiments 271-274 and 276-280, wherein the F protein or the biologically active portion thereof is a NiV-F protein or a biologically active portion thereof.

282. The combination of embodiment 281, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

283. The combination of any of embodiments 271-274 and 276-282, wherein the F protein or the biologically active portion is a truncated NiV-F that lacks amino acids 525-546 of SEQ ID NO:2.

284. The combination of any of embodiments 271-274 and 276-283, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

285. The combination of any of embodiments 271-274 and 276-284, wherein the Niv-G protein comprises the amino acid sequence set forth in SEQ ID NO: 19, and the Niv-F protein comprises the amino acid sequence set forth in SEQ ID NO:12.

286. The combination of any of embodiments 254-285, wherein the fusogen is a re-targeted fusogen that binds to a target cell.

287. The combination of embodiment 286, wherein the fusogen comprises a targeting moiety that binds to the target cell.

288. The combination of embodiment 287, wherein the paramyxovirus G, paramyxovirus H, and/or paramyxovirus HN protein further comprises a targeting moiety.

289. The combination of embodiment 287, wherein the targeting moiety comprises a binding agent.

290. The combination of embodiment 289, wherein the binding agent is targeted against CD3, CD4, CD8, CD34, CD90, CD19, CD20, CD22, CD16, or CD56.

291. The combination of any of embodiments 286-290, wherein the target cell is a T cell, a B cell, an NK cell, a macrophage, a monocyte, a dendritic cell, a hematopoietic stem cell or a CD34+progenitor cell.

292. The combination of any of embodiments 286-291, wherein the target cell is a T cell.

293. The combination of any of embodiments 287-292, wherein the targeting moiety binds to CD4 or CD8.

294. The combination of any of embodiments 287-293, wherein the targeting moiety is a Design ankyrin repeat proteins (DARPin), a single domain antibody (sdAb), a single chain variable fragment (scFv), or an antigen-binding fibronectin type III (Fn3) scaffold.

295. The combination of any of embodiments 287-294, wherein the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker.

296. The combination of any of embodiments 287-294, wherein the CD8 binding agent is a VHH having the sequence set forth in SEQ ID NO: 128.

297. The combination of embodiment 295 or embodiment 296, wherein the CD8 binding agent is linked to the C-terminus of a truncated NiV-G set forth in SEQ ID NO: 19 for retargeting of the lipid particle or lentiviral vector to CD8+ T cells.

298. The combination of embodiment 297, wherein the lipid particle or lentiviral vector comprising the retargeted NiV-G is pseudotyped with the a NiV-F of a biologically active portion thereof, optionally wherein the NiV-F or biologically active portion is set forth in SEQ ID NO:12.

299. The combination of any of embodiments 251-298, wherein the lipid particle is a viral vector and the composition comprises from 1×10⁸ to 1×10¹¹ infectious units (IU), 1×10⁸ to 1×10¹⁰ IU, 1×108 to 1×109 IU, 1×109 to 1×10¹¹ IU, 1×109 to 1×10¹⁰ IU, 1×10¹⁰ to 1×10¹¹ IU.

300. The combination of any of embodiments 251-299, wherein the volume of the composition comprising lipid particles is between 100 mL and 1000 mL, inclusive, optionally between 100 mL and 400 mL, inclusive.

301. The combination of any of embodiments 251-300, wherein the composition comprising lipid particles further comprises a cytokine receptor agonist.

302. The combination of any of embodiments 251-300, wherein the combination further comprises a container comprising a composition comprising a cytokine receptor agonist.

303. The combination of embodiment 301 or embodiment 302, wherein the cytokine receptor agonist is a recombinant protein, a chemically synthesized protein or a conjugate.

304. The combination of any of embodiments 301-303, wherein the cytokine receptor agonist binds to a cytokine receptor on a T cell.

305. The combination of embodiment 304, wherein the cytokine receptor is selected from the group consisting of an IL-2 receptor (IL-2R), an IL-15 receptor (IL-15R), an IL-7 receptor (IL-7R), or an IL-21 receptor (IL-21R).

306. The combination of any of embodiments 301-305, wherein the cytokine receptor agonist comprises a T cell stimulating cytokine or a T cell stimulating cytokine mutein, a T cell stimulating cytokine mimetic, an antibody or antigen-binding fragment that binds a cytokine receptor on a T cell, or an antibody or antigen-binding fragment that binds a T cell stimulating cytokine.

307. The combination of any of embodiments 301-306, wherein the cytokine receptor agonist is a conjugate comprising (1) a T cell stimulating cytokine, a T cell stimulating cytokine mutein or a T cell stimulating cytokine mimetic and (2) a water soluble polymer.

308. The combination of any of embodiments 301-306, wherein the cytokine receptor agonist is a fusion protein comprising (1) a T cell stimulating cytokine, a T cell stimulating cytokine mutein or a T cell stimulating cytokine mimetic and (2) a half-life extending moiety.

309. The combination of any of embodiments 306-308, wherein the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21), or a T cell stimulating cytokine mutein of any of the foregoing.

310. The combination of any of embodiments 306-309, wherein the T cell stimulating cytokine mutein comprises at least one amino acid modification relative to a wild-type T cell stimulating cytokine.

311. The combination of any of embodiments 306-310, wherein the T cell stimulating cytokine mutein is an IL-2 cytokine mutein comprising one or more amino acid modifications relative to wild-type human IL-2.

312. The combination of embodiment 311, wherein the IL-2 mutein exhibits increased affinity for the IL-2 Rβ, relative to wild-type human IL-2.

313. The combination of embodiment 311 or embodiment 312, wherein the IL-2 mutein exhibits increased IL-2 activity for the intermediate affinity IL-2 receptor composed of IL-2Rbeta and IL-2Rgamma (IL-2R β/γ), relative to wild-type human IL-2.

314. The combination any of embodiments 311-313, wherein the IL-2 mutein exhibits reduced binding to IL-2Ralpha, relative to wild-type human IL-2.

315. The combination of any of embodiments 312-314, wherein the IL-2 mutein exhibits reduced IL-2 activity for the high-affinity IL-2 receptor composed of IL-2Ralpha, IL-2Rbeta and IL-2Rgamma (IL-2R α/β/γ).

316. The combination of any of embodiments 306-310, wherein the T cell stimulating cytokine or cytokine mutein is IL-15 or an IL-15 cytokine mutein and the IL-15 or IL-15 cytokine mutein is bound to IL-15Rα or a portion thereof comprising the sushi domain.

317. The combination of any of embodiments 306-310 and 316, wherein the T cell stimulating cytokine mutein is a IL-15 cytokine mutein comprising one more amino acid modifications relative to human IL-15.

318. The combination of embodiment 316 or embodiment 317, wherein the IL-15 mutein exhibits reduced binding to IL-15Rα.

319. The combination of any of embodiments 306-308, wherein the cytokine receptor agonist comprises a T cell stimulating cytokine mimetic and the mimetic is a IL-2Rα ligand, a IL-2RP ligand, a IL-2Rγ ligand, a common γc receptor (Rγc) ligand, and/or IL-7Rα ligand.

320. The combination of any of embodiments 307 and 309-319, wherein one, two, three, four, five or six water-soluble polymers are attached to the T cell stimulating cytokine.

321. The combination of any of embodiments 307 and 309-320, wherein the water soluble polymer is a polymer selected from the group consisting of poly(alkylene oxide), poly(vinyl pyrrolidone), poly(vinyl alcohol), polyoxazoline, and poly(acryloylmorpholine).

322. The combination of any of embodiments 307 and 309-321, wherein the water-soluble polymer has a weight-average molecular weight in a range of from about 500 Daltons to about 100,000 Daltons.

323. The combination of any of embodiments 307 and 309-322, wherein the water-soluble polymer is a poly(alkylene oxide).

324. The combination of embodiment 323, wherein the poly(alkylene oxide) is a poly(ethylene glycol).

325. The combination of embodiment 324, wherein the cytokine receptor agonist is a human IL-2 or IL-2 mutein covalently attached to one or more poly(ethylene glycol) polymers.

326. The combination of embodiment 324, wherein the cytokine receptor agonist is a human IL-15 or IL-15 mutein covalently attached to one or more poly(ethylene glycol) polymers.

327. The combination of embodiment 324, wherein the cytokine receptor agonist is a human IL-7 or IL-7 mutein covalently attached to one or more poly(ethylene glycol) polymers.

328. The combination of embodiment 324, wherein the cytokine receptor agonist is a human IL-21 or IL-21 mutein covalently attached to one or more poly(ethylene glycol) polymers.

329. The combination of any of embodiments 308-319, wherein the half-life extending moiety is an Fc region of an immunoglobulin, human serum albumin, an albumin binding moiety, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof.

330. The combination of any of embodiments 309-319 and 329, wherein the half-life extending moiety is an albumin binding moiety.

331. The combination of any of embodiment 330, wherein the cytokine receptor agonist is a fusion protein comprising a human IL-2 or IL-2 mutein fused an albumin binding moiety.

332. The combination of embodiment 330, wherein the cytokine receptor agonist is a fusion protein comprising a human IL-15 or IL-15 mutein fused to an albumin binding moiety.

333. The combination of embodiment 330, wherein the cytokine receptor agonist is a fusion protein comprising a human IL-7 or IL-7 mutein fused to an albumin binding moiety.

334. The combination of embodiment 330, wherein the cytokine receptor agonist is a fusion protein comprising a human IL-21 or IL-21 mutein fused to an albumin binding moiety.

335. The combination of any of embodiments 330-334, wherein the albumin binding moiety is a single domain antibody (sdAb) that specifically binds to albumin.

336. The combination of any of embodiments 309-319 and 329, wherein the half-life extending moiety is an Fc region of an immunoglobulin.

337. The combination of any of embodiment 336, wherein the cytokine receptor agonist is a fusion protein comprising a human IL-2 or IL-2 mutein fused an Fc region of an immunoglobulin.

338. The combination of embodiment 336, wherein the cytokine receptor agonist is a fusion protein comprising a human IL-15 or IL-15 mutein fused to an Fc region of an immunoglobulin.

339. The combination of embodiment 336, wherein the cytokine receptor agonist is a fusion protein comprising a human IL-7 or IL-7 mutein fused to an Fc region of an immunoglobulin.

340. The combination of embodiment 336, wherein the cytokine receptor agonist is a fusion protein comprising a human IL-21 or IL-21 mutein fused to an Fc region of an immunoglobulin.

341. The combination of any of embodiments 329 and 337-340, wherein the Fc of an immunoglobulin is an Fc of human IgG1.

342. The combination of any of embodiments 329 and 337-340, wherein the Fc of an immunoglobulin is an Fc of human IgG4.

343. The combination of any of embodiments 309, 310, 320-324, 327, 329, 330, 333, 336, 339, 341 and 342, wherein the T cell stimulating cytokine or mutein is an IL-7 or IL-7 mutein that is glycosylated.

344. The combination of embodiment 343, wherein the T cell stimulating cytokine or mutein is hyperglycosylated, relative to wild-type human IL-7. 345. The combination of embodiment 343 or embodiment 344, wherein the T cell stimulating cytokine or mutein is produced from Chinese Hamster Ovary (CHO) cells.

346. The combination of any of embodiments 309, 310, 320-324, 327, 329, 330, 333, 336, 339, 341 and 342, wherein the T cell stimulating cytokine or mutein is an IL-7 conformer, wherein said conformer comprises the following three disulfide bridges: Cys: 1-4 (Cys2-Cys92); 2-5 (Cys34-Cys129) and 3-6 (Cys47-Cys141).

347. The combination of any of embodiments 301-306, wherein the cytokine receptor agonist is an antibody or antigen-binding fragment that binds a T cell stimulating cytokine and the T cell stimulating cytokine is human IL-2.

348. The combination of embodiment 347, wherein the antibody or antigen binding fragment inhibits binding of IL-2 with an IL-2 receptor alpha (IL-2 Rα) subunit, inhibits IL-2 signaling through IL-2 Rαβγ and through IL-2 Rβγ and/or inhibits IL-2 signaling through IL-2 Rαβγto a greater extent than through IL-2 Rβγ.

349. The combination of any of embodiments 301-306, wherein the cytokine receptor agonist is an antibody or antigen-binding fragment that binds a T cell stimulating agent and the T cell stimulating agent is human IL-21.

350. The combination of embodiment 349, wherein the antibody or antigen binding fragment enhances human IL-21 activity through the IL-21 receptor.

351. The combination of any of embodiments 301-350, wherein the cytokine receptor agonist is selected from the group consisting of NL-201, SAR444245 (IL-2 Synthorin™), STK-012, BPT-143, AU-007, IL-15 Synthorin™, PIO-001, bempegaldesleukin (NKTR-214), SHR-1916, ARK102, 8MW-2311, NKTR-255, Exenokine-2, MDNA-11, GX-I7/NT-I7, SHR-1501, ASKG-215, BCD-225, Exenokine-21, MK-1169, Hu-Mikβ1, JS08-1, CYT-107, AM0015 and KW-007.

352. A sterile composition comprising between 1×10⁶ cells/mL to 1×108 cells/mL of peripheral blood mononuclear cells (PBMCs) or subset thereof and viral vector composition consisting of 1×108 to 1×10¹¹ infectious units (IU).

353. The sterile composition of embodiment 352, wherein the composition has a volume of between 100 mL to 1000 mL.

354. A sterile composition comprising peripheral blood mononuclear cells (PBMCs) from a 100 mL to 400 mL leukapheresis product and a viral vector composition comprising 1×108 to 1×10” infectious units (IU).

355. The sterile composition of embodiment 354, wherein the volume of the composition is between 100 mL to 1000 mL.

356. The sterile composition of any of embodiment 352-355, wherein the viability of the PBMCs or subset thereof is greater than 95%, e.g. between 95% and 100%, between 96% and 100%, between 97% and 100%, between 98% and 100% or between 99% and 100%,

357. The sterile composition of any of embodiment 352-356, wherein the viability of cells of the PBMCs or subset thereof is greater than 95%, e.g. between 95% and 100%, between 96% and 100%, between 97% and 100%, between 98% and 100% or between 99% and 100%,

358. The sterile composition of any of embodiments 352-357, wherein the virial vector is a lentiviral vector.

359. The sterile composition of any of embodiments 352-358, wherein the viral vector comprises a fusogen embedded in the lipid bilayer.

360. The sterile composition of embodiment 359, wherein the fusogen is a viral fusogen selected from a Class I viral membrane fusion protein, a Class II viral membrane protein, a Class II viral membrane fusion protein, a viral membrane glycoprotein, or a viral envelope protein.

361. The sterile composition of embodiment 359 or embodiment 360, wherein the fusogen is endogenous to the virus.

362. The sterile composition of embodiment 359 or embodiment 360, wherein the fusogen is a pseudotyped fusogen.

363. The sterile composition of any of embodiments 359-362, wherein the fusogen is a viral envelope protein.

364. The sterile composition of any of embodiments 359-362, wherein the fusogen is a vesicular stomatitis virus envelope glycoprotein (VSV-G).

365. The sterile composition of any of embodiments 359-362, wherein the fusogen is a baboon endogenous virus (BaEV) envelope glycoprotein.

366. The sterile composition of any of embodiments 359-362, wherein the fusogen is a Cocal virus envelope glycoprotein.

367. The sterile composition of any of embodiments 359-362, wherein the fusogen is an Alphavirus fusion protein (e.g. Sindbis virus) or a functional variant thereof.

368. The sterile composition of any of embodiments 359-362, wherein the fusogen is a Paramyxoviridae fusion protein (e.g., a Morbillivirus or a Henipavirus) or a functional variant thereof.

369. The sterile composition of any of embodiments 359-362 and 368, wherein the fusogen is a Morbillivirus fusion protein (e.g., measles virus (MeV), canine distemper virus, Cetacean morbillivirus, Peste-des-petits-ruminants virus, Phocine distemper virus, Rinderpest virus) or a functional variant thereof.

370. The sterile composition of any of embodiments 359-362, 368 and 369, wherein the fusogen is a Henipavirus fusion protein (e.g., Nipah virus, Hendra virus, Cedar virus, Kumasi virus, Mójiāng virus) or a functional variant thereof.

371. The sterile composition of any of embodiments 359-362 and 368-370, wherein the fusogen is a Nipah virus fusion protein or a functional variant thereof.

372. The sterile composition of any of embodiments 359-362 and 368-370, wherein the fusogen comprises a paramyxovirus F protein, or a biologically active portion thereof.

373. The sterile composition of any of embodiments 359-362 and 368-372, wherein the fusogen comprises a paramyxovirus G, paramyxovirus H, and/or paramyxovirus HN protein, or a biologically active portion thereof.

374. The sterile composition of embodiment 373, wherein the paramyxovirus G, paramyxovirus H, and/or paramyxovirus HN protein further comprises a targeting moiety.

375. The sterile composition of any of embodiments 352-374, wherein the viral vector comprises a nucleic acid encoding a payload gene.

376. The sterile composition of embodiment 375, wherein the nucleic acid encoding a payload gene encodes a chimeric antigen receptor (CAR).

377. The sterile composition of any of embodiments 374-376, wherein the targeting moiety comprises a binding agent.

378. The sterile composition of embodiment 377, wherein the binding agent is targeted against CD3, CD4, CD8, CD34, CD90, CD19, CD20, CD22, CD16, or CD56. 379. The sterile composition of any of embodiments 359-362 and 368-378, wherein the fusogen comprises an F protein molecule or a biologically active portion thereof from a Paramyxovirus and a glycoprotein G (G protein) or a biologically active portion thereof from a Paramyxovirus.

380. The sterile composition of embodiment 379, wherein the Paramyxovirus is a henipavirus.

381. The sterile composition of embodiment 379 or embodiment 380, wherein the Paramyxovirus is Nipah virus.

382. The sterile composition of any of embodiments 359-362 and 368-381, wherein the fusogen comprises a Nipah virus F glycoprotein (NiV-F) or a biologically active portion thereof and a Nipah virus G glycoprotein (NiV-G) or a biologically active portion thereof.

383. The sterile composition of embodiment 379 or embodiment 380, wherein the Paramyxovirus is Hendra virus.

384. The sterile composition of any of embodiments 379-382, wherein the G protein or the biologically active portion thereof is a mutant NiV-G protein or biologically active portion thereof that exhibits reduced binding to Ephrin B2 or Ephrin B3. 385. The sterile composition of embodiment 384, wherein the mutant NiV-G protein comprises one or more amino acid substitutions corresponding to amino acid substitutions selected from the group consisting of E501A, W504A, Q530A and E533A with reference to numbering set forth in SEQ ID NO:14. 386. The sterile composition of any of embodiments 379-382, 384 and 385, wherein the G protein or biologically active portion is a biologically active portion of wild-type NiV-G that has a deletion of up to 40 amino acids at or near the N-terminus, optionally not including the initial methionine.

387. The sterile composition of any of embodiments 379-382 and 384-386, wherein the G protein is a biologically active portion that is a truncated NiV-G that has a deletion of amino acids 2-34 at or near the N-terminus of wild-type NiV-G set forth in SEQ ID NO:14.

388. The sterile composition of any of embodiments 379-382 and 384-387, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

389. The sterile composition of any of embodiments 379-382 and 384-388, wherein the F protein or the biologically active portion thereof is a NiV-F protein or a biologically active portion thereof.

390. The sterile composition of embodiment 389, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

391. The sterile composition of any of embodiments 379-382 and 384-390, wherein the F protein or the biologically active portion is a truncated NiV-F that lacks amino acids 525-546 of SEQ ID NO:2.

392. The sterile composition of any of embodiments 379-382 and 384-391, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

393. The sterile composition of any of embodiments 379-382 and 384-392, wherein the Niv-G protein comprises the amino acid sequence set forth in SEQ ID NO: 19, and the Niv-F protein comprises the amino acid sequence set forth in SEQ ID NO:12.

394. The sterile composition of any of embodiments 359-393, wherein the fusogen is a re-targeted fusogen that binds to a target cell.

395. The sterile composition of embodiment 394, wherein the fusogen comprises a targeting moiety that binds to the target cell.

396. The sterile composition of embodiment 394 or embodiment 395, wherein the target cell is a T cell, a B cell, an NK cell, a macrophage, a monocyte, a dendritic cell, a hematopoietic stem cell or a CD34+progenitor cell.

397. The sterile composition of any of embodiments 394-396, wherein the target cell is a T cell.

398. The sterile composition of any of embodiments 394-397, wherein the targeting moiety binds to CD4 or CD8. 399. The sterile composition of any of embodiments 394-397, wherein the targeting moiety is a Design ankyrin repeat proteins (DARPin), a single domain antibody (sdAb), a single chain variable fragment (scFv), or an antigen-binding fibronectin type III (Fn3) scaffold.

400. The sterile composition of any of embodiments 394-399, wherein the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker.

401. The sterile composition of any of embodiments 394-399, wherein the CD8 binding agent is a VHH having the sequence set forth in SEQ ID NO: 128.

402. The sterile composition of embodiment 400 or embodiment 401, wherein the CD8 binding agent is linked to the C-terminus of a truncated NiV-G set forth in SEQ ID NO: 19 for retargeting of the lipid particle or lentiviral vector to CD8+ T cells.

403. The sterile composition of embodiment 402, wherein the viral vector comprising the retargeted NiV-G is pseudotyped with the a NiV-F of a biologically active portion thereof, optionally wherein the NiV-F or biologically active portion is set forth in SEQ ID NO:12.

404. The sterile composition of any of embodiments 352-403, wherein the composition comprises from 1×10⁸ to 1×10¹¹ infectious units (IU), 1×10⁸ to 1×10¹⁰ IU, 1×10⁸ to 1×10⁹ IU, 1×10⁹ to 1×10¹¹ IU, 1×10⁹ to 1×10¹⁰ IU, 1×10¹⁰ to 1×10¹¹ IU.

405. The sterile composition of any of embodiments 352-404, further comprising a cytokine receptor agonist.

406. The sterile composition of embodiment 405, wherein the cytokine receptor agonist is a recombinant protein, a chemically synthesized protein or a conjugate.

407. The sterile composition of embodiment 405 or embodiment 406, wherein the cytokine receptor agonist binds to a cytokine receptor on a T cell.

408. The sterile composition of embodiment 407, wherein the cytokine receptor is selected from the group consisting of an IL-2 receptor (IL-2R), an IL-15 receptor (IL-15R), an IL-7 receptor (IL-7R), or an IL-21 receptor (IL-21R).

409. The sterile composition of any of embodiments 405-408, wherein the cytokine receptor agonist comprises a T cell stimulating cytokine or a T cell stimulating cytokine mutein, a T cell stimulating cytokine mimetic, an antibody or antigen-binding fragment that binds a cytokine receptor on a T cell, or an antibody or antigen-binding fragment that binds a T cell stimulating cytokine.

410. The sterile composition of any of embodiments 405-409, wherein the cytokine receptor agonist is a conjugate comprising (1) a T cell stimulating cytokine, a T cell stimulating cytokine mutein or a T cell stimulating cytokine mimetic and (2) a water soluble polymer.

411. The sterile composition of any of embodiments 405-490, wherein the cytokine receptor agonist is a fusion protein comprising (1) a T cell stimulating cytokine, a T cell stimulating cytokine mutein or a T cell stimulating cytokine mimetic and (2) a half-life extending moiety.

412. The sterile composition of any of embodiments 409-411, wherein the T cell stimulating cytokine or a T cell stimulating cytokine mutein is selected from the group consisting of interleukin-2 (IL-2), an interleukin-15 (IL-15), an interleukin-7 (IL-7), an interleukin-21 (IL-21), or a T cell stimulating cytokine mutein of any of the foregoing.

413. The sterile composition of any of embodiments 409-412, wherein the T cell stimulating cytokine mutein comprises at least one amino acid modification relative to a wild-type T cell stimulating cytokine.

414. The sterile composition of any of embodiments 409-413, wherein the T cell stimulating cytokine mutein is an IL-2 cytokine mutein comprising one or more amino acid modifications relative to wild-type human IL-2.

415. The sterile composition of embodiment 414, wherein the IL-2 mutein exhibits increased affinity for the IL-2 Rβ, relative to wild-type human IL-2.

416. The sterile composition of embodiment 414 or embodiment 415, wherein the IL-2 mutein exhibits increased IL-2 activity for the intermediate affinity IL-2 receptor composed of IL-2Rbeta and IL-2Rgamma (IL-2R β/γ), relative to wild-type human IL-2.

417. The sterile composition any of any of embodiments 414-416, wherein the IL-2 mutein exhibits reduced binding to IL-2Ralpha, relative to wild-type human IL-2.

418. The sterile composition of any of embodiments 414-417, wherein the IL-2 mutein exhibits reduced IL-2 activity for the high-affinity IL-2 receptor composed of IL-2Ralpha, IL-2Rbeta and IL-2Rgamma (IL-2R α/β/γ).

419. The sterile composition of any of embodiments 409-413, wherein the T cell stimulating cytokine or cytokine mutein is IL-15 or an IL-15 cytokine mutein and the IL-15 or IL-15 cytokine mutein is bound to IL-15Rα or a portion thereof comprising the sushi domain.

420. The sterile composition of any of embodiments 409-413 and 419, wherein the T cell stimulating cytokine mutein is a IL-15 cytokine mutein comprising one more amino acid modifications relative to human IL-15.

421. The sterile composition of embodiment 419 or embodiment 420, wherein the IL-15 mutein exhibits reduced binding to IL-15Rα.

422. The sterile composition of any of embodiments 409-413, wherein the cytokine receptor agonist comprises a T cell stimulating cytokine mimetic and the mimetic is a IL-2Rα ligand, a IL-2RP ligand, a IL-2Rγ ligand, a common γc receptor (Rγc) ligand, and/or IL-7Rα ligand.

423. The sterile composition of any of embodiments 410 and 412-422, wherein one, two, three, four, five or six water-soluble polymers are attached to the T cell stimulating cytokine.

424. The sterile composition of any of embodiments 410 and 412-423, wherein the water soluble polymer is a polymer selected from the group consisting of poly(alkylene oxide), poly(vinyl pyrrolidone), poly(vinyl alcohol), polyoxazoline, and poly(acryloylmorpholine).

425. The sterile composition of any of embodiments 410 and 412-424, wherein the water-soluble polymer has a weight-average molecular weight in a range of from about 500 Daltons to about 100,000 Daltons.

426. The sterile composition of any of embodiments 410 and 412-425, wherein the water-soluble polymer is a poly(alkylene oxide).

427. The sterile composition of embodiment 426, wherein the poly(alkylene oxide) is a poly(ethylene glycol).

428. The sterile composition of embodiment 427, wherein the cytokine receptor agonist is a human IL-2 or IL-2 mutein covalently attached to one or more poly(ethylene glycol) polymers.

429. The sterile composition of embodiment 427 wherein the cytokine receptor agonist is a human IL-15 or IL-15 mutein covalently attached to one or more poly(ethylene glycol) polymers.

430. The sterile composition of embodiment 427, wherein the cytokine receptor agonist is a human IL-7 or IL-7 mutein covalently attached to one or more poly(ethylene glycol) polymers.

431. The sterile composition of embodiment 427, wherein the cytokine receptor agonist is a human IL-21 or IL-21 mutein covalently attached to one or more poly(ethylene glycol) polymers.

432. The sterile composition of any of embodiments 411-422, wherein the half-life extending moiety is an Fc region of an immunoglobulin, human serum albumin, an albumin binding moiety, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a sterile composition thereof.

433. The sterile composition of any of embodiments 411-422 and 432, wherein the half-life extending moiety is an albumin binding moiety.

434. The sterile composition of any of embodiment 433, wherein the cytokine receptor agonist is a fusion protein comprising a human IL-2 or IL-2 mutein fused an albumin binding moiety.

435. The sterile composition of embodiment 434, wherein the cytokine receptor agonist is a fusion protein comprising a human IL-15 or IL-15 mutein fused to an albumin binding moiety.

436. The sterile composition of embodiment 434, wherein the cytokine receptor agonist is a fusion protein comprising a human IL-7 or IL-7 mutein fused to an albumin binding moiety.

437. The sterile composition of embodiment 434, wherein the cytokine receptor agonist is a fusion protein comprising a human IL-21 or IL-21 mutein fused to an albumin binding moiety.

438. The sterile composition of any of embodiments 432-437, wherein the albumin binding moiety is a single domain antibody (sdAb) that specifically binds to albumin.

439. The sterile composition of any of embodiments 411-422 and 432, wherein the half-life extending moiety is an Fc region of an immunoglobulin.

440. The sterile composition of any of embodiment 439, wherein the cytokine receptor agonist is a fusion protein comprising a human IL-2 or IL-2 mutein fused an Fc region of an immunoglobulin.

441. The sterile composition of embodiment 439, wherein the cytokine receptor agonist is a fusion protein comprising a human IL-15 or IL-15 mutein fused to an Fc region of an immunoglobulin.

442. The sterile composition of embodiment 439, wherein the cytokine receptor agonist is a fusion protein comprising a human IL-7 or IL-7 mutein fused to an Fc region of an immunoglobulin.

443. The sterile composition of embodiment 439, wherein the cytokine receptor agonist is a fusion protein comprising a human IL-21 or IL-21 mutein fused to an Fc region of an immunoglobulin.

444. The sterile composition of any of embodiments 432 and 439-443, wherein the Fc of an immunoglobulin is an Fc of human IgG1.

445. The sterile composition of any of embodiments 432 and 439-449, wherein the Fc of an immunoglobulin is an Fc of human IgG4.

446. The sterile composition of any of embodiments 412, 413, 423-427, 430, 432, 433, 436, 439, 442, 444 and 445, wherein the T cell stimulating cytokine or mutein is an IL-7 or IL-7 mutein that is glycosylated.

447. The sterile composition of embodiment 446, wherein the T cell stimulating cytokine or mutein is hyperglycosylated, relative to wild-type human IL-7.

448. The sterile composition of embodiment 446 or embodiment 447, wherein the T cell stimulating cytokine or mutein is produced from Chinese Hamster Ovary (CHO) cells.

449. The sterile composition of any of embodiments 412, 413, 423-427, 430, 432, 433, 436, 439, 442, 444 and 445, wherein the T cell stimulating cytokine or mutein is an IL-7 conformer, wherein said conformer comprises the following three disulfide bridges: Cys: 1-4 (Cys2-Cys92); 2-5 (Cys34-Cys129) and 3-6 (Cys47-Cys141).

450. The sterile composition of any of embodiments 405-409, wherein the cytokine receptor agonist is an antibody or antigen-binding fragment that binds a T cell stimulating cytokine and the T cell stimulating cytokine is human IL-2.

451. The sterile composition of embodiment 450, wherein the antibody or antigen binding fragment inhibits binding of IL-2 with an IL-2 receptor alpha (IL-2 Rα) subunit, inhibits IL-2 signaling through IL-2 Rαβγ and through IL-2 Rβγ and/or inhibits IL-2 signaling through IL-2 Rαβγto a greater extent than through IL-2 Rβγ.

452. The sterile composition of any of embodiments 405-409, wherein the cytokine receptor agonist is an antibody or antigen-binding fragment that binds a T cell stimulating agent and the T cell stimulating agent is human IL-21.

453. The sterile composition of embodiment 452, wherein the antibody or antigen binding fragment enhances human IL-21 activity through the IL-21 receptor.

454. The sterile composition of any of embodiments 405-453, wherein the cytokine receptor agonist is selected from the group consisting of NL-201, SAR444245 (IL-2 Synthorin™), STK-012, BPT-143, AU-007, IL-15 Synthorin™, PIO-001, bempegaldesleukin (NKTR-214), SHR-1916, ARK102, 8MW-2311, NKTR-255, Exenokine-2, MDNA-11, GX-17/NT-17, SHR-1501, ASKG-215, BCD-225, Exenokine-21, MK-1169, Hu-Mikβ1, JS08-1, CYT-107, AM0015 and KW-007.

455. The sterile composition of any of embodiments 405-454, wherein the composition comprises an anticoagulant.

456. The sterile composition of embodiment 455, wherein the anticoagulant is a citrate.

457. A container comprising the sterile composition of any of embodiments 405-456.

458. The container of embodiment 457, wherein the container is a bag.

459. A method of treating a disease or condition in a subject comprising administering a lipid particle or payload gene by the method of any of embodiments 1-8 and 18-128 and 137-226 to a subject in need thereof.

460. A method of treating a disease or condition comprising infusing the composition of any of embodiments 405-456 into a subject in need thereof.

461. A method of treating a disease or condition comprising administering the sterile composition of any of embodiments 405-456 to a subject having a disease or condition in need of treatment thereof.

462. The method of embodiment 460 or embodiment 460, wherein the infusion or administration is by in-line infusion of the composition to the subject.

463. The method of embodiment 462, wherein the in-line infusion comprises an apheresis device.

464. The method of any of embodiments 459-463, wherein the disease or disorder is treatable by administration of the lipid particle or the payload agent.

465. The 1 method of embodiment 464, wherein the payload agent is a CAR.

466. A method of treating a disease or condition comprising infusing the lentiviral vector encoding the chimeric antigen receptor (CAR) by the method of any of embodiments 9-133 and 137-226 to a subject having a disease or condition in need of treatment thereof.

467. The method of embodiment 465 or embodiment 466, wherein the CAR comprises an extracellular antigen binding domain specific for an antigen associated with the disease or condition.

468. The method of any of embodiments 465-467, wherein the CAR is an anti-CD19 CAR, an anti-CD22 CAR, an anti-CD20 CAR or an anti-BCMA CAR.

469. The method of any of embodiments 465-468, wherein the CAR is an anti-CD19 CAR.

470. The method of any of embodiments 459-469, wherein the disease or condition is a cancer.

471. The method of any of embodiments 134-226 and 459-469, wherein the cancer is a solid tumor, a lymphoma or a leukemia.

472. The method of any of embodiments 134-226 and 459-469, wherein the cancer is a B cell Lymphoma.

473. The method of embodiment 472, wherein the B cell lymphoma is a Non-Hodgkin lymphoma (NHL), DLBCL, or follicular lymphoma.

474. The method of any of embodiments 135-226 and 459-473, wherein the cancer is a relapsed/refractory cancer.

475. The method of any of embodiments 135-226 and 459-474, wherein the cancer is a relapsed and/or refractory Large B-cell Lymphoma (LBCL).

476. The method of embodiment 475, wherein the LBCL comprises Non-Hodgkin's lymphoma (NHL).

477. The method of any of embodiments 135-226 and 459-476, wherein the subject has received 2 prior lines of systemic therapy for treating the cancer.

478. The method of any of embodiments 135-226 and 459-477, wherein the subject has received 2 prior chemotherapy regimens comprising 1 or more regimens comprising anthracycline and/or one or more regimens comprising anti-CD20 antibody.

479. The method of any of embodiments 135-226 and 459-478, wherein the subject has received an autologous stem cell transplant (ASCT).

480. The method of any of embodiments 473-479, wherein the NHL comprises a lymphoma selected from the group consisting of diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma.

481. The method of any of embodiments 1-226 and 459-480, wherein the subject has not received a lymphodepleting regimen or therapy.

482. The method of any of embodiments 1-226 and 459-481, wherein the subject has not received a lymphodepleting regimen or therapy within 30 days prior to the use or administration.

483. The method of any of embodiments 1-226 and 459-482, prior to performing the therapy or method, further comprising administering to the subject one or more agents to mobilize peripheral blood hematopoietic stem cells or CD34+progenitor cells, optionally wherein the agent is G-CSF.

484. A lipid particle therapy for use in treating a subject having a disease or condition in need of treatment, wherein the lipid particle therapy particle therapy is administered to the subject by the method of any of embodiments 1-8 and 18-128 and 137-226.

485. A lipid particle therapy for use in treating a subject having a disease or condition in need of treatment, wherein the lipid particle therapy comprises a leukapheresis cell composition contacted with a composition comprising lipid particles, and wherein the lipid particle therapy is administered to the subject via an apheresis device.

486. A lentiviral vector therapy for use in treating a subject having a disease or condition in need of treatment, wherein the lentiviral vector therapy is for administration to the subject by the method of any of embodiments 8-84 and 87-226.

487. The sterile composition of any of embodiments 405-456 for use in treating a subject having a disease or condition in need of treatment by in-line infusion of the composition to a subject.

488. The sterile composition of embodiment 487, wherein the in-line infusion comprises an apheresis device.

489. The lipid particle therapy for use of embodiment 487 or embodiment 485, the lentiviral vector therapy for use of embodiment 486 or the sterile composition for use of embodiment 487 or embodiment 488, wherein the disease or condition is a cancer.

490. The lipid particle therapy for use, the lentiviral vector therapy for use or the sterile composition for use of embodiment 489, wherein the cancer is a solid tumor, a lymphoma or a leukemia.

491. The lipid particle therapy for use, the lentiviral vector therapy for use or the sterile composition for use of embodiment 489 or embodiment 490, wherein the cancer is a B cell Lymphoma.

492. The lipid particle therapy for use, the lentiviral vector therapy for use or the sterile composition for use of embodiment 491, wherein the B cell lymphoma is a Non-Hodgkin lymphoma (NHL), DLBCL, or follicular lymphoma.

493. The lipid particle therapy for use, the lentiviral vector therapy for use or the sterile composition for use of any of embodiments 489-492, wherein the cancer is a relapsed/refractory cancer.

494. The lipid particle therapy for use, the lentiviral vector therapy for use or the sterile composition for use of any of embodiments 487-493, wherein the cancer is a relapsed and/or refractory Large B-cell Lymphoma (LBCL).

495. The lipid particle therapy for use, the lentiviral vector therapy for use or the sterile composition for use of embodiment 494, wherein the LBCL comprises Non-Hodgkin's lymphoma (NHL).

496. The lipid particle therapy for use, the lentiviral vector therapy for use or the sterile composition for use of any of embodiments 484-495, wherein the subject has received 2 prior lines of systemic therapy for treating the cancer.

497. The lipid particle therapy for use, the lentiviral vector therapy for use or the sterile composition for use of any of embodiments 484-496, wherein the subject has received 2 prior chemotherapy regimens comprising 1 or more regimens comprising anthracycline and/or one or more regimens comprising anti-CD20 antibody.

498. The lipid particle therapy for use, the lentiviral vector therapy for use or the sterile composition for use of any of embodiments 484-497, wherein the subject has received an autologous stem cell transplant (ASCT).

499. The lipid particle therapy for use, the lentiviral vector therapy for use or the sterile composition for use of any of embodiments 492-498, wherein the NHL comprises a lymphoma selected from the group consisting of diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma.

500. The lipid particle therapy for use, the lentiviral vector therapy for use or the sterile composition for use of any of embodiments 484-499, wherein the subject has not received a lymphodepleting regimen or therapy.

501. The lipid particle therapy for use, the lentiviral vector therapy for use or the sterile composition for use of any of embodiments 484-500, wherein the subject has not received a lymphodepleting regimen or therapy within 30 days prior to the use or administration.

502. The lipid particle therapy for use, the lentiviral vector therapy for use or the sterile composition for use of any of embodiments 484-501, prior to the use, the subject has received one or more agents to mobilize peripheral blood hematopoietic stem cells or CD34+progenitor cells, optionally wherein the agent is G-CSF.

503. The method of embodiment 483 or the lipid particle therapy for use, the lentiviral vector therapy for use or the sterile composition for use of embodiment 502, wherein the one or more agents that stimulate mobilization are selected from the group consisting of stem cell factor (SCF), small molecule VLA-4 inhibitor BI05192, BOP (N-(benzenesulfonyl)-L-prolyl-L-O-(1-pyrrolidinylcarbonyl)tyrosine), heparin, granulocyte colony-stimulating factor (G-CSF), MGTA-145, and plerixafor (AMD3100).

504. The method of embodiment 483 or embodiment 403 or the lipid particle therapy for use, the lentiviral vector therapy for use or the sterile composition for use of embodiment 492 or embodiment

494, wherein the one or more agents that stimulate mobilization comprise G-CSF.

505. The method or the lipid particle therapy for use, the lentiviral vector therapy for use or the sterile composition for use of embodiment 504, wherein the G-CSF is administered to the subject daily on the two days, three days, four days, or five days prior to obtaining the whole blood.

506. The method or the lipid particle therapy for use, the lentiviral vector therapy for use or the sterile composition for use of embodiment 504 or embodiment 505, wherein the G-CSF is administered to the subject on the day of obtaining the whole blood.

507. The method or the lipid particle therapy for use, the lentiviral vector therapy for use or the sterile composition for use of embodiment 504 or embodiment 505, wherein the G-CSF is administered to the subject on the day of reinfusing the contacted PBMCs to the subject.

508. The method or the lipid particle therapy for use, the lentiviral vector therapy for use or the sterile composition for use of any of embodiments 483 and 502-507, wherein the one or more agents that stimulate mobilization comprise plerixafor.

509. The method or the lipid particle therapy for use, the lentiviral vector therapy for use or the sterile composition for use of embodiment 508, wherein the plerixafor is administered to the subject on the day of reinfusing the contacted PBMCs to the subject.

510. The method or the lipid particle therapy for use, the lentiviral vector therapy for use or the sterile composition for use of any of embodiments 483 and 502-509, wherein the one or more agents that stimulate mobilization are G-CSF and plerixafor.

511. The method or the lipid particle therapy for use, the lentiviral vector therapy for use or the sterile composition for use of embodiment 510, wherein:
the G-CSF is administered to the subject daily on the four days prior to obtaining the blood; and
the plerixafor is administered to the subject on the day of reinfusing the contacted PBMCs to the subject.

### EXAMPLES

The following examples are included for illustrative purposes only and are not intended to limit the scope of the invention.

### Example 1 Ex Vivo Dosing of Human PBMCs for Tumor Control in Mice

To assess the feasibility of ex vivo dosing of a viral vector, human PBMCs were contacted with viral vector particles encoding an anti-CD19 CAR and the transduced cells were administered to CD19-expressing Nalm6 tumor-bearing mice within 2 or 4 hours after the initial contacting with the viral vector as described below.

For viral vector production, HEK293 producer cells were transfected with plasmids expressing viral vector proteins (gag/pol, rev) and a transfer plasmid encoding an anti-CD19 CAR (containing an FMC63-derived scFv extracellular antigen binding domain, a CD8α transmembrane domain, and an intracellular signaling domain containing a 4-1BB costimulatory signaling domain and a CD3zeta signaling domain). Envelope proteins were provided as plasmids expressing Nipah F protein and a CD8-retargeted Nipah G protein (see US 2019/0144885, incorporated by reference herein). The CD8-retargered Nipah G (NiV-G) protein contained an anti-CD8 scFv as a fusion with the exemplary NiV-G sequence GcΔ34 (Bender et al. 2016 PLoS Pathol 12(6):e1005641; set forth in SEQ ID NO:19), and the Nipah F (NiV-F) protein was the exemplary NiV-F sequence NivFdel22 (SEQ ID NO:11; or SEQ ID NO:12 without a signal sequence; Bender et al. 2016 PLoS). Following viral vector production, the cell culture was centrifuged to pellet the cells and the supernatant containing crude virus was collected.

### a. 4 Hour Incubation Protocol

NSG mice bearing were infused intravenously with NALM6 tumor cells (1×10⁷tum or cells/mouse) and the tumor was allowed to grow for about 72 hours. The tumor cells administered were additionally engineered to contain a luciferase marker. About 6×10⁷human PBMCs from a healthy donor were thawed and incubated with viral vector such that cells would be exposed to vector at a dose ranging from 1×10⁶ IU to 1×10⁷ IU. Following incubation for 4 hours, the transfected PBMCs were pelleted at 500×g for 5 minutes, resuspended in 1200 μL saline, and then directly injected into the mice on D-1. Live imaging of the mice was conducted starting on study D1, and bleeds for flow cytometric analysis were collected on D14 and D28. Animals were sacrificed on study D28 for study of the spleen.

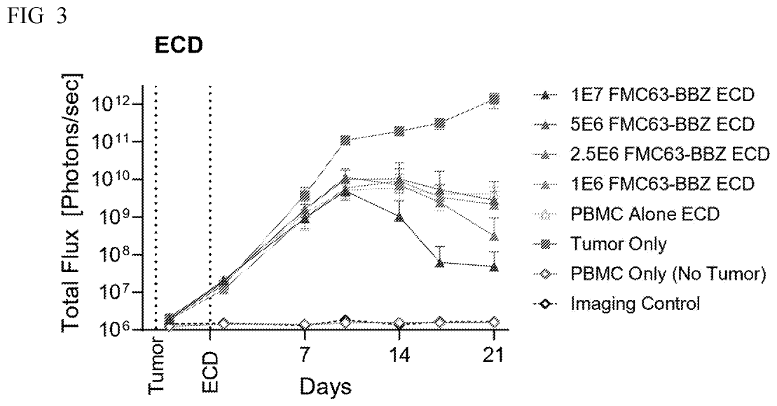

The number of tumor cells was monitored over the study period as Total Flux via flow cytometry. Tumor growth as monitored by Total Flux is shown in FIG. 3 for each of the viral vector doses. Also depicted are several controls, including a tumor only control, PBMC only control, as well as a naive imaging control. As shown, each of the viral vector doses reduced tumor growth in this model compared to tumor control mice that were not administered ex vivo viral vector.

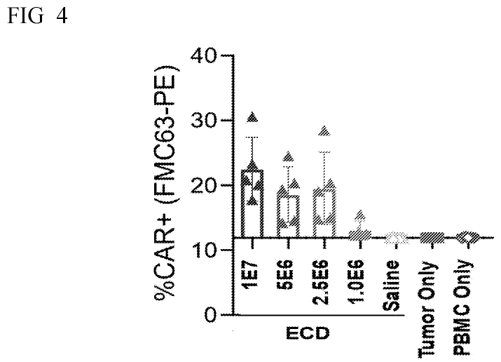

CD8+ T cells expressing the CAR were detected in the peripheral blood in mice, as shown by flow cytometric analysis of CD8+cells in the peripheral blood from study D14 depicted in FIG. 4. The CAR was not detected on T cells from peripheral blood of control mice.

### b. 2 Hour Incubation Protocol

NSG mice bearing were infused intravenously with NALM6 tumor cells (1×106tumor cells/mouse) and the tumor was allowed to grow for about 72 hours. The tumor cells administered were additionally engineered to contain a luciferase marker. About 1×10⁷human PBMCs from a single healthy donor were obtained.

In an in vivo delivery control condition, 1×10⁷ huPBMC were injected into each animal, followed by I.V. injection of the exemplary viral vector about 24 hours later.

In conditions designed to assess extracorporeal delivery, about 1×10⁷ human PBMCs from a single healthy donor were obtained and incubated with viral vector such that cells would be exposed to vector at a dose ranging from 1.25×10⁶-5×10⁶ IU. Following incubation for only 2 hours, the transfected PBMCs were pelleted at 500×g for 5 minutes resuspended in 1200 μL saline, and then injected into the mice on D-1. In a third condition designated “combined injection”, PBMCs and viral vector are incubated as described above and injected directly, with no resuspension to remove unbound viral vector.

Live imaging of the mice was conducted starting on study D1, and bleeds for flow cytometric analysis were collected on D14 and D28. Animals were sacrificed on study D28 for study of the spleen.

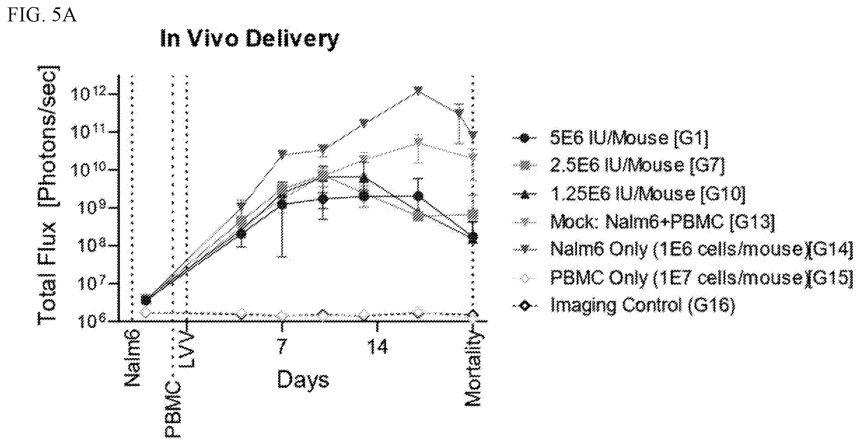

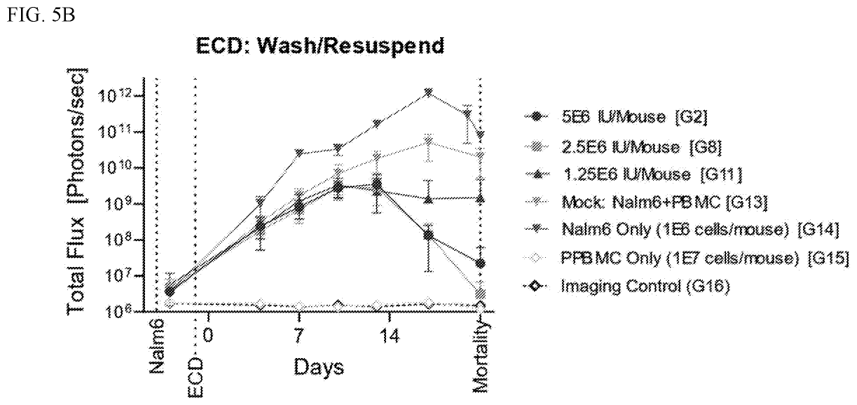

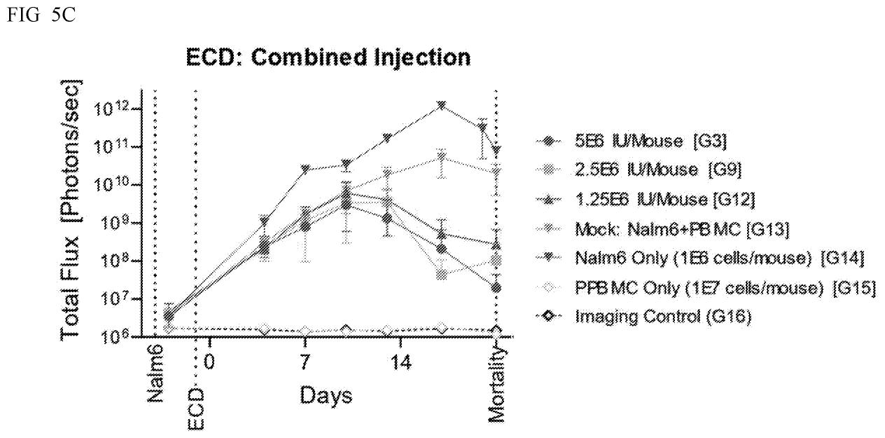

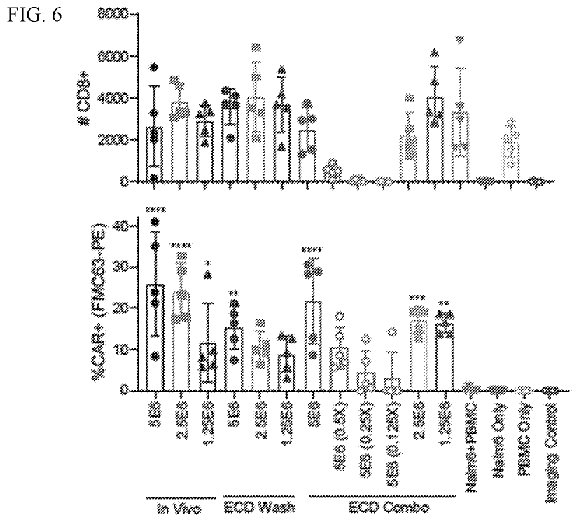

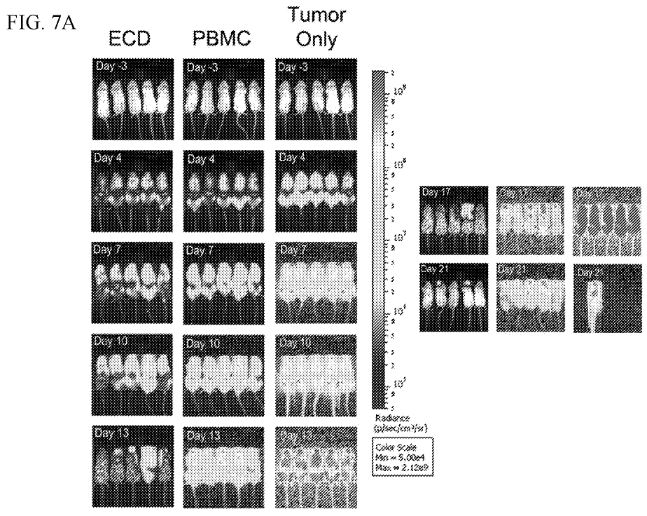

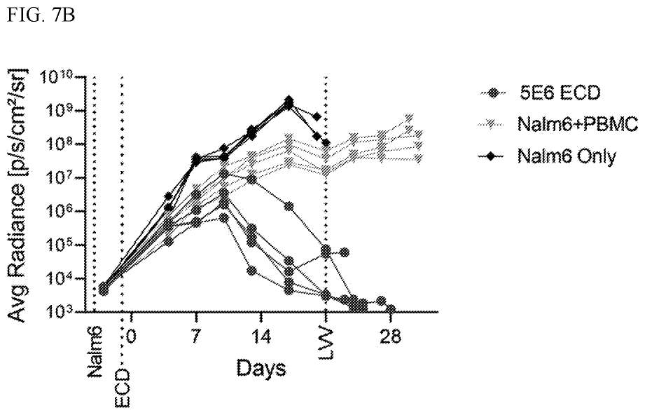

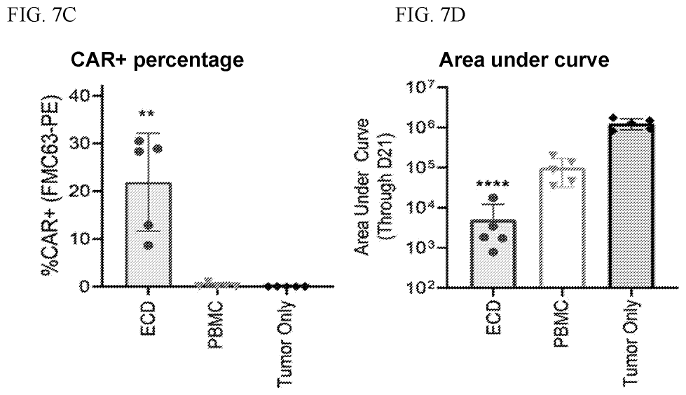

The number of tumor cells was monitored over the study period as Total Flux via flow cytometry. Tumor growth as monitored by Total Flux is shown for each of the administration conditions described above in FIG. 5A (In vivo delivery), FIG. 5B (ECD with a wash/resuspend), and FIG. 5C (combined injection). Also depicted are several controls, including a tumor only control (e.g., Nalm6 only), PBMC only control, as well as a naive imaging control. As shown, the ECD delivery of viral vector effectively reduced tumor growth in this model compared to tumor control mice that were administered viral vector via IV, which was as efficient or improved compared to the other delivery methods. Following delivery, CAR-T cells were detected within CD8+cells in peripheral blood at day 14 (FIG. 6).

In the Nalm6 model, tumor bioluminescence (BLI) was measured daily in mice that had been delivered 5×10⁶ IU CD8-retargeted viral vector by ECD, or that had received PBMCs only (untreated) or Nalm6 tumor cells only. CAR T frequency in peripheral blood was also assessed. FIG. 7A depicts tumor luciferase activity at indicated timepoints, and FIG. 7B depicts radiance of individual mice at indicated time points. Short-term exposure of PBMCs to CD8 re-targeted viral vector, followed by injection into CD19+Nalm-6 tumor-bearing animals, showed increased CAR T cell frequency (FIG. 7C), and elimination of tumor as tumor size was significantly reduced as assessed by area under the curve (AUC) showing cumulative tumor size through day 17 (FIG. 7D).

### c. 1 Hour Incubation Protocol

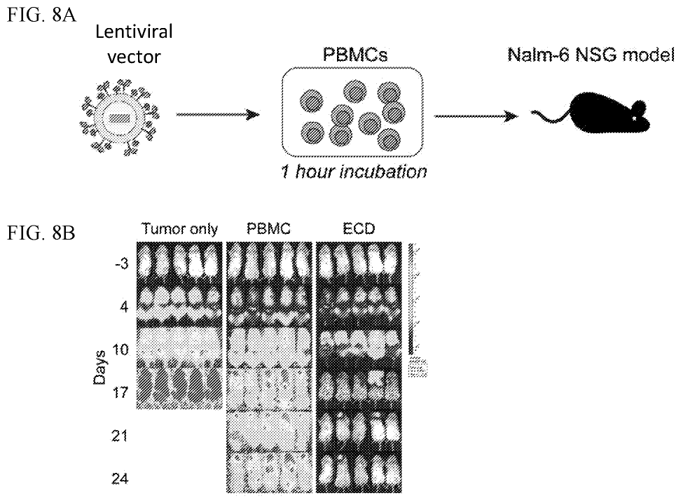

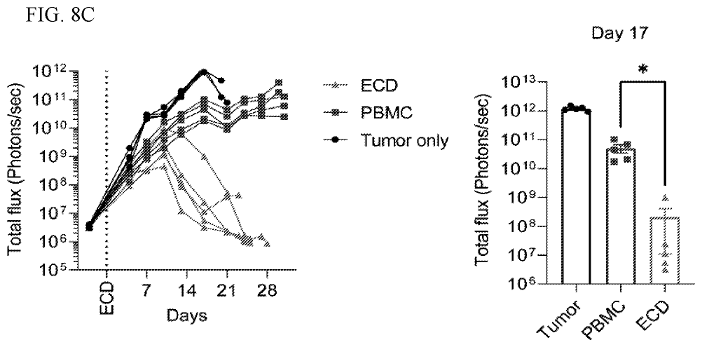

In vivo efficacy was measured using immune-deficient NSG mice engrafted with CD19+Nalm-6 tumors, substantially as described in Example 1. Freshly thawed healthy or patient (DLBCL) PBMCs were incubated for 1 hour with CD8-targeted CD19 CAR lentiviral vector and subsequently infused via i.v. injection (FIG. 8A). Tumor bioluminescence (BLI) was measured weekly, and CAR T frequency in peripheral blood was assessed. Short-term exposure of PBMCs, followed by intravenous injection into CD19+Nalm-6 tumor-bearing animals showed elimination of tumor (FIG. 8B and FIG. 8C).

These results show that lentiviral vector encoding a CAR transgene and pseudotyped with a CD8-targeted fusogen can specifically target resting CD8+ T cells. These results support rapid, ex vivo production of functional CAR T cells from apheresis collections after a brief incubation period. Extracorporeal delivery also allows for the optimization of viral vector and T cell exposure at a given dose to maximize CAR T transduction of freshly isolated lymphocytes (i.e., apheresis). This method has the potential for rapid production of CAR T cells for patients at lower viral vector doses. ECD may allow bedside CAR T applications while providing pharmacokinetic, safety and efficacy data for direct in vivo vector administration, such as by direct intravenous administration of CD8-targeted viral vectors delivering a CD19 CAR transgene.

### Example 2: Ex Vivo Dosing of CD8-Directed CD19 CAR-T 2Ene Therapy

A viral vector particle encoding a CD8-directed CD19 CAR-T gene is administered by ex vivo dosing to patients with relapsed and/or refractory Large B-cell Lymphoma (LBCL). Patients include patients with Non-Hodgkin's lymphoma undergoing 2+lines of systemic therapy, including diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma. Readouts from the administration include objective response and duration of response per Lugano classification criteria, MRD negativity; pharmacokinetics-related parameters; and cellular kinetics-related parameters. Patients are monitored for any toxicities (e.g., dose-limiting toxicities) and any adverse events (e.g., treatment-emergent adverse events or treatment-related adverse events).

For viral vector production, HEK293 producer cells are transfected with plasmids expressing viral vector proteins (gag/pol, rev) and a transfer plasmid encoding an anti-CD19 CAR (containing an FMC63-derived scFv extracellular antigen binding domain (SEQ ID NO:40), CD8 hinge (SEQ ID NO:27) and transmembrane (SEQ ID NO:33) domains, and an intracellular signaling domain containing a 4-1BB costimulatory signaling domain (SEQ ID NO:36) and a CD3zeta signaling domain (SEQ ID NO:38)). Envelope proteins are provided as plasmids expressing Nipah F protein and a CD8-retargeted Nipah G protein (see U.S. provisional application No. 63/172,518, incorporated by reference herein). The CD8-retargered Nipah G (NiV-G) protein has an anti-CD8 scFv as a fusion with the exemplary NiV-G sequence GcΔ34 (Bender et al. 2016 PLoS Pathol 12(6):e1005641; set forth in SEQ ID NO:19), and the Nipah F (NiV-F) protein has the exemplary NiV-F sequence NivFdel22 (SEQ ID NO:11; or SEQ ID NO:12 without a signal sequence; Bender et al. 2016 PLoS). Following viral vector production, the cell culture is centrifuged to pellet the cells and the supernatant containing crude virus is collected.

Patients are not required to be pre-conditioned with either of cyclophosphamide and fludarabine prior to apheresis. Vascular access to the patient for apheresis is established via peripheral or central IV access for 60-120 mL/min flow rate. Prior to apheresis, the patient is monitored for complete blood count (CBC), blood chemistry, and blood coagulation. Further eligibility criteria are summarized in the Table E1 below.

TABLE E1
Patient Eligibility Criteria

| Inclusion Criteria | Exclusion Criteria |
|---|---|
| Histologically confirmed relapsed or refractory | Prior treatment with anti-CD19 therapy, any |
| large B-cell lymphoma | gene therapy, any genetically modified cell |
| | therapy or adoptive T-cell therapy |
| ≥2 prior chemotherapy containing regimens (≥1 | Ineligible or unable to receive CAR T therapy |
| anthracycline, ≥1 anti-CD20 mAb (unless CD20 | |
| negative) or after autologous stem | |
| cell transplant (ASCT) | |
| ≥1 measurable PET-positive lesion per Lugano | Active systemic infections, coagulation |
| Classification | disorders, or cardiopulmonary disease |
| Adequate Organ Function | Any disease requiring systemic steroid treatment |
| | or steroid inhalant. |
| ECOG performance status of 0 or 1 | CNS lymphoma or untreated brain metastases |

Patients are subject to apheresis in the collection of a Peripheral Blood Mononuclear Cells (PBMCs) separated from whole blood. The collected PBMCs are enriched in leukocytes (e.g., B and T lymphocytes). Briefly, patient whole blood is pumped into a tubing set and spun in a centrifuge at the speed to target packing factor of 4.5. Cells flow into the collect port where mononuclear cells are pumped into the collection bag. Red blood cells, platelets, and plasma are pumped into the reservoir and returned to the patient via the apheresis device. An average of 10-12 liters of blood is processed per patient, yielding an average of 10×10⁹ (about 1×10⁸ to 30×10⁹) nucleated cells, 4×10′ (about 4×108 to 20 x 10⁹) CD3+ T cells, and 1.5×10⁹ CD8+ T cells in a median volume of 240 mL.

PBMCs are contacted with the viral vector particles for an incubation period of 30-60 mins and the transduced cells are reinfused into the patient via a side port which allows the collection bag to operate on a closed system with no disconnection from the patient. Patients are monitored for vital signs (e.g., blood pressure, heart rate, O₂, oxygen saturation, etc.), fluid balance, platelet counts, hemolysis, and optionally an electrocardiogram (ECG).

In a first dose escalation study, patients are administered the viral particles encoding the CD8-directed CD19 CAR-T gene therapy by ex vivo dosing according to the Table E2. In this protocol, the viral particles are administered at a constant MOI relative to the target cells.

TABLE E2
Dose Design for Ex Vivo Administration of Lentiviral
Particles Encoding CD8 directed anti-CD19 CAR

| | | | Estimated |
|---|---|---|---|
| Dose | Vector Dose | Target CD8+ T Cells | CAR-T |
| Level | (1 × 10⁷ IU/mL) | (estimated apheresis vol) | Cells |
| 1 | 100 mL | 5 × 10⁸ (100 mL) | 10-25 × 10⁶ |
| 2 | 200 mL | 10 × 10⁸ (200 mL) | 20-50 × 10⁶ |
| 3 | 400 mL | 20 × 10⁸ (400 mL) | 40-100 × 10⁶ |

An alternative dosing schedule involving a fixed number of cells (e.g., 100 mL white blood cells) and increasing Multiplicity of infection (MOI) of vector virus as follows (e.g., 100-400 ml virus) may be administered.

After ex vivo dosing, the patient is assessed for transduction efficiency, vector copy number, on-target gene delivery, target cell population, and humoral immunogenicity. Biomarkers to be evaluated with ex vivo vector delivery of CAR T therapy include tumor burden, peak of CAR T cell number (Cₘₐₓ), cellular kinetics (AUC), cytokine profile and kinetics thereof (e.g., IL-4, IL-6, IL-8, IL-10, IL-15, TNF-α, INF-γ, IL2Rα, MIP-1α, etc.), immunogenicity, CAR T cell dose, durability and/or persistence or response, and impact of previous treatment and other patient criteria.

### Example 3 Transduction of T Cells Using a CD8 Targeted Fusogen to Generate CAR T Cells Via Lentiviral Vector Following Treatment with Cytokines

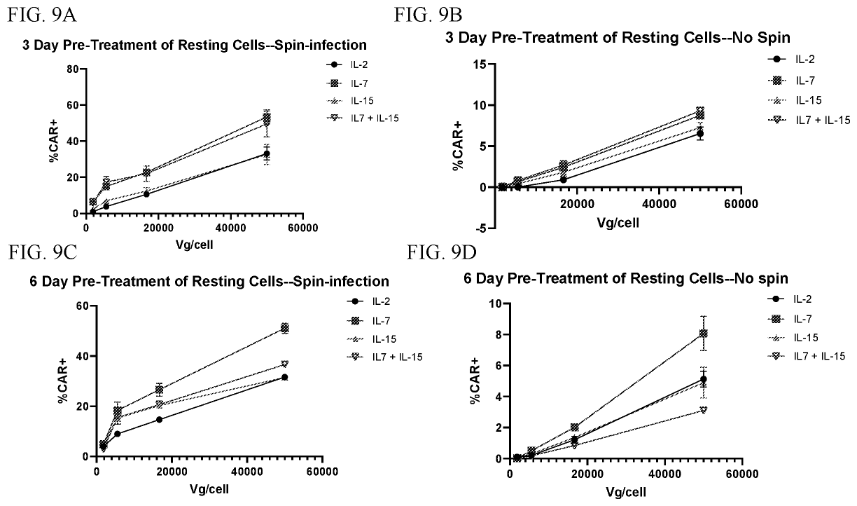

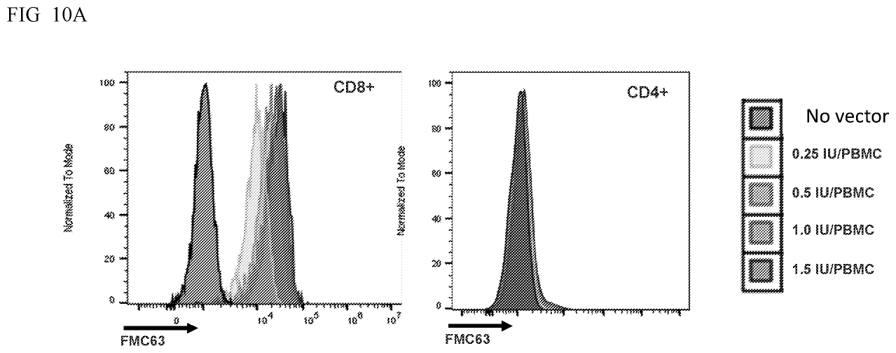

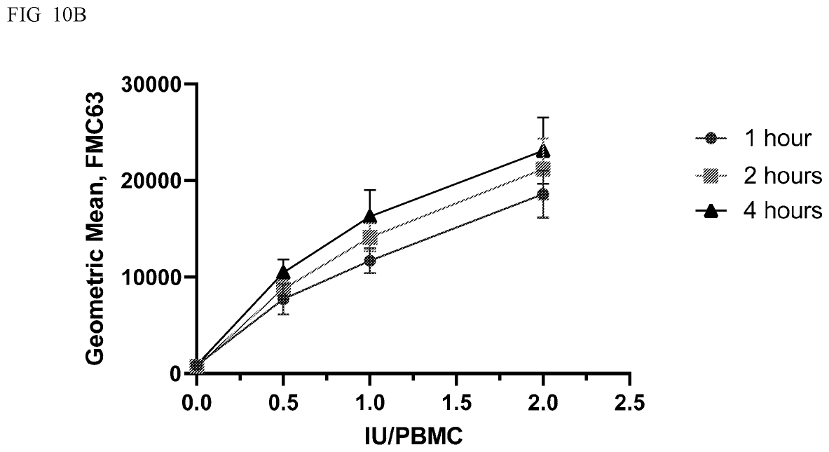

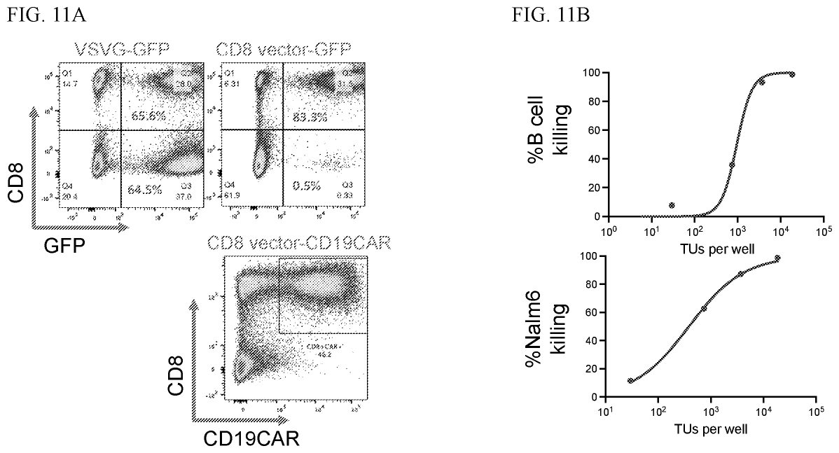

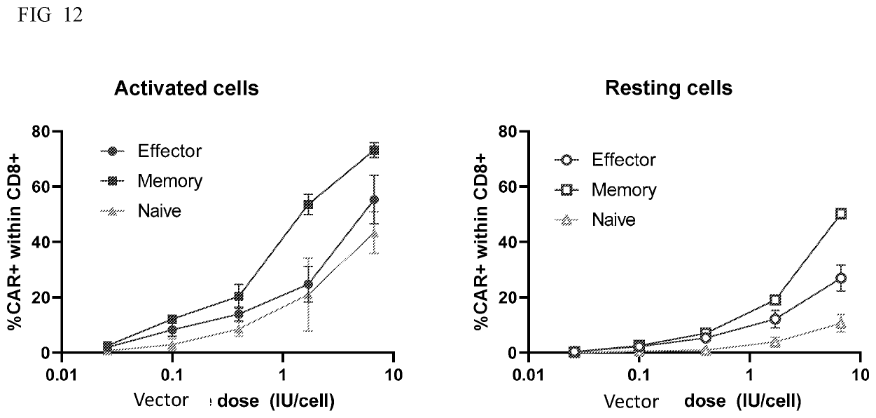

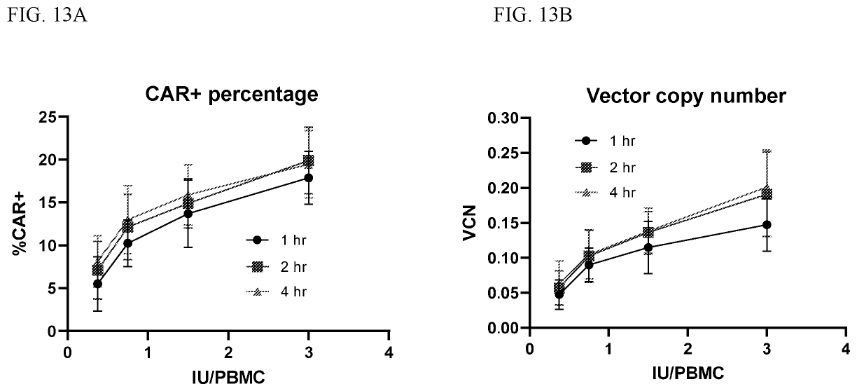

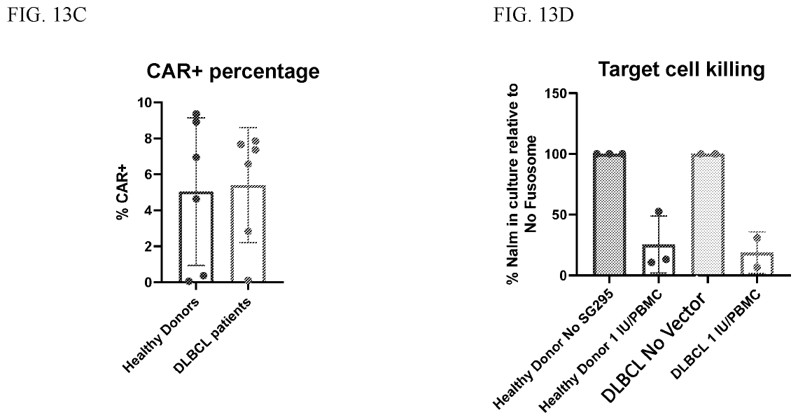

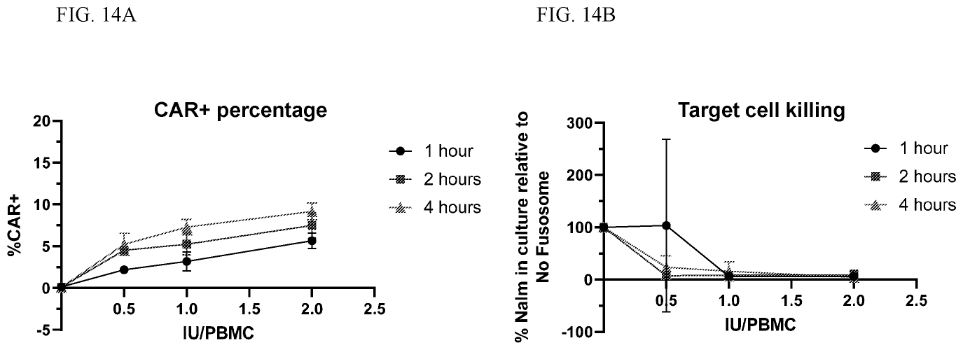

This example describes the analysis of cytokine treatment on T cell transduction by targeted lentiviral vectors. PBMCs from three human donors were thawed and rested for 3 or 6 days with cytokines (100 U/ml IL-2; 15 ng/ml IL-7; 10 ng/ml IL-15; or 15 ng/ml IL-7+10 ng; ml IL-15). Following resting of the cells with cytokines, the cells were transduced with a lentiviral vector pseudotyped with a CD8-retargeted Nipah fusogen and a CD19 CAR transgene, plus or minus spin-fection at 3-fold dilutions of 5e4 vector genomes/cell. The next day, the cells were washed to remove unbound lentiviral vector and the cells were activated with CD3/CD28 DYNABEADS human T activator CD3/CD28 (Invitrogen). Six days later, the cells were analyzed for CAR expression by flow cytometry and the presence of transgene by VCN analysis. In general, the percent of cells expressing CAR increased with increasing vector concentration (FIGS. 9A-9D). IL-7 pretreatment resulted in increased transduction as compared to pretreatment with IL-2 or IL-15 (FIGS. 9A-9D). Whereas the combination of IL-7 and IL-15 provided roughly equivalent CAR expression to IL-7 alone after three days pre-treatment (FIGS. 9A-9B), the combination of IL-7 and IL-15 resulted in reduced CAR expression after six days pre-treatment (FIGS. 9C-9D).

In summary, this example demonstrates measurable transduction of human T cells using a targeted vector following treatment with various cytokines.

### Example 4 Transduction of CD8+ T Cells Via Viral Vectors with CD8-Targeted Viral Vector

The ability of the CD8-retargeted viral vector prepared as described above to specifically bind or transduce CD8+ T cells was assessed.

Cells were transduced with an exemplary lentiviral vector encoding a GFP transgene or a CD19 CAR transgene (containing an FMC63-derived scFv extracellular antigen binding domain, a CD8α transmembrane domain, and an intracellular signaling domain containing a 4-1BB costimulatory signaling domain and a CD3zeta signaling domain). The lentiviral vectors were pseudotyped with VSV-G or a CD8-retargeted Nipah fusogen composed of a Nipah F protein and a CD8-retargeted Nipah G protein (see US 2019/0144885, incorporated by reference herein). The CD8-retargeted Nipah G (NiV-G) protein contained an anti-CD8 scFv targeting human CD8α as a fusion with the exemplary NiV-G sequence GcΔ34 that was mutated to ablate its native tropism (Bender et al. 2016 PLoS Pathol 12(6):e1005641; set forth in SEQ ID NO:19), and the Nipah F (NiV-F) protein was the exemplary NiV-F sequence NivFdel22 (SEQ ID NO:11; or SEQ ID NO:12 without a signal sequence; Bender et al. 2016 PLoS). Viral vectors were produced substantially as described in Example 1.

CD4+ or CD8+ T cells from apheresis were exposed to CD8 retargeted viral vector for 1, 2 or 4 hours. After viral vector exposure, CD4+ or CD8+ T cells were washed and stained with a panel of antibodies to assess vector binding by measuring passively packaged CAR with the anti-idiotype FMC63 antibody. It was observed that the viral vector successfully bound to the surface of CD8+ T cells but not CD4+ T cells FIG. 10A. As shown in FIG. 10B, binding of the viral vector to target CD8 cells was confirmed to be dose dependent and was detected by anti-FMC63 antibody after exposure for as little as 1 hour.

Transduction of CD8+ T cell with the exemplary CD8 targeted vector compared to VSVg-pseudotyped lentiviral vector also was assessed by short term incubations of viral vector with isolated PBMCs or apheresis samples at various multiplicity-of-infection (MOI; 0.25 to 3 IU/cell calculated from infections of SupTI cells). Transduction was followed by measurement of GFP or CAR transduction and ability to kill CD19+target cells. As shown in FIG. 11A, the CD8 retargeted viral vector resulted in specific transduction of CD8+ T cells as compared to VSVg-pseudotyped lentiviral vector, as determined by expression that was analyzed after 7 days by flow cytometry demonstrating delivery of GFP (top) or CD19 CAR (bottom) transgene in CD3/CD28 bead-activated human PBMCs. CD8+ T cells transduced with CD8/CD19CAR vector eliminated CD19+B cells and Nalm6 tumor cells in a dose-dependent manner as shown in FIG. 11B..These data support that the exemplary CD8-targeted vector described can specifically transduce CD8 T cells.

The phenotype of activated CD8+ T cells (CD3/CD28 beads) or resting CD8+ T cell transduced with CD8 retargeted viral vector was assessed by flow cytometry for naive (CD45RA+CCR7+), memory (CD45RO+), and effector (CD45RA-CCR7−) CD8+ T cells. Transduction was determined by analyzing CD19CAR expression 7 days after transduction by flow cytometry. As shown in FIG. 12, it was observed that transduction was more pronounced in memory compared to effector or naive cells, and in activated compared to resting cells. The results indicate that CD8-targeted delivery targets less differentiated T cells.

### Example 5 Transduction of Resting T Cells Via Viral Vectors with CD8-Targeted Viral Vector

Resting T cells collected as an apheresis product were also assessed for transduction with a CD8-targeted viral vector, as well as for functional activity. Vector copy number (VCN) also was assessed.

Cells were transduced with an exemplary lentiviral vector encoding a GFP transgene or a CD19 CAR transgene (containing an FMC63-derived scFv extracellular antigen binding domain, a CD8α transmembrane domain, and an intracellular signaling domain containing a 4-1BB costimulatory signaling domain and a CD3zeta signaling domain). The lentiviral vectors were pseudotyped with VSV-G or a CD8-retargeted Nipah fusogen composed of a Nipah F protein and a CD8-retargeted Nipah G protein (see US 2019/0144885, incorporated by reference herein). The CD8-retargeted Nipah G (NiV-G) protein contained an anti-CD8 scFv targeting human CD8α as a fusion with the exemplary NiV-G sequence GcΔ34 that was mutated to ablate its native tropism (Bender et al. 2016 PLoS Pathol 12(6):e1005641; set forth in SEQ ID NO:19), and the Nipah F (NiV-F) protein was the exemplary NiV-F sequence NivFdel22 (SEQ ID NO:11; or SEQ ID NO:12 without a signal sequence; Bender et al. 2016 PLoS). Viral vectors were produced substantially as described in Example 1.

Resting or activated (anti-CD3/anti-CD28 beads) apheresis-collected lymphocytes were transduced via short-term exposure (30 minutes to 4 hours) with CD8-targeted lentiviral vectors at various multiplicity-of-infection (MOI; 0.5 to 3 IU/cell). CAR transduction and ability to kill CD19+target cells was assessed. CD8-targeted lentiviral vectors showed specific transduction of CD8+activated T cells, and further, demonstrated the ability to quickly bind (within 1 hour) and transduce resting CD8+ T cells (up to 19.9±3.8% CAR+CD8 T cells at 3 IU/PBMC and VCN<0.5) resulting in functional CD19 CAR T cells. Flow cytometry and cell sorting indicated that CD8-targeted lentiviral vector can target naive CD8+ T cells (CD45RA+CCR7+; 10.6±3.1%), memory (CD45RO+; 50.2±0.5%), and effector (CD45RA-CCR7-; 26.9±4.8%).

In a related experiment, a dose-dependent increase in CAR expression and integration was observed after the short-term exposure (1 to 4 hours) as assessed at day 8 of coculture as shown based on the percentage of CAR+CD8+ T cells (FIG. 13A and integration as assessed via Vector Copy Number (VCN; FIG. 13B).A positive correlation was observed between CAR expression and VCN.

To further assess transduction feasibility of resting T cells, freshly thawed healthy or patient (DLBCL) PBMCs were transduced via short-term exposure (1 hour to 4 hours) with CD8-targeted lentiviral vectors. , and then were used to assess cytotoxic killing of Nalm-6 CD19+tumor cells. Transduction of both healthy and patient (DLBCL) PBMCs was vector- and time-dependent, and specific for CD8+ T cells, resulting in the generation of cytotoxic, CAR T cells (e.g. at 1 IU/PBMC, healthy donors: 5.0±4.1% CAR+CD8 T cells vs. DLBCL patients: 5.4±3.2% CAR+CD8 T cells) capable of lysing CD19+tumor cells (healthy donors: 74.5±23.5% vs. DLBCL patients: 81.4±17.1% reduction in Nalm-6 representation within co-culture versus cells not exposed to the CD8-targeted lentiviral vector). Exemplary results are depicted in FIG. 13C (CAR+percentage of CD8+ T cells at day 8 of culture) and FIG. 13D (Nalm6 target cell killing after 72 hour coculture).

Taken together, these results show short-term incubation of healthy or DLBCL patient lymphocytes with an exemplary CD8-targeted CD19 CAR viral vector results in the generation of highly functional CD8+CAR T cells capable of eliminating CD19+tumor cells in vitro.

### Example 6 Transduction of Cells from Fresh Leukopaks with CD8-Targeted Viral Vector

In order to further assess functional CAR T generation by extracorporeal delivery of viral vector, CD8-targeted CD19CAR exemplary vector as described above was incubated by short-term exposure (1 to 4 hours) with cells directly sampled from fresh leukopaks, followed by activation and culture. The CD8-targeted viral vectors carrying a CD19 CAR transgene were generated as described in Example 1. The percentage of CAR+cells in the CD8+ T cell population is shown in FIG. 14A and Nalm6 target cell killing initiated at day 8 of culture is shown in FIG. 14B. The results confirm generation of CAR T cells by ECD in a clinically relevant setting.

### Example 7 Transduction of T Cells Using a CD8 Targeted Lentiviral Vector Following Treatment with Interleukin 7

Human PBMC were exposed to IL-7 for 3 days before transduction with a lentiviral vector pseudotyped with a CD8-retargeted Nipah fusogen and a CD19 CAR transgene (“fusosome”). Cell expansion and transgene expression levels were analyzed during 7-10 day cultures by flow cytometry. To test post-treatment effects, fusosome-generated CD19 CAR-T cells were cultured with Nalm6 cells and IL-7 for 10 days, and cell expansion and tumor killing were monitored by Incucyte. For in vivo testing, NSG mice were engrafted with Nalm6-ffluc tumors (5E5 cells, IV) on day -4, and with human PBMC (1E7 cells, IV) on day -1. CD8/CD19CAR fusosome was dosed on day 0 (IV). IL-7 (1-5 ug/mouse) was administered SC twice weekly. Tumor growth was monitored by bioluminescence imaging.

Pre-treatment was observed to have a moderate effect (-1.5x) on transduction efficiency with CD8 targeted fusosomes compared to untreated cells. IL-7 treatment promoted the expansion of both fusosome-generated CD8+CAR-T cells and non-transduced T cells in vitro. However, in the presence of tumor antigen, IL-7 treatment resulted in selective expansion of CAR-T cells over bystander T cells (40× vs 10×, respectively) in vitro. Importantly, systemic IL-7 treatment following CD8/CD19CAR fusosome administration increased efficacy in Nalm6 tumor bearing NSG mice compared to those treated with fusosome only.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID NO | SEQUENCE | DESCRIPTION |
|---|---|---|
| 1 | MATQEVRLKCLLCGIIVLVLSLEGLGILHYEKLSKIGLVKGI | Hendra F |
| | TRKYKIKSNPLTKDIVIKMIPNVSNVSKCTGTVMENYKSRL | |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILYV | |
| | LSIAALCIGLITFISFVIVEKKRGNYSRLDDRQVRPVSNGDL | |
| | YYIGT | |
| 2 | MVVILDKRCYCNLLILILMISECSVGILHYEKLSKIGLVKGV | Nipah F0 |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNTYSRLEDRRVRPTSSGDLYYI | |
| | GT | |
| 3 | MSNKRTTVLIIISYTLFYLNNAAIVGFDFDKLNKIGVVQGRV | CedV F |
| | LNYKIKGDPMTKDLVLKFIPNIVNITECVREPLSRYNETVRR | |
| | LLLPIHNMLGLYLNNTNAKMTGLMIAGVIMGGIAIGIATAA | |
| | QITAGFALYEAKKNTENIQKLTDSIMKTQDSIDKLTDSVGT | |
| | SILILNKLQTYINNQLVPNLELLSCRQNKIEFDLMLTKYLVD | |
| | LMTVIGPNINNPVNKDMTIQSLSLLFDGNYDIMMSELGYTP | |
| | QDFLDLIESKSITGQIIYVDMENLYVVIRTYLPTLIEVPDAQI | |
| | YEFNKITMSSNGGEYLSTIPNFILIRGNYMSNIDVATCYMTK | |
| | ASVICNQDYSLPMSQNLRSCYQGETEYCPVEAVIASHSPRF | |
| | ALTNGVIFANCINTICRCQDNGKTITQNINQFVSMIDNSTCN | |
| | DVMVDKFTIKVGKYMGRKDINNINIQIGPQIIIDKVDLSNEI | |
| | NKMNQSLKDSIFYLREAKRILDSVNISLISPSVQLFLIIISVLS | |
| | FIILLIIIVYLYCKSKHSYKYNKFIDDPDYYNDYKRERINGK | |
| | ASKSNNIYYVGD | |
| 4 | MALNKNMFSSLFLGYLLVYATTVQSSIHYDSLSKVGVIKGL | Mojiang F |
| | TYNYKIKGSPSTKLMVVKLIPNIDSVKNCTQKQYDEYKNL | |
| | VRKALEPVKMAIDTMLNNVKSGNNKYRFAGAIMAGVALG | |
| | VATAATVTAGIALHRSNENAQAIANMKSAIQNTNEAVKQL | |
| | QLANKQTLAVIDTIRGEINNNIIPVINQLSCDTIGLSVGIRLT | |
| | QYYSEIITAFGPALQNPVNTRITIQAISSVFNGNFDELLKIMG | |
| | YTSGDLYEILHSELIRGNIIDVDVDAGYIALEIEFPNLTLVPN | |
| | AVVQELMPISYNIDGDEWVTLVPRFVLTRTTLLSNIDTSRC | |
| | TITDSSVICDNDYALPMSHELIGCLQGDTSKCAREKVVSSY | |
| | VPKFALSDGLVYANCLNTICRCMDTDTPISQSLGATVSLLD | |
| | NKRCSVYQVGDVLISVGSYLGDGEYNADNVELGPPIVIDKI | |
| | DIGNQLAGINQTLQEAEDYIEKSEEFLKGVNPSIITLGSMVV | |
| | LYIFMILIAIVSVIALVLSIKLTVKGNVVRQQFTYTQHVPSM | |
| | ENINYVSH | |
| 5 | MKKKTDNPTISKRGHNHSRGIKSRALLRETDNYSNGLIVEN | Bat PV F |
| | LVRNCHHPSKNNLNYTKTQKRDSTIPYRVEERKGHYPKIK | |
| | HLIDKSYKHIKRGKRRNGHNGNIITIILLLILILKTQMSEGAI | |
| | HYETLSKIGLIKGITREYKVKGTPSSKDIVIKLIPNVTGLNKC | |
| | TNISMENYKEQLDKILIPINNIIELYANSTKSAPGNARFAGVI | |
| | IAGVALGVAAAAQITAGIALHEARQNAERINLLKDSISATN | |
| | NAVAELQEATGGIVNVITGMQDYINTNLVPQIDKLQCSQIK | |
| | TALDISLSQYYSEILTVFGPNLQNPVTTSMSIQAISQSFGGNI | |
| | DLLLNLLGYTANDLLDLLESKSITGQITYINLEHYFMVIRVY | |
| | YPIMTTISNAYVQELIKISFNVDGSEWVSLVPSYILIRNSYLS | |
| | NIDISECLITKNSVICRHDFAMPMSYTLKECLTGDTEKCPRE | |
| | AVVTSYVPRFAISGGVIYANCLSTTCQCYQTGKVIAQDGSQ | |
| | TLMMIDNQTCSIVRIEEILISTGKYLGSQEYNTMHVSVGNPV | |
| | FTDKLDITSQISNINQSIEQSKFYLDKSKAILDKINLNLIGSVP | |
| | ISILFIIAILSLILSIITFVIVMIIVRRYNKYTPLINSDPSSRRSTIQ | |
| | DVYIIPNPGEHSIRSAARSIDRDRD | |
| 6 | ILHYEKLSKIGLVKGITRKYKIKSNPLTKDIVIKMIPNVSNVS | Hendra virus F |
| | KCTGTVMENYKSRLTGILSPIKGAIELYNNNTHDLVGDVKL | Protein, Without |
| | AGVVMAGIAIGIATAAQITAGVALYEAMKNADNINKLKSSI | signal sequence |
| | ESTNEAVVKLQETAEKTVYVLTALQDYINTNLVPTIDQISC | |
| | KQTELALDLALSKYLSDLLFVFGPNLQDPVSNSMTIQAISQ | |
| | AFGGNYETLLRTLGYATEDFDDLLESDSIAGQIVYVDLSSY | |
| | YIIVRVYFPILTEIQQAYVQELLPVSFNNDNSEWISIVPNFVLI | |
| | RNTLISNIEVKYCLITKKSVICNQDYATPMTASVRECLTGST | |
| | DKCPRELVVSSHVPRFALSGGVLFANCISVTCQCQTTGRAI | |
| | SQSGEQTLLMIDNTTCTTVVLGNIIISLGKYLGSINYNSESIA | |
| | VGPPVYTDKVDISSQISSMNQSLQQSKDYIKEAQKILDTVN | |
| | PSLISMLSMIILYVLSIAALCIGLITFISFVIVEKKRGNYSRLD | |
| | DRQVRPVSNGDLYYIGT | |
| 7 | ILHYEKLSKIGLVKGVTRKYKIKSNPLTKDIVIKMIPNVSNM | Nipah virus F |
| | SQCTGSVMENYKTRLNGILTPIKGALEIYKNNTHDLVGDVR | Protein, without |
| | LAGVIMAGVAIGIATAAQITAGVALYEAMKNADNINKLKS | signal sequence |
| | SIESTNEAVVKLQETAEKTVYVLTALQDYINTNLVPTIDKIS | |
| | CKQTELSLDLALSKYLSDLLFVFGPNLQDPVSNSMTIQAISQ | |
| | AFGGNYETLLRTLGYATEDFDDLLESDSITGQIIYVDLSSYY | |
| | IIVRVYFPILTEIQQAYIQELLPVSFNNDNSEWISIVPNFILVR | |
| | NTLISNIEIGFCLITKRSVICNQDYATPMTNNMRECLTGSTE | |
| | KCPRELVVSSHVPRFALSNGVLFANCISVTCQCQTTGRAIS | |
| | QSGEQTLLMIDNTTCPTAVLGNVIISLGKYLGSVNYNSEGIA | |
| | IGPPVFTDKVDISSQISSMNQSLQQSKDYIKEAQRLLDTVNP | |
| | SLISMLSMIILYVLSIASLCIGLITFISFIIVEKKRNTYSRLEDR | |
| | RVRPTSSGDLYYIGT | |
| 8 | TVLIIISYTLFYLNNAAIVGFDFDKLNKIGVVQGRVLNYKIK | Cedar Virus F |
| | GDPMTKDLVLKFIPNIVNITECVREPLSRYNETVRRLLLPIH | Protein, without |
| | NMLGLYLNNTNAKMTGLMIAGVIMGGIAIGIATAAQITAG | signal sequence |
| | FALYEAKKNTENIQKLTDSIMKTQDSIDKLTDSVGTSILILN | |
| | KLQTYINNQLVPNLELLSCRQNKIEFDLMLTKYLVDLMTVI | |
| | GPNINNPVNKDMTIQSLSLLFDGNYDIMMSELGYTPQDFLD | |
| | LIESKSITGQIIYVDMENLYVVIRTYLPTLIEVPDAQIYEFNKI | |
| | TMSSNGGEYLSTIPNFILIRGNYMSNIDVATCYMTKASVICN | |
| | QDYSLPMSQNLRSCYQGETEYCPVEAVIASHSPRFALTNGV | |
| | IFANCINTICRCQDNGKTITQNINQFVSMIDNSTCNDVMVD | |
| | KFTIKVGKYMGRKDINNINIQIGPQIIIDKVDLSNEINKMNQS | |
| | LKDSIFYLREAKRILDSVNISLISPSVQLFLIIISVLSFIILLIIIV | |
| | YLYCKSKHSYKYNKFIDDPDYYNDYKRERINGKASKSNNI | |
| | YYVGD | |
| 9 | IHYDSLSKVGVIKGLTYNYKIKGSPSTKLMVVKLIPNIDSVK | Mojiang virus, |
| | NCTQKQYDEYKNLVRKALEPVKMAIDTMLNNVKSGNNK | Tongguan 1 F |
| | YRFAGAIMAGVALGVATAATVTAGIALHRSNENAQAIAN | Protein, without |
| | MKSAIQNTNEAVKQLQLANKQTLAVIDTIRGEINNNIIPVIN | signal sequence |
| | QLSCDTIGLSVGIRLTQYYSEIITAFGPALQNPVNTRITIQAIS | |
| | SVFNGNFDELLKIMGYTSGDLYEILHSELIRGNIIDVDVDAG | |
| | YIALEIEFPNLTLVPNAVVQELMPISYNIDGDEWVTLVPRFV | |
| | LTRTTLLSNIDTSRCTITDSSVICDNDYALPMSHELIGCLQG | |
| | DTSKCAREKVVSSYVPKFALSDGLVYANCLNTICRCMDTD | |
| | TPISQSLGATVSLLDNKRCSVYQVGDVLISVGSYLGDGEYN | |
| | ADNVELGPPIVIDKIDIGNQLAGINQTLQEAEDYIEKSEEFLK | |
| | GVNPSIITLGSMVVLYIFMILIAIVSVIALVLSIKLTVKGNVV | |
| | RQQFTYTQHVPSMENINYVSH | |
| 10 | SRALLRETDNYSNGLIVENLVRNCHHPSKNNLNYTKTQKR | Bat |
| | DSTIPYRVEERKGHYPKIKHLIDKSYKHIKRGKRRNGHNGN | Paramyxovirus F |
| | IITIILLLILILKTQMSEGAIHYETLSKIGLIKGITREYKVKGTP | Protein, without |
| | SSKDIVIKLIPNVTGLNKCTNISMENYKEQLDKILIPINNIIEL | signal sequence |
| | YANSTKSAPGNARFAGVIIAGVALGVAAAAQITAGIALHEA | |
| | RQNAERINLLKDSISATNNAVAELQEATGGIVNVITGMQDY | |
| | INTNLVPQIDKLQCSQIKTALDISLSQYYSEILTVFGPNLQNP | |
| | VTTSMSIQAISQSFGGNIDLLLNLLGYTANDLLDLLESKSIT | |
| | GQITYINLEHYFMVIRVYYPIMTTISNAYVQELIKISFNVDGS | |
| | EWVSLVPSYILIRNSYLSNIDISECLITKNSVICRHDFAMPMS | |
| | YTLKECLTGDTEKCPREAVVTSYVPRFAISGGVIYANCLST | |
| | TCQCYQTGKVIAQDGSQTLMMIDNQTCSIVRIEEILISTGKY | |
| | LGSQEYNTMHVSVGNPVFTDKLDITSQISNINQSIEQSKFYL | |
| | DKSKAILDKINLNLIGSVPISILFIIAILSLILSIITFVIVMIIVRR | |
| | YNKYTPLINSDPSSRRSTIQDVYIIPNPGEHSIRSAARSIDRD | |
| | RD | |
| 11 | MVVILDKRCY CNLLILILMI SECSVGILHY EKLSKIGLVK | Truncated NiV |
| | GVTRKYKIKS NPLTKDIVIK MIPNVSNMSQ | fusion |
| | CTGSVMENYK TRLNGILTPI KGALEIYKNN | glycoprotein |
| | THDLVGDVRL AGVIMAGVAI GIATAAQITA | (FcDelta22) at |
| | GVALYEAMKN ADNINKLKSS IESTNEAVVK | cytoplasmic tail |
| | LQETAEKTVY VLTALQDYIN TNLVPTIDKI SCKQTELSLD | (with signal |
| | LALSKYLSDL LFVFGPNLQD PVSNSMTIQA ISQAFGGNYE | sequence) |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 12 | ILHY EKLSKIGLVK GVTRKYKIKS NPLTKDIVIK | Truncated mature |
| | MIPNVSNMSQ CTGSVMENYK TRLNGILTPI KGALEIYKNN | NiV fusion |
| | THDLVGDVRL AGVIMAGVAI GIATAAQITA | glycoprotein |
| | GVALYEAMKN ADNINKLKSS IESTNEAVVK | (FcDelta22) at |
| | LQETAEKTVY VLTALQDYIN TNLVPTIDKI SCKQTELSLD | cytoplasmic tail |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFIL VRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 13 | MMADSKLVSLNNNLSGKIKDQGKVIKNYYGTMDIKKIND | Hendra virus G |
| | GLLDSKILGAFNTVIALLGSIIIIVMNIMIIQNYTRTTDNQALI | protein Uniprot |
| | KESLQSVQQQIKALTDKIGTEIGPKVSLIDTSSTITIPANIGLL | O89343 |
| | GSKISQSTSSINENVNDKCKFTLPPLKIHECNISCPNPLPFRE | |
| | YRPISQGVSDLVGLPNQICLQKTTSTILKPRLISYTLPINTRE | |
| | GVCITDPLLAVDNGFFAYSHLEKIGSCTRGIAKQRIIGVGEV | |
| | LDRGDKVPSMFMTNVWTPPNPSTIHHCSSTYHEDFYYTLC | |
| | AVSHVGDPILNSTSWTESLSLIRLAVRPKSDSGDYNQKYIAI | |
| | TKVERGKYDKVMPYGPSGIKQGDTLYFPAVGFLPRTEFQY | |
| | NDSNCPIIHCKYSKAENCRLSMGVNSKSHYILRSGLLKYNL | |
| | SLGGDIILQFIEIADNRLTIGSPSKIYNSLGQPVFYQASYSWD | |
| | TMIKLGDVDTVDPLRVQWRNNSVISRPGQSQCPRFNVCPE | |
| | VCWEGTYNDAFLIDRLNWVSAGVYLNSNQTAENPVFAVF | |
| | KDNEILYQVPLAEDDTNAQKTITDCFLLENVIWCISLVEIYD | |
| | TGDSVIRPKLFAVKIPAQCSES | |
| 14 | MPAENKKVRFENTTSDKGKIPSKVIKSYYGTMDIKKINEGL | gb:AF212302\| |
| | LDSKILSAFNTVIALLGSIVIIVMNIMIIQNYTRSTDNQAVIK | Organism: Nipah |
| | DALQGIQQQIKGLADKIGTEIGPKVSLIDTSSTITIPANIGLLG | virus\|Strain |
| | SKISQSTASINENVNEKCKFTLPPLKIHECNISCPNPLPFREY | Name: UNKNOWN- |
| | RPQTEGVSNLVGLPNNICLQKTSNQILKPKLISYTLPVVGQS | AF212302\|Protein |
| | GTCITDPLLAMDEGYFAYSHLERIGSCSRGVSKQRIIGVGEV | Name: attachment |
| | LDRGDEVPSLFMTNVWTPPNPNTVYHCSAVYNNEFYYVL | glycoprotein\|Gene |
| | CAVSTVGDPILNSTYWSGSLMMTRLAVKPKSNGGGYNQH | Symbol: G |
| | QLALRSIEKGRYDKVMPYGPSGIKQGDTLYFPAVGFLVRTE | Uniprot Q9IH62 |
| | FKYNDSNCPITKCQYSKPENCRLSMGIRPNSHYILRSGLLK | |
| | YNLSDGENPKVVFIEISDQRLSIGSPSKIYDSLGQPVFYQASF | |
| | SWDTMIKFGDVLTVNPLVVNWRNNTVISRPGQSQCPRENT | |
| | CPEICWEGVYNDAFLIDRINWISAGVFLDSNQTAENPVFTV | |
| | FKDNEILYRAQLASEDTNAQKTITNCFLLKNKIWCISLVEIY | |
| | DTGDNVIRPKLFAVKIPEQCT | |
| 15 | MLSQLQKNYLDNSNQQGDKMNNPDKKLSVNFNPLELDKG | gb: JQ001776: |
| | QKDLNKSYYVKNKNYNVSNLLNESLHDIKFCIYCIFSLLIIIT | 8170-10275\| |
| | IINIITISIVITRLKVHEENNGMESPNLQSIQDSLSSLTNMINTE | Organism: |
| | ITPRIGILVTATSVTLSSSINYVGTKTNQLVNELKDYITKSCG | Cedar virus\|Strain |
| | FKVPELKLHECNISCADPKISKSAMYSTNAYAELAGPPKIFC | Name: CG1a\|Protein |
| | KSVSKDPDFRLKQIDYVIPVQQDRSICMNNPLLDISDGFFTY | Name: attachment |
| | IHYEGINSCKKSDSFKVLLSHGEIVDRGDYRPSLYLLSSHYH | glycoprotein\|Gene |
| | PYSMQVINCVPVTCNQSSFVFCHISNNTKTLDNSDYSSDEY | Symbol: G |
| | YITYFNGIDRPKTKKIPINNMTADNRYIHFTFSGGGGVCLGE | |
| | EFIIPVTTVINTDVFTHDYCESFNCSVQTGKSLKEICSESLRS | |
| | PTNSSRYNLNGIMIISQNNMTDFKIQLNGITYNKLSFGSPGR | |
| | LSKTLGQVLYYQSSMSWDTYLKAGFVEKWKPFTPNWMN | |
| | NTVISRPNQGNCPRYHKCPEICYGGTYNDIAPLDLGKDMY | |
| | VSVILDSDQLAENPEITVFNSTTILYKERVSKDELNTRSTTTS | |
| | CFLFLDEPWCISVLETNRFNGKSIRPEIYSYKIPKYC | |
| 16 | MPQKTVEFINMNSPLERGVSTLSDKKTLNQSKITKQGYFGL | gb:NC_025256: |
| | GSHSERNWKKQKNQNDHYMTVSTMILEILVVLGIMENLIV | 9117-11015\| |
| | LTMVYYQNDNINQRMAELTSNITVLNLNLNQLTNKIQREII | Organism: Bat |
| | PRITLIDTATTITIPSAITYILATLTTRISELLPSINQKCEFKTPT | Paramyxovirus |
| | LVLNDCRINCTPPLNPSDGVKMSSLATNLVAHGPSPCRNFS | Eid_hel/GH- |
| | SVPTIYYYRIPGLYNRTALDERCILNPRLTISSTKFAYVHSEY | M74a/GHA/2009\| |
| | DKNCTRGFKYYELMTFGEILEGPEKEPRMFSRSFYSPTNAV | Strain |
| | NYHSCTPIVTVNEGYFLCLECTSSDPLYKANLSNSTFHL VIL | Name: BatPV/Eid |
| | RHNKDEKIVSMPSFNLSTDQEYVQIIPAEGGGTAESGNLYF | hel/GH- |
| | PCIGRLLHKRVTHPLCKKSNCSRTDDESCLKSYYNQGSPQH | M74a/GHA/2009\| |
| | QVVNCLIRIRNAQRDNPTWDVITVDLTNTYPGSRSRIFGSFS | Protein |
| | KPMLYQSSVSWHTLLQVAEITDLDKYQLDWLDTPYISRPG | Name: glycoprotein\| |
| | GSECPFGNYCPTVCWEGTYNDVYSLTPNNDLFVTVYLKSE | Gene Symbol: G |
| | QVAENPYFAIFSRDQILKEFPLDAWISSARTTTISCFMENNEI | |
| | WCIAALEITRLNDDIIRPIYYSFWLPTDCRTPYPHTGKMTRV | |
| | PLRSTYNY | |
| 17 | MATNRDNTITSAEVSQEDKVKKYYGVETAEKVADSISGNK | gb:NC_025352: |
| | VFILMNTLLILTGAIITITLNITNLTAAKSQQNMLKIIQDDVN | 8716-11257\| |
| | AKLEMFVNLDQLVKGEIKPKVSLINTAVSVSIPGQISNLQTK | Organism: |
| | FLQKYVYLEESITKQCTCNPLSGIFPTSGPTYPPTDKPDDDT | Mojiang |
| | TDDDKVDTTIKPIEYPKPDGCNRTGDHFTMEPGANFYTVPN | virus\|Strain |
| | LGPASSNSDECYTNPSFSIGSSIYMFSQEIRKTDCTAGEILSIQ | Name: Tongguan1\| |
| | IVLGRIVDKGQQGPQASPLLVWAVPNPKIINSCAVAAGDE | Protein |
| | MGWVLCSVTLTAASGEPIPHMFDGFWLYKLEPDTEVVSYR | Name:a ttachment |
| | ITGYAYLLDKQYDSVFIGKGGGIQKGNDLYFQMYGLSRNR | glycoprotein\|Gene |
| | QSFKALCEHGSCLGTGGGGYQVLCDRAVMSFGSEESLITN | Symbol: G |
| | AYLKVNDLASGKPVIIGQTFPPSDSYKGSNGRMYTIGDKYG | |
| | LYLAPSSWNRYLRFGITPDISVRSTTWLKSQDPIMKILSTCT | |
| | NTDRDMCPEICNTRGYQDIFPLSEDSEYYTYIGITPNNGGTK | |
| | NFVAVRDSDGHIASIDILQNYYSITSATISCFMYKDEIWCIAI | |
| | TEGKKQKDNPQRIYAHSYKIRQMCYNMKSATVTVGNAKN | |
| | ITIRRY | |
| 18 | MLSQLQKNYLDNSNQQGDKMNNPDKKLSVNFNPLELDKG | gb:JQ001776: |
| | QKDLNKSYYVKNKNYNVSNLLNESLHDIKFCIYCIFSLLIIIT | 8170-10275\| |
| | IINIITISIVITRLKVHEENNGMESPNLQSIQDSLSSLTNMINTE | Organism: |
| | ITPRIGILVTATSVTLSSSINYVGTKTNQLVNELKDYITKSCG | Cedar virus\|Strain |
| | FKVPELKLHECNISCADPKISKSAMYSTNAYAELAGPPKIFC | Name: CG1a\|Protein |
| | KSVSKDPDFRLKQIDYVIPVQQDRSICMNNPLLDISDGFFTY | Name: attachment |
| | IHYEGINSCKKSDSFKVLLSHGEIVDRGDYRPSLYLLSSHYH | glycoprotein\|Gene |
| | PYSMQVINCVPVTCNQSSFVFCHISNNTKTLDNSDYSSDEY | Symbol: G |
| | YITYFNGIDRPKTKKIPINNMTADNRYIHFTFSGGGGVCLGE | |
| | EFIIPVTTVINTDVFTHDYCESFNCSVQTGKSLKEICSESLRS | |
| | PTNSSRYNLNGIMIISQNNMTDFKIQLNGITYNKLSFGSPGR | |
| | LSKTLGQVLYYQSSMSWDTYLKAGFVEKWKPFTPNWMN | |
| | NTVISRPNQGNCPRYHKCPEICYGGTYNDIAPLDLGKDMY | |
| | VSVILDSDQLAENPEITVFNSTTILYKERVSKDELNTRSTTTS | |
| | CFLFLDEPWCISVLETNRFNGKSIRPEIYSYKIPKYC | |
| 19 | MKKINE GLLDSKILSA FNTVIALLGS IVIIVMNIMI | (E501 A, W504A, |
| | IQNYTRSTDN QAVIKDALQG IQQQIKGLAD KIGTEIGPKV | Q530A, E533A) |
| | SLIDTSSTIT IPANIGLLGS KISQSTASIN ENVNEKCKFT | NiV G protein (Gc |
| | LPPLKIHECN ISCPNPLPFR EYRPQTEGVS NLVGLPNNIC | Δ 34) |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST | |
| | VGDPILNSTY WSGSLMMTRL AVKPKSNGGG | |
| | YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRFNTC | |
| | PAICAEGVYN DAFLIDRINW ISAGVFLDSN ATAANPVFTV | |
| | FKDNEILYRA QLASEDTNAQ KTITNCFLLK NKIWCISLVE | |
| | IYDTGDNVIR PKLFAVKIPE QC | |
| 20 | GGGGS | Linker |
| 21 | GGGGGS | Linker |
| 22 | (GGGGS)n | Linker |
| 23 | (GGGGGS)n | Linker |
| 24 | MALPVTALLLPLALLLHAARP | CD8α signal |
| | | peptide |
| 25 | METDTLLLWVLLLWVPGSTG | IgK signal peptide |
| 26 | MLLLVTSLLLCELPHPAFLLIP | GMCSFR-α |
| | | (CSF2RA) signal |
| | | peptide |
| 27 | TTTPAPRPPTPAPTIASQPLSLRPEACRPAAGGAVHTRGLDF | CD8α hinge |
| | ACD | domain |
| 28 | IEVMYPPPYLDNEKSNGTIIHVKGKHLCPSPLFPGPSKP | CD28 hinge |
| | | domain |
| 29 | AAAIEVMYPPPYLDNEKSNGTIIHVKGKHLCPSPLFPGPSKP | CD28 hinge |
| | | domain |
| 30 | ESKYGPPCPPCP | IgG4 hinge |
| | | domain |
| 31 | ESKYGPPCPSCP | IgG4 hinge |
| | | domain |
| 32 | ESKYGPPCPPCPAPEFLGGPSVFLFPPKPKDTLMISRTPEVTC | IgG4 hinge-CH2- |
| | VVVDVSQEDPEVQFNWYVDGVEVHNAKTKPREEQFNSTY | CH3 domain |
| | RVVSVLTVLHQDWLNGKEYKCKVSNKGLPSSIEKTISKAK | |
| | GQPREPQVYTLPPSQEEMTKNQVSLTCLVKGFYPSDIAVE | |
| | WESNGQPENNYKTTPPVLDSDGSFFLYSRLTVDKSRWQEG | |
| | NVFSCSVMHEALHNHYTQKSLSLSLGK | |
| 33 | IYIWAPLAGTCGVLLLSLVITLYC | CD8α |
| | | transmembrane |
| | | domain |
| 34 | FWVLVVVGGVLACYSLLVTVAFIIFWV | CD28 |
| | | transmembrane |
| | | domain |
| 35 | MFWVLVVVGGVLACYSLLVTVAFIIFWV | CD28 |
| | | transmembrane |
| | | domain |
| 36 | KRGRKKLLYIFKQPFMRPVQTTQEEDGCSCRFPEEEEGGCE | 4-1BB |
| | L | costimulatory |
| | | domain |
| 37 | RSKRSRLLHSDYMNMTPRRPGPTRKHYQPYAPPRDFAAYR | CD28 |
| | S | costimulatory |
| | | domain |
| 38 | RVKFSRSADAPAYQQGQNQLYNELNLGRREEYDVLDKRR | CD3ζ signaling |
| | GRDPEMGGKPRRKNPQEGLYNELQKDKMAEAYSEIGMKG | domain |
| | ERRRGKGHDGLYQGLSTATKDTYDALHMQALPPR | |
| 39 | RVKFSRSADAPAYKQGQNQLYNELNLGRREEYDVLDKRR | CD3ζ signaling |
| | GRDPEMGGKPRRKNPQEGLYNELQKDKMAEAYSEIGMKG | domain (with Q to |
| | ERRRGKGHDGLYQGLSTATKDTYDALHMQALPPR | K mutation at |
| | | position 14) |
| 40 | DIQMTQTTSSLSASLGDRVTISCRASQDISKYLNWYQQKPD | Anti-CD19 |
| | GTVKLLIYHTSRLHSGVPSRFSGSGSGTDYSLTISNLEQEDI | FMC63 scFv |
| | ATYFCQQGNTLPYTFGGGTKLEITGSTSGSGKPGSGEGSTK | entire sequence, |
| | GEVKLQESGPGLVAPSQSLSVTCTVSGVSLPDYGVSWIRQP | with Whitlow |
| | PRKGLEWLGVIWGSETTYYNSALKSRLTIIKDNSKSQVFLK | linker |
| | MNSLQTDDTAIYYCAKHYYYGGSYAMDYWGQGTSVTVS | |
| | S | |
| 41 | DIQMTQTTSSLSASLGDRVTISCRASQDISKYLNWYQQKPD | Anti-CD19 |
| | GTVKLLIYHTSRLHSGVPSRFSGSGSGTDYSLTISNLEQEDI | FMC63 scFv light |
| | ATYFCQQGNTLPYTFGGGTKLEIT | chain variable |
| | | region |
| 42 | QDISKY | Anti-CD19 |
| | | FMC63 scFv light |
| | | chain CDR1 |
| 43 | HTS | Anti-CD19 |
| | | FMC63 scFv light |
| | | chain CDR2 |
| 44 | QQGNTLPYT | Anti-CD19 |
| | | FMC63 scFv light |
| | | chain CDR3 |
| 45 | GSTSGSGKPGSGEGSTKG | Whitlow linker |
| 46 | EVKLQESGPGLVAPSQSLSVTCTVSGVSLPDYGVSWIRQPP | Anti-CD19 |
| | RKGLEWLGVIWGSETTYYNSALKSRLTIIKDNSKSQVFLKM | FMC63 scFv |
| | NSLQTDDTAIYYCAKHYYYGGSYAMDYWGQGTSVTVSS | heavy chain |
| | | variable region |
| 47 | GVSLPDYG | Anti-CD19 |
| | | FMC63 scFv |
| | | heavy chain |
| | | CDR1 |
| 48 | IWGSETT | Anti-CD19 |
| | | FMC63 scFv |
| | | heavy chain |
| | | CDR2 |
| 49 | AKHYYYGGSYAMDY | Anti-CD19 |
| | | FMC63 scFv |
| | | heavy chain |
| | | CDR3 |
| 50 | DIQMTQTTSSLSASLGDRVTISCRASQDISKYLNWYQQKPD | Anti-CD19 |
| | GTVKLLIYHTSRLHSGVPSRFSGSGSGTDYSLTISNLEQEDI | FMC63 scFv |
| | ATYFCQQGNTLPYTFGGGTKLEITGGGGSGGGGSGGGGSE | entire sequence, |
| | VKLQESGPGLVAPSQSLSVTCTVSGVSLPDYGVSWIRQPPR | with 3xG₄S linker |
| | KGLEWLGVIWGSETTYYNSALKSRLTIIKDNSKSQVFLKM | |
| | NSLQTDDTAIYYCAKHYYYGGSYAMDYWGQGTSVTVSS | |
| 51 | GGGGSGGGGSGGGGS | 3xG₄S linker |
| 52 | atggccttaccagtgaccgccttgctcctgccgctggccttgctgctccacgccgccaggcc | Exemplary CD19 |
| | ggacatccagatgacacagactacatcctccctgtctgcctctctgggagacagagtcacca | CAR nucleotide |
| | tcagttgcagggcaagtcaggacattagtaaatatttaaattggtatcagcagaaaccagat | sequence |
| | ggaactgttaaactcctgatctaccatacatcaagattacactcaggagtcccatcaaggtt | |
| | cagtggcagtgggtctggaacagattattctctcaccattagcaacctggagcaagaagata | |
| | ttgccacttacttttgccaacagggtaatacgcttccgtacacgttcggaggggggaccaag | |
| | ctggagatcacaggctccacctctggatccggcaagcccggatctggcgagggatccaccaa | |
| | gggcgaggtgaaactgcaggagtcaggacctggcctggtggcgccctcacagagcctgtccg | |
| | tcacatgcactgtctcaggggtctcattacccgactatggtgtaagctggattcgccagcct | |
| | ccacgaaagggtctggagtggctgggagtaatatggggtagtgaaaccacatactataattc | |
| | agctctcaaatccagactgaccatcatcaaggacaactccaagagccaagttttcttaaaaa | |
| | tgaacagtctgcaaactgatgacacagccatttactactgtgccaaacattattactacggt | |
| | ggtagctatgctatggactactggggccaaggaacctcagtcaccgtctcctcaaccacgac | |
| | gccagcgccgcgaccaccaacaccggcgcccaccatcgcgtcgcagcccctgtccctgcgcc | |
| | cagaggcgtgccggccagcggggggggcgcagtgcacacgagggggctggacttcgcctgtg | |
| | atatctacatctgggcgcccttggccgggacttgtggggtccttctcctgtcactggttatc | |
| | accctttactgcaaacggggcagaaagaaactcctgtatatattcaaacaaccatttatgag | |
| | accagtacaaactactcaagaggaagatggctgtagctgccgatttccagaagaagaagaag | |
| | gaggatgtgaactgagagtgaagttcagcaggagcgcagacgcccccgcgtaccagcagggc | |
| | cagaaccagctctataacgagctcaatctaggacgaagagaggagtacgatgttttggacaa | |
| | gagacgtggccgggaccctgagatggggggaaagccgagaaggaagaaccctcaggaaggcc | |
| | tgtacaatgaactgcagaaagataagatggcggaggcctacagtgagattgggatgaaaggc | |
| | gagcgccggaggggcaaggggcacgatggcctttaccagggtctcagtacagccaccaagga | |
| | cacctacgacgcccttcacatgcaggccctgccccctcgc | |
| 53 | MALPVTALLLPLALLLHAARPDIQMTQTTSSLSASLGDRVT | Exemplary CD19 |
| | ISCRASQDISKYLNWYQQKPDGTVKLLIYHTSRLHSGVPSR | CAR amino acid |
| | FSGSGSGTDYSLTISNLEQEDIATYFCQQGNTLPYTFGGGTK | sequence |
| | LEITGSTSGSGKPGSGEGSTKGEVKLQESGPGLVAPSQSLSV | |
| | TCTVSGVSLPDYGVSWIRQPPRKGLEWLGVIWGSETTYYN | |
| | SALKSRLTIIKDNSKSQVFLKMNSLQTDDTAIYYCAKHYYY | |
| | GGSYAMDYWGQGTSVTVSSTTTPAPRPPTPAPTIASQPLSL | |
| | RPEACRPAAGGAVHTRGLDFACDIYIWAPLAGTCGVLLLS | |
| | LVITLYCKRGRKKLLYIFKQPFMRPVQTTQEEDGCSCRFPE | |
| | EEEGGCELRVKFSRSADAPAYQQGQNQLYNELNLGRREEY | |
| | DVLDKRRGRDPEMGGKPRRKNPQEGLYNELQKDKMAEA | |
| | YSEIGMKGERRRGKGHDGLYQGLSTATKDTYDALHMQAL | |
| | PPR | |
| 54 | atggccttaccagtgaccgccttgctcctgccgctggccttgctgctccacgccgccaggc | Tisagenlecleucel |
| | cggacatccagatgacacagactacatcctccctgtctgcctctctgggagacagagtcacc | CD19 CAR |
| | atcagttgcagggcaagtcaggacattagtaaatatttaaattggtatcagcagaaaccaga | nucleotide |
| | tggaactgttaaactcctgatctaccatacatcaagattacactcaggagtcccatcaaggt | sequence |
| | tcagtggcagtgggtctggaacagattattctctcaccattagcaacctggagcaagaagat | |
| | attgccacttacttttgccaacagggtaatacgcttccgtacacgttcggaggggggaccaa | |
| | gctggagatcacaggtggcggtggctcgggcggtggtgggtcgggtggcggcggatctgagg | |
| | tgaaactgcaggagtcaggacctggcctggtggcgccctcacagagcctgtccgtcacatgc | |
| | actgtctcaggggtctcattacccgactatggtgtaagctggattcgccagcctccacgaaa | |
| | gggtctggagtggctgggagtaatatggggtagtgaaaccacatactataattcagctctca | |
| | aatccagactgaccatcatcaaggacaactccaagagccaagttttcttaaaaatgaacagt | |
| | ctgcaaactgatgacacagccatttactactgtgccaaacattattactacggtggtagcta | |
| | tgctatggactactggggccaaggaacctcagtcaccgtctcctcaaccacgacgccagcgc | |
| | cgcgaccaccaacaccggcgcccaccatcgcgtcgcagcccctgtccctgcgcccagaggcg | |
| | tgccggccagcggcggggggcgcagtgcacacgagggggctggacttcgcctgtgatatcta | |
| | catctgggcgcccttggccgggacttgtggggtccttctcctgtcactggttatcacccttt | |
| | actgcaaacggggcagaaagaaactcctgtatatattcaaacaaccatttatgagaccagta | |
| | caaactactcaagaggaagatggctgtagctgccgatttccagaagaagaagaaggaggatg | |
| | tgaactgagagtgaagttcagcaggagcgcagacgcccccgcgtacaagcagggccagaacc | |
| | agctctataacgagctcaatctaggacgaagagaggagtacgatgttttggacaagagacgt | |
| | ggccgggaccctgagatggggggaaagccgagaaggaagaaccctcaggaaggcctgtacaa | |
| | tgaactgcagaaagataagatggcggaggcctacagtgagattgggatgaaaggcgagcgcc | |
| | ggaggggcaaggggcacgatggcctttaccagggtctcagtacagccaccaaggacacctac | |
| | gacgcccttcacatgcaggccctgccccctcgc | |
| 55 | MALPVTALLLPLALLLHAARPDIQMTQTTSSLSASLGDRVT | Tisagenlecleucel |
| | ISCRASQDISKYLNWYQQKPDGTVKLLIYHTSRLHSGVPSR | CD19 CAR amino |
| | FSGSGSGTDYSLTISNLEQEDIATYFCQQGNTLPYTFGGGTK | acid sequence |
| | LEITGGGGSGGGGSGGGGSEVKLQESGPGLVAPSQSLSVTC | |
| | TVSGVSLPDYGVSWIRQPPRKGLEWLGVIWGSETTYYNSA | |
| | LKSRLTIIKDNSKSQVFLKMNSLQTDDTAIYYCAKHYYYG | |
| | GSYAMDYWGQGTSVTVSSTTTPAPRPPTPAPTIASQPLSLR | |
| | PEACRPAAGGAVHTRGLDFACDIYIWAPLAGTCGVLLLSL | |
| | VITLYCKRGRKKLLYIFKQPFMRPVQTTQEEDGCSCRFPEE | |
| | EEGGCELRVKFSRSADAPAYKQGQNQLYNELNLGRREEYD | |
| | VLDKRRGRDPEMGGKPRRKNPQEGLYNELQKDKMAEAYS | |
| | EIGMKGERRRGKGHDGLYQGLSTATKDTYDALHMQALPP | |
| | R | |
| 56 | atgctgctgctggtgaccagcctgctgctgtgcgagctgccccaccccgcctttctgctgat | Lisocabtagene |
| | ccccgacatccagatgacccagaccacctccagcctgagcgccagcctgggcgaccgggtga | maraleucel CD19 |
| | ccatcagctgccgggccagccaggacatcagcaagtacctgaactggtatcagcagaagcc | CAR nucleotide |
| | cgacggcaccgtcaagctgctgatctaccacaccagccggctgcacagcggcgtgcccag | sequence |
| | ccggtttagcggcagcggctccggcaccgactacagcctgaccatctccaacctggaacag | |
| | gaagatatcgccacctacttttgccagcagggcaacacactgccctacacctttggcggcgg | |
| | aacaaagctggaaatcaccggcagcacctccggcagcggcaagcctggcagcggcgaggg | |
| | cagcaccaagggcgaggtgaagctgcaggaaagcggccctggcctggtggcccccagcc | |
| | agagcctgagcgtgacctgcaccgtgagcggcgtgagcctgcccgactacggcgtgagct | |
| | ggatccggcagccccccaggaagggcctggaatggctgggcgtgatctggggcagcgag | |
| | accacctactacaacagcgccctgaagagccggctgaccatcatcaaggacaacagcaaga | |
| | gccaggtgttcctgaagatgaacagcctgcagaccgacgacaccgccatctactactgcgcc | |
| | aagcactactactacggcggcagctacgccatggactactggggccagggcaccagcgtga | |
| | ccgtgagcagcgaatctaagtacggaccgccctgccccccttgccctatgttctgggtgctg | |
| | gtggtggtcggaggcgtgctggcctgctacagcctgctggtcaccgtggccttcatcatctt | |
| | ttgggtgaaacggggcagaaagaaactcctgtatatattcaaacaaccatttatgagaccag | |
| | tacaaactactcaagaggaagatggctgtagctgccgatttccagaagaagaagaaggagga | |
| | tgtgaactgcgggtgaagttcagcagaagcgccgacgcccctgcctaccagcagggccagaa | |
| | tcagctgtacaacgagctgaacctgggcagaagggaagagtacgacgtcctggataagcgga | |
| | gaggccgggaccctgagatgggcggcaagcctcggcggaagaacccccaggaaggcct | |
| | gtataacgaactgcagaaagacaagatggccgaggcctacagcgagatcggcatgaaggg | |
| | cgagcggaggcggggcaagggccacgacggcctgtatcagggcctgtccaccgccacca | |
| | aggatacctacgacgccctgcacatgcaggccctgcccccaagg | |
| 57 | MLLLVTSLLLCELPHPAFLLIPDIQMTQTTSSLSASLGDRVTI | Lisocabtagene |
| | SCRASQDISKYLNWYQQKPDGTVKLLIYHTSRLHSGVPSRF | maraleucel CD19 |
| | SGSGSGTDYSLTISNLEQEDIATYFCQQGNTLPYTFGGGTKL | CAR amino acid |
| | EITGSTSGSGKPGSGEGSTKGEVKLQESGPGLVAPSQSLSVT | sequence |
| | CTVSGVSLPDYGVSWIRQPPRKGLEWLGVIWGSETTYYNS | |
| | ALKSRLTIIKDNSKSQVFLKMNSLQTDDTAIYYCAKHYYY | |
| | GGSYAMDYWGQGTSVTVSSESKYGPPCPPCPMFWVLVVV | |
| | GGVLACYSLLVTVAFIIFWVKRGRKKLLYIFKQPFMRPVQT | |
| | TQEEDGCSCRFPEEEEGGCELRVKFSRSADAPAYQQGQNQ | |
| | LYNELNLGRREEYDVLDKRRGRDPEMGGKPRRKNPQEGL | |
| | YNELQKDKMAEAYSEIGMKGERRRGKGHDGLYQGLSTAT | |
| | KDTYDALHMQALPPR | |
| 58 | atgcttctcctggtgacaagccttctgctctgtgagttaccacacccagcattcctcctgat | Axicabtagene |
| | cccagacatccagatgacacagactacatcctccctgtctgcctctctgggagacagagtca | ciloleucel CD19 |
| | ccatcagttgcagggcaagtcaggacattagtaaatatttaaattggtatcagcagaaacca | CAR nucleotide |
| | gatggaactgttaaactcctgatctaccatacatcaagattacactcaggagtcccatcaag | sequence |
| | gttcagtggcagtgggtctggaacagattattctctcaccattagcaacctggagcaagaag | |
| | atattgccacttacttttgccaacagggtaatacgcttccgtacacgttcggaggggggact | |
| | aagttggaaataacaggctccacctctggatccggcaagcccggatctggcgagggatccac | |
| | caagggcgaggtgaaactgcaggagtcaggacctggcctggtggcgccctcacagagcctgt | |
| | ccgtcacatgcactgtctcaggggtctcattacccgactatggtgtaagctggattcgccag | |
| | cctccacgaaagggtctggagtggctgggagtaatatggggtagtgaaaccacatactataa | |
| | ttcagctctcaaatccagactgaccatcatcaaggacaactccaagagccaagttttcttaa | |
| | aaatgaacagtctgcaaactgatgacacagccatttactactgtgccaaacattattactac | |
| | ggtggtagctatgctatggactactggggtcaaggaacctcagtcaccgtctcctcagcggc | |
| | cgcaattgaagttatgtatcctcctccttacctagacaatgagaagagcaatggaaccatta | |
| | tccatgtgaaagggaaacacctttgtccaagtcccctatttcccggaccttctaagcccttt | |
| | tgggtgctggtggtggttgggggagtcctggcttgctatagcttgctagtaacagtggcctt | |
| | tattattttctgggtgaggagtaagaggagcaggctcctgcacagtgactacatgaacatga | |
| | ctccccgccgccccgggcccacccgcaagcattaccagccctatgccccaccacgcgacttc | |
| | gcagcctatcgctccagagtgaagttcagcaggagcgcagacgcccccgcgtaccagcaggg | |
| | ccagaaccagctctataacgagctcaatctaggacgaagagaggagtacgatgttttggaca | |
| | agagacgtggccgggaccctgagatggggggaaagccgagaaggaagaaccctcaggaaggc | |
| | ctgtacaatgaactgcagaaagataagatggcggaggcctacagtgagattgggatgaaagg | |
| | cgagcgccggaggggcaaggggcacgatggcctttaccagggtctcagtacagccaccaagg | |
| | acacctacgacgcccttcacatgcaggccctgccccctcgc | |
| 59 | MLLLVTSLLLCELPHPAFLLIPDIQMTQTTSSLSASLGDRVTI | Axicabtagene |
| | SCRASQDISKYLNWYQQKPDGTVKLLIYHTSRLHSGVPSRF | ciloleucel CD19 |
| | SGSGSGTDYSLTISNLEQEDIATYFCQQGNTLPYTFGGGTKL | CAR amino acid |
| | EITGSTSGSGKPGSGEGSTKGEVKLQESGPGLVAPSQSLSVT | sequence |
| | CTVSGVSLPDYGVSWIRQPPRKGLEWLGVIWGSETTYYNS | |
| | ALKSRLTIIKDNSKSQVFLKMNSLQTDDTAIYYCAKHYYY | |
| | GGSYAMDYWGQGTSVTVSSAAAIEVMYPPPYLDNEKSNG | |
| | TIIHVKGKHLCPSPLFPGPSKPFWVLVVVGGVLACYSLLVT | |
| | VAFIIFWVRSKRSRLLHSDYMNMTPRRPGPTRKHYQPYAPP | |
| | RDFAAYRSRVKFSRSADAPAYQQGQNQLYNELNLGRREE | |
| | YDVLDKRRGRDPEMGGKPRRKNPQEGLYNELQKDKMAE | |
| | AYSEIGMKGERRRGKGHDGLYQGLSTATKDTYDALHMQA | |
| | LPPR | |
| 60 | DIVLTQSPAILSASPGEKVTMTCRASSSVNYMDWYQKKPG | Anti-CD20 Leu16 |
| | SSPKPWIYATSNLASGVPARFSGSGSGTSYSLTISRVEAEDA | scFv entire |
| | ATYYCQQWSFNPPTFGGGTKLEIKGSTSGSGKPGSGEGSTK | sequence, with |
| | GEVQLQQSGAELVKPGASVKMSCKASGYTFTSYNMHWVK | Whitlow linker |
| | QTPGQGLEWIGAIYPGNGDTSYNQKFKGKATLTADKSSST | |
| | AYMQLSSLTSEDSADYYCARSNYYGSSYWFFDVWGAGTT | |
| | VTVSS | |
| 61 | DIVLTQSPAILSASPGEKVTMTCRASSSVNYMDWYQKKPG | Anti-CD20 Leu16 |
| | SSPKPWIYATSNLASGVPARFSGSGSGTSYSLTISRVEAEDA | scFv light chain |
| | ATYYCQQWSFNPPTFGGGTKLEIK | variable region |
| 62 | RASSSVNYMD | Anti-CD20 Leu16 |
| | | scFv light chain |
| | | CDR1 |
| 63 | ATSNLAS | Anti-CD20 Leu16 |
| | | scFv light chain |
| | | CDR2 |
| 64 | QQWSFNPPT | Anti-CD20 Leu16 |
| | | scFv light chain |
| | | CDR3 |
| 65 | EVQLQQSGAELVKPGASVKMSCKASGYTFTSYNMHWVKQ | Anti-CD20 Leu16 |
| | TPGQGLEWIGAIYPGNGDTSYNQKFKGKATLTADKSSSTA | scFv heavy chain |
| | YMQLSSLTSEDSADYYCARSNYYGSSYWFFDVWGAGTTV | |
| | TVSS | |
| 66 | SYNMH | Anti-CD20 Leu16 |
| | | scFv heavy chain |
| | | CDR1 |
| 67 | AIYPGNGDTSYNQKFKG | Anti-CD20 Leu16 |
| | | scFv heavy chain |
| | | CDR2 |
| 68 | SNYYGSSYWFFDV | Anti-CD20 Leu16 |
| | | scFv heavy chain |
| | | CDR3 |
| 69 | QVQLQQSGPGLVKPSQTLSLTCAISGDSVSSNSAAWNWIRQ | Anti-CD22 m971 |
| | SPSRGLEWLGRTYYRSKWYNDYAVSVKSRITINPDTSKNQ | scFv entire |
| | FSLQLNSVTPEDTAVYYCAREVTGDLEDAFDIWGQGTMVT | sequence, with |
| | VSSGGGGSGGGGSGGGGSDIQMTQSPSSLSASVGDRVTITC | 3xG4S linker |
| | RASQTIWSYLNWYQQRPGKAPNLLIYAASSLQSGVPSRFSG | |
| | RGSGTDFTLTISSLQAEDFATYYCQQSYSIPQTFGQGTKLEI | |
| | K | |
| 70 | QVQLQQSGPGLVKPSQTLSLTCAISGDSVSSNSAAWNWIRQ | Anti-CD22 m971 |
| | SPSRGLEWLGRTYYRSKWYNDYAVSVKSRITINPDTSKNQ | scFv heavy chain |
| | FSLQLNSVTPEDTAVYYCAREVTGDLEDAFDIWGQGTMVT | variable region |
| | VSS | |
| 71 | GDSVSSNSAA | Anti-CD22 m971 |
| | | scFv heavy chain |
| | | CDR1 |
| 72 | TYYRSKWYN | Anti-CD22 m971 |
| | | scFv heavy chain |
| | | CDR2 |
| 73 | AREVTGDLEDAFDI | Anti-CD22 m971 |
| | | scFv heavy chain |
| | | CDR3 |
| 74 | DIQMTQSPSSLSASVGDRVTITCRASQTIWSYLNWYQQRPG | Anti-CD22 m971 |
| | KAPNLLIYAASSLQSGVPSRFSGRGSGTDFTLTISSLQAEDF | scFv light chain |
| | ATYYCQQSYSIPQTFGQGTKLEIK | |
| 75 | QTIWSY | Anti-CD22 m971 |
| | | scFv light chain |
| | | CDR1 |
| 76 | AAS | Anti-CD22 m971 |
| | | scFv light chain |
| | | CDR2 |
| 77 | QQSYSIPQT | Anti-CD22 m971 |
| | | scFv light chain |
| | | CDR3 |
| 78 | QVQLQQSGPGMVKPSQTLSLTCAISGDSVSSNSVAWNWIR | Anti-CD22 m971- |
| | QSPSRGLEWLGRTYYRSTWYNDYAVSMKSRITINPDTNKN | L7 scFv entire |
| | QFSLQLNSVTPEDTAVYYCAREVTGDLEDAFDIWGQGTMV | sequence, with |
| | TVSSGGGGSGGGGSGGGGSDIQMIQSPSSLSASVGDRVTIT | 3xG₄S linker |
| | CRASQTIWSYLNWYRQRPGEAPNLLIYAASSLQSGVPSRFS | |
| | GRGSGTDFTLTISSLQAEDFATYYCQQSYSIPQTFGQGTKLE | |
| | IK | |
| 79 | QVQLQQSGPGMVKPSQTLSLTCAISGDSVSSNSVAWNWIR | Anti-CD22 m971- |
| | QSPSRGLEWLGRTYYRSTWYNDYAVSMKSRITINPDTNKN | L7 scFv heavy |
| | QFSLQLNSVTPEDTAVYYCAREVTGDLEDAFDIWGQGTMV | chain variable |
| | TVSS | region |
| 80 | GDSVSSNSVA | Anti-CD22 m971- |
| | | L7 scFv heavy |
| | | chain CDR1 |
| 81 | TYYRSTWYN | Anti-CD22 m971- |
| | | L7 scFv heavy |
| | | chain CDR2 |
| 82 | AREVTGDLEDAFDI | Anti-CD22 m971- |
| | | L7 scFv heavy |
| | | chain CDR3 |
| 83 | DIQMIQSPSSLSASVGDRVTITCRASQTIWSYLNWYRQRPG | Anti-CD22 m971- |
| | EAPNLLIYAASSLQSGVPSRFSGRGSGTDFTLTISSLQAEDF | L7 scFv light |
| | ATYYCQQSYSIPQTFGQGTKLEIK | chain variable |
| | | region |
| 84 | QTIWSY | Anti-CD22 m971- |
| | | L7 scFv light |
| | | chain CDR1 |
| 85 | AAS | Anti-CD22 m971- |
| | | L7 scFv light |
| | | chain CDR2 |
| 86 | QQSYSIPQT | Anti-CD22 m971- |
| | | L7 scFv light |
| | | chain CDR3 |
| 87 | DIVLTQSPASLAMSLGKRATISCRASESVSVIGAHLIHWYQ | Anti-BCMA |
| | QKPGQPPKLLIYLASNLETGVPARFSGSGSGTDFTLTIDPVE | C11D5.3 scFv |
| | EDDVAIYSCLQSRIFPRTFGGGTKLEIKGSTSGSGKPGSGEG | entire sequence, |
| | STKGQIQLVQSGPELKKPGETVKISCKASGYTFTDYSINWV | with Whitlow |
| | KRAPGKGLKWMGWINTETREPAYAYDFRGRFAFSLETSAS | linker |
| | TAYLQINNLKYEDTATYFCALDYSYAMDYWGQGTSVTVS | |
| | S | |
| 88 | DIVLTQSPASLAMSLGKRATISCRASESVSVIGAHLIHWYQ | Anti-BCMA |
| | QKPGQPPKLLIYLASNLETGVPARFSGSGSGTDFTLTIDPVE | C11D5.3 scFv |
| | EDDVAIYSCLQSRIFPRTFGGGTKLEIK | light chain |
| | | variable region |
| 89 | RASESVSVIGAHLIH | Anti-BCMA |
| | | C11D5.3 scFv |
| | | light chain CDR1 |
| 90 | LASNLET | Anti-BCMA |
| | | C11D5.3 scFv |
| | | light chain CDR2 |
| 91 | LQSRIFPRT | Anti-BCMA |
| | | C11D5.3 scFv |
| | | light chain CDR3 |
| 92 | QIQLVQSGPELKKPGETVKISCKASGYTFTDYSINWVKRAP | Anti-BCMA |
| | GKGLKWMGWINTETREPAYAYDFRGRFAFSLETSASTAYL | C11D5.3 scFv |
| | QINNLKYEDTATYFCALDYSYAMDYWGQGTSVTVSS | heavy chain |
| | | variable region |
| 93 | DYSIN | Anti-BCMA |
| | | C11D5.3 scFv |
| | | heavy chain |
| | | CDR1 |
| 94 | WINTETREPAYAYDFRG | Anti-BCMA |
| | | C11D5.3 scFv |
| | | heavy chain |
| | | CDR2 |
| 95 | DYSYAMDY | Anti-BCMA |
| | | C11D5.3 scFv |
| | | heavy chain |
| | | CDR3 |
| 96 | DIVLTQSPPSLAMSLGKRATISCRASESVTILGSHLIYWYQQ | Anti-BCMA |
| | KPGQPPTLLIQLASNVQTGVPARFSGSGSRTDFTLTIDPVEE | C12A3.2 scFv |
| | DDVAVYYCLQSRTIPRTFGGGTKLEIKGSTSGSGKPGSGEG | entire sequence, |
| | STKGQIQLVQSGPELKKPGETVKISCKASGYTFRHYSMNW | with Whitlow |
| | VKQAPGKGLKWMGRINTESGVPIYADDFKGRFAFSVETSA | linker |
| | STAYLVINNLKDEDTASYFCSNDYLYSLDFWGQGTALTVS | |
| | S | |
| 97 | DIVLTQSPPSLAMSLGKRATISCRASESVTILGSHLIYWYQQ | Anti-BCMA |
| | KPGQPPTLLIQLASNVQTGVPARFSGSGSRTDFTLTIDPVEE | C12A3.2 scFv |
| | DDVAVYYCLQSRTIPRTFGGGTKLEIK | light chain |
| | | variable region |
| 98 | RASESVTILGSHLIY | Anti-BCMA |
| | | C12A3.2 scFv |
| | | light chain CDR1 |
| 99 | LASNVQT | Anti-BCMA |
| | | C12A3.2 scFv |
| | | light chain CDR2 |
| 100 | LQSRTIPRT | Anti-BCMA |
| | | C12A3.2 scFv |
| | | light chain CDR3 |
| 101 | QIQLVQSGPELKKPGETVKISCKASGYTFRHYSMNWVKQA | Anti-BCMA |
| | PGKGLKWMGRINTESGVPIYADDFKGRFAFSVETSASTAYL | C12A3.2 scFv |
| | VINNLKDEDTASYFCSNDYLYSLDFWGQGTALTVSS | heavy chain |
| | | variable region |
| 102 | HYSMN | Anti-BCMA |
| | | C12A3.2 scFv |
| | | heavy chain |
| | | CDR1 |
| 103 | RINTESGVPIYADDFKG | Anti-BCMA |
| | | C12A3.2 scFv |
| | | heavy chain |
| | | CDR2 |
| 104 | DYLYSLDF | Anti-BCMA |
| | | C12A3.2 scFv |
| | | heavy chain |
| | | CDR3 |
| 105 | EVQLLESGGGLVQPGGSLRLSCAASGFTFSSYAMSWVRQA | Anti-BCMA |
| | PGKGLEWVSSISGSGDYIYYADSVKGRFTISRDISKNTLYLQ | FHVH33 entire |
| | MNSLRAEDTAVYYCAKEGTGANSSLADYRGQGTLVTVSS | sequence |
| 106 | GFTFSSYA | Anti-BCMA |
| | | FHVH33 CDR1 |
| 107 | ISGSGDYI | Anti-BCMA |
| | | FHVH33 CDR2 |
| 108 | AKEGTGANSSLADY | Anti-BCMA |
| | | FHVH33 CDR3 |
| 109 | DIQMTQSPSSLSASVGDRVTITCRASQSISSYLNWYQQKPG | Anti-BCMA |
| | KAPKLLIYAASSLQSGVPSRFSGSGSGTDFTLTISSLQPEDFA | CT103A scFv |
| | TYYCQQKYDLLTFGGGTKVEIKGSTSGSGKPGSGEGSTKG | entire sequence, |
| | QLQLQESGPGLVKPSETLSLTCTVSGGSISSSSYYWGWIRQP | with Whitlow |
| | PGKGLEWIGSISYSGSTYYNPSLKSRVTISVDTSKNQFSLKL | linker |
| | SSVTAADTAVYYCARDRGDTILDVWGQGTMVTVSS | |
| 110 | DIQMTQSPSSLSASVGDRVTITCRASQSISSYLNWYQQKPG | Anti-BCMA |
| | KAPKLLIYAASSLQSGVPSRFSGSGSGTDFTLTISSLQPEDFA | CT103A scFv |
| | TYYCQQKYDLLTFGGGTKVEIK | light chain |
| | | variable region |
| 111 | QSISSY | Anti-BCMA |
| | | CT103A scFv |
| | | light chain CDR1 |
| 112 | AAS | Anti-BCMA |
| | | CT103A scFv |
| | | light chain CDR2 |
| 113 | QQKYDLLT | Anti-BCMA |
| | | CT103A scFv |
| | | light chain CDR3 |
| 114 | QLQLQESGPGLVKPSETLSLTCTVSGGSISSSSYYWGWIRQP | Anti-BCMA |
| | PGKGLEWIGSISYSGSTYYNPSLKSRVTISVDTSKNQFSLKL | CT103A scFv |
| | SSVTAADTAVYYCARDRGDTILDVWGQGTMVTVSS | heavy chain |
| | | variable region |
| 115 | GGSISSSSYY | Anti-BCMA |
| | | CT103A scFv |
| | | heavy chain |
| | | CDR1 |
| 116 | ISYSGST | Anti-BCMA |
| | | CT103A scFv |
| | | heavy chain |
| | | CDR2 |
| 117 | ARDRGDTILDV | Anti-BCMA |
| | | CT103A scFv |
| | | heavy chain |
| | | CDR3 |
| 118 | atggccttaccagtgaccgccttgctcctgccgctggccttgctgctccacgccgccaggc | Exemplary BCMA |
| | cggacatccagatgacccagtctccatcctccctgtctgcatctgtaggagacagagtcacc | CAR nucleotide |
| | atcacttgccgggcaagtcagagcattagcagctatttaaattggtatcagcagaaaccagg | sequence |
| | gaaagcccctaagctcctgatctatgctgcatccagtttgcaaagtggggtcccatcaaggt | |
| | tcagtggcagtggatctgggacagatttcactctcaccatcagcagtctgcaacctgaagat | |
| | tttgcaacttactactgtcagcaaaaatacgacctcctcacttttggcggagggaccaaggt | |
| | tgagatcaaaggcagcaccagcggctccggcaagcctggctctggcgagggcagcacaaagg | |
| | gacagctgcagctgcaggagtcgggcccaggactggtgaagccttcggagaccctgtccctc | |
| | acctgcactgtctctggtggctccatcagcagtagtagttactactggggctggatccgcca | |
| | gcccccagggaaggggctggagtggattgggagtatctcctatagtgggagcacctactaca | |
| | acccgtccctcaagagtcgagtcaccatatccgtagacacgtccaagaaccagttctccctg | |
| | aagctgagttctgtgaccgccgcagacacggcggtgtactactgcgccagagatcgtggaga | |
| | caccatactagacgtatggggtcagggtacaatggtcaccgtcagctcattcgtgcccgtgt | |
| | tcctgcccgccaaacctaccaccacccctgcccctagacctcccaccccagccccaacaatc | |
| | gccagccagcctctgtctctgcggcccgaagcctgtagacctgctgccggcggagccgtgca | |
| | caccagaggcctggacttcgcctgcgacatctacatctgggcccctctggccggcacctgtg | |
| | gcgtgctgctgctgagcctggtgatcaccctgtactgcaaccaccggaacaaacggggcaga | |
| | aagaaactcctgtatatattcaaacaaccatttatgagaccagtacaaactactcaagagga | |
| | agatggctgtagctgccgatttccagaagaagaagaaggaggatgtgaactgagagtgaagt | |
| | tcagcagatccgccgacgcccctgcctaccagcagggacagaaccagctgtacaacgagctg | |
| | aacctgggcagacgggaagagtacgacgtgctggacaagcggagaggccgggaccccgagat | |
| | gggcggaaagcccagacggaagaacccccaggaaggcctgtataacgaactgcagaaagaca | |
| | agatggccgaggcctacagcgagatcggcatgaagggcgagcggaggcgcggcaagggcc | |
| | acgatggcctgtaccagggcctgagcaccgccaccaaggacacctacgacgccctgcacat | |
| | gcaggccctgccccccaga | |
| 119 | MALPVTALLLPLALLLHAARPDIQMTQSPSSLSASVGDRVT | Exemplary BCMA |
| | ITCRASQSISSYLNWYQQKPGKAPKLLIYAASSLQSGVPSRF | CAR amino acid |
| | SGSGSGTDFTLTISSLQPEDFATYYCQQKYDLLTFGGGTKV | sequence |
| | EIKGSTSGSGKPGSGEGSTKGQLQLQESGPGLVKPSETLSLT | |
| | CTVSGGSISSSSYYWGWIRQPPGKGLEWIGSISYSGSTYYNP | |
| | SLKSRVTISVDTSKNQFSLKLSSVTAADTAVYYCARDRGDT | |
| | ILDVWGQGTMVTVSSFVPVFLPAKPTTTPAPRPPTPAPTIAS | |
| | QPLSLRPEACRPAAGGAVHTRGLDFACDIYIWAPLAGTCG | |
| | VLLLSLVITLYCNHRNKRGRKKLLYIFKQPFMRPVQTTQEE | |
| | DGCSCRFPEEEEGGCELRVKFSRSADAPAYQQGQNQLYNE | |
| | LNLGRREEYDVLDKRRGRDPEMGGKPRRKNPQEGLYNEL | |
| | QKDKMAEAYSEIGMKGERRRGKGHDGLYQGLSTATKDTY | |
| | DALHMQALPPR | |
| 120 | QVQLVQSGAEVKKPGASVKVSCKASGGTFSSYAISWVRQA | CD8_1 VH |
| | PGQGLEWMGIIDPSDGNTNYAQNFQGRVTMTRDTSTSTVY | |
| | MELSSLRSEDTAVYYCAKERAAAGYYYYMDVWGQGTTV | |
| | TVSS | |
| 121 | DIQMTQSPSSLSASVGDRVTITCRASQSISSYLNWYQQKPG | CD8_1 VL |
| | KAPKLLIYAASSLQSGVPSRFSGSGSGTDFTLTISSLQPEDFA | |
| | TYYCQQSYSTPLTFGGGTKVEIKR | |
| 122 | QVQLVQSGAEVKKPGASVKVSCKASGYTFTDYYIQWVRQ | CD8_2 VH |
| | APGQGLEWMGWINPNSGGTSYAQKFQGRVTMTRDTSTST | |
| | VYMELSSLRSEDTAVYYCAKEGDYYYGMDAWGQGTMVT | |
| | VSS | |
| 123 | DIVMTQSPLSLPVTPGEPASISCRSSQSLLHSNGYNYLDWY | CD8_2 VL |
| | LQKPGQSPQLLIYLGSNRASGVPDRFSGSGSGTDFTLKISRV | |
| | EAEDVGVYYCMQGLQTPHTFGQGTKVEIKR | |
| 124 | QVQLVQSGAEVKKPGASVKVSCKASGYTFTSYYMHWVRQ | CD8_3 VH |
| | APGQGLEWMGGFDPEDGETIYAQKFQGRVTMTRDTSTSTV | |
| | YMELSSLRSEDTAVYYCARDQGWGMDVWGQGTTVTVSS | |
| 125 | DIQMTQSPSSLSASVGDRVTITCRASQSISSYLNWYQQKPG | CD8_3 VL |
| | KAPKLLIYAASSLQSGVPSRFSGSGSGTDFTLTISSLQPEDFA | |
| | TYYCQQTYSTPYTFGQGTKLEIKR | |
| 126 | QVQLVQSGAEVKKPGASVKVSCKASGYTFTNHYMHWVR | CD8_4 VH |
| | QAPGQGLEWMGWMNPNSGNTGYAQKFQGRVTMTRDTST | |
| | STVYMELSSLRSEDTAVYYCASSESGSDLDYWGQGTLVTV | |
| | SS | |
| 127 | DIQMTQSPSSLSASVGDRVTITCRASQTIGNYVNWYQQKPG | CD8_4 VL |
| | KAPKLLIYGASNLHTGVPSRFSGSGSGTDFTLTISSLQPEDF | |
| | ATYYCQQTYSAPLTFGGGTKVEIKR | |
| 128 | QVQLVESGGGLVQAGGSLRLSCAASGRTFSGYVMGWFRQ | CD8_VHH |
| | APGKQRKFVAAISRGGLSTSYADSVKGRFTISRDNAKNTVF | |
| | LQMNTLKPEDTAVYYCAADRSDLYEITAASNIDSWGQGTL | |
| | VTVSS | |
| 129 | APTSSSTKKTQLQLEHLLLDLQMILNGINNYKNPKLTRMLT | IL-2 (mature) |
| | FKFYMPKKATELKHLQCLEEELKPLEEVLNLAQSKNFHLRP | |
| | RDLISNINVIVLELKGSETTFMCEYADETATIVEFLNRWITF | |
| | CQSIISTLT | |
| 130 | MYRMQLLSCIALSLALVTNSAPTSSSTKKTQLQLEHLLLDL | IL-2 (precursor) |
| | QMILNGINNYKNPKLTRMLTFKFYMPKKATELKHLQCLEE | |
| | ELKPLEEVLNLAQSKNFHLRPRDLISNINVIVLELKGSETTF | |
| | MCEYADETATIVEFLNRWITFCQSIISTLT | |
| 131 | PTSSSTKKTQLQLEHLLLDLQMILNGINNYKNPKLTRMLTF | Aldesleukin/ |
| | KFYMPKKATELKHLQCLEEELKPLEEVLNLAQSKNFHLRP | proleukin |
| | RDLISNINVIVLELKGSETTFMCEYADETATIVEFLNRWITFS | |
| | QSIISTLT | |
| 132 | GFTFSYYG | 17 |
| 133 | ISNDGSNK | 18 |
| 134 | RPEDTAVYYCAT | 19 |
| 135 | GYNFDTYW | 20 |
| 136 | IYPADSDT | 21 |
| 137 | ARFSSSAYDI | 22 |
| 138 | GYNFDTYW | 23 |
| 139 | IYPADSDT | 24 |
| 140 | SSAYDIARFS | 25 |
| 141 | GFTFNRYA | 26 |
| 142 | ISFDGGNQ | 27 |
| 143 | VRSPAGDWVAYFDY | 28 |
| 144 | ALPKRF | 29 |
| 145 | EDN | 30 |
| 146 | YSTDNTGGLWV | 31 |
| 147 | ALPRQF | 32 |
| 148 | KDT | 33 |
| 149 | DSSGTYHQSA | 34 |
| 150 | ALPRKF | 35 |
| 151 | QDT | 36 |
| 152 | QSADRSDSYV | 37 |
| 153 | EDISNH | 38 |
| 154 | DAS | 39 |
| 155 | HQYHDYPSYT | 40 |
| 156 | MFHVSFRYIF GLPPLIL VLL PVASSDCDIE GKDGKQYESV | Genbank |
| | LMVSIDQLLD SMKEIGSNCL NNEFNFFKRH | Accession No. |
| | ICDANKEGMF LFRAARKLRQ FLKMNSTGDF | P13232 (human |
| | DLHLLKVSEG TTILLNCTGQ VKGRKPAALG EAQPTKSLEE | IL-7) |
| | NKSLKEQKKL NDLCFLKRLL QEIKTCWNKI LMGTKEH | |
| 157 | MRISKPHLRSISIQCYLCLLLNSHFLTEAGIHVFILGCFSAGL | IL-15 LSP |
| | PKTEANWVNVISDLKKIEDLIQSMHIDATLYTESDVHPSCK | |
| | VTAMKCFLLELQVISLESGDASIHDTVENLIILANNSLSSNG | |
| | NVTESGCKECEELEEKNIKEFLQSFVHIVQMFINTS | |
| 158 | MVLGTIDLCSCFSAGLPKTEANWVNVISDLKKIEDLIQSMHI | IL-15 SSP |
| | DATLYTESDVHPSCKVTAMKCFLLELQVISLESGDASIHDT | |
| | VENLIILANNSLSSNGNVTESGCKECEELEEKNIKEFLQSFV | |
| | HIVQMFINTS | |
| 159 | NWVNVISDLKKIEDLIQSMHIDATLYTESDVHPSCKVTAMK | IL-15 mature |
| | CFLLELQVISLESGDASIHDTVENLIILANNSLSSNGNVTESG | |
| | CKECEELEEKNIKEFLQSFVHIVQMFINTS | |
| 160 | QVQLVQSGAEVKKPGASVKVSCKAS | 24 |
| 161 | IHWVRQAPGQGLELIGW | 25 |
| 162 | NYAQKFQVKATMTRDTSISTAYMELSRLRSDDTAVYYC | 26 |
| 163 | WGQGTTVTVSS | 27 |
| 164 | DVVMTQSPLSLPVTLGQPASISCKSS | 28 |
| 165 | LNWLQQRPGQSPRRLI | 29 |
| 166 | KLDSGVPDRFSGSGSGTDFTLKISRVEAEDVGVYYC | 30 |
| 167 | FGQGTKLEIK | 31 |
| 168 | QVQLVQSGAEVKKPGASVKVSCKASGFNIK | 32 |
| 169 | WVRQAPGQGLELIG | 33 |
| 170 | KATMTRDTSISTAYMELSRLRSDDTAVYYCND | 34 |
| 171 | WGQGTTVTVSS | 35 |
| 172 | DVVMTQSPLSLPVTLGQPASISC | 36 |
| 173 | WLQQRPGQSPRRLIS | 37 |
| 174 | GVPDRFSGSGSGTDFTLKISRVEAEDVGVYYC | 38 |
| 175 | FGQGTKLEIK | 39 |
| 176 | QGQDRHMIRMRQLIDIVDQLKNYVNDLVPEFLPAPEDVET | IL-21 (156) |
| | NCEWSAFSCFQKAQLKSANTGNNERIINVSIKKLKRKPPST | |
| | NAGRRQKHRLTCPSCDSYEKKPPKEFLERFKSLLQKMIHQ | |
| | HLSSRTHGSEDS | |
| 177 | QGQDEHMIRMRQLIDIVDQLKNYVNDLVPEFLPAPEDVET | IL-21 (157) |
| | NCEWSAFSCFQKAQLKSANTGNNERIINVSIKKLKRKPPST | |
| | NAGRRQKHRLTCPSCDSYEKKPPKEFLERFKSLLQKMIHQ | |
| | HLSSRTHGSEDS | |
| 178 | QGQDRHMIRMRQLIDIVDQLKNYVNDLVPEFLPAPEDVET | IL-21 (158) |
| | NCEWSAFSCFQKAQLKSANTGNNERIINVSIKKLKRKPPST | |
| | NAGRRQKHRLTCPSCDSYEKKPPKEFLERFKSLLQKMIHQ | |
| | HL | |
| 179 | MDCDIEGKDGKQYESVLMVSIDQLLDSMKEIGSNCLNNEF | IL-7 (1) |
| | NFFKRHICDANKEGMFLFRAARKLRQFLKMNSTGDFDLHL | |
| | LKVSEGTTILLNCTGQVKGRKPAALGEAQPTKSLEENKSLK | |
| | EQKKLNDLCFLKRLLQEIKTCWNKILMGTKEH | |
| 180 | DCDIEGKDGKQYESVLMVSIDQLLDSMKEIGSNCLNNEFNF | IL-7 (2) |
| | FKRHICDANKEGMFLFRAARKLRQFLKMNSTGDFDLHLLK | |
| | VSEGTTILLNCTGQVKGRKPAALGEAQPTKSLEENKSLKEQ | |
| | KKLNDLCFLKRLLQEIKTCWNKILMGTKEH | |
| 181 | DCDIEGKDGKQYESVLMVSIDQLLDSMKEIGSNCLNNEFNF | IL-7 (3) |
| | FKRHICDANKEGMFLFRAARKLRQFLKMNSTGDFDLHLLK | |
| | VSEGTTILLNCTGQEENKSLKEQKKLNDLCFLKRLLQEIKT | |
| | CWNKILMGTKEH | |
| 182 | DCDIEGKDGKQYESVLMVSIDQLLDSMKEIGSNCLNNEFNF | IL-7 (4) |
| | FKRHICDANKEGMFLFRAARKLRQFLKMNSTGDFDLHLLK | |
| | VSEGTTILLKEQKKLNDLCFLKRLLQEIKTCWNKILMGTKE | |
| | H | |
| 183 | DCDIEGKDGKQYESVLMVSIDQLLDSMKEIGSNCLNNEFNF | IL-7 (5) |
| | FKRHICDANKVKGRKPAALGEAQPTKSLEENESLKEQKKL | |
| | NDLCFLKRLLQEIKTCWNKILMGTKEH | |
| 184 | DCDIEGKDGKQYESVLMVSIDQLLDSMKEIGSNCLNNEFNF | IL-7 (6) |
| | FKRHICDANKEENESLKEQKKLNDLCFLKRLLQEIKTCWN | |
| | KILMGTKEH | |
| 185 | DCDIEGKDGKQYESVLMVSIDQLLVKGRKPAALGEAQPTK | IL-7 (7) |
| | SLEENESLKEQKKLNDLCFLKRLLQEIKTCWNKILMGTKEH | |
| 186 | DCDIEGKDGKQYESVLMVSIDQLLEENKSLKEQKKLNDLC | IL-7 (8) |
| | FLKRLLQEIKTCWNKILMGTKEH | |
| 187 | DCDIEGKDGKQYESVLMVSIDQLLDSMKEIGSNCL | IL-7 (mature |
| | NNEFNFFKRHICDANKVKGRKPAALGEAQPTKSLEENKSL | human IL-7) |
| | KEQKKLNDLCFLKRLLQEIK | |
| | TCWNKILMGTKEH | |
| 188 | HKSSSQGQDRHMIRMRQLIDIVDQLKNYVNDLVPEF | IL-21 (mature |
| | LPAPEDVETNCEWSAFSCFQKAQLKSANTGNNERIINVSIK | human IL-21) |
| | KLKRKPPSTNAGRRQKHRL | |
| | TCPSCDSYEKKPPKEFLERFKSLLQKMIHQHLSSRTHGSEDS | |
| 189 | GFTFSGYW | CDR-H1 |
| 190 | ISPGGGST | CDR-H2 |
| 191 | ASSLTATHTYEYDY | CDR-H3 |
| 192 | EVQLVESGGGLVQSGGSLRLSCAASGFTFSGYWMYWVRQ | VHH |
| | APGKGLEWVSAISPGGGSTYYPDSVKGRFTISRDNAKNTLY | |
| | LQMNSLEPEDTALYYCASSLTATHTYEYDYWGQGTQVTV | |
| | SS | |
| 193 | GYTFSNYW | CDR-H1 |
| 194 | ILPGSGST | CDR-H2 |
| 195 | ARRGYGYDEGFDY | CDR-H3 |
| 196 | QDINSY | CDR-L1 |
| 197 | RAN | CDR-L2 |
| 198 | LQYDEFPPT | CDR-L3 |
| 199 | QVQLQQSGAELMKPGASVKMSCKATGYTFSNYWIEWVKQ | VH |
| | RPGHGLEWIGEILPGSGSTSYNEKFKGKATFTADTSSSTAY | |
| | MQLSSLTSEDSAVYYCARRGYGYDEGFDYWGQGSTLTVS | |
| | S | |
| 200 | DIKMTQSPSSMYASLGERVTITCKASQDINSYLSWFQQKPG | VL |
| | KSPKTLIYRANRLVDGVPSRFSGSGSGQDYSLTISSLEYED | |
| | MGIYYCLQYDEFPPTFGAGTKLELKR | |
| 201 | QVQLQQSGAELMKPGASVKMSCKATGYTFSNYWIEWVKQ | scFv |
| | RPGHGLEWIGEILPGSGSTSYNEKFKGKATFTADTSSSTAY | |
| | MQLSSLTSEDSAVYYCARRGYGYDEGFDYWGQGSTLTVS | |
| | SGGGGSGGGGSGGGGSDIKMTQSPSSMYASLGERVTITCK | |
| | ASQDINSYLSWFQQKPGKSPKTLIYRANRLVDGVPSRFSGS | |
| | GSGQDYSLTISSLEYEDMGIYYCLQYDEFPPTFGAGTKLEL | |
| | KR | |
| 202 | GYTFTDYV | CDR-H1 |
| 203 | IYPGSGSS | CDR-H2 |
| 204 | ARPGDLGFAY | CDR-H3 |
| 205 | QSVDYDGDSY | CDR-L1 |
| 206 | AAS | CDR-L2 |
| 207 | QQSNKDPFT | CDR-L3 |
| 208 | QVQLQQSGPELVKPGASVKMSCKASGYTFTDYVISWVRQ | VH |
| | APGQGLEWIGEIYPGSGSSYYNEKFKGRATLTADKSSNTAY | |
| | MQLSSLRSEDSAVYFCARPGDLGFAYWGQGTLVTVSS | |
| 209 | DIVLTQSPSSLAVSLGQRATISCKASQSVDYDGDSYMNWY | VL |
| | QQKPGQPPKLLIYAASNLESGIPARFSGSGSGTDFTLTIHPV | |
| | EEEDAATYYCQQSNKDPFTFGGGTKLELKR | |
| 210 | QVQLQQSGPELVKPGASVKMSCKASGYTFTDYVISWVRQ | scFv |
| | APGQGLEWIGEIYPGSGSSYYNEKFKGRATLTADKSSNTAY | |
| | MQLSSLRSEDSAVYFCARPGDLGFAYWGQGTLVTVSSGG | |
| | GGSGGGGSGGGGSDIVLTQSPSSLAVSLGQRATISCKASQS | |
| | VDYDGDSYMNWYQQKPGQPPKLLIYAASNLESGIPARFSG | |
| | SGSGTDFTLTIHPVEEEDAATYYCQQSNKDPFTFGGGTKLE | |
| | LKR | |
| 211 | GGTFSSYA | CDR-H1 |
| 212 | INPNSGGT | CDR-H2 |
| 213 | ARDGYSGSYSD | CDR-H3 |
| 214 | QSVLSSSYNKNY | CDR-L1 |
| 215 | WAS | CDR-L2 |
| 216 | QQYYSTPWT | CDR-L3 |
| 217 | QVQLVQSGAEVKKPGASVKVSCKASGGTFSSYAISWVRQA | VH |
| | PGQGLEWMGWINPNSGGTNYAQKFQGRVTMTRDTSTSTV | |
| | YMELSSLRSEDTAVYYCARDGYSGSYSDWGQGTLVTVSS | |
| 218 | DIVMTQSPDSLAVSLGERATINCKSSQSVLSSSYNKNYLAW | VL |
| | YQQKPGQPPKLLIYWASTRESGVPDRFSGSGSGTDFTLTISS | |
| | LQAEDVAVYYCQQYYSTPWTFGQGTKVEIK | |
| 219 | QVQLVQSGAEVKKPGASVKVSCKASGGTFSSYAISWVRQA | scFv |
| | PGQGLEWMGWINPNSGGTNYAQKFQGRVTMTRDTSTSTV | |
| | YMELSSLRSEDTAVYYCARDGYSGSYSDWGQGTLVTVSS | |
| | GGGGSGGGGSGGGGSDIVMTQSPDSLAVSLGERATINCKS | |
| | SQSVLSSSYNKNYLAWYQQKPGQPPKLLIYWASTRESGVP | |
| | DRFSGSGSGTDFTLTISSLQAEDVAVYYCQQYYSTPWTFGQ | |
| | GTKVEIK | |
| 220 | GYTFTSYD | CDR-H1 |
| 221 | IIPLSGAP | CDR-H2 |
| 222 | ARGALYNWNDGWFDP | CDR-H3 |
| 223 | QDIGDY | CDR-L1 |
| 224 | DAS | CDR-L2 |
| 225 | QQANSFPLT | CDR-L3 |
| 226 | QVQLVQSGAEVKKPGSSVKVSCKASGYTFTSYDINWVRQA | VH |
| | PGQGLEWMGGIIPLSGAPNYAHKFQGRVTITADESTSTAY | |
| | MELSSLRSEDTAVYYCARGALYNWNDGWFDPWGQGTLV | |
| | TVSS | |
| 227 | DIQMTQSPSSLSASVGDRVTITCRASQDIGDYLAWYQQKPG | VL |
| | KAPKLLIYDASSLQSGVPSRFSGSGSGTDFTLTISSLQPEDFA | |
| | TYYCQQANSFPLTFGGGTKVEIK | |
| 228 | QVQLVQSGAEVKKPGSSVKVSCKASGYTFTSYDINWVRQA | scFi |
| | PGQGLEWMGGIIPLSGAPNYAHKFQGRVTITADESTSTAY | |
| | MELSSLRSEDTAVYYCARGALYNWNDGWFDPWGQGTLV | |
| | TVSSGGGGSGGGGSGGGGSDIQMTQSPSSLSASVGDRVTIT | |
| | CRASQDIGDYLAWYQQKPGKAPKLLIYDASSLQSGVPSRFS | |
| | GSGSGTDFTLTISSLQPEDFATYYCQQANSFPLTFGGGTKVE | |
| | IK | |
| 229 | GDTFSDHA | CDR-H1 |
| 230 | MNPKIGNT | CDR-H2 |
| 231 | VYDSSGYDAFDI | CDR-H3 |
| 232 | QSVLSTSYNRNF | CDR-L1 |
| 233 | QQYYSTPYT | CDR-L3 |
| 234 | QVQLVQSGAEVKKPGASVKVSCKASGDTFSDHAINWVRQ | VH |
| | APGQGLEWMGWMNPKIGNTGYAQKFQGRVTMTRDTSTS | |
| | TVYMELSSLRSEDTAVYYCVYDSSGYDAFDIWGQGTTVTV | |
| | SS | |
| 235 | DIVMTQSPDSLAVSLGERATINCKSSQSVLSTSYNRNFLAW | VL |
| | YQQKPGQPPKLLIYWASTRQSGVPDRFSGSGSGTDFTLTISS | |
| | LQAEDVAVYYCQQYYSTPYTFGQGTKLEIK | |
| 236 | QVQLVQSGAEVKKPGASVKVSCKASGDTFSDHAINWVRQ | scFv |
| | APGQGLEWMGWMNPKIGNTGYAQKFQGRVTMTRDTSTS | |
| | TVYMELSSLRSEDTAVYYCVYDSSGYDAFDIWGQGTTVTV | |
| | SSGGGGSGGGGSGGGGSDIVMTQSPDSLAVSLGERATINC | |
| | KSSQSVLSTSYNRNFLAWYQQKPGQPPKLLIYWASTRQSG | |
| | VPDRFSGSGSGTDFTLTISSLQAEDVAVYYCQQYYSTPYTF | |
| | GQGTKLEIK | |
| 237 | GYSLITHW | CDR-H1 |
| 238 | INPSDGVT | CDR-H2 |
| 239 | AREYYGEGFDY | CDR-H3 |
| 240 | QGISNY | CDR-L1 |
| 241 | SAS | CDR-L2 |
| 242 | QQSYSTPLT | CDR-L3 |
| 243 | QVQLVQSGAEVKKPGASVKVSCKASGYSLITHWMHWVRQ | VH |
| | APGQGLEWMGMINPSDGVTYYAQTFQGRVTMTRDTSTST | |
| | VYMELSSLRSEDTAVYYCAREYYGEGFDYWGQGTLVTVS | |
| | S | |
| 244 | DIQMTQSPSSLSASVGDRVTITCRASQGISNYLAWYQQKPG | VL |
| | KAPKLLIYSASNLQSGVPSRFSGSGSGTDFTLTISSLQPEDFA | |
| | TYYCQQSYSTPLTFGGGTKVEIKR | |
| 245 | QVQLVQSGAEVKKPGASVKVSCKASGYSLITHWMHWVRQ | scFv |
| | APGQGLEWMGMINPSDGVTYYAQTFQGRVTMTRDTSTST | |
| | VYMELSSLRSEDTAVYYCAREYYGEGFDYWGQGTLVTVS | |
| | SGGGGSGGGGSGGGGSDIQMTQSPSSLSASVGDRVTITCRA | |
| | SQGISNYLAWYQQKPGKAPKLLIYSASNLQSGVPSRFSGSG | |
| | SGTDFTLTISSLQPEDFATYYCQQSYSTPLTFGGGTKVEIKR | |
| 246 | GYWMY | CDR-H1 |
| 247 | AISPGGGSTYYPDSVKG | CDR-H2 |
| 248 | SLTATHTYEYDY | CDR-H3 |
| 249 | GFTFSGY | CDR-H1 |
| 250 | SPGGGS | CDR-H2 |
| 251 | NYWIE | CDR-H1 |
| 252 | EILPGSGSTSYNEKFKG | CDR-H2 |
| 253 | RGYGYDEGFDY | CDR-H3 |
| 254 | KASQDINSYLS | CDR-L1 |
| 255 | RANRLVD | CDR-L2 |
| 256 | GYTFSNY | CDR-H1 |
| 257 | LPGSGS | CDR-H2 |
| 258 | DYVIS | CDR-H1 |
| 259 | EIYPGSGSSYYNEKFKG | CDR-H2 |
| 260 | PGDLGFAY | CDR-H3 |
| 261 | KASQSVDYDGDSYMN | CDR-L1 |
| 262 | AASNLES | CDR-L2 |
| 263 | GYTFTDY | CDR-H1 |
| 264 | YPGSGS | CDR-H2 |
| 265 | SYAIS | CDR-H1 |
| 266 | WINPNSGGTNYAQKFQG | CDR-H2 |
| 267 | DGYSGSYSD | CDR-H3 |
| 268 | KSSQSVLSSSYNKNYLA | CDR-L1 |
| 269 | WASTRES | CDR-L2 |
| 270 | GGTFSSY | CDR-H1 |
| 271 | NPNSGG | CDR-H2 |
| 272 | SYDIN | CDR-H1 |
| 273 | GIIPLSGAPNYAHKFQG | CDR-H2 |
| 274 | GALYNWNDGWFDP | CDR-H3 |
| 275 | RASQDIGDYLA | CDR-L1 |
| 276 | DASSLQS | CDR-L2 |
| 277 | GYTFTSY | CDR-H1 |
| 278 | IPLSGA | CDR-H2 |
| 279 | DHAIN | CDR-H1 |
| 280 | WMNPKIGNTGYAQKFQG | CDR-H2 |
| 281 | DSSGYDAFDI | CDR-H3 |
| 282 | KSSQSVLSTSYNRNFLA | CDR-L1 |
| 283 | WASTRQS | CDR-L2 |
| 284 | GDTFSDH | CDR-H1 |
| 285 | NPKIGN | CDR-H2 |
| 286 | THWMH | CDR-H1 |
| 287 | MINPSDGVTYYAQTFQG | CDR-H2 |
| 288 | EYYGEGFDY | CDR-H3 |
| 289 | RASQGISNYLA | CDR-L1 |
| 290 | SASNLQS | CDR-L2 |
| 291 | GYSLITH | CDR-H1 |
| 292 | NPSDGV | CDR-H2 |
| 293 | GFDDPRKAIELVQKRYGRPCDCSGGQVSEPPSDRVSQVTCS | Full BaEV protein |
| | GKTAYLMPDQRWKCKSIPKDTSPSGPLQECPCNSYQSSVH | without signal |
| | SSCYTSYQQCRSGNKTYYTATLLKTQTGGTSDVQVLGSTN | sequence |
| | KLIQSPCNGIKGQSICWSTTAPIHVSDGGGPLDTTRIKSVQR | (cytoplasmic tail, |
| | KLEEIHKALYPELQYHPLAIPKVRDNLMVDAQTLNILNATY | bold and |
| | NLLLMSNTSLVDDCWLCLKLGPPTPLAIPNFLLSYVTRSSD | underline) |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQQYQVL | |
| | RTDEEAQD | |
| 294 | VLTQQYQVLRTDEEAQD | Full R peptide |
| | | only |
| 295 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+3 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLT | |
| 296 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+4 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQ | |
| 297 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+5 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQQ | |
| 298 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+6 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQQY | |
| 299 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+7 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQQYQ | |
| 300 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+8 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQQYQV | |
| 301 | GFDDPRKAIELVQKRYGRPCDCSGGQVSEPPSDRVSQVTCS | BaEv envelope |
| | GKTAYLMPDQRWKCKSIPKDTSPSGPLQECPCNSYQSSVH | glycoprotein R+9 |
| | SSCYTSYQQCRSGNKTYYTATLLKTQTGGTSDVQVLGSTN | |
| | KLIQSPCNGIKGQSICWSTTAPIHVSDGGGPLDTTRIKSVQR | |
| | KLEEIHKALYPELQYHPLAIPKVRDNLMVDAQTLNILNATY | |
| | NLLLMSNTSLVDDCWLCLKLGPPTPLAIPNFLLSYVTRSSD | |
| | NISCLIIPPLLVQPMQFSNSSCLFSPSYNSTEEIDLGHVAFSN | |
| | CTSITNVTGPICAVNGSVFLCGNNMAYTYLPTNWTGLCVL | |
| | ATLLPDIDIIPGDEPVPIPAIDHFIYRPKRAIQFIPLLAGLGITA | |
| | AFTTGATGLGVSVTQYTKLSNQLISDVQILSSTIQDLQDQV | |
| | DSLAEVVLQNRRGLDLLTAEQGGICLALQEKCCFYVNKSGI | |
| | VRDKIKTLQEELERRRKDLASNPLWTGLQGLLPYLLPFLGP | |
| | LLTLLLLLTIGPCIFNRLTAFINDKLNIIHAMVLTQQYQVL | |
| 302 | MGFTTKIIFLYNLVLVYAGFDDPRKAIELVQKRYGRPCDCS | BaEVRLess |
| | GGQVSEPPSDRVSQVTCSGKTAYLMPDQRWKCKSIPKDTS | |
| | PSGPLQECPCNSYQSSVHSSCYTSYQQCRSGNKTYYTATLL | |
| | KTQTGGTSDVQVLGSTNKLIQSPCNGIKGQSICWSTTAPIHV | |
| | SDGGGPLDTTRIKSVQRKLEEIHKALYPELQYHPLAIPKVR | |
| | DNLMVDAQTLNILNATYNLLLMSNTSLVDDCWLCLKLGPP | |
| | TPLAIPNFLLSYVTRSSDNISCLIIPPLLVQPMQFSNSSCLFSP | |
| | SYNSTEEIDLGHVAFSNCTSITNVTGPICAVNGSVFLCGNN | |
| | MAYTYLPTNWTGLCVLATLLPDIDIIPGDEPVPIPAIDHFIYR | |
| | PKRAIQFIPLLAGLGITAAFTTGATGLGVSVTQYTKLSNQLI | |
| | SDVQILSSTIQDLQDQVDSLAEVVLQNRRGLDLLTAEQGGI | |
| | CLALQEKCCFYVNKSGIVRDKIKTLQEELERRRKDLASNPL | |
| | WTGLQGLLPYLLPFLGPLLTLLLLLTIGPCIFNRLTAFINDKL | |
| | NIIHAM | |
| 303 | MGFTTKIIFLYNLVLVYAGFDDPRKAIELVQKRYGRPCDCS | BaEVTR |
| | GGQVSEPPSDRVSQVTCSGKTAYLMPDQRWKCKSIPKDTS | |
| | PSGPLQECPCNSYQSSVHSSCYTSYQQCRSGNKTYYTATLL | |
| | KTQTGGTSDVQVLGSTNKLIQSPCNGIKGQSICWSTTAPIHV | |
| | SDGGGPLDTTRIKSVQRKLEEIHKALYPELQYHPLAIPKVR | |
| | DNLMVDAQTLNILNATYNLLLMSNTSLVDDCWLCLKLGPP | |
| | TPLAIPNFLLSYVTRSSDNISCLIIPPLLVQPMQFSNSSCLFSP | |
| | SYNSTEEIDLGHVAFSNCTSITNVTGPICAVNGSVFLCGNN | |
| | MAYTYLPTNWTGLCVLATLLPDIDIIPGDEPVPIPAIDHFIYR | |
| | PKRAIQFIPLLAGLGITAAFTTGATGLGVSVTQYTKLSNQLI | |
| | SDVQILSSTIQDLQDQVDSLAEVVLQNRRGLDLLTAEQGGI | |
| | CLALQEKCCFYVNKSGIVRDKIKTLQEELERRRKDLASNPL | |
| | WTGLQGLLPYLLPFLGPLLTLLLLLTIGPCIFNRLVQFVKDR | |
| | ISVVQALVLTQQYHQLKPLEYEP | |
| 304 | RYTMH | CD3 CDR-H1 |
| 305 | YINPSRGYTNYNQKFKD | CD3 CDR-H2 |
| 306 | YYDDHYCLDY | CD3 CDR-H3 |
| 307 | RASSSVSYMN | CD3 CDR-L1 |
| 308 | DTSKVAS | CD3 CDR-L2 |
| 309 | QQWSSNPLT | CD3 CDR-L3 |
| 310 | DIKLQQSGAELARPGASVKMSCKTSGYTFTRYTMHWVKQ | CD3 VH |
| | RPGQGLEWIGYINPSRGYTNYNQKFKDKATLTTDKSSSTA | |
| | YMQLSSLTSEDSAVYYCARYYDDHYCLDYWGQGTTLTVS | |
| | SVE | |
| 311 | DIQLTQSPAIMSASPGEKVTMTCRASSSVSYMNWYQQKSG | CD3 VL |
| | TSPKRWIYDTSKVASGVPYRFSGSGSGTSYSLTISSMEAED | |
| | AATYYCQQWSSNPLTFGAGTKLELK | |
| 312 | DIKLQQSGAELARPGASVKMSCKTSGYTFTRYTMHWVKQ | CD3 scFv |
| | RPGQGLEWIGYINPSRGYTNYNQKFKDKATLTTDKSSSTA | |
| | YMQLSSLTSEDSAVYYCARYYDDHYCLDYWGQGTTLTVS | |
| | SVEGGSGGSGGSGGSGGVDDIQLTQSPAIMSASPGEKVTM | |
| | TCRASSSVSYMNWYQQKSGTSPKRWIYDTSKVASGVPYRF | |
| | SGSGSGTSYSLTISSMEAEDAATYYCQQWSSNPLTFGAGTK | |
| | LELK | |

What is claimed:

1. A method for administration of a lipid particle to a subject, the method comprising: a) obtaining whole blood from a subject; b) collecting the fraction of blood containing peripheral blood mononuclear cells (PBMCs) or a subset thereof; c) contacting the collected PBMCs or subset with a composition comprising lipid particles to create a transfection mixture; and d) reinfusing the transfection mixture to the subject, thereby administering the lipid particle to the subject.

2. The method of claim 1, wherein the lipid particle comprises a nucleic acid encoding a payload gene.

3. A method for administration of a payload gene to a subject, the method comprising: a) obtaining whole blood from a subject; b) collecting the fraction of blood containing peripheral blood mononuclear cells (PBMCs) or subset thereof; c) contacting the PBMCs or subset with a composition comprising lipid particles comprising a nucleic acid encoding a payload gene to create a transfection mixture; and d) reinfusing the transfection mixture to the subject, thereby administering the payload gene to the subject.

4. A method for administration of a lipid particle to a subject, the method comprising: a) obtaining whole blood from a subject; b) collecting the fraction of blood containing peripheral blood mononuclear cells (PBMCs) subset thereof; c) contacting the PBMCs or subset thereof with a composition comprising lipid particles to create a transfection mixture; and d) reinfusing the transfection mixture to the subject, thereby administering the lipid particle to the subject, wherein the method is characterized by one or more of:
(i) wherein the method is characterized by the collecting in (b) not comprising a selection step for target cells, optionally wherein the selection step is immunoaffinity selection;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, collected PBMCs or subset thereof, and transfection mixture are not subjected to cryopreservation or freezing; and/or
(v) steps (a)-(d) are carried out for a time that is no more than 24 hours.

5. The method of claim 4, wherein the lipid particle comprises a nucleic acid encoding a payload gene.

6. A method for delivering a payload gene to a subject, the method comprising: a) obtaining whole blood from a subject; b) collecting the fraction of blood containing peripheral blood mononuclear cells (PBMCs) or leukocyte components; c) contacting the PBMCs or subset thereof with a composition comprising lipid particles comprising a nucleic acid encoding a payload gene to create a transfection mixture; and d) reinfusing the transfection mixture to the subject, thereby administering the lipid particle to the subject, wherein the method is characterized by one or more of:
(i) wherein the method is characterized by the collecting in (b) not comprising a selection step for target cells, optionally wherein the selection step is immunoaffinity selection;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

7. The method of any of claims 1-6, wherein the method is carried out in a single in-line procedure to maintain a closed or functionally closed fluid circuit.

8. The method of any of claims 1-7 wherein:
two or more of steps (a)-(d) are carried out in-line in a closed fluid circuit;
three or more of steps (a)-(d) are carried out in-line in a closed fluid circuit; or wherein
all of steps (a)-(d) are carried out in-line in a closed fluid circuit.

9. The method of claim 8, wherein, between at least two steps, the method includes separating the subject from the in-line closed fluid circuit and then reconnecting the subject prior to the next step.

10. The method of claim 8 or claim 9, wherein steps (a)-(c) are carried out in-line in a closed fluid circuit, and wherein the method comprises separating the subject from the closed fluid circuit after step (c) and reconnecting the subject to the closed fluid circuit before step (d).

11. The method of any of claims 6-10, wherein the method is characterized by at least two of (i)-(v), at least three of (i)-(v), at least four of (i)-(v), or (i)-(v).

12. The method of any of claims 1-11, wherein the method is characterized by the contacting in step (c) being initiated within 24 hours after collecting the fraction of blood containing PBMCs or subset thereof, optionally wherein the contacting in step (c) is initiated 0 to 12 hours, 0 to 6 hours, 0 to 4 hours, 0 to 2 hours or 0 to 1 hour, or 0 to 30 minutes, or within at or about 12 hours, within at or about 6 hours, within at or about 2 hours, within at or about 1 hour, within at or about 30 minutes or within at or about 15 minutes after collecting the fraction of blood containing PBMCs or subset thereof.

13. The method of any of claims 1-12, wherein the contacting in step (c) is initiated immediately after collecting the fraction of blood containing PBMCs or subset thereof following transfer to a contacting chamber.

14. The method of any of claims 1-13, wherein the method is characterized by the contacting in step (c) being no more than 24 hours prior to the reinfusing in step (d).

15. The method of any of claims 1-14, wherein the contacting in step (c) is for at or about 15 minutes, at or about 30 minutes, at or about 1 hour, or at or about 2 hours, or any value between any of the foregoing.

16. The method of any of claims 1-15, wherein at least a portion of the contacting in (c) is carried out under centrifugation.

17. The method of any of claims 1-16, wherein the transfection mixture is directly reinfused to the subject, optionally without any further processing or washing steps.

18. The method of any of claims 1-17, wherein the method is characterized by steps (a)-(d) being carried out for a time that is no more than 24 hours.

19. The method of any of claims 1-18, wherein the steps (a)-(d) are carried out for a time that is between 2 hours and 6 hours, and/or wherein the steps (a)-(d) are carried out for a time that is between 2 hours and 4 hours or between 3 hours and 4 hours.

20. The method of any of claims 7-19, wherein the closed fluid circuit comprises one or more of a blood processing set for obtaining the whole blood from the subject, a separation chamber for the separating the PBMCS or subset from the blood to collect the PBMCs or subset, a contacting container for the contacting the collected PBMCs or subset thereof with the composition comprising lipid particles, and a transfer container containing the contacted PBMCs or subset thereof and/or the transfection mixture for reinfusion to the subject.

21. The method of claim 20, wherein the closed fluid circuit further comprises a collection container operably connected to the separation chamber to collect the PBMCs or subset, optionally wherein the collection container is a bag, more optionally a sterile bag.

22. The method of claim 20 or claim 21, wherein the contacting chamber and the transfer container are the same container, optionally wherein the container is a bag, more optionally a sterile bag.

23. The method of any of claims 20-22, wherein the collecting container, the contacting chamber, and the transfer container are the same container, wherein the container is a bag, more optionally a sterile bag.

24. The method of any of claims 7-19, wherein the closed fluid circuit comprises one or more of a blood processing set for obtaining the whole blood from the subject, a separation chamber for the separating the PBMCs or subset from the blood to collect the PBMCs or subset, and a container, wherein the container is configured as a collection container for collecting the PBMCs or subset from the separation chamber, a contacting chamber for contacting with the lipid particles to create a transfection mixture, and a transfer container for reinfusing the transfer mixture to the subject, optionally wherein the container is a bag, further optionally a sterile bag.

25. The method of any of claims 20-24, wherein the container or the collecting container is operably connected to a source container comprising the composition comprising lipid particles.

26. The method of any of claims 20-25, wherein the container or the transfer container is operably connected to a return processing unit for reinfusion of contacted PBMCs or the transfection mixture to the subject.

27. The method of any of claims 20-26, wherein the transfer container is separably connected form the closed fluid circuit for reinfusion.

28. The method of any of claims 20-27, wherein the transfer container is not disengaged from the closed fluid circuit during reinfusion to the subject.

29. The method of any of claims 20-28, wherein the transfer container is part of a return processing unit comprised by the closed fluid circuit, said return processing unit configured to reinfuse the PBMCs or subset thereof or the transfection mixture to the subject.

30. The method of any of claims 7-29, wherein the closed fluid circuit is characterized by:
(a) a blood processing set for obtaining the whole blood from the subject,
(b) a separation chamber for the separating the PBMCs or subset from the blood to collect the PBMCs or subset;
(c) a collecting container for collecting the PBMCs or subset from the separation chamber,
(d) a source container comprising the composition comprising lipid particles, wherein the collecting container is configured as a contacting container and is operably connected to the source container for contacting of the collected PBMCs or subset with a composition comprising lipid particles to create the transfection mixture; and
(e) a return processing unit configured to reinfuse to the subject the transfection mixture, wherein the collecting container containing the transfection mixture is configured as a transfer container and is operably connected to the return processing unit for reinfusion to the subject,
wherein (a)-(e) are operably connected in-line in a closed fluid circuit, optionally wherein the container is a bag, further optionally a sterile bag.

31. The method of any of claims 7-30, wherein the closed fluid circuit is sterile.

32. The method of any of claims 20-31, wherein during at least a portion of the contacting in (c) the method comprises mixing the transfection mixture comprising the PBMCs or subset and the composition comprising lipid particles.

33. The method of any of claims 1-32, wherein the collected fraction of blood contains PBMCs or subset thereof separated from other blood components.

34. The method of any of claims 1-33, wherein collecting the fraction of blood is by apheresis via an apheresis device, optionally wherein the apheresis device comprises membrane apheresis or centrifugal apheresis.

35. The method of any of claims 1-34, wherein the collected fraction comprises leukocytes or precursors thereof, optionally wherein the precursors thereof comprise hematopoietic stem cells or a CD34+progenitor cell.

36. The method of any of claims 1-34, wherein collecting the fraction of blood is by leukapheresis and/or the collected fraction is a leukapheresis composition obtained from whole blood by leukapheresis, and/or wherein the collected fraction of blood contains leukocytes.

37. The method of any of claims 1-36, wherein the transfection mixture comprises an anticoagulant, optionally wherein the anticoagulant is a citrate.

38. The method of any of claim 1-37, wherein the viability of cells of the collected fraction and/or of the transfection mixture is greater than 95%, optionally between 95% and 100%, between 96% and 100%, between 97% and 100%, between 98% and 100% or between 99% and 100%.

39. The method of any of claims 1-38, wherein the lipid particle is a viral vector or viral-like particle, optionally wherein the viral vector or viral-like particle is a retroviral vector or retroviral-like particle.

40. The method of claim 39, wherein the viral vector or viral-like particle is a lentiviral vector.

41. The method of claim 40, wherein the lentiviral vector is pseudotyped for targeting to a T cell.

42. The method of claim 41, wherein the T cell is a CD3+ T cell, a CD4+ T cell or a CD8+T cell, optionally wherein the T cell is a CD8+ T cell.

43. The method of any of claims 1-42, wherein the lipid particle comprises a fusogen embedded in the lipid bilayer.

44. The method of claim 43, wherein the fusogen is a viral fusogen selected from a Class I viral membrane fusion protein, a Class II viral membrane protein, a Class II viral membrane fusion protein, a viral membrane glycoprotein, or a viral envelope protein.

45. The method of claim 43 or claim 44, wherein the fusogen is endogenous to the virus.

46. The method of claim 43 or claim 44, wherein the fusogen is a pseudotyped fusogen.

47. The method of any of claims 44-46, wherein the fusogen is a viral envelope protein.

48. The method of any of claims 43-47, wherein the fusogen is
(i) a vesicular stomatitis virus envelope glycoprotein (VSV-G),
(ii) a baboon endogenous virus (BaEV) envelope glycoprotein,
(iii) a Cocal virus envelope glycoprotein,
(iv) an Alphavirus fusion protein or a functional variant thereof, optionally wherein the fusogen is a Sindbis virus fusion protein or functional variant thereof or
(v) a Paramyxoviridae fusion protein or a functional variant thereof, optionally wherein the fusogen is a Morbillivirus or a Henipavirus fusion protein or functional variant thereof.

49. The method of any of claims 43-48, wherein the fusogen is a Morbillivirus fusion protein or a functional variant thereof, optionally wherein the fusogen is a measles virus (MeV), canine distemper virus, Cetacean morbillivirus, Peste-des-petits-ruminants virus, Phocine distemper virus, Rinderpest virus fusion protein or a functional variant thereof.

50. The method of any of claims 43-48, wherein the fusogen is a Henipavirus fusion protein or a functional variant thereof, optionally wherein the fusogen is a Nipah virus, Hendra virus, Cedar virus, Kumasi virus, Mójiāng virus fusion protein or a functional variant thereof.

51. The method of any of claims 43-48, and 50, wherein the fusogen is a Nipah virus fusion protein or a functional variant thereof.

52. The method of any of claims 43-51, wherein the fusogen comprises a paramyxovirus F protein, or a biologically active portion thereof, and wherein the fusogen comprises a paramyxovirus G, paramyxovirus H, and/or paramyxovirus HN protein, or a biologically active portion thereof.

53. The method of any of claims 1-52, wherein the lipid particle or lentiviral vector comprises a nucleic acid encoding a payload gene.

54. The method of any of claims 43-53, wherein the fusogen comprises an F protein molecule or a biologically active portion thereof from a Paramyxovirus and a glycoprotein G (G protein) or a biologically active portion thereof from a Paramyxovirus, optionally wherein the Paramyxovirus is a Nipah virus and the fusogen comprises a Nipah virus F glycoprotein (NiV-F) or a biologically active portion thereof and a Nipah virus G glycoprotein (NiV-G) or a biologically active portion thereof..

55. The method of claim 54, wherein the G protein or biologically active portion is a biologically active portion of wild-type NiV-G that has a deletion of up to 40 amino acids at or near the N-terminus, optionally not including the initial methionine.

56. The method of claim 54 or claim 55, wherein the G protein is a biologically active portion that is a truncated NiV-G that has a deletion of amino acids 2-34 at or near the N-terminus of wild-type NiV-G set forth in SEQ ID NO:14.

57. The method of any of claims 54-56, wherein the G protein or the biologically active portion thereof is a NiV-G protein or biologically active portion thereof that is further mutated to exhibit reduced binding to Ephrin B2 or Ephrin B3.

58. The method of claim 57, wherein the NiV-G protein or a biologically active portion thereof comprises one or more amino acid substitutions corresponding to amino acid substitutions selected from the group consisting of E501A, W504A, Q530A and E533A with reference to numbering set forth in SEQ ID NO:14.

59. The method of any of claims 54-58, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

60. The method of any of claims 54-59, wherein the F protein or the biologically active portion thereof is a NiV-F protein or a biologically active portion thereof.

61. The method of claim 60, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

62. The method of any of claims 54-61, wherein the F protein or the biologically active portion is a truncated NiV-F that lacks amino acids 525-546 of SEQ ID NO:2.

63. The method of any of claims 54-62, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

64. The method of any of claims 54-63, wherein the Niv-G protein is a biologically active portion set forth by the amino acid sequence set forth in SEQ ID NO: 19, and the Niv-F protein is a biologically active portion set forth by the amino acid sequence set forth in SEQ ID NO:12.

65. The method of any of claims 43-64, wherein the fusogen is a re-targeted fusogen comprising a targeting moiety that binds to a target cell.

66. The method of claim 65, wherein the fusogen comprises a paramyxovirus G, paramyxovirus H and/or paramyxovirus HN protein and wherein the targeting moiety is linked to the paramyxovirus G, paramyxovirus H and/or paramyxovirus HN protein.

67. The method of any of claims 54-64, wherein the fusogen is a re-targeted Nipah fusogen and the NiV-G protein or a biologically active portion thereof is further linked to a targeting moiety that binds to a target cell.

68. The method of any of claims 4-67, wherein the target cell is a T cell, a B cell, an NK cell, a macrophage, a monocyte, a dendritic cell, a hematopoietic stem cell or a CD34+progenitor cell, optionally wherein the target cell is a T cell.

69. The method of claim 65-68, wherein the targeting moiety binds to CD4, CD8 or CD3.

70. The method of any of claims 65-69, wherein the targeting moiety comprises a binding agent that is a Design ankyrin repeat proteins (DARPin), a single domain antibody (sdAb), a single chain variable fragment (scFv), or an antigen-binding fibronectin type III (Fn3) scaffold.

71. A method for administration of a lentiviral vector to a subject, the method comprising:
a) obtaining whole blood from a subject;
b) collecting the fraction of blood containing peripheral blood mononuclear cells (PBMCs) or subset thereof;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the transfection mixture to the subject, thereby administering the lentiviral vector to the subject.

72. A method for administrating a lentiviral vector to a subject, the method comprising:
a) obtaining whole blood from a subject;
b) collecting the fraction of blood containing peripheral blood mononuclear cells (PBMCs) or leukocyte components;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR), and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the transfection mixture to the subject, thereby administering the lentiviral particle to the subject, wherein the method is characterized by one or more of:
(i) wherein the method is characterized by the collecting in (b) not comprising a selection step for target cells, optionally wherein the selection step is immunoaffinity selection;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

73. The method of claim 71 or claim 72, wherein the CAR binds to or recognizes a protein or antigen expressed by or on cells associated with a disease or condition, optionally wherein the disease or condition is a cancer.

74. A method for treating cancer in a subject, the method comprising:
a) obtaining whole blood from a subject;
b) collecting the fraction of blood containing peripheral blood mononuclear cells (PBMCs) or subset thereof;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the transfection mixture to the subject, thereby administering the lentiviral vector to the subject.

75. A method for treating a cancer in a subject, the method comprising:
a) obtaining whole blood from a subject;
b) collecting the fraction of blood containing peripheral blood mononuclear cells (PBMCs) or leukocyte components;
c) contacting the PBMCs or subset with a composition comprising a lentiviral vector pseudotyped with a re-targeted Nipah virus fusogen to create a transfection mixture, wherein the lentiviral vector comprises a nucleic acid encoding a chimeric antigen receptor (CAR) that binds to or recognizes a protein or antigen expressed by or on tumor cells, and wherein the re-targeted Nipah virus fusogen comprises (i) a re-targeted Nipah virus G glycoprotein (NiV-G) that is a truncated NiV-G set forth in SEQ ID NO:19 linked to a CD8 binding agent, and (ii) a truncated Nipah virus F glycoprotein (NiV-F) set forth in SEQ ID NO:12; and
d) reinfusing the transfection mixture to the subject, thereby administering the lentiviral particle to the subject, wherein the method is characterized by one or more of:
(i) wherein the method is characterized by the collecting in (b) not comprising a selection step for target cells, optionally wherein the selection step is immunoaffinity selection;
(ii) the contacting in step (c) is initiated within 24 hours after collecting the fraction of blood containing PBMCs or subset thereof;
(iii) the contacting in step (c) is for no more than 24 hours prior to the reinfusing in step (d);
(iv) the whole blood, PBMCs or subset thereof, and transfection mixture are not subjected to cryopreservation or freezing; and/or
(v) steps (a)-(d) are carried out for a time that is no more than 24 hours.

76. The method of any of claims 65-76, wherein the targeting moiety is a CD8 binding agent that is an scFv comprising the VH and VL set forth in SEQ ID NO:120 and 121, SEQ ID NOS: 122 and 123, SEQ ID NOS: 124 and 125 or SEQ ID NOS: 126 and 127, optionally wherein the VH and VL are separated by a linker.

77. The method of any of claims 65-76, wherein the targeting moiety is a CD8 binding agent that is a VHH having the sequence set forth in SEQ ID NO: 128.

78. The method of claim 76 or claim 77, wherein the CD8 binding agent is linked to the C-terminus of a truncated NiV-G set forth in SEQ ID NO: 19 for retargeting of the lipid particle or lentiviral vector to CD8+ T cells.

79. The method of claim 78, wherein the lipid particle or lentiviral vector comprising the retargeted NiV-G is pseudotyped with a NiV-F of a biologically active portion thereof, optionally wherein the NiV-F or biologically active portion is set forth in SEQ ID NO:12.

80. The method of any of claims 39-79, wherein the composition comprising the lipid particle is a viral vector and the composition comprising the lipid particle or the composition comprising the lentiviral vector comprises from 1×10⁸ to 1×10¹¹ infectious units (IU), 1×10⁸ to 1×10¹⁰ IU, 1×10⁸ to 1×10⁹ IU, 1×10⁹ to 1×10¹¹ IU, 1×10⁹ to 1×10¹⁰ IU, 1×10¹⁰ to 1×10¹¹ IU.

81. The method of any of claims 1-80, wherein the volume of the composition comprising lipid particles or lentiviral vector and/or the volume of the collected PBMCs or subset thereof is between 100 mL and 400 mL, inclusive.

82. The method of any of claims 39-81, wherein the collected PBMCs or subset thereof comprises from 1×10⁸ to 1×10¹⁰ nucleated cells, 1×10⁸ to 5×10⁹ nucleated cells, 1×10⁸ to 2×10⁹ nucleated cells, 1×10⁸ to 1×10⁹ nucleated cells, 1×10⁸ to 5×10⁸ nucleated cells, 5×10⁸ to 1×10¹⁰ nucleated cells, 5×10⁸ to 5×10⁹ nucleated cells, 5×10⁸ to 2×10⁹ nucleated cells, 5×10⁸ to 1×10⁹ nucleated cells, 1×10⁹ to 1×10¹⁰ nucleated cells, 1×10⁸ to 5×10⁹ nucleated cells, 1×10⁸ to 2×10⁹ nucleated cells, 2×10⁹ to 1×10¹⁰ nucleated cells, 2×10⁸ to 5×10⁹ nucleated cells, or 5×10⁹ to 1×10¹⁰ nucleated cells.

83. The method of any of claims 1-82, wherein the concentration of the PBMCs or subset during the contacting is from 1×10⁶ cells/mL to 1×10⁸ cells/mL, from 1×10⁶ cells/mL to 5×10⁷ cells/mL, from 1×10⁶ cells/mL to 1×10⁷ cells/mL, from 1×10⁶ cells/mL to 5×10⁶ cells/mL, from 5×10⁶ cells/mL to 1×10⁸ cells/mL, from 5×10⁶ cells/mL to 5×10⁷ cells/mL, from 5×10⁶ cells/mL to 1×10⁷ cells/mL, from 1×10⁷ cells/mL to 1×10⁸ cells/mL, from 1×10⁷ cells/mL to 5×10⁷ cells/mL, from 5×10⁷ cells/mL to 1×10⁸ cells/mL

84. The method of any of claims 1-83, wherein the method does not include a lymphodepleting regimen prior to obtaining the whole blood from the subject.

85. The method of any of claims 4-84, wherein the payload agent is or encodes a therapeutic agent and/or wherein the payload agent is a nucleic acid comprising a gene for correcting a genetic deficiency.

86. The method of any of claims 4-85, wherein the payload agent encodes a membrane protein, optionally wherein the membrane protein is an antigen receptor for targeting cells expressed by or associated with a disease or condition.

87. The method of claim 86, wherein the membrane protein is a chimeric antigen receptor (CAR), optionally wherein the CAR is an anti-CD19 CAR, an anti-CD22 CAR or an anti-CD22 CAR, more optionally wherein the CAR is an anti-CD19 CAR.

88. The method of claim 87, wherein the anti-CD19 CAR comprises an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36, and a CD3zeta signaling domain set forth in SEQ ID NO: 38.

89. The method of any of claims 1-88, further comprising administering a cytokine receptor agonist to the subject, optionally wherein the cytokine receptor agonist is a recombinant protein, a chemically synthesized protein or a conjugate.

90. The method of claim 89, wherein the cytokine receptor agonist binds to a cytokine receptor on a T cell, optionally wherein the cytokine receptor is selected from the group consisting of an IL-2 receptor (IL-2R), an IL-15 receptor (IL-15R), an IL-7 receptor (IL-7R), or an IL-21 receptor (IL-21R).

91. The method of claim 89 or claim 90, wherein the transfection mixture further comprises the cytokine receptor agonist, and wherein reinfusing the transfection mixture to the subject further administers the cytokine receptor agonist to the subject by the in-line method of administration.

92. The method of claim 91, wherein the collected PBMCs or subset are contacted with the cytokine receptor agonist to produce the transfection mixture comprising the cytokine receptor agonist, wherein the contacting with the cytokine receptor agonist is carried out prior to the reinfusing of step (d).

93. The method of claim 92, wherein the contacting with the cytokine receptor agonist is carried out prior to, concurrently with or after the contacting with the composition comprising lipid particles or lentiviral vector.

94. The method of claim 92 or claim 93, wherein the contacting with the cytokine receptor agonist is performed in-line in the closed fluid circuit.

95. The method of any of claims 1-94, wherein the volume of the transfection mixture is between 100 mL and 1000 mL, inclusive, optionally between 100 mL and 400 mL, inclusive.

96. The method of any of claims 91-95, further comprising:
administering one or more doses of the cytokine receptor agonist to the subject after the in-line administration of the lipid particle or lentiviral vector; and/or
administering one or more doses of the cytokine receptor agonist to the subject prior to the in-line administration of the lipid particle or lentiviral vector.

97. The method of any of claims 89, 90, or 96, wherein a first dose of the cytokine receptor agonist is administered prior to the in-line administration of the lipid particle or the lentiviral vector, optionally wherein the first dose of the cytokine receptor agonist is administered within one month, within one week or within three days of the in-line administration of the lipid particle or the lentiviral vector.

98. The method of any of claims 89, 90, or 96, wherein the first dose of the cytokine receptor agonist is administered on the same day as the in-line administration of the lipid particle or the lentiviral vector.

99. The method of any of claims 89, 90, or 96, wherein the first dose of the cytokine receptor agonist is administered after the in-line administration of the lipid particle or the lentiviral vector, optionally wherein the first dose of the cytokine receptor agonist is administered no more than one month, no more than 21 days, no more than 14 days or no more than 7 days after the in-line administration of the lipid particle or the lentiviral vector.

100. A system for infusion of lipid particles into a subject, the system comprising:
(a) an incoming processing unit for obtaining whole blood from the circulatory system of a subject;
(b) a separation chamber for collecting peripheral blood mononuclear cells (PBMCs) or subset thereof from the blood fraction; and
(c) a contacting chamber container for transfection of the PBMCs or subset thereof with a composition comprising lipid particles to create a transfection mixture; and
(d) a transfer container for reinfusing the contacted PBMCs or subset thereof or the transfection mixture to the same subject.

101. A system for delivering a payload agent into a subject, the system comprising:
(a) an incoming processing unit for obtaining whole blood from the circulatory system of a subject;
(b) a separation chamber for collecting peripheral blood mononuclear cells (PBMCs) or subset thereof from the blood fraction; and
(c) a contacting chamber container for transfection of the PBMCs or subset thereof with a composition comprising lipid particles to create a transfection mixture; and
(d) a transfer container for reinfusing the contacted PBMCs or subset thereof or the transfection mixture to the same subject.

102. The system of claim 100 or 101, wherein the lipid particle is a viral vector, optionally wherein the viral vector is a lentiviral vector.

103. The system of any of claims 100-102, wherein the contacting chamber and the transfer container are configured as part of the same container or are the same container, and/or wherein the collecting container, the contacting chamber and the transfer container are configured as part of the same container or are the same container.

104. The system of any of claims 100-103, wherein the system is a closed fluid circuit to operate in-line.

105. The system of claim 104, wherein the transfer container is configured to be separably connected from the closed fluid circuit for reinfusion, or wherein the transfer container is configured not to be disengaged from the closed fluid circuit during reinfusion to the subject.

106. The system of any of claims 100-104, wherein the transfer container is part of a return processing unit comprised by the system, optionally the closed fluid circuit, said return processing unit configured to reinfuse the PBMCs or subset thereof or the transfection mixture to the subject.

107. The system of any of claims 100-106, wherein the transfer container is configured to be operably connected to the closed fluid circuit and/or donor subject, optionally via one or more tubing lines, during the reinfusion to the subject.

108. The system of any of claims 100-107, wherein the separation chamber is an apheresis device and/or wherein the separation chamber is a leukapheresis device.

109. A combination comprising the system of any of claims 100-108 and a container comprising a composition comprising lipid particles.

110. The combination of claim 109, wherein the lipid particle is a viral vector, optionally wherein the viral vector is a lentiviral vector.

111. The combination of claim 109 or claim 110, wherein the viral vector comprises a fusogen embedded in the lipid bilayer.

112. The combination of claim 111, wherein the fusogen is
(i) a vesicular stomatitis virus envelope glycoprotein (VSV-G),
(ii) a baboon endogenous virus (BaEV) envelope glycoprotein,
(iii) a Cocal virus envelope glycoprotein,
(iv) an Alphavirus fusion protein or a functional variant thereof, optionally a Sindbis virus fusion protein or a functional variant thereof, or
(v) a Paramyxoviridae fusion protein or a functional variant thereof, optionally a Morbillivirus or a Henipavirus fusion protein or a functional variant thereof.

113. The combination of any of claims 111 or 112, wherein the fusogen comprises a paramyxovirus F protein, or a biologically active portion thereof, and wherein the fusogen comprises a paramyxovirus G, paramyxovirus H, and/or paramyxovirus HN protein, or a biologically active portion thereof.

114. The combination of any of claims 111-113, wherein the fusogen comprises an F protein molecule or a biologically active portion thereof from a Paramyxovirus and a glycoprotein G (G protein) or a biologically active portion thereof from a Paramyxovirus, optionally wherein the Paramyxovirus is a Nipah virus and the fusogen comprises a Nipah virus F glycoprotein (NiV-F) or a biologically active portion thereof and a Nipah virus G glycoprotein (NiV-G) or a biologically active portion thereof.

115. The combination of claim 114, wherein the G protein or biologically active portion is a biologically active portion of wild-type NiV-G that has a deletion of up to 40 amino acids at or near the N-terminus, optionally not including the initial methionine.

116. The combination of claim 114 or claim 115, wherein the G protein is a biologically active portion that is a truncated NiV-G that has a deletion of amino acids 2-34 at or near the N-terminus of wild-type NiV-G set forth in SEQ ID NO:14.

117. The combination of any of claims 114-116, wherein the G protein or the biologically active portion thereof is a mutant NiV-G protein or biologically active portion thereof that exhibits reduced binding to Ephrin B2 or Ephrin B3.

118. The combination of claim 117, wherein the mutant NiV-G protein comprises one or more amino acid substitutions corresponding to amino acid substitutions selected from the group consisting of E501A, W504A, Q530A and E533A with reference to numbering set forth in SEQ ID NO:14.

119. The combination of any of claims 114-118, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

120. The combination of any of claims 114-119, wherein the F protein or the biologically active portion thereof is a NiV-F protein or a biologically active portion thereof.

121. The combination of claim 120, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

122. The combination of any of claims 114-121, wherein the F protein or the biologically active portion is a truncated NiV-F that lacks amino acids 525-546 of SEQ ID NO:2.

123. The combination of any of claims 114-122, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

124. The combination of any of claims 114-123, wherein the Niv-G protein is a biologically active portion set forth by the amino acid sequence set forth in SEQ ID NO: 19, and the Niv-F protein is a biological active portion set forth by the amino acid sequence set forth in SEQ ID NO:12.

125. The combination of any of claims 111-124, wherein the fusogen is a re-targeted fusogen comprising a targeting moiety that binds to a target cell.

126. The method of claim 125, wherein the fusogen comprises a paramyxovirus G, paramyxovirus H and/or paramyxovirus HN protein and wherein the targeting moiety is linked to the paramyxovirus G, paramyxovirus H and/or paramyxovirus HN protein.

127. The method of any of claims 111-126, wherein the fusogen is a re-targeted Nipah fusogen and the NiV-G protein or a biologically active portion thereof is further linked to a targeting moiety that binds to a target cell.

128. The combination of any of claims 125-127, wherein the targeting moiety comprises a binding agent, optionally wherein the binding agent is targeted against CD3, CD4, CD8, CD34, CD90, CD19, CD20, CD22, CD16, or CD56.

129. The combination of any of claims 125-128, wherein the target cell is a T cell, a B cell, an NK cell, a macrophage, a monocyte, a dendritic cell, a hematopoietic stem cell or a CD34+progenitor cell, optionally wherein the target cell is a T cell, further optionally wherein the targeting moiety binds to CD4 or CD8.

130. The combination of claim 126-129, wherein the lipid particle or lentiviral vector comprising the retargeted NiV-G is pseudotyped with the a NiV-F of a biologically active portion thereof, optionally wherein the NiV-F or biologically active portion is set forth in SEQ ID NO:12.

131. The combination of any of claims 109-130, wherein the lipid particle or lentiviral vector comprises a nucleic acid encoding a payload gene, optionally wherein the nucleic acid encoding a payload gene encodes a chimeric antigen receptor (CAR).

132. The combination of any of claims 109-131, wherein the composition comprising lipid particles further comprises a cytokine receptor agonist, optionally wherein the cytokine receptor is selected from the group consisting of an IL-2 receptor (IL-2R), an IL-15 receptor (IL-15R), an IL-7 receptor (IL-7R), or an IL-21 receptor (IL-21R).

133. A sterile composition comprising between 1×10⁶ cells/mL to 1×108 cells/mL of peripheral blood mononuclear cells (PBMCs) or subset thereof and viral vector composition consisting of 1×10⁸ to 1×10¹¹ infectious units (IU), optionally wherein the composition has a volume of between 100 mL to 1000 mL.

134. A sterile composition comprising peripheral blood mononuclear cells (PBMCs) from a 100 mL to 400 mL leukapheresis product and a viral vector composition comprising 1×10⁸ to 1×10¹¹ infectious units (IU), optionally wherein the volume of the composition is between 100 mL to 1000 mL.

135. The sterile composition of any of claims 133-134, wherein the virial vector is a lentiviral vector.

136. The sterile composition of any of claims 133-135, wherein the viral vector comprises a fusogen embedded in the lipid bilayer.

137. The sterile composition of claim 136, wherein the fusogen is
(i) a vesicular stomatitis virus envelope glycoprotein (VSV-G),
(ii) a baboon endogenous virus (BaEV) envelope glycoprotein,
(iii) a Cocal virus envelope glycoprotein,
(iv) an Alphavirus fusion protein or a functional variant thereof, optionally a Sindbis virus fusion protein or a functional variant thereof,
(v) a Paramyxoviridae fusion protein or a functional variant thereof, optionally a Morbillivirus or a Henipavirus fusion protein or a functional variant thereof,
(vi) a Morbillivirus fusion protein, optionally measles virus (MeV), canine distemper virus, Cetacean morbillivirus, Peste-des-petits-ruminants virus, Phocine distemper virus, Rinderpest virus, or a functional variant thereof, or
(vii) a Henipavirus fusion protein, optionally Nipah virus, Hendra virus, Cedar virus, Kumasi virus, Mójiāng virus, or a functional variant thereof.

138. The sterile composition of claim 136 or claim 137, wherein the fusogen comprises a paramyxovirus F protein, or a biologically active portion thereof and wherein the fusogen comprises a paramyxovirus G, paramyxovirus H, and/or paramyxovirus HN protein, or a biologically active portion thereof.

139. The sterile composition of claim 138, wherein the paramyxovirus G, paramyxovirus H, and/or paramyxovirus HN protein further comprises a targeting moiety, optionally wherein the targeting moiety comprises a binding agent, further optionally wherein the binding agent is targeted against CD3, CD4, CD8, CD34, CD90, CD19, CD20, CD22, CD16, or CD56.

140. The sterile composition of any of claims 136-139, wherein the fusogen comprises a paramyxovirus G, paramyxovirus H and/or paramyxovirus HN protein and wherein the targeting moiety is linked to the paramyxovirus G, paramyxovirus H and/or paramyxovirus HN protein.

141. The sterile composition of any of claims 136-140, wherein the fusogen is a re-targeted Nipah fusogen and the NiV-G protein or a biologically active portion thereof is further linked to a targeting moiety that binds to a target cell.

142. The sterile composition of any of claims 138-141, wherein the G protein or the biologically active portion thereof is a mutant NiV-G protein or biologically active portion thereof that exhibits reduced binding to Ephrin B2 or Ephrin B3.

143. The sterile composition of claim 142, wherein the mutant NiV-G protein comprises one or more amino acid substitutions corresponding to amino acid substitutions selected from the group consisting of E501A, W504A, Q530A and E533A with reference to numbering set forth in SEQ ID NO:14.

144. The sterile composition of any of claims 138-143, wherein the G protein or biologically active portion is a biologically active portion of wild-type NiV-G that has a deletion of up to 40 amino acids at or near the N-terminus, optionally not including the initial methionine.

145. The sterile composition of any of claims 138-144, wherein the G protein is a biologically active portion that is a truncated NiV-G that has a deletion of amino acids 2-34 at or near the N-terminus of wild-type NiV-G set forth in SEQ ID NO:14.

146. The sterile composition of any of claims 138-145, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

147. The sterile composition of any of claims 138-146, wherein the F protein or the biologically active portion thereof is a NiV-F protein or a biologically active portion thereof.

148. The sterile composition of claim 147, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

149. The sterile composition of any of claims 138-148, wherein the F protein or the biologically active portion is a truncated NiV-F that lacks amino acids 525-546 of SEQ ID NO:2.

150. The sterile composition of any of claims 138-149, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

151. The sterile composition of any of claims 138-150, wherein the Niv-G protein comprises the amino acid sequence set forth in SEQ ID NO: 19, and the Niv-F protein comprises the amino acid sequence set forth in SEQ ID NO:12.

152. The sterile composition of any of claims 133-151, wherein the viral vector comprises a nucleic acid encoding a payload gene, optionally wherein the nucleic acid encoding a payload gene encodes a chimeric antigen receptor (CAR).

153. A container comprising the sterile composition of any of claims 133-152, optionally wherein the container is a bag.

154. A method of treating a disease or condition in a subject comprising administering a lipid particle or payload gene by the method of any of claims 1-70 and 76-99 to a subject in need thereof.

155. A method of treating a disease or condition comprising infusing and/or administering the sterile composition of any of claims 133-152 into or to a subject having a disease or condition and/or in need thereof, optionally wherein the infusion or administration is by in-line infusion of the composition to the subject.

156. A method of treating a disease or condition comprising infusing the lentiviral vector encoding the chimeric antigen receptor (CAR) by the method of any of claims 8-73 and 74-99 to a subject having a disease or condition in need of treatment thereof, optionally wherein the CAR comprises an extracellular antigen binding domain specific for an antigen associated with the disease or condition, further optionally wherein the CAR is an anti-CD19 CAR, an anti-CD22 CAR, an anti-CD20 CAR or an anti-BCMA CAR.

157. The method of any of claims 154-156, wherein the disease or condition is a cancer, optionally wherein
(i) the cancer is a solid tumor, a lymphoma or a leukemia,
(ii) the cancer is a B cell Lymphoma, optionally a Non-Hodgkin lymphoma (NHL), DLBCL, or follicular lymphoma,
(iii) the cancer is a relapsed/refractory cancer, and/or
(iv) the cancer is a relapsed and/or refractory Large B-cell Lymphoma (LBCL), optionally wherein the LBCL comprises Non-Hodgkin's lymphoma (NHL).

158. The method of any of claims 1-99 and 154-157, wherein the subject has not received a lymphodepleting regimen or therapy, optionally wherein the subject has not received a lymphodepleting regimen or therapy within 30 days prior to the use or administration.

159. The method of any of claims 1-99 and 154-158, prior to performing the therapy or method, further comprising administering to the subject one or more agents to mobilize peripheral blood hematopoietic stem cells or CD34+progenitor cells, optionally wherein the agent is G-CSF.

160. A lipid particle therapy for use in treating a subject having a disease or condition in need of treatment, wherein the lipid particle therapy particle therapy is administered to the subject by the method of any of claims 1-70 and 76-99.

161. A lentiviral vector therapy for use in treating a subject having a disease or condition in need of treatment, wherein the lentiviral vector therapy is for administration to the subject by the method of any of claims 6-38 and 40-99.

162. The sterile composition of any of claims 133-152 for use in treating a subject having a disease or condition in need of treatment by in-line infusion of the composition to a subject, optionally wherein the in-line infusion comprises an apheresis device.

163. The lipid particle therapy for use of claim 160, the lentiviral vector therapy for use of claim 161 or the sterile composition for use of claim 162, wherein the disease or condition is a cancer.

164. The lipid particle therapy for use, the lentiviral vector therapy for use or the sterile composition for use of any of claims 160-163, prior to the use, the subject has received one or more agents to mobilize peripheral blood hematopoietic stem cells or CD34+progenitor cells, optionally wherein the agent is G-CSF.

165. The method of claim 159 or the lipid particle therapy for use, the lentiviral vector therapy for use or the sterile composition for use of claim 164, wherein the one or more agents that stimulate mobilization are selected from the group consisting of stem cell factor (SCF), small molecule VLA-4 inhibitor BI05192, BOP (N-(benzenesulfonyl)-L-prolyl-L-0-(1-pyrrolidinylcarbonyl)tyrosine), heparin, granulocyte colony-stimulating factor (G-CSF), MGTA-145, and plerixafor (AMD3100).

166. The method or the lipid particle therapy for use, the lentiviral vector therapy for use or the sterile composition for use of any of claims 159 and 164-165, wherein the one or more agents that stimulate mobilization are G-CSF and plerixafor.


SEQUENCE LISTING (312 sequences)

SEQ ID NO: 1 - protein, 546 aa, synthetic construct
  REGION 1..546: note = Hendra F
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 2 - protein, 546 aa, synthetic construct
  REGION 1..546: note = Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 3 - protein, 557 aa, synthetic construct
  REGION 1..557: note = CedV F
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 4 - protein, 545 aa, synthetic construct
  REGION 1..545: note = Mojiang F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 5 - protein, 662 aa, synthetic construct
  REGION 1..662: note = Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 6 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 7 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 8 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 9 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 10 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 11 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 12 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 13 - protein, 604 aa, synthetic construct
  REGION 1..604: note = Hendra virus G protein Uniprot O89343
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 14 - protein, 602 aa, synthetic construct
  REGION 1..602: note = Uniprot Q9IH62
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 15 - protein, 622 aa, synthetic construct
  REGION 1..622: note = gb:JQ001776:8170-10275
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 16 - protein, 632 aa, synthetic construct
  REGION 1..632: note = gb:NC_025256:9117-11015
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 17 - protein, 625 aa, synthetic construct
  REGION 1..625: note = gb:NC_025352:8716-11257
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 18 - protein, 622 aa, synthetic construct; identical to SEQ ID NO: 15
  REGION 1..622: note = gb:JQ001776:8170-10275

SEQ ID NO: 19 - protein, 568 aa, synthetic construct
  REGION 1..568: note = (E501 A, W504A, Q530A, E533A) NiV G protein (Gc del 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQC

SEQ ID NO: 20 - protein, 5 aa, synthetic construct
  REGION 1..5: note = Linker
GGGGS

SEQ ID NO: 21 - protein, 6 aa, synthetic construct
  REGION 1..6: note = Linker
GGGGGS

SEQ ID NO: 22 - protein, 5 aa, synthetic construct
  REGION 1..5: note = Linker
  REPEAT 1..5: note = repeat any number of times
GGGGS

SEQ ID NO: 23 - protein, 6 aa, synthetic construct
  REGION 1..6: note = Linker
  REPEAT 1..6: note = Repeat any number of times
GGGGGS

SEQ ID NO: 24 - protein, 21 aa, synthetic construct
  REGION 1..21: note = CD8alpha signal peptide
MALPVTALLLPLALLLHAARP

SEQ ID NO: 25 - protein, 20 aa, synthetic construct
  REGION 1..20: note = IgK signal peptide
METDTLLLWVLLLWVPGSTG

SEQ ID NO: 26 - protein, 22 aa, synthetic construct
  REGION 1..22: note = CSF2RA signal peptide
MLLLVTSLLLCELPHPAFLLIP

SEQ ID NO: 27 - protein, 45 aa, synthetic construct
  REGION 1..45: note = CD8alpha hinge domain
TTTPAPRPPTPAPTIASQPLSLRPEACRPAAGGAVHTRGLDFACD

SEQ ID NO: 28 - protein, 39 aa, synthetic construct
  REGION 1..39: note = CD28 hinge domain
IEVMYPPPYLDNEKSNGTIIHVKGKHLCPSPLFPGPSKP

SEQ ID NO: 29 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 30 - protein, 12 aa, synthetic construct
  REGION 1..12: note = IgG4 hinge domain
ESKYGPPCPPCP

SEQ ID NO: 31 - protein, 12 aa, synthetic construct
  REGION 1..12: note = IgG4 hinge domain
ESKYGPPCPSCP

SEQ ID NO: 32 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 24 aa, synthetic construct
  REGION 1..24: note = CD8alpha transmembrane domain
IYIWAPLAGTCGVLLLSLVITLYC

SEQ ID NO: 34 - protein, 27 aa, synthetic construct
  REGION 1..27: note = CD28 transmembrane domain
FWVLVVVGGVLACYSLLVTVAFIIFWV

SEQ ID NO: 35 - protein, 28 aa, synthetic construct
  REGION 1..28: note = CD28 transmembrane domain
MFWVLVVVGGVLACYSLLVTVAFIIFWV

SEQ ID NO: 36 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 37 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 38 - protein, 112 aa, synthetic construct
  REGION 1..112: note = CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 112 aa, synthetic construct
  REGION 1..112: note = CD3z signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 245 aa, synthetic construct
  REGION 1..245: note = Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 41 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 42 - protein, 6 aa, synthetic construct
  REGION 1..6: note = Anti-CD19 FMC63 scFv light chain CDR1
QDISKY

SEQ ID NO: 43 - skipped

SEQ ID NO: 44 - protein, 9 aa, synthetic construct
  REGION 1..9: note = Anti-CD19 FMC63 scFv light chain CDR3
QQGNTLPYT

SEQ ID NO: 45 - protein, 18 aa, synthetic construct
  REGION 1..18: note = Whitlow linker
GSTSGSGKPGSGEGSTKG

SEQ ID NO: 46 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 47 - protein, 8 aa, synthetic construct
  REGION 1..8: note = Anti-CD19 FMC63 scFv heavy chain CDR1
GVSLPDYG

SEQ ID NO: 48 - protein, 7 aa, synthetic construct
  REGION 1..7: note = Anti-CD19 FMC63 scFv heavy chain CDR2
IWGSETT

SEQ ID NO: 49 - protein, 14 aa, synthetic construct
  REGION 1..14: note = Anti-CD19 FMC63 scFv heavy chain CDR3
AKHYYYGGSYAMDY

SEQ ID NO: 50 - protein, 242 aa, synthetic construct
  REGION 1..242: note = Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 51 - protein, 15 aa, synthetic construct
  REGION 1..15: note = 3xG4S linker
GGGGSGGGGSGGGGS

SEQ ID NO: 52 - DNA, 1467 bp, synthetic construct
  source 1..1467: mol_type = other DNA
  misc_feature 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 53 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1458 bp, synthetic construct
  source 1..1458: mol_type = other DNA
  misc_feature 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 1383 bp, synthetic construct
  source 1..1383: mol_type = other DNA
  misc_feature 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 57 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1467 bp, synthetic construct
  source 1..1467: mol_type = other DNA
  misc_feature 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 59 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 61 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 62 - protein, 10 aa, synthetic construct
  REGION 1..10: note = Anti-CD20 Leu16 scFv light chain CDR1
RASSSVNYMD

SEQ ID NO: 63 - protein, 7 aa, synthetic construct
  REGION 1..7: note = Anti-CD20 Leu16 scFv light chain CDR2
ATSNLAS

SEQ ID NO: 64 - protein, 9 aa, synthetic construct
  REGION 1..9: note = Anti-CD20 Leu16 scFv light chain CDR3
QQWSFNPPT

SEQ ID NO: 65 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 66 - protein, 5 aa, synthetic construct
  REGION 1..5: note = Anti-CD20 Leu16 scFv heavy chain CDR1
SYNMH

SEQ ID NO: 67 - protein, 17 aa, synthetic construct
  REGION 1..17: note = Anti-CD20 Leu16 scFv heavy chain CDR2
AIYPGNGDTSYNQKFKG

SEQ ID NO: 68 - protein, 13 aa, synthetic construct
  REGION 1..13: note = Anti-CD20 Leu16 scFv heavy chain CDR3
SNYYGSSYWFFDV

SEQ ID NO: 69 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 70 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 71 - protein, 10 aa, synthetic construct
  REGION 1..10: note = Anti-CD22 m971 scFv heavy chain CDR1
GDSVSSNSAA

SEQ ID NO: 72 - protein, 9 aa, synthetic construct
  REGION 1..9: note = Anti-CD22 m971 scFv heavy chain CDR2
TYYRSKWYN

SEQ ID NO: 73 - protein, 14 aa, synthetic construct
  REGION 1..14: note = Anti-CD22 m971 scFv heavy chain CDR3
AREVTGDLEDAFDI

SEQ ID NO: 74 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 75 - protein, 6 aa, synthetic construct
  REGION 1..6: note = Anti-CD22 m971 scFv light chain CDR1
QTIWSY

SEQ ID NO: 76 - skipped

SEQ ID NO: 77 - protein, 9 aa, synthetic construct
  REGION 1..9: note = Anti-CD22 m971 scFv light chain CDR3
QQSYSIPQT

SEQ ID NO: 78 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 79 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 80 - protein, 10 aa, synthetic construct
  REGION 1..10: note = Anti-CD22 m971-L7 scFv heavy chain CDR1
GDSVSSNSVA

SEQ ID NO: 81 - protein, 9 aa, synthetic construct
  REGION 1..9: note = Anti-CD22 m971-L7 scFv heavy chain CDR2
TYYRSTWYN

SEQ ID NO: 82 - protein, 14 aa, synthetic construct
  REGION 1..14: note = Anti-CD22 m971-L7 scFv heavy chain CDR3
AREVTGDLEDAFDI

SEQ ID NO: 83 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 84 - protein, 6 aa, synthetic construct
  REGION 1..6: note = Anti-CD22 m971-L7 scFv light chain CDR1
QTIWSY

SEQ ID NO: 85 - skipped

SEQ ID NO: 86 - protein, 9 aa, synthetic construct
  REGION 1..9: note = Anti-CD22 m971-L7 scFv light chain CDR3
QQSYSIPQT

SEQ ID NO: 87 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 88 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 89 - protein, 15 aa, synthetic construct
  REGION 1..15: note = Anti-BCMA C11D5.3 scFv light chain CDR1
RASESVSVIGAHLIH

SEQ ID NO: 90 - protein, 7 aa, synthetic construct
  REGION 1..7: note = Anti-BCMA C11D5.3 scFv light chain CDR2
LASNLET

SEQ ID NO: 91 - protein, 9 aa, synthetic construct
  REGION 1..9: note = Anti-BCMA C11D5.3 scFv light chain CDR3
LQSRIFPRT

SEQ ID NO: 92 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 93 - protein, 5 aa, synthetic construct
  REGION 1..5: note = Anti-BCMA C11D5.3 scFv heavy chain CDR1
DYSIN

SEQ ID NO: 94 - protein, 17 aa, synthetic construct
  REGION 1..17: note = Anti-BCMA C11D5.3 scFv heavy chain CDR2
WINTETREPAYAYDFRG

SEQ ID NO: 95 - protein, 8 aa, synthetic construct
  REGION 1..8: note = Anti-BCMA C11D5.3 scFv heavy chain CDR3
DYSYAMDY

SEQ ID NO: 96 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 97 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 98 - protein, 15 aa, synthetic construct
  REGION 1..15: note = Anti-BCMA C12A3.2 scFv light chain CDR1
RASESVTILGSHLIY

SEQ ID NO: 99 - protein, 7 aa, synthetic construct
  REGION 1..7: note = Anti-BCMA C12A3.2 scFv light chain CDR2
LASNVQT

SEQ ID NO: 100 - protein, 9 aa, synthetic construct
  REGION 1..9: note = Anti-BCMA C12A3.2 scFv light chain CDR3
LQSRTIPRT

SEQ ID NO: 101 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 102 - protein, 5 aa, synthetic construct
  REGION 1..5: note = Anti-BCMA C12A3.2 scFv heavy chain CDR1
HYSMN

SEQ ID NO: 103 - protein, 17 aa, synthetic construct
  REGION 1..17: note = Anti-BCMA C12A3.2 scFv heavy chain CDR2
RINTESGVPIYADDFKG

SEQ ID NO: 104 - protein, 8 aa, synthetic construct
  REGION 1..8: note = Anti-BCMA C12A3.2 scFv heavy chain CDR3
DYLYSLDF

SEQ ID NO: 105 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 106 - protein, 8 aa, synthetic construct
  REGION 1..8: note = Anti-BCMA FHVH33 CDR1
GFTFSSYA

SEQ ID NO: 107 - protein, 8 aa, synthetic construct
  REGION 1..8: note = Anti-BCMA FHVH33 CDR2
ISGSGDYI

SEQ ID NO: 108 - protein, 14 aa, synthetic construct
  REGION 1..14: note = Anti-BCMA FHVH33 CDR3
AKEGTGANSSLADY

SEQ ID NO: 109 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 110 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 111 - protein, 6 aa, synthetic construct
  REGION 1..6: note = Anti-BCMA CT103A scFv light chain CDR1
QSISSY

SEQ ID NO: 112 - skipped

SEQ ID NO: 113 - protein, 8 aa, synthetic construct
  REGION 1..8: note = Anti-BCMA CT103A scFv light chain CDR3
QQKYDLLT

SEQ ID NO: 114 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 115 - protein, 10 aa, synthetic construct
  REGION 1..10: note = Anti-BCMA CT103A scFv heavy chain CDR1
GGSISSSSYY

SEQ ID NO: 116 - protein, 7 aa, synthetic construct
  REGION 1..7: note = Anti-BCMA CT103A scFv heavy chain CDR2
ISYSGST

SEQ ID NO: 117 - protein, 11 aa, synthetic construct
  REGION 1..11: note = Anti-BCMA CT103A scFv heavy chain CDR3
ARDRGDTILDV

SEQ ID NO: 118 - DNA, 1503 bp, synthetic construct
  source 1..1503: mol_type = other DNA
  misc_feature 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 119 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 122 aa, synthetic construct
  REGION 1..122: note = CD8_1 VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 121 - protein, 108 aa, synthetic construct
  REGION 1..108: note = CD8_1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 122 - protein, 119 aa, synthetic construct
  REGION 1..119: note = CD8_2 VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 123 - protein, 113 aa, synthetic construct
  REGION 1..113: note = CD8_2 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 124 - protein, 117 aa, synthetic construct
  REGION 1..117: note = CD8_3 VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 125 - protein, 108 aa, synthetic construct
  REGION 1..108: note = CD8_3 VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 126 - protein, 118 aa, synthetic construct
  REGION 1..118: note = CD8_4 VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 127 - protein, 108 aa, synthetic construct
  REGION 1..108: note = CD8_4 VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 128 - protein, 125 aa, synthetic construct
  REGION 1..125: note = CD8_VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 129 - protein, 133 aa, synthetic construct [sequence omitted]

SEQ ID NO: 130 - protein, 153 aa, synthetic construct
  REGION 1..153: note = IL-2 (precursor)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 131 - protein, 132 aa, synthetic construct
  REGION 1..132: note = Aldesleukin/proleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 132 - protein, 8 aa, synthetic construct
  REGION 1..8: note = 17
GFTFSYYG

SEQ ID NO: 133 - protein, 8 aa, synthetic construct
  REGION 1..8: note = 18
ISNDGSNK

SEQ ID NO: 134 - protein, 12 aa, synthetic construct
  REGION 1..12: note = 19
RPEDTAVYYCAT

SEQ ID NO: 135 - protein, 8 aa, synthetic construct
  REGION 1..8: note = 20
GYNFDTYW

SEQ ID NO: 136 - protein, 8 aa, synthetic construct
  REGION 1..8: note = 21
IYPADSDT

SEQ ID NO: 137 - protein, 10 aa, synthetic construct
  REGION 1..10: note = 22
ARFSSSAYDI

SEQ ID NO: 138 - protein, 8 aa, synthetic construct
  REGION 1..8: note = 23
GYNFDTYW

SEQ ID NO: 139 - protein, 8 aa, synthetic construct
  REGION 1..8: note = 24
IYPADSDT

SEQ ID NO: 140 - protein, 10 aa, synthetic construct
  REGION 1..10: note = 25
SSAYDIARFS

SEQ ID NO: 141 - protein, 8 aa, synthetic construct
  REGION 1..8: note = 26
GFTFNRYA

SEQ ID NO: 142 - protein, 8 aa, synthetic construct
  REGION 1..8: note = 27
ISFDGGNQ

SEQ ID NO: 143 - protein, 14 aa, synthetic construct
  REGION 1..14: note = 28
VRSPAGDWVAYFDY

SEQ ID NO: 144 - protein, 6 aa, synthetic construct
  REGION 1..6: note = 29
ALPKRF

SEQ ID NO: 145 - skipped

SEQ ID NO: 146 - protein, 11 aa, synthetic construct
  REGION 1..11: note = 31
YSTDNTGGLWV

SEQ ID NO: 147 - protein, 6 aa, synthetic construct
  REGION 1..6: note = 32
ALPRQF

SEQ ID NO: 148 - skipped

SEQ ID NO: 149 - protein, 10 aa, synthetic construct
  REGION 1..10: note = 34
DSSGTYHQSA

SEQ ID NO: 150 - protein, 6 aa, synthetic construct
  REGION 1..6: note = 35
ALPRKF

SEQ ID NO: 151 - skipped

SEQ ID NO: 152 - protein, 10 aa, synthetic construct
  REGION 1..10: note = 37
QSADRSDSYV

SEQ ID NO: 153 - protein, 6 aa, synthetic construct
  REGION 1..6: note = 38
EDISNH

SEQ ID NO: 154 - skipped

SEQ ID NO: 155 - protein, 10 aa, synthetic construct
  REGION 1..10: note = 40
HQYHDYPSYT

SEQ ID NO: 156 - protein, 177 aa, synthetic construct
  REGION 1..177: note = Genbank Accession No. P13232 (human IL-7)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 157 - protein, 162 aa, synthetic construct
  REGION 1..162: note = IL-15 LSP
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 158 - protein, 135 aa, synthetic construct
  REGION 1..135: note = IL-15 SSP
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 159 - protein, 114 aa, synthetic construct
  REGION 1..114: note = IL-15 mature
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 160 - protein, 25 aa, synthetic construct [sequence omitted]

SEQ ID NO: 161 - protein, 17 aa, synthetic construct
  REGION 1..17: note = 25
IHWVRQAPGQGLELIGW

SEQ ID NO: 162 - protein, 38 aa, synthetic construct
  REGION 1..38: note = 26
NYAQKFQVKATMTRDTSISTAYMELSRLRSDDTAVYYC

SEQ ID NO: 163 - protein, 11 aa, synthetic construct
  REGION 1..11: note = 27
WGQGTTVTVSS

SEQ ID NO: 164 - protein, 26 aa, synthetic construct
  REGION 1..26: note = 28
DVVMTQSPLSLPVTLGQPASISCKSS

SEQ ID NO: 165 - protein, 16 aa, synthetic construct
  REGION 1..16: note = 29
LNWLQQRPGQSPRRLI

SEQ ID NO: 166 - protein, 36 aa, synthetic construct
  REGION 1..36: note = 30
KLDSGVPDRFSGSGSGTDFTLKISRVEAEDVGVYYC

SEQ ID NO: 167 - protein, 10 aa, synthetic construct
  REGION 1..10: note = 31
FGQGTKLEIK

SEQ ID NO: 168 - protein, 30 aa, synthetic construct
  REGION 1..30: note = 32
QVQLVQSGAEVKKPGASVKVSCKASGFNIK

SEQ ID NO: 169 - protein, 14 aa, synthetic construct
  REGION 1..14: note = 33
WVRQAPGQGLELIG

SEQ ID NO: 170 - protein, 32 aa, synthetic construct
  REGION 1..32: note = 34
KATMTRDTSISTAYMELSRLRSDDTAVYYCND

SEQ ID NO: 171 - protein, 11 aa, synthetic construct
  REGION 1..11: note = 35
WGQGTTVTVSS

SEQ ID NO: 172 - protein, 23 aa, synthetic construct
  REGION 1..23: note = 36
DVVMTQSPLSLPVTLGQPASISC

SEQ ID NO: 173 - protein, 15 aa, synthetic construct
  REGION 1..15: note = 37
WLQQRPGQSPRRLIS

SEQ ID NO: 174 - protein, 32 aa, synthetic construct
  REGION 1..32: note = 38
GVPDRFSGSGSGTDFTLKISRVEAEDVGVYYC

SEQ ID NO: 175 - protein, 10 aa, synthetic construct
  REGION 1..10: note = 39
FGQGTKLEIK

SEQ ID NO: 176 - protein, 133 aa, synthetic construct [sequence omitted]

SEQ ID NO: 177 - protein, 133 aa, synthetic construct
  REGION 1..133: note = IL-21 (157)
QGQDEHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 178 - protein, 123 aa, synthetic construct
  REGION 1..123: note = IL-21 (158)
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHL

SEQ ID NO: 179 - protein, 153 aa, synthetic construct
  REGION 1..153: note = IL-7 (1)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 180 - protein, 152 aa, synthetic construct
  REGION 1..152: note = IL-7 (2)
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 181 - protein, 134 aa, synthetic construct
  REGION 1..134: note = IL-7 (3)
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQEENKSLKEQKKLNDLCFLKRLLQEIKTCWNKILMGTKEH

SEQ ID NO: 182 - protein, 123 aa, synthetic construct
  REGION 1..123: note = IL-7 (4)
DCDIEGKDGKQYESVLMVSIDQLLDSMKEIGSNCLNNEFNFFKRHICDANKEGMFLFRAARKLRQFLKMNSTGDFDLHLLKVSEGTTILLKEQKKLNDLCFLKRLLQEIKTCWNKILMGTKEH

SEQ ID NO: 183 - protein, 108 aa, synthetic construct
  REGION 1..108: note = IL-7 (5)
DCDIEGKDGKQYESVLMVSIDQLLDSMKEIGSNCLNNEFNFFKRHICDANKVKGRKPAALGEAQPTKSLEENESLKEQKKLNDLCFLKRLLQEIKTCWNKILMGTKEH

SEQ ID NO: 184 - protein, 90 aa, synthetic construct
  REGION 1..90: note = IL-7 (6)
DCDIEGKDGKQYESVLMVSIDQLLDSMKEIGSNCLNNEFNFFKRHICDANKEENESLKEQKKLNDLCFLKRLLQEIKTCWNKILMGTKEH

SEQ ID NO: 185 - protein, 81 aa, synthetic construct
  REGION 1..81: note = IL-7 (7)
DCDIEGKDGKQYESVLMVSIDQLLVKGRKPAALGEAQPTKSLEENESLKEQKKLNDLCFLKRLLQEIKTCWNKILMGTKEH

SEQ ID NO: 186 - protein, 63 aa, synthetic construct
  REGION 1..63: note = IL-7 (8)
DCDIEGKDGKQYESVLMVSIDQLLEENKSLKEQKKLNDLCFLKRLLQEIKTCWNKILMGTKEH

SEQ ID NO: 187 - protein, 108 aa, synthetic construct
  REGION 1..108: note = IL-7 (mature human IL-7)
DCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 188 - protein, 138 aa, synthetic construct
  REGION 1..138: note = IL-21 (mature human IL-21)
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 189 - protein, 8 aa, synthetic construct
  REGION 1..8: note = CDR-H1
GFTFSGYW

SEQ ID NO: 190 - protein, 8 aa, synthetic construct
  REGION 1..8: note = CDR-H2
ISPGGGST

SEQ ID NO: 191 - protein, 14 aa, synthetic construct
  REGION 1..14: note = CDR-H3
ASSLTATHTYEYDY

SEQ ID NO: 192 - protein, 121 aa, synthetic construct
  REGION 1..121: note = VHH
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 193 - protein, 8 aa, synthetic construct
  REGION 1..8: note = CDR-H1
GYTFSNYW

SEQ ID NO: 194 - protein, 8 aa, synthetic construct
  REGION 1..8: note = CDR-H2
ILPGSGST

SEQ ID NO: 195 - protein, 13 aa, synthetic construct
  REGION 1..13: note = CDR-H3
ARRGYGYDEGFDY

SEQ ID NO: 196 - protein, 6 aa, synthetic construct
  REGION 1..6: note = CDR-L1
QDINSY

SEQ ID NO: 197 - skipped

SEQ ID NO: 198 - protein, 9 aa, synthetic construct
  REGION 1..9: note = CDR-L3
LQYDEFPPT

SEQ ID NO: 199 - protein, 120 aa, synthetic construct
  REGION 1..120: note = VH
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSS

SEQ ID NO: 200 - protein, 108 aa, synthetic construct
  REGION 1..108: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 201 - protein, 243 aa, synthetic construct
  REGION 1..243: note = scFv
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 202 - protein, 8 aa, synthetic construct
  REGION 1..8: note = CDR-H1
GYTFTDYV

SEQ ID NO: 203 - protein, 8 aa, synthetic construct
  REGION 1..8: note = CDR-H2
IYPGSGSS

SEQ ID NO: 204 - protein, 10 aa, synthetic construct
  REGION 1..10: note = CDR-H3
ARPGDLGFAY

SEQ ID NO: 205 - protein, 10 aa, synthetic construct
  REGION 1..10: note = CDR-L1
QSVDYDGDSY

SEQ ID NO: 206 - skipped

SEQ ID NO: 207 - protein, 9 aa, synthetic construct
  REGION 1..9: note = CDR-L3
QQSNKDPFT

SEQ ID NO: 208 - protein, 117 aa, synthetic construct
  REGION 1..117: note = VH
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 209 - protein, 112 aa, synthetic construct
  REGION 1..112: note = VL
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 210 - protein, 244 aa, synthetic construct
  REGION 1..244: note = scFv
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSSGGGGSGGGGSGGGGSDIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 211 - protein, 8 aa, synthetic construct
  REGION 1..8: note = CDR-H1
GGTFSSYA

SEQ ID NO: 212 - protein, 8 aa, synthetic construct
  REGION 1..8: note = CDR-H2
INPNSGGT

SEQ ID NO: 213 - protein, 11 aa, synthetic construct
  REGION 1..11: note = CDR-H3
ARDGYSGSYSD

SEQ ID NO: 214 - protein, 12 aa, synthetic construct
  REGION 1..12: note = CDR-L1
QSVLSSSYNKNY

SEQ ID NO: 215 - skipped

SEQ ID NO: 216 - protein, 9 aa, synthetic construct
  REGION 1..9: note = CDR-L3
QQYYSTPWT

SEQ ID NO: 217 - protein, 118 aa, synthetic construct
  REGION 1..118: note = VH
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSS

SEQ ID NO: 218 - protein, 113 aa, synthetic construct
  REGION 1..113: note = VL
DIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 219 - protein, 246 aa, synthetic construct
  REGION 1..246: note = scFv
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSSGGGGSGGGGSGGGGSDIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 220 - protein, 8 aa, synthetic construct
  REGION 1..8: note = CDR-H1
GYTFTSYD

SEQ ID NO: 221 - protein, 8 aa, synthetic construct
  REGION 1..8: note = CDR-H2
IIPLSGAP

SEQ ID NO: 222 - protein, 15 aa, synthetic construct
  REGION 1..15: note = CDR-H3
ARGALYNWNDGWFDP

SEQ ID NO: 223 - protein, 6 aa, synthetic construct
QDIGDY

SEQ ID NO: 224 - skipped

SEQ ID NO: 225 - protein, 9 aa, synthetic construct
  REGION 1..9: note = CDR-L3
QQANSFPLT

SEQ ID NO: 226 - protein, 122 aa, synthetic construct
  REGION 1..122: note = VH
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSS

SEQ ID NO: 227 - protein, 107 aa, synthetic construct
  REGION 1..107: note = VL
DIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 228 - protein, 244 aa, synthetic construct
  REGION 1..244: note = scFv
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSSGGGGSGGGGSGGGGSDIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 229 - protein, 8 aa, synthetic construct
  REGION 1..8: note = CDR-H1
GDTFSDHA

SEQ ID NO: 230 - protein, 8 aa, synthetic construct
  REGION 1..8: note = CDR-H2
MNPKIGNT

SEQ ID NO: 231 - protein, 12 aa, synthetic construct
  REGION 1..12: note = CDR-H3
VYDSSGYDAFDI

SEQ ID NO: 232 - protein, 12 aa, synthetic construct
  REGION 1..12: note = CDR-L1
QSVLSTSYNRNF

SEQ ID NO: 233 - protein, 9 aa, synthetic construct
  REGION 1..9: note = CDR-L3
QQYYSTPYT

SEQ ID NO: 234 - protein, 119 aa, synthetic construct
  REGION 1..119: note = VH
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSS

SEQ ID NO: 235 - protein, 113 aa, synthetic construct
  REGION 1..113: note = VL
DIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 236 - protein, 247 aa, synthetic construct
  REGION 1..247: note = scFv
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSSGGGGSGGGGSGGGGSDIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 237 - protein, 8 aa, synthetic construct
  REGION 1..8: note = CDR-H1
GYSLITHW

SEQ ID NO: 238 - protein, 8 aa, synthetic construct
  REGION 1..8: note = CDR-H2
INPSDGVT

SEQ ID NO: 239 - protein, 11 aa, synthetic construct
  REGION 1..11: note = CDR-H3
AREYYGEGFDY

SEQ ID NO: 240 - protein, 6 aa, synthetic construct
  REGION 1..6: note = CDR-L1
QGISNY

SEQ ID NO: 241 - skipped

SEQ ID NO: 242 - protein, 9 aa, synthetic construct
  REGION 1..9: note = CDR-L3
QQSYSTPLT

SEQ ID NO: 243 - protein, 118 aa, synthetic construct
  REGION 1..118: note = VH
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 244 - protein, 108 aa, synthetic construct
  REGION 1..108: note = VL
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 245 - protein, 241 aa, synthetic construct
  REGION 1..241: note = scFv
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSSGGGGSGGGGSGGGGSDIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 246 - protein, 5 aa, synthetic construct
  REGION 1..5: note = CDR-H1
GYWMY

SEQ ID NO: 247 - protein, 17 aa, synthetic construct
  REGION 1..17: note = CDR-H2
AISPGGGSTYYPDSVKG

SEQ ID NO: 248 - protein, 12 aa, synthetic construct
  REGION 1..12: note = CDR-H3
SLTATHTYEYDY

SEQ ID NO: 249 - protein, 7 aa, synthetic construct
  REGION 1..7: note = CDR-H1
GFTFSGY

SEQ ID NO: 250 - protein, 6 aa, synthetic construct
  REGION 1..6: note = CDR-H2
SPGGGS

SEQ ID NO: 251 - protein, 5 aa, synthetic construct
  REGION 1..5: note = CDR-H1
NYWIE

SEQ ID NO: 252 - protein, 17 aa, synthetic construct
  REGION 1..17: note = CDR-H2
EILPGSGSTSYNEKFKG

SEQ ID NO: 253 - protein, 11 aa, synthetic construct
  REGION 1..11: note = CDR-H3
RGYGYDEGFDY

SEQ ID NO: 254 - protein, 11 aa, synthetic construct
  REGION 1..11: note = CDR-L1
KASQDINSYLS

SEQ ID NO: 255 - protein, 7 aa, synthetic construct
  REGION 1..7: note = CDR-L2
RANRLVD

SEQ ID NO: 256 - protein, 7 aa, synthetic construct
  REGION 1..7: note = CDR-H1
GYTFSNY

SEQ ID NO: 257 - protein, 6 aa, synthetic construct
  REGION 1..6: note = CDR-H2
LPGSGS

SEQ ID NO: 258 - protein, 5 aa, synthetic construct
  REGION 1..5: note = CDR-H1
DYVIS

SEQ ID NO: 259 - protein, 17 aa, synthetic construct
  REGION 1..17: note = CDR-H2
EIYPGSGSSYYNEKFKG

SEQ ID NO: 260 - protein, 8 aa, synthetic construct
  REGION 1..8: note = CDR-H3
PGDLGFAY

SEQ ID NO: 261 - protein, 15 aa, synthetic construct
  REGION 1..15: note = CDR-L1
KASQSVDYDGDSYMN

SEQ ID NO: 262 - protein, 7 aa, synthetic construct
  REGION 1..7: note = CDR-L2
AASNLES

SEQ ID NO: 263 - protein, 7 aa, synthetic construct
  REGION 1..7: note = CDR-H1
GYTFTDY

SEQ ID NO: 264 - protein, 6 aa, synthetic construct
  REGION 1..6: note = CDR-H2
YPGSGS

SEQ ID NO: 265 - protein, 5 aa, synthetic construct
  REGION 1..5: note = CDR-H1
SYAIS

SEQ ID NO: 266 - protein, 17 aa, synthetic construct
  REGION 1..17: note = CDR-H2
WINPNSGGTNYAQKFQG

SEQ ID NO: 267 - protein, 9 aa, synthetic construct
  REGION 1..9: note = CDR-H3
DGYSGSYSD

SEQ ID NO: 268 - protein, 17 aa, synthetic construct
  REGION 1..17: note = CDR-L1
KSSQSVLSSSYNKNYLA

SEQ ID NO: 269 - protein, 7 aa, synthetic construct
  REGION 1..7: note = CDR-L2
WASTRES

SEQ ID NO: 270 - protein, 7 aa, synthetic construct
  REGION 1..7: note = CDR-H1
GGTFSSY

SEQ ID NO: 271 - protein, 6 aa, synthetic construct
  REGION 1..6: note = CDR-H2
NPNSGG

SEQ ID NO: 272 - protein, 5 aa, synthetic construct
  REGION 1..5: note = CDR-H1
SYDIN

SEQ ID NO: 273 - protein, 17 aa, synthetic construct
  REGION 1..17: note = CDR-H2
GIIPLSGAPNYAHKFQG

SEQ ID NO: 274 - protein, 13 aa, synthetic construct
  REGION 1..13: note = CDR-H3
GALYNWNDGWFDP

SEQ ID NO: 275 - protein, 11 aa, synthetic construct
  REGION 1..11: note = CDR-L1
RASQDIGDYLA

SEQ ID NO: 276 - protein, 7 aa, synthetic construct
  REGION 1..7: note = CDR-L2
DASSLQS

SEQ ID NO: 277 - protein, 7 aa, synthetic construct
  REGION 1..7: note = CDR-H1
GYTFTSY

SEQ ID NO: 278 - protein, 6 aa, synthetic construct
  REGION 1..6: note = CDR-H2
IPLSGA

SEQ ID NO: 279 - protein, 5 aa, synthetic construct
  REGION 1..5: note = CDR-H1
DHAIN

SEQ ID NO: 280 - protein, 17 aa, synthetic construct
  REGION 1..17: note = CDR-H2
WMNPKIGNTGYAQKFQG

SEQ ID NO: 281 - protein, 10 aa, synthetic construct
  REGION 1..10: note = CDR-H3
DSSGYDAFDI

SEQ ID NO: 282 - protein, 17 aa, synthetic construct
  REGION 1..17: note = CDR-L1
KSSQSVLSTSYNRNFLA

SEQ ID NO: 283 - protein, 7 aa, synthetic construct
  REGION 1..7: note = CDR-L2
WASTRQS

SEQ ID NO: 284 - protein, 7 aa, synthetic construct
  REGION 1..7: note = CDR-H1
GDTFSDH

SEQ ID NO: 285 - protein, 6 aa, synthetic construct
  REGION 1..6: note = CDR-H2
NPKIGN

SEQ ID NO: 286 - protein, 5 aa, synthetic construct
  REGION 1..5: note = CDR-H1
THWMH

SEQ ID NO: 287 - protein, 17 aa, synthetic construct
  REGION 1..17: note = CDR-H2
MINPSDGVTYYAQTFQG

SEQ ID NO: 288 - protein, 9 aa, synthetic construct
  REGION 1..9: note = CDR-H3
EYYGEGFDY

SEQ ID NO: 289 - protein, 11 aa, synthetic construct
  REGION 1..11: note = CDR-L1
RASQGISNYLA

SEQ ID NO: 290 - protein, 7 aa, synthetic construct
  REGION 1..7: note = CDR-L2
SASNLQS

SEQ ID NO: 291 - protein, 7 aa, synthetic construct
  REGION 1..7: note = CDR-H1
GYSLITH

SEQ ID NO: 292 - protein, 6 aa, synthetic construct
  REGION 1..6: note = CDR-H2
NPSDGV

SEQ ID NO: 293 - protein, 545 aa, synthetic construct
  REGION 1..545: note = Full BaEV protein without signal sequence
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 294 - protein, 17 aa, synthetic construct
  REGION 1..17: note = Full R peptide only
VLTQQYQVLRTDEEAQD

SEQ ID NO: 295 - protein, 531 aa, synthetic construct
  REGION 1..531: note = BaEv envelope glycoprotein R'
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLT

SEQ ID NO: 296 - protein, 532 aa, synthetic construct
  REGION 1..532: note = BaEv envelope glycoprotein R∝
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQ

SEQ ID NO: 297 - protein, 533 aa, synthetic construct
  REGION 1..533: note = BaEv envelope glycoprotein R≡
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQ

SEQ ID NO: 298 - protein, 534 aa, synthetic construct
  REGION 1..534: note = BaEv envelope glycoprotein R≢
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQY

SEQ ID NO: 299 - protein, 535 aa, synthetic construct
  REGION 1..535: note = BaEv envelope glycoprotein R∂
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQ

SEQ ID NO: 300 - protein, 536 aa, synthetic construct
  REGION 1..536: note = BaEv envelope glycoprotein R[
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQV

SEQ ID NO: 301 - protein, 537 aa, synthetic construct
  REGION 1..537: note = BaEv envelope glycoprotein R]
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVL

SEQ ID NO: 302 - protein, 546 aa, synthetic construct
  REGION 1..546: note = BaEVRLess
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 303 - protein, 562 aa, synthetic construct
  REGION 1..562: note = BaEVTR
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLVQFVKDRISVVQALVLTQQYHQLKPLEYEP

SEQ ID NO: 304 - protein, 5 aa, synthetic construct
  REGION 1..5: note = CD3 CDR-H1
RYTMH

SEQ ID NO: 305 - protein, 17 aa, synthetic construct
  REGION 1..17: note = CD3 CDR-H2
YINPSRGYTNYNQKFKD

SEQ ID NO: 306 - protein, 10 aa, synthetic construct
  REGION 1..10: note = CD3 CDR-H3
YYDDHYCLDY

SEQ ID NO: 307 - protein, 10 aa, synthetic construct
  REGION 1..10: note = CD3 CDR-L1
RASSSVSYMN

SEQ ID NO: 308 - protein, 7 aa, synthetic construct
  REGION 1..7: note = CD3 CDR-L2
DTSKVAS

SEQ ID NO: 309 - protein, 9 aa, synthetic construct
  REGION 1..9: note = CD3 CDR-L3
QQWSSNPLT

SEQ ID NO: 310 - protein, 121 aa, synthetic construct
  REGION 1..121: note = CD3 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 311 - protein, 106 aa, synthetic construct
  REGION 1..106: note = CD3 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 312 - protein, 243 aa, synthetic construct
  REGION 1..243: note = CD3 scFv
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK